(19) United States
(12) Patent Application Publication
(10) Pub. No.: US 2025/0304535 A1
(43) Pub. Date: Oct. 2, 2025

(54) KAT6 Inhibitors

(71) Applicant: BeiGene Switzerland GmbH, Basel (CH)

(72) Inventors: Yayi WANG, Beijing (CN); Qiuwen WANG, Beijing (CN); Yunhang GUO, Beijing (CN); Zhiwei WANG, Beijing (CN)

(73) Assignee: Beigene Switzerland GmbH, Basel (CH)

(21) Appl. No.: 19/093,521

(22) Filed: Mar. 28, 2025

(30) Foreign Application Priority Data
Mar. 29, 2024 (WO) PCT/CN2024/084840
Sep. 6, 2024 (WO) PCT/CN2024/117463
Jan. 14, 2025 (WO) PCT/CN2025/072209

(51) Int. Cl. C07D 231/12; C07D 213/56; C07D 213/71; C07D 401/06; C07D 401/12; C07D 401/14; C07D 405/10; C07D 405/12; C07D 405/14; C07D 413/14; C07D 493/10; C07D 498/08

(52) U.S. Cl. CPC C07D 231/12; C07D 213/56; C07D 213/71; C07D 401/06; C07D 401/12; C07D 401/14; C07D 405/10; C07D 405/12; C07D 405/14; C07D 413/14; C07D 493/10; C07D 498/08

(57) ABSTRACT

Disclosed herein are compounds used as inhibitors of KAT6 and pharmaceutical compositions comprising the compounds. Also disclosed are methods for treating a disorder or disease responsive to the inhibition of KAT6 activity in a subject. In an embodiment, the compounds are of formula (I):

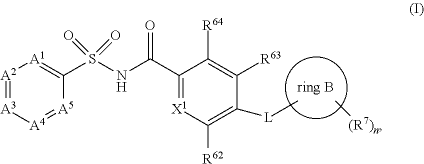

or a pharmaceutically acceptable salt thereof, or a deuterated analog thereof, or an N-oxide thereof, or a tautomer thereof, wherein values for the variables (e.g., ring B, A¹, A², A³, A⁴, A⁵, X¹, L, R⁶², R⁶³, R⁶⁴, R⁷, and n) are disclosed herein.

### CROSS-REFERENCE TO RELATED APPLICATIONS

This application claims the right of priority of International Application No. PCT/CN2024/084840, filed Mar. 29, 2024, International Application No. PCT/CN2024/117463, filed Sep. 6, 2024, and International Application No. PCT/CN2025/072209, filed Jan. 14, 2025. The contents of each of the above-mentioned applications are hereby incorporated by reference in their entireties.

### FIELD OF THE INVENTION

Disclosed herein are compounds used as inhibitors of KAT6. Also disclosed herein is the use of such compounds for inhibiting KAT6 activity, and for treating breast cancer.

### SEQUENCE LISTING

This application contains a Sequence Listing, which has been submitted electronically in XML format. The XML file is entitled “F24W1544PCT-sequence listing.xml,” was created on Mar. 20, 2025, and is 1,982 bytes in size. The Sequence Listing is incorporated herein by reference in its entirety.

### BACKGROUND OF THE INVENTION

Epsilon (ε)-lysine acetylation is one of the key mechanisms governing the ability of cells to respond to intracellular and extracellular signals. Levels of lysine acetylation are closely controlled by lysine acetyltransferases (KATs) and lysine deacetylases (KDACs). KATs, a highly diverse group of enzymes, transfer an acetyl moiety from acetyl-CoA to lysine residues, whereas KDACs remove these moieties. To date, 37 mammalian proteins have been suggested to possess endogenous KAT activity, including proteins of the MYST (Moz, Ybf2/Sas3, Sas2, Tip60) family, the p300 and CBP family, the SRC/p160 family, and the GCN5 and PCAF family (Sheikh, B. N., & Akhtar, A. (2019). The many lives of KATs—detectors, integrators and modulators of the cellular environment. Nat Rev Genet, 20(1), 7-23). KAT6 proteins (KAT6A and KAT6B) belong to the MYST family of acetyltransferases, which includes five members: KAT5 (also known as TIP60), KAT6A (also known as MOZ, MYST3), KAT6B (also known as MORF, MYST4), KAT7 (also known as HBO1) and KAT8 (also known as MOF) (Sheikh, B. N., & Akhtar, A. (2019).

KAT6A is overexpressed in a variety of tumor types (Wiesel-Motiuk, N., & Assaraf, Y. G. (2020). The lysine acetyltransferases KAT6A and KAT6B play key roles in physiology and pathology. Drug Resist Updat, 53, 100729.). Dysregulation of the expression of KAT6 proteins is known to support tumor progression, and aberrant histone acetylation may lead to tumorigenesis and cancer progression.

For example, in breast cancer, KAT6A was found to be amplified and/or overexpressed in 10-15% of breast cancers and in about 22% ER+HER2− breast cancers (Yu, L., Liang, Y., Cao, X., Wang, X., Gao, H., Lin, S. Y., Li, K. (2017). Identification of MYST3 as a novel epigenetic activator of ERalpha frequently amplified in breast cancer. Oncogene, 36(20), 2910-2918. doi:10.1038/onc.2016.433).

Thus, KAT6 inhibitors have potential use in the treatment of breast cancer.

### SUMMARY OF THE INVENTION

In one embodiment, disclosed herein are compounds of Formula (I):

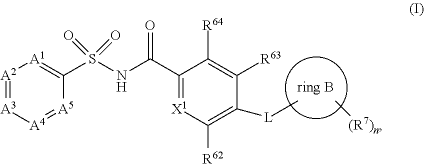

or a pharmaceutically acceptable salt thereof, or a deuterated analog thereof, or an N-oxide thereof, or a tautomer thereof, wherein values for the variables (e.g., A¹, A², A³, A⁴, A⁵, X¹, ring B, L, R⁶², R⁶³, R⁶⁴, R⁷, and n) are as described herein.

Also disclosed herein are pharmaceutical compositions comprising a compound of Formula (I), or a pharmaceutically acceptable salt thereof, or a deuterated analog thereof, or an N-oxide thereof, or a tautomer thereof.

Also disclosed herein is a method of treating breast cancer, comprising administering to a subject in need thereof a compound of Formula (I), or a pharmaceutically acceptable salt, or a deuterated analog thereof, or an N-oxide thereof, or a tautomer thereof.

Also disclosed herein is the use of a compound of Formula (I), or a pharmaceutically acceptable salt, or a deuterated analog thereof, or an N-oxide thereof, or a tautomer thereof for the treatment of breast cancer in a subject in need thereof, comprising administering to the subject a therapeutically effective amount of the compound.

Also disclosed herein is a compound of Formula (I), or a pharmaceutically acceptable salt, or a deuterated analog thereof, or an N-oxide thereof, or a tautomer thereof for use in the treatment of breast cancer in a subject in need thereof, the treatment comprising administering to the subject a therapeutically effective amount of the compound.

Also disclosed herein is the use of a compound of Formula (I), or a pharmaceutically acceptable salt, or a deuterated analog thereof, or an N-oxide thereof, or a tautomer thereof for the manufacture of a medicament useful in the treatment of breast cancer in a subject in need thereof.

### DETAILED DESCRIPTION OF THE INVENTION

The following terms have the indicated meanings throughout the specification:

As used herein, including the appended Aspects, the singular forms of words such as “a”, “an”, and “the”, include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 5, or from 1 to 4, or from 1 to 3, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl groups. An alkyl group defined herein is optionally deuterated or tritiated.

The term “propyl” refers to 1-propyl or n-propyl (“n-Pr”), 2-propyl or isopropyl (“i-Pr”).

The term “butyl” refers to 1-butyl or n-butyl (“n-Bu”), 2-methyl-1-propyl or isobutyl (“i-Bu”), 1-methylpropyl or s-butyl (“s-Bu”), 1, 1-dimethylethyl ort-butyl (“t-Bu”).

The term “pentyl” refers to 1-pentyl, 2-pentyl, 3-pentyl, 2-methyl-2-butyl, 3-methyl-2-butyl, 3-methyl-1-butyl, 2-methyl-1-butyl.

The term “hexyl” refers to 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl.

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈alkyl, haloC₁₋₆alkyl, haloC₁₋₅alkyl, halo C₁₋₄alkyl or haloC₁₋₃alkyl, but not limited to —CF₃, —CH₂Cl, —CH₂CF₃, —CHCl₂, —CF₃, and the like.

The term “deuteroalkyl” refers to an alkyl group in which one or more hydrogens are replaced by one or more deuterium atoms (²H). Examples of deuteroalkyl include but are not limited to C₁₋₈deuteroalkyl, C₁₋₆deuteroalkyl, C₁₋₄deuteroalkyl, —CD₃, —CHD₂, —CH₂D, and the like.

The term “alkoxy” refers to an alkyl group which is attached to a molecule via oxygen. This includes moieties where the alkyl part may be linear or branched. For example, the term “C₁₋₆ alkoxy” refers to an alkyl group which is attached to a molecule via oxygen containing 1, 2, 3, 4, 5 or 6 carbon atoms, for example methyl, ethyl, n-propyl, iso-propyl, n-butyl, sec-butyl, tert-butyl, n-pentyl and n-hexyl. Therefore, the alkoxy group may be methoxy, ethoxy, n-propoxy, iso-propoxy, n-butoxy, sec-butoxy, tert-butoxy, n-pentoxy and n-hexoxy.

The term “alkenyl” refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1, 3-dienyl, 2-methylbuta-1, 3-dienyl, hex-1-enyl, hex-2-enyl, hex-3-enyl, hex-4-enyl, and hexa-1, 3-dienyl groups.

The term “alkynyl” refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4, 4], [4, 5], [5, 5], [5, 6] and [6, 6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5, 6] and [6, 6] ring systems.

The term “spiro cycloalkyl” refers to a cyclic structure which contains carbon atoms and is formed by at least two rings sharing one atom. The term “7 to 12 membered spiro cycloalkyl” refers to a cyclic structure which contains 7 to 12 carbon atoms and is formed by at least two rings sharing one atom.

The term “fused cycloalkyl” refers to a bicyclic cycloalkyl group as defined herein which is saturated and is formed by two or more rings sharing two adjacent atoms.

The term “bridged cycloalkyl” refers to a cyclic structure which contains carbon atoms and is formed by two rings sharing two atoms which are not adjacent to each other. The term “7 to 10 membered bridged cycloalkyl” refers to a cyclic structure which contains 7 to 12 carbon atoms and is formed by two rings sharing two atoms which are not adjacent to each other.

The term “cycloalkenyl” refers to non-aromatic cyclic alkyl groups of from 3 to 10 carbon atoms having single or multiple rings and having at least one double bond and preferably from 1 to 2 double bonds. In one embodiment, the cycloalkenyl is cyclopentenyl or cyclohexenyl, 1-cyclopent-1-enyl, 1-cyclopent-2-enyl, 1-cyclopent-3-enyl, 1-cyclohex-1-enyl, 1-cyclohex-2-enyl, 1-cyclohex-3-enyl, cyclohexadienyl, preferably cyclohexenyl.

The term “cycloalkynyl” refers to non-aromatic cycloalkyl groups of from 5 to 10 carbon atoms having single or multiple rings and having at least one triple bond.

The term “aryl” used alone or in combination with other terms refers to a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2, 4-pyrimidinyl, 3, 5-pyrimidinyl, 2, 4-imidazolyl, imidazopyridinyl, isoxazolyl, oxazolyl, thiazolyl, isothiazolyl, thiadiazolyl (such as 1, 2, 3-thiadiazolyl, 1, 2, 4-thiadiazolyl, or 1, 3, 4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, oxadiazolyl (such as 1, 2, 3-oxadiazolyl, 1, 2, 4-oxadiazolyl, or 1, 3, 4-oxadiazolyl), phthalazinyl, pyrazinyl, pyridazinyl, pyrrolyl, triazolyl (such as 1, 2, 3-triazolyl, 1, 2, 4-triazolyl, or 1, 3, 4-triazolyl), quinolinyl, isoquinolinyl, pyrazolyl, pyrrolopyridinyl (such as 1H-pyrrolo[2, 3-b]pyridin-5-yl), pyrazolopyridinyl (such as 1H-pyrazolo[3, 4-b]pyridin-5-yl), benzoxazolyl (such as benzo[d]oxazol-6-yl), pteridinyl, purinyl, 1-oxa-2, 3-diazolyl, 1-oxa-2, 4-diazolyl, 1-oxa-2, 5-diazolyl, 1-oxa-3, 4-diazolyl, 1-thia-2, 3-diazolyl, 1-thia-2, 4-diazolyl, 1-thia-2, 5-diazolyl, 1-thia-3, 4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), and indazolyl (such as 1H-indazol-5-yl).

“Heterocycloalkyl”, “heterocyclyl” or “heterocyclic” are interchangeable and refer to a non-aromatic heterocycloalkyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “optionally oxidized sulfur,” used herein, refers to S, SO or SO₂.

The term “monocyclic heterocyclyl” refers to monocyclic groups in which at least one ring member (e.g., 1-3 heteroatoms, 1 or 2 heteroatoms) is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2, 5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1, 2-dithietanyl, 1, 3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1, 4-oxathianyl, 1, 4-dioxepanyl, 1, 4-oxathiepanyl, 1, 4-oxaazepanyl, 1, 4-dithiepanyl, 1, 4-thiazepanyl and 1, 4-diazepanyl, 1, 4-dithianyl, 1, 4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1, 4-dioxanyl, 1, 3-dioxolanyl, pyrazolinyl, pyrazolidinyl, dithianyl, dithiolanyl, pyrazolidinyl, imidazolinyl, pyrimidinonyl, or 1, 1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/3-membered, 4-membered/4-membered, 3-membered/5-membered, 4-membered/5-membered, 4-membered/6-membered, 5-membered/5-membered, or 5-membered/6-membered mono-spiro heterocyclyl. Representative examples of spiro heterocyclyls include, but not limited to the following groups: 2, 3-dihydrospiro[indene-1, 2′-pyrrolidine](e.g., 2, 3-dihydrospiro[indene-1, 2′-pyrrolidine]-1′-yl), 1, 3-dihydrospiro[indene-2, 2′-pyrrolidine](e.g., 1, 3-dihydrospiro[indene-2, 2′-pyrrolidine]-1′-yl), azaspiro[2.4]heptane (e.g., 5-azaspiro[2.4]heptane-5-yl), 2-oxa-6-azaspiro[3.3]heptane (e.g., 2-oxa-6-azaspiro[3.3]heptan-6-yl), azaspiro[3.4]octane (e.g., 6-azaspiro[3.4]octane-6-yl), 2-oxa-6-azaspiro[3.4]octane (e.g., 2-oxa-6-azaspiro[3.4]octane-6-yl), azaspiro[3.4]octane (e.g., 6-azaspiro[3.4]octan-6-yl), azaspiro[3.4]octane (e.g., 6-azaspiro[3.4]octan-6-yl), 1, 7-dioxaspiro[4.5]decane, 2-oxa-7-aza-spiro[4.4]nonane (e.g., 2-oxa-7-aza-spiro[4.4]non-7-yl), 7-oxa-spiro[3.5]nonyl and 5-oxa-spiro[2.4]heptyl.

The term “fused heterocyclyl” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but the fused heterocyclic group does not have a completely conjugated pi-electron system. Preferably, a fused heterocyclyl is 6 to 14-membered, and more preferably 7 to 12-membered, or 7- to 10-membered. According to the number of membered rings, a fused heterocyclyl is divided into bicyclic, tricyclic, tetracyclic, or polycyclic fused heterocyclyl. The group can be attached to the remainder of the molecule through either ring.

Specifically, the term “bicyclic fused heterocyclyl” refers to a 7 to 12-membered, preferably 7- to 10-membered, more preferably 9- or 10-membered fused heterocyclyl as defined herein comprising two fused rings and comprising 1 to 4 heteroatoms selected from nitrogen, oxygen or optionally oxidized sulfur as ring members. Typically, a bicyclic fused heterocyclyl is 5-membered/5-membered, 5-membered/6-membered, 6-membered/6-membered, or 6-membered/7-membered bicyclic fused heterocyclyl. Representative examples of (bicyclic) fused heterocycles include, but not limited to, the following groups octahydrocyclopenta[c]pyrrole, octahydropyrrolo[3, 4-c]pyrrolyl, octahydroisoindolyl, isoindolinyl, octahydro-benzo[b][1, 4]dioxin, indolinyl, isoindolinyl, benzopyranyl, dihydrothiazolopyrimidinyl, tetrahydroquinolyl, tetrahydroisoquinolyl (or tetrahydroisoquinolinyl), dihydrobenzofuranyl, dihydrobenzoxazinyl, dihydrobenzoimidazolyl, tetrahydrobenzothienyl, tetrahydrobenzofuranyl, benzodioxolyl, benzodioxonyl, chromanyl, chromenyl, octahydrochromenyl, dihydrobenzodioxynyl, dihydrobenzoxezinyl, dihydrobenzodioxepinyl, dihydrothienodioxynyl, dihydrobenzooxazepinyl, tetrahydrobenzooxazepinyl, dihydrobenzoazepinyl, tetrahydrobenzoazepinyl, isochromanyl, chromanyl, or tetrahydropyrazolopyrimidinyl (e.g., 4, 5, 6, 7-tetrahydropyrazolo[1, 5-a]pyrimidin-3-yl).

The term a “benzo fused heterocyclyl” is a bicyclic fused heterocyclyl in which a monocyclic 4 to 9-membered heterocyclyl as defined herein (preferably 5- or 6-membered) fused to a benzene ring.

The term “bridged heterocyclyl” refers to a 5 to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

If amine is substituted by R⁵, it means that the nitrogen atom in structures of

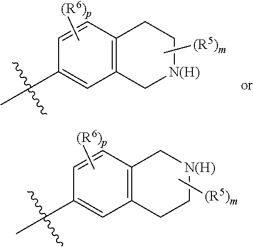

is not bonded to a hydrogen.

The term “at least one substituents” disclosed herein includes, for example, from 1 to 4, such as from 1 to 3, further as 1 or 2, substituents, provided the theory of valence is met. For example, “at least one substituents R^{6″}” disclosed herein includes from 1 to 4, such as from 1 to 3, further as 1 or 2, substituents selected from the list of R⁶ as disclosed herein.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, the reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired product of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Pharmaceutically acceptable salt”” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and/or water and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The term “subject” herein means a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject.

The pharmaceutical composition comprising the compound disclosed herein can be administrated via oral, administration to a subject in need thereof. The pharmaceutical composition may be a regular solid formulation such as tablets, capsules and the like.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation.

The term “disease” refers to any disease, discomfort, illness, symptoms or indications, and can be interchangeable with the term “disorder” or “condition”.

Throughout this specification and the Aspects which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the Aspects which follow, the term “Cₙ-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### Aspects and Embodiments

The following aspects and embodiments are not intended to be an explicit or implicit admission that these aspects or embodiments are independent or distinct nor should it be interpreted as such. Rather, it is intended to convey information so that the full breadth of the present invention can be understood. Furthermore, the following aspects and embodiments are not meant to be limiting on the full breadth of the invention as recited by the structure of Formula I.

Aspect 1. A compound of Formula (I):

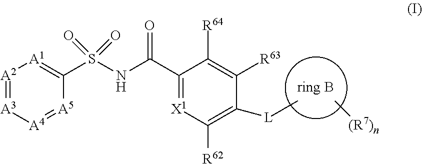

or a pharmaceutically acceptable salt thereof, or a deuterated analog thereof, or an N-oxide thereof, or a tautomer thereof, wherein:
A¹ is CR¹ or N, A² is CR² or N, A³ is CR³ or N, A⁴ is CR⁴ or N, A⁵ is CR⁵ or N, provided that at most two of A¹, A², A³, A⁴ and A⁵ are N;
R¹, R², R³, R⁴ and R⁵ are each independently hydrogen, deuterium, halogen, —C₁₋₁₀alkyl, —C₂₋₁₀alkenyl, —C₂₋₁₀alkynyl, cycloalkyl, cycloalkenyl, cycloalkynyl, heterocyclyl, aryl, heteroaryl, —CN, —NO₂, —OR^{1a}, —SO₂R^{1a}, —COR^{1a}, —CO₂R^{1a}, —CONR^{1a}R^{1b}, —C(═NR^{1a})NR^{1b}R^{1c}, —NR^{1a}R^{1b}, —NR^{1a}COR^{1b}, —NR^{1a}CONR^{1b}R^{1c}, —NR^{1a}CO₂R^{1b}, —NR^{1a}SONR^{1b}R^{1c}, —NR^{1a}SO₂NR^{1b}R^{1c}, —P(═O)R^{1a}R^{1b}, —NR^{1a}SO₂R^{1b} or —SOR^{1a}(═NR^{1b}), wherein each of said —C₁₋₁₀alkyl, —C₂₋₁₀alkenyl, —C₂₋₁₀alkynyl, cycloalkyl, cycloalkenyl, cycloalkynyl, heterocyclyl, aryl or heteroaryl is unsubstituted or substituted with 1, 2, or 3 R^{1d};
or (R³ and R⁴) or (R⁴ and R⁵), together with the atoms to which they are attached, independently form a C₃₋₁₀ cycloalkyl, C₃₋₁₀ cycloalkenyl, 5- to 12-membered heterocyclyl ring containing 1, 2 or 3 heteroatoms independently selected from nitrogen, oxygen and optionally oxidized sulfur, 5- to 12-membered aryl, or 5- to 12-membered heteroaryl ring containing 1, 2 or 3 heteroatoms independently selected from nitrogen, oxygen and optionally oxidized sulfur, wherein each of said C₃₋₁₀ cycloalkyl, C₃₋₁₀ cycloalkenyl, 5- to 12-membered heterocyclyl ring, 5- to 12-membered aryl, or 5- to 12-membered heteroaryl ring is unsubstituted or substituted with 1, 2, 3, or 4 R^{1d};
R^{1a}, R^{1b}, and R^{1c} are each independently hydrogen, deuterium, —C₁₋₁₀alkyl, —C₁₋₁₀deuteroalkyl, —C₂₋₁₀alkenyl, —C₂₋₁₀alkynyl, cycloalkyl, cycloalkenyl, cycloalkynyl, heterocyclyl, aryl, or heteroaryl, wherein each of said —C₁₋₁₀alkyl, —C₂₋₁₀alkenyl, —C₂₋₁₀alkynyl, cycloalkyl, heterocyclyl, aryl, or heteroaryl is unsubstituted or substituted with 1, 2, or 3 R^{1j};
R^{1d} and R^{1j} are each independently hydrogen, deuterium, halogen, —C₁₋₁₀alkyl, —C₂₋₁₀alkenyl, —C₂₋₁₀alkynyl, cycloalkyl, cycloalkenyl, cycloalkynyl, heterocyclyl, aryl, heteroaryl, —CN, —NO₂, —OR^{1e}, —SO₂R^{1e}, —COR^{1e}, —CO₂R^{1e}, —CONR^{1e}R^{1f}, —C(═NR^{1e})NR^{1f}R^{1g}, —NR^{1e}R^{1f}, —NR^{1e}COR^{1f}, NR^{1e}CONR^{1f}R^{1g}, —NR^{1e}CO₂R^{1f}, —NR^{1e}SONR^{1f}R^{1g}, —NR^{1e}SO2NR^{1f}R^{1g}, —P(═O)R^{1e}R^{1f}, —NR^{1e}SO₂R^{1f}, or —SOR^{1e}(═NR^{1f}), wherein each of said —C₁₋₁₀alkyl, —C₂₋₁₀alkenyl, —C₂₋₁₀alkynyl, cycloalkyl, heterocyclyl, aryl, or heteroaryl is unsubstituted or substituted with at least one substituent selected from halogen, —CN, —C₁₋₁₀alkyl, —OR^{1h}, —NR^{1h}R¹ⁱ, cycloalkyl, heterocyclyl, aryl, and heteroaryl; or
two R^{1d} or two R^{1j} substituted at same atom form an oxo; or
two R^{1d} substituted at same or different atom(s), together with the atom(s) to which they are attached and any intervening atoms, form a C₃₋₁₀ cycloalkyl, 4- to 12-membered heterocyclyl ring containing 1, 2 or 3 heteroatoms independently selected from nitrogen, oxygen and optionally oxidized sulfur, 6- to 12-membered aryl, or 5- to 12-membered heteroaryl ring containing 1, 2 or 3 heteroatoms independently selected from nitrogen, oxygen and optionally oxidized sulfur; wherein said C₃₋₁₀ cycloalkyl, 4- to 12-membered heterocyclyl ring, 6- to 12-membered aryl, or 5- to 12-membered heteroaryl ring is unsubstituted or substituted with 1, 2, or 3 R^{1k};
R^{1e}, R^{1f}, R^{1g}, R^{1h} and R¹ⁱ are each independently hydrogen, —C₁₋₁₀alkyl, -haloC₁₋₁₀alkyl, —C₂₋₁₀alkenyl, —C₂₋₁₀alkynyl, cycloalkyl, heterocyclyl, aryl, or heteroaryl;
each R^{1k} is independently hydrogen, halogen, —C₁₋₁₀alkyl, -haloC₁₋₁₀alkyl, or —C₁₋₁₀alkoxy;
   X¹ is CR⁶¹ or N;
   L is a direct bond, —(CR^{L1}R^{L2})ₚ—, —O—, —S— or —NR^{L3}—;
   ring B is isoxazolyl, thiazolyl, imidazolyl, oxazolyl, pyrazolyl, pyridinyl, pyridazinyl, pyrizinyl or pyrimidinyl;
   R⁶¹, R⁶², R⁶³, R⁶⁴, R⁷, R^{L1} and R^{L2} are each independently hydrogen, deuterium, halogen, —C₁₋₁₀alkyl, —OC₁₋₁₀deuteroalkyl, —C₂₋₁₀alkenyl, —C₂₋₁₀alkynyl, cycloalkyl, cycloalkenyl, cycloalkynyl, heterocyclyl, aryl, heteroaryl, —CN, —NO₂, —OR^{3a}, —SO₂R^{3a}, —COR^{3a}, —CO₂R^{3a}, —CONR^{3a}R^{3b}, —C(═NR^{3a})NR^{3b}R^{3c}, —NR^{3a}R^{3b}, —NR^{3a}COR^{3b}, —NR^{3a}CONR^{3b}R^{3c}, —NR^{3a}CO₂R^{3b}, —NR^{3a}SONR^{3b}R^{3c}, —NR^{3a}SO₂NR^{3b}R^{3c}, —P(═O)R^{3a}R^{3b}, —NR^{3a}SO₂R^{3b}, or —SOR^{3a}(═NR^{3b}), wherein each of said —C₁₋₁₀alkyl, —C₂₋₁₀alkenyl, —C₂₋₁₀alkynyl, cycloalkyl, heterocyclyl, aryl, or heteroaryl is unsubstituted or substituted with 1, 2, or 3 R^{3d};
   R^{3a}, R^{3b} and R^{3c} are each independently hydrogen, deuterium, —C₁₋₁₀alkyl, —C₂₋₁₀alkenyl, —C₂₋₁₀alkynyl, cycloalkyl, cycloalkenyl, cycloalkynyl, heterocyclyl, aryl, or heteroaryl; wherein each of said —C₁₋₁₀alkyl, —C₂₋₁₀alkenyl, —C₂₋₁₀alkynyl, cycloalkyl, heterocyclyl, aryl, or heteroaryl is unsubstituted or substituted with R^{3j}.
   R^{3d} and R^{3j} are each independently hydrogen, deuterium, halogen, —C₁₋₁₀alkyl, —C₂₋₁₀alkenyl, —C₂₋₁₀alkynyl, cycloalkyl, cycloalkenyl, cycloalkynyl, heterocyclyl, aryl, heteroaryl, —CN, —NO₂, —OR^{3e}, —SO₂R^{3e}, —COR^{3e}, —CO₂R^{3e}, —CONR^{3e}R^{3f}, —C(═NR³e)NR^{3f}R^{3g}, —NR^{3e}R^{3f}, —NR^{3e}COR^{3f}, —NR^{3e}CONR^{3f}R^{3g}, —NR^{3e}CO₂R^{3f}, —NR^{3e}SONR^{3f}R^{3g}, —NR^{3e}SO₂NR^{3f}R^{3g}, —P(═O)R^{3e}R^{3f}, —NR^{3e}SO₂R^{3f}, or —SOR^{3e}(═NR^{3f}), wherein each of said —C₁₋₁₀alkyl, —C₂₋₁₀alkenyl, —C₂₋₁₀alkynyl, cycloalkyl, heterocyclyl, aryl, or heteroaryl is unsubstituted or substituted with at least one substituent selected from halogen, —C₁₋₁₀alkyl, —OR^{3h}, —NR^{3h}R³ⁱ, cycloalkyl, heterocyclyl, aryl, or heteroaryl; or
two R^{3d} or two R^{3j} substituted at same atom form an oxo; or
   R^{3e}, R^{3f}, R^{3g}, R^{3h} and R³ⁱ are each independently hydrogen, —C₁₋₁₀alkyl, -haloC₁₋₁₀alkyl, —C₂₋₁₀alkenyl, —C₂₋₁₀alkynyl, cycloalkyl, heterocyclyl, alkoxy substituted benzyl, aryl, or heteroaryl;
   R^{L3} is hydrogen, —C₁₋₁₀alkyl, —C₂₋₁₀alkenyl, —C₂₋₁₀alkynyl, cycloalkyl, cycloalkenyl, cycloalkynyl, heterocyclyl, aryl, or heteroaryl;
   p is 0, 1, 2, or 3 (optionally, p is 1 or 2; further optionally, p is 1); and
   n is 0, 1, 2, or 3 (optionally, n is 1 or 2; further optionally n is 1).

In an embodiment of Aspect 1, A³ is CR³, A⁴ is CR⁴, A⁵ is CR⁵. Optionally, R¹ is —C₁₋₅alkoxy(optionally —OC₁₋₅deuteroalkyl), R², R³, and R⁵ are hydrogen, R⁴ is —C₁₋₁₀alkyl, —C₁₋₁₀alkoxy, cycloalkyl, heterocyclyl, aryl, or heteroaryl, wherein each of said —C₁₋₁₀alkyl, cycloalkyl, heterocyclyl, aryl, or heteroaryl is unsubstituted or substituted with at least one substituent R^{1d}; each R^{1d} is independently halogen, —C₁₋₁₀alkyl, —C₁₋₁₀alkylene-C₁₋₁₀alkoxy, —C₁₋₁₀alkoxy, or —COOC₁₋₁₀alkyl.

In a preferred embodiment, R⁴ is selected from —C₁₋₆alkyl; —C₁₋₆alkylene-C₁₋₃alkoxy; C₃₋₁₀ cycloalkyl substituted with 1, 2, or 3 substituents independently selected from F, Cl, Br, —C₁₋₆alkyl, and —C₁₋₆alkoxy; and 3- to 7-membered monocyclic or bicyclic (optionally fused-, spiro-, or bridged-bicyclic ring) heterocyclyl containing 1, 2 or 3 heteroatoms independently selected from nitrogen, oxygen and optionally oxidized sulfur and unsubstituted or substituted with 1, 2, or 3 substituents independently selected from F, Cl, Br, —C₁₋₆alkyl, and —C₁₋₆alkoxy.

In a further preferred embodiment, R⁴ is selected from —C₁₋₆alkyl, —C₁₋₆alkylene-C₁₋₃alkoxy, cyclopropanyl, 3-azabicyclo[3.1.0]hexanyl, and oxetanyl; wherein each of said —C₁₋₆alkyl, —C₁₋₆alkylene-C₁₋₃alkoxy, cyclopropanyl, 3-azabicyclo[3.1.0]hexanyl, and oxetanyl is unsubstituted or substituted with 1, 2, or 3 substituents independently selected from methyl, ethyl, propyl, F, Cl, Br, methoxy, ethoxy, or —COOCH₃.

In an embodiment of Aspect 1, R¹ is —C₁₋₅alkoxy(optionally —OC₁₋₅deuteroalkyl), R² is hydrogen, A³ is CR³, A⁴ is CR⁴, A⁵ is CR⁵. Optionally, R¹ is —C₁₋₅alkoxy, R² is hydrogen, (R³ and R⁴), or (R⁴ and R⁵), together with the atoms to which they are attached, form a C₃₋₁₀ cycloalkyl, C₃₋₁₀ cycloalkenyl, 5- to 12-membered heterocyclyl ring containing 1, 2 or 3 heteroatoms independently selected from nitrogen, oxygen and optionally oxidized sulfur, 6- to 12-membered aryl, or 5- to 12-membered heteroaryl ring containing 1, 2 or 3 heteroatoms independently selected from nitrogen, oxygen and optionally oxidized sulfur, and unsubstituted or substituted with 1, 2, 3, or 4 R^{1d}; each R^{1d} is independently halogen, —C₁₋₁₀alkyl, —C₁₋₁₀alkoxy, or —COOC₁₋₁₀alkyl. In a preferred embodiment, (R³ and R⁴), or (R⁴ and R⁵), together with the atoms to which they are attached, form a dihydrofuran ring, or pyrrole ring; wherein each of said dihydrofuran ring, or pyrrole is unsubstituted or substituted with 1, 2, 3, or 4 substituents selected from methyl, ethyl, propyl, F, Cl, Br, methoxy, ethoxy, or —COOCH₃.

In a preferred embodiment, R¹ is —C₁₋₅alkoxy, R² is hydrogen, A³ is CR³, A⁴ is CR⁴, A⁵ is CR⁵. Optionally, R¹ is —C₁₋₅alkoxy(optionally —OC₁₋₅deuteroalkyl), R² is hydrogen, R³ is hydrogen, (R⁴ and R⁵), together with the atoms to which they are attached, form a C₃₋₁₀ cycloalkyl, C₃₋₁₀ cycloalkenyl, 5- to 12-membered heterocyclyl ring containing 1, 2 or 3 heteroatoms independently selected from nitrogen, oxygen and optionally oxidized sulfur, 6- to 12-membered aryl, or 5- to 12-membered heteroaryl ring containing 1, 2 or 3 heteroatoms independently selected from nitrogen, oxygen and optionally oxidized sulfur, and unsubstituted or substituted with 1, 2, 3, or 4 R^{1d}; each R^{1d} is independently halogen, —C₁₋₁₀alkyl, —C₁₋₁₀alkoxy, heterocyl, or —COOC₁₋₁₀alkyl.

In a preferred embodiment, R¹ is —C₁₋₅alkoxy(optionally —OC₁₋₅deuteroalkyl), R² is hydrogen, A³ is CR³, A⁴ is CR⁴, A⁵ is CR⁵. Optionally, R¹ is —C₁₋₅alkoxy, R² is hydrogen, R⁵ is hydrogen, (R³ and R⁴), together with the atoms to which they are attached, form a C₃₋₁₀ cycloalkyl, C₃₋₁₀ cycloalkenyl, 5- to 12-membered heterocyclyl ring containing 1, 2 or 3 heteroatoms independently selected from nitrogen, oxygen and optionally oxidized sulfur, 6- to 12-membered aryl, or 5- to 12-membered heteroaryl ring containing 1, 2 or 3 heteroatoms independently selected from nitrogen, oxygen and optionally oxidized sulfur, and unsubstituted or substituted with 1, 2, 3, or 4 R^{1d}; each R^{1d} is independently halogen, —C₁₋₁₀alkyl, —C₁₋₁₀alkoxy, heterocyl, or —COOC₁₋₁₀alkyl.

In an embodiment of Aspect 1, R¹ is —C₁₋₅alkoxy(optionally —OC₁₋₅deuteroalkyl), R² is hydrogen, A³ is CR³, A⁴ is CR⁴, A⁵ is N. Optionally, R¹ is —C₁₋₅alkoxy(optionally —OC₁₋₅deuteroalkyl), R², R³, and R⁵ are hydrogen, R⁴ is —C₁₋₁₀alkyl, —C₁₋₁₀alkoxy, cycloalkyl, heterocyclyl, aryl, or heteroaryl, wherein each of said —C₁₋₁₀alkyl, cycloalkyl, heterocyclyl, aryl, or heteroaryl is unsubstituted or substituted with at least one substituent R^{1d}; each R^{1d} is independently halogen, —C₁₋₁₀alkyl, —C₁₋₁₀alkylene-C₁₋₁₀alkoxy, —C₁₋₁₀alkoxy, or —COOC₁₋₁₀alkyl.

Aspect 2. The compound of Aspect 1, wherein

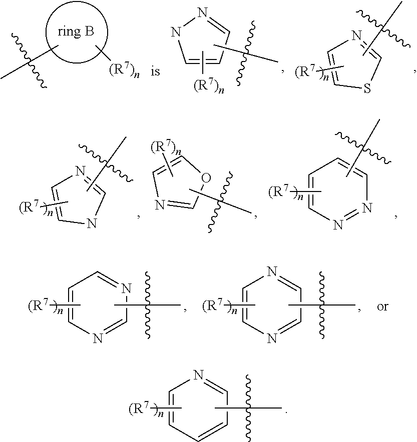

In an embodiment of Aspect 2,

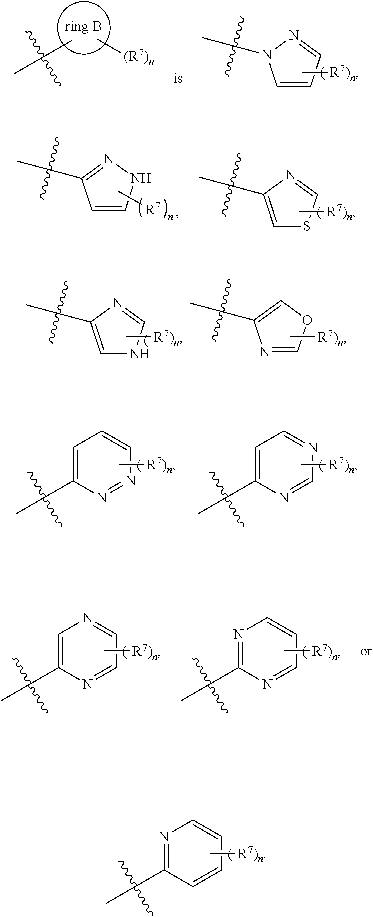

In an embodiment of Aspect 2,

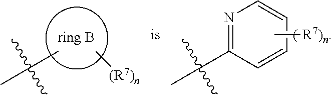

In an embodiment of Aspect 2,

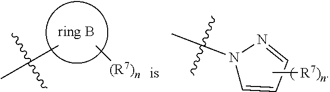

In first embodiment of Aspect 2, ring B is unsubstituted or substituted with 1, or 2 R⁷ (i.e., n is 0, 1 or 2). In an embodiment of Aspect 2, n is 0 or 1. In an embodiment of Aspect 2, ring B is unsubstituted (i.e., n is 0).

In second embodiment of Aspect 2,

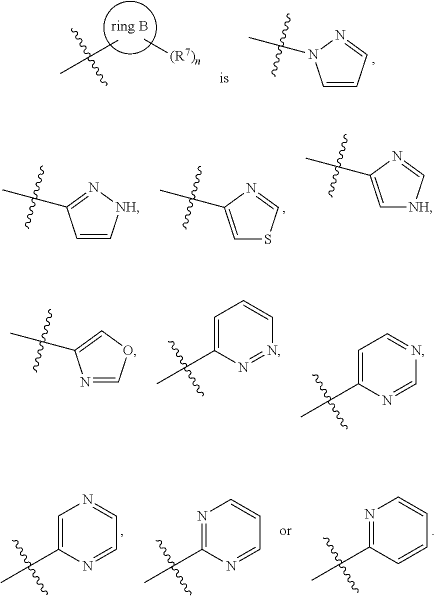

In third embodiment of Aspect 2,

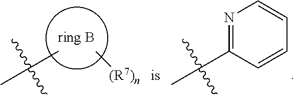

In fourth embodiment of Aspect 2,

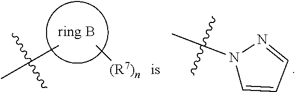

Aspect 2′. the compound of Formula (I) is the compound of formula (II-1):

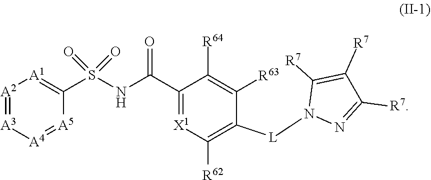

In some embodiments, the compound of formula (II-1), A¹ is CR¹; R¹ is —OR^{1a}; and R^{1a} is hydrogen, —C₁₋₁₀deuteroalkyl(preferably, —C₁₋₅deuteroalkyl), —C₁₋₁₀alkyl(preferably, —C₁₋₅alkyl) or -haloC₁₋₁₀alkyl(preferably, -haloC₁₋₅alkyl);
A² is CR²; and R² is hydrogen, deuterium, halogen, —C₁₋₃alkyl or -haloC₁₋₃alkyl;
A³ is CR³ or N, A⁴ is CR⁴ or N, A⁵ is CR⁵ or N, provided that at most one of A³, A⁴ and A⁵ are N;
if present, R³, R⁴ and R⁵ are each independently hydrogen, deuterium, halogen, —C₁₋₁₀alkyl, —C₂₋₁₀alkenyl, —C₂₋₁₀alkynyl, cycloalkyl, cycloalkenyl, cycloalkynyl, heterocyclyl, aryl, heteroaryl, —CN, —NO₂, —OR^{1a}, —SO₂R^{1a}, —COR^{1a}, —CO₂R^{1a}, —CONR^{1a}R^{1b}, —C(═NR^{1a})NR^{1b}R^{1c}, —NR^{1a}R^{1b}, —NR^{1a}COR^{1b}, —NR^{1a}CONR^{1b}R^{1c}, —NR^{1a}CO₂R^{1b}, —NR^{1a}SONR^{1b}R^{1c}, —NR^{1a}SO₂NR^{1b}R^{1c}, —P(═O)R^{1a}R^{1b}, —NR^{1a}SO₂R^{1b}, or —SOR^{1a}(═NR^{1b}), wherein each of said —C₁₋₁₀alkyl, —C₂₋₁₀alkenyl, —C₂₋₁₀alkynyl, cycloalkyl, cycloalkenyl, cycloalkynyl, heterocyclyl, aryl, or heteroaryl is unsubstituted or substituted with 1, 2, or 3 R^{1d};
or (R³ and R⁴) or (R⁴ and R⁵), together with the atoms to which they are attached, independently form a C₃₋₁₀ cycloalkyl, C₃₋₁₀ cycloalkenyl, 5- to 12-membered heterocyclyl ring containing 1, 2 or 3 heteroatoms independently selected from nitrogen, oxygen and optionally oxidized sulfur, 5- to 12-membered aryl, or 5- to 12-membered heteroaryl ring containing 1, 2 or 3 heteroatoms independently selected from nitrogen, oxygen and optionally oxidized sulfur, wherein each of said C₃₋₁₀ cycloalkyl, C₃₋₁₀ cycloalkenyl, 5- to 12-membered heterocyclyl ring, 5- to 12-membered aryl, or 5- to 12-membered heteroaryl ring is unsubstituted or substituted with 1, 2, 3, or 4 R^{1d};
R^{1a}, R^{1b}, and R^{1c} are each independently hydrogen, —C₁₋₁₀alkyl, —C₂₋₁₀alkenyl, —C₂₋₁₀alkynyl, cycloalkyl, cycloalkenyl, cycloalkynyl, heterocyclyl, aryl, or heteroaryl, wherein each of said —C₁₋₁₀alkyl, —C₂₋₁₀alkenyl, —C₂₋₁₀alkynyl, cycloalkyl, heterocyclyl, aryl, or heteroaryl is unsubstituted or substituted with 1, 2, or 3 R^{J};
R^{1d} and R^{1j} are each independently hydrogen, deuterium, halogen, —C₁₋₁₀alkyl, —C₂₋₁₀alkenyl, —C₂₋₁₀alkynyl, cycloalkyl, cycloalkenyl, cycloalkynyl, heterocyclyl, aryl, heteroaryl, —CN, —NO₂, —OR^{1e}, —SO₂R^{1e}, —COR^{1e}, —CO₂R^{1e}, —CONR^{1e}R^{1f}, —C(═NR^{1e})NR^{1f}R^{1g}, —NR^{1e}R^{1f}, —NR^{1e}COR^{1f}, —NR^{1e}CONR^{1f}R^{1g}, —NR^{1e}CO₂R^{1f}, —NR^{1e}SONR^{1f}R^{1g}, —NR^{1e}SO₂NR^{1f}R^{1g}, —P(═O)R^{1e}R^{1f}, —NR^{1e}SO₂R^{1f}, or —SOR^{1e}(═NR^{1f}), wherein each of said —C₁₋₁₀alkyl, —C₂₋₁₀alkenyl, —C₂₋₁₀alkynyl, cycloalkyl, heterocyclyl, aryl, or heteroaryl is unsubstituted or substituted with at least one substituent selected from halogen, —CN, —C₁₋₁₀alkyl, —OR^{1h}, —NR^{1h}R¹ⁱ, cycloalkyl, heterocyclyl, aryl, and heteroaryl; or
two R^{1d} or two R^{1j} substituted at same atom form an oxo; or
two R^{1d} substituted at same or different atom(s), together with the atom(s) to which they are attached and any intervening atoms, form a C₃₋₁₀ cycloalkyl, 4- to 12-membered heterocyclyl ring containing 1, 2 or 3 heteroatoms independently selected from nitrogen, oxygen and optionally oxidized sulfur, 6- to 12-membered aryl, or 5- to 12-membered heteroaryl ring containing 1, 2 or 3 heteroatoms independently selected from nitrogen, oxygen and optionally oxidized sulfur; wherein each of said C₃₋₁₀ cycloalkyl, 4- to 12-membered heterocyclyl ring, 6- to 12-membered aryl, or 5- to 12-membered heteroaryl ring is unsubstituted or substituted with 1, 2, or 3 R^{1k};
R^{1e}, R^{1f}, R^{1g}, and R¹ⁱ are each independently hydrogen, —C₁₋₁₀alkyl, -haloC₁₋₁₀alkyl, —C₂₋₁₀alkenyl, —C₂₋₁₀alkynyl, cycloalkyl, heterocyclyl, aryl, or heteroaryl;
R^{1k} is independently hydrogen, halogen, —C₁₋₁₀alkyl, -haloC₁₋₁₀alkyl, or —C₁₋₁₀alkoxy.

In some embodiments, the compound of formula (II-1), X¹ is N or CR⁶¹,
R⁶¹, R⁶², R⁶³ and R⁶⁴ are each independently hydrogen, deuterium, halogen, —C₁₋₁₀alkyl, —SC₁₋₁₀alkyl, —OC₁₋₁₀deuteroalkyl, —C₂₋₁₀alkenyl, —C₂₋₁₀alkynyl, —C₁₋₁₀alkoxy, C₃₋₁₀ monocyclic cycloalkyl, C₃₋₁₀ monocyclic cycloalkenyl, C₃₋₁₀ monocyclic cycloalkynyl, 4- to 10-membered monocyclic heterocyclyl containing 1, 2 or 3 heteroatoms independently selected from nitrogen, oxygen and optionally oxidized sulfur, 6-membered monocyclic aryl, 5- or 6-membered monocyclic heteroaryl, —CN, or —NR^{3a}R^{3b}; wherein each of said —C₁₋₁₀alkyl, —C₂₋₁₀alkenyl, —C₂₋₁₀alkynyl, —C₁₋₁₀alkoxy, C₁₋₁₀ monocyclic cycloalkyl, C₁₋₁₀ monocyclic cycloalkenyl, C₁₋₁₀ monocyclic cycloalkynyl, 4- to 10-membered monocyclic heterocyclyl containing 1, 2 or 3 heteroatoms independently selected from nitrogen, oxygen and optionally oxidized sulfur, 6-membered monocyclic aryl, or 5- or 6-membered monocyclic heteroaryl is unsubstituted or substituted with 1, 2 or 3 R^{3d};
R^{3a} and R^{3b} are each independently hydrogen, —C₁₋₅alkyl, —C₂₋₅alkenyl, —C₂₋₅alkynyl, or C₃₋₆ monocyclic cycloalkyl;
each R^{3d} is independently hydrogen, halogen, —C₁₋₁₀alkyl, —C₂₋₁₀alkenyl, —C₁₋₁₀alkoxy, —C₂₋₁₀alkynyl or C₃₋₆ monocyclic cycloalkyl.

In some embodiments, the compound of formula (II-1), wherein X¹ is N or CR⁶¹,
if R⁶¹ is present, R⁶¹, R⁶², R⁶³ and R⁶⁴ are each independently hydrogen, methyl, —OCD₃, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, pyrrolidinyl, furanyl, pyrazolyl, oxazolyl, oxetanyl, azetidinyl, —NH₂, —NH(C₁₋₆alkyl), —N(C₁₋₆alkyl)₂, —CN, F, Cl, Br or I; wherein each of said methyl, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, pyrrolidinyl, furanyl, pyrazolyl, oxazolyl, oxetanyl or azetidinyl is unsubstituted or substituted with 1, 2 or 3 R^{3d};
each R^{3d} is independently hydrogen, methyl, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, oxiranyl, oxetanyl, F, Cl, Br or I.

In some embodiments, the compound of formula (II-1), wherein X¹ is N or CR⁶¹,
if R⁶¹ is present, R⁶¹, R⁶², R⁶³ and R⁶⁴ are each independently hydrogen, methyl, —OCD₃, ethyl, n-propyl, isopropyl, ethenyl, prop-1-enyl, prop-2-enyl, ethynyl, 1-propynyl, cyclopropyl, —CN, F, Cl, Br, I, —N(CH₃)₂, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂, —CH(CH₃)OCH₃, —OCHF₂ or —CH₂OCH₃.

In some embodiments, the compound of formula (II-1), X¹ is N or CR⁶¹,
if R⁶¹ is present, R⁶¹, R⁶³, and R⁶⁴ are each independently hydrogen or deuterium;
   R⁶² is hydrogen, methyl, —OCD₃, ethyl, n-propyl, isopropyl, ethenyl, prop-1-enyl, prop-2-enyl, ethynyl, 1-propynyl, cyclopropyl, F, Cl, Br, I, —N(CH₃)₂, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂ or —CH₂OCH₃.

In some embodiments, the compound of formula (II-1), X¹ is N or CR⁶¹,
if R⁶¹ is present, R⁶¹, R⁶³ and R⁶⁴ are each independently hydrogen;
   R⁶² is each independently methyl, ethyl, cyclopropyl, ethyl or methoxy.

Aspect 3, The compound of Aspect 1 or Aspect 2, L is —CH₂—, or —(CH₂)₂.

In an embodiment of Aspect 3, L is —CH₂—.

Aspect 4, The compound of any one of Aspects 1-3, X¹ is CR⁶¹,
R⁶¹, R⁶², R⁶³ and R⁶⁴ are each independently hydrogen, deuterium, halogen, —C₁₋₁₀alkyl, —OC₁₋₁₀deuteroalkyl, —C₂₋₁₀alkenyl, —C₂₋₁₀alkynyl, —C₁₋₁₀alkoxy, C₃₋₁₀ monocyclic cycloalkyl, C₃₋₁₀ monocyclic cycloalkenyl, C₃₋₁₀ monocyclic cycloalkynyl, 4- to 10-membered monocyclic heterocyclyl containing 1, 2 or 3 heteroatoms independently selected from nitrogen, oxygen and optionally oxidized sulfur, 6-membered monocyclic aryl, 5- or 6-membered monocyclic heteroaryl, —CN, or —NR^{3a}R^{3b}; wherein each of said —C₁₋₁₀alkyl, —C₂₋₁₀alkenyl, —C₂₋₁₀alkynyl, —C₁₋₁₀alkoxy, C₁₋₁₀ monocyclic cycloalkyl, C₁₋₁₀ monocyclic cycloalkenyl, C₁₋₁₀ monocyclic cycloalkynyl, 4- to 10-membered monocyclic heterocyclyl containing 1, 2 or 3 heteroatoms independently selected from nitrogen, oxygen and optionally oxidized sulfur, 6-membered monocyclic aryl, or 5- or 6-membered monocyclic heteroaryl is unsubstituted or substituted with 1, 2 or 3 R^{3a};
R^{3a}, and R^{3b} are each independently hydrogen, —C₁₋₅alkyl, —C₂₋₅alkenyl, —C₂₋₅alkynyl, or C₃₋₆ monocyclic cycloalkyl;
each R^{3d} is independently hydrogen, halogen, —C₁₋₁₀alkyl, —C₂₋₁₀alkenyl, —C₁₋₁₀alkoxy, —C₂₋₁₀alkynyl or C₃₋₆ monocyclic cycloalkyl.

In a preferred embodiment, R⁶¹, R⁶³ and R⁶⁴ are each independently hydrogen. In a further preferred embodiment, R⁶² is halogen, —C₁₋₅alkyl, —OC₁₋₅deuteroalkyl, —C₂₋₅alkenyl, —C₁₋₅alkoxy, or C₃₋₆ monocyclic cycloalkyl; wherein each of said C₁₋₅alkyl, —C₂₋₅alkenyl, —C₁₋₅alkoxy, or C₃₋₆ monocyclic cycloalkyl is unsubstituted or substituted with 1, 2 or 3 R^{3a};

Each R^{3d} is independently hydrogen, F, Cl, Br, I, methoxy, ethoxy, methyl, or ethyl.

In a preferred embodiment, R⁶¹, and R⁶³ are each independently hydrogen.

In a preferred embodiment, R⁶², R⁶³ and R⁶⁴ are each independently hydrogen.

In a preferred embodiment, R⁶³ and R⁶⁴ are each independently hydrogen.

In a preferred embodiment, R⁶¹, R⁶², and R⁶⁴ are each independently hydrogen.

In a first embodiment of Aspect 4, X¹ is CR⁶¹,
R⁶¹, R⁶², R⁶³ and R⁶⁴ are each independently hydrogen, methyl, —OCD₃, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, pyrrolidinyl, furanyl, pyrazolyl, oxazolyl, oxetanyl, azetidinyl, —NH₂, —NH(C₁₋₅alkyl), —N(C₁₋₅alkyl)₂, —CN, F, Cl, Br or I; wherein each of said methyl, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, pyrrolidinyl, furanyl, pyrazolyl, oxazolyl, oxetanyl or azetidinyl is unsubstituted or substituted with 1, 2 or 3 R^{3a};
each R^{3d} is independently hydrogen, methyl, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, oxiranyl, oxetanyl, F, Cl, Br or I.

In a second embodiment of Aspect 4, X¹ is CR⁶¹,
R⁶¹, R⁶², R⁶³ and R⁶⁴ are each independently hydrogen, deuterium, methyl, —OCD₃, ethyl, n-propyl, isopropyl, ethenyl, prop-1-enyl, prop-2-enyl, ethynyl, 1-propynyl, cyclopropyl, —CN, F, Cl, Br, I, —N(CH₃)₂, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂, —CH(CH₃)OCH₃, —OCHF₂ or —CH₂OCH₃.

In a third embodiment of Aspect 4, X¹ is CR⁶¹,
R⁶¹, R⁶³, and R⁶⁴ are hydrogen;
R⁶² is hydrogen, methyl, —OCD₃, ethyl, n-propyl, isopropyl, ethenyl, prop-1-enyl, prop-2-enyl, ethynyl, 1-propynyl, cyclopropyl, F, Cl, Br, I, —N(CH₃)₂, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂ or —CH₂OCH₃.

In a fourth embodiment of Aspect 4, X¹ is CR⁶¹,
R⁶¹, R⁶³ and R⁶⁴ are each independently hydrogen;
R⁶² is each independently methyl, ethyl, I, cyclopropyl, ethyl or methoxy.

Aspect 5, The compound of any one of Aspects 1-3, X¹ is N,
R⁶², R⁶³ and R⁶⁴ are each independently hydrogen, halogen, —C₁₋₁₀alkyl, —OC₁₋₁₀deuteroalkyl, —C₂₋₁₀alkenyl, —C₂₋₁₀alkynyl, —C₁₋₁₀alkoxy, C₃₋₁₀ monocyclic cycloalkyl, C₃₋₁₀ monocyclic cycloalkenyl, C₃₋₁₀ monocyclic cycloalkynyl, 4- or 10-membered monocyclic heterocyclyl containing 1, 2 or 3 heteroatoms independently selected from nitrogen, oxygen and optionally oxidized sulfur, 6-membered monocyclic aryl, 5- or 6-membered monocyclic heteroaryl containing 1, 2 or 3 heteroatoms independently selected from nitrogen, oxygen and optionally oxidized sulfur, or —NR^{3a}R^{3b}; wherein each of said —C₁₋₁₀alkyl, —C₂₋₁₀alkenyl, —C₂₋₁₀alkynyl, —C₁₋₁₀alkoxy, C₃₋₁₀ monocyclic cycloalkyl, C₃₋₁₀ monocyclic cycloalkenyl, C₃₋₁₀ monocyclic cycloalkynyl, 4- or 10-membered monocyclic heterocyclyl containing 1, 2 or 3 heteroatoms independently selected from nitrogen, oxygen and optionally oxidized sulfur, 6-membered monocyclic aryl, 5- or 6-membered monocyclic heteroaryl containing 1, 2 or 3 heteroatoms independently selected from nitrogen, oxygen and optionally oxidized sulfur is unsubstituted or substituted with 1, 2 or 3 R^{3a};
R^{3a}, and R^{3b} are each independently hydrogen, —C₁₋₁₀alkyl, —C₂₋₁₀alkenyl, —C₂₋₁₀alkynyl, or cycloalkyl;
each R^{3d} is independently hydrogen, halogen, —C₁₋₁₀alkyl, —C₂₋₁₀alkenyl, —C₁₋₁₀alkoxy, —C₂₋₁₀alkynyl, or cycloalkyl.
   In a preferred embodiment, R⁶³ and R⁶⁴ are each independently hydrogen. In a preferred embodiment, R⁶² is C₃₋₆ monocyclic cycloalkyl or —C₁₋₅alkoxy, wherein each of said C₃₋₆ monocyclic cycloalkyl or —C₁₋₅alkoxy is unsubstituted or substituted with 1, 2 or 3 R^{3d};
each R^{3d} is independently hydrogen, F, Cl, Br, I, methoxy, ethoxy, methyl, or ethyl.

In a first embodiment of Aspect 5, X¹ is N,
R⁶², R⁶³, and R⁶⁴ are each independently hydrogen, methyl, —OCD₃, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, pyrrolidinyl, furanyl, pyrazolyl, oxazolyl, oxetanyl, pyrrolidinyl, —NH₂, —NH(C₁₋₆ alkyl), —N(C₁₋₆alkyl)₂, F, Cl, Br or I; wherein each of said methyl, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, pyrrolidinyl, furanyl, pyrazolyl, oxazolyl, oxetanyl, pyrrolidinyl is unsubstituted or substituted with 1, 2 or 3 R^{3d};
each R^{3d} is independently hydrogen, methyl, ethyl, 1-propyl, 2-propyl, 1-butyl, 2-methyl-1-propyl, 1-methylpropyl, 1,1-dimethylethyl, ethenyl, prop-1-enyl, prop-2-enyl, ethynyl, 1-propynyl, 2-propynyl, methoxy, ethoxy, propoxy, cyclopropyl, cyclobutyl, cyclopentyl, cyclohexyl, cycloheptyl, cyclooctyl, oxiranyl, oxetanyl, F, Cl, Br or I.

In a second embodiment of Aspect 5, X¹ is N,
R⁶², R⁶³, and R⁶⁴ are each independently hydrogen, methyl, —OCD₃, ethyl, n-propyl, isopropyl, ethenyl, prop-1-enyl, prop-2-enyl, ethynyl, 1-propynyl, cyclopropyl, F, Cl, Br, I, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂, or —CH₂OCH₃.

In a third embodiment of Aspect 5, X¹ is N,
R⁶³ and R⁶⁴ are hydrogen;
R⁶² is hydrogen, methyl, —OCD₃, ethyl, n-propyl, isopropyl, ethenyl, prop-1-enyl, prop-2-enyl, ethynyl, 1-propynyl, cyclopropyl, F, Cl, Br, I, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂, or —CH₂OCH₃.

In a fourth embodiment of Aspect 5, X¹ is N,
R⁶³ and R⁶⁴ are hydrogen;
R⁶² is cyclopropyl or methoxy.

In a fifth embodiment of Aspect 5, X¹ is N,
R⁶³ and R⁶⁴ are hydrogen;
R⁶² is —OCD₃.

Aspect 6, The compound of any one of Aspects 1-5, R⁷ is each independently hydrogen, —C₁₋₁₀alkyl, —NH₂, —NH(—C₁₋₆alkyl) or —N(—C₁₋₆alkyl)₂.

In a first embodiment of Aspect 6, n is 0, 1, 2, or 3; R⁷ is each independently hydrogen, methyl, ethyl, 1-propyl, 2-propyl, or —NH₂. In a second embodiment of Aspect 6, n is 2, or 3; one of R⁷ is each independently methyl; the other R⁷ is hydrogen. In a third embodiment of Aspect 6, n is 2, or 3; one of R⁷ is —NH₂; and the other R⁷ is hydrogen. In a fourth embodiment of Aspect 6, R⁷ is hydrogen.

Aspect 7, The compound of any one of Aspects 1-6, R¹ is —OR^{1a}; and R^{1a} is hydrogen, —C₁₋₁₀deuteroalkyl(preferably, —C₁₋₅deuteroalkyl), —C₁₋₁₀alkyl(preferably, —C₁₋₅alkyl) or -haloC₁₋₁₀alkyl(preferably, -haloC₁₋₅alkyl).

In some embodiments of Aspect 7, the compound of formula (I) is a compound of formula (III-1):

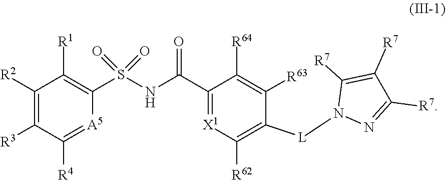

In some embodiments of the compound of formula (I) or formula (III-1),
A⁵ is N or CR⁵; if present, R⁵ is hydrogen, deuterium, halogen, —C₁₋₃alkyl or -haloC₁₋₃alkyl;
R¹ is —OR^{1a}; and R^{1a} is hydrogen, —C₁₋₁₀deuteroalkyl (preferably, —C₁₋₅deuteroalkyl; more preferably, -CD₃), —C₁₋₁₀alkyl (preferably, —C₁₋₅alkyl; more preferably, methyl) or -haloC₁₋₁₀alkyl(preferably, -haloC₁₋₅alkyl; more preferably, —CF₃, —CH₂F, or —CHF₂);
R² is hydrogen, deuterium, halogen, —C₁₋₃alkyl or -haloC₁₋₃alkyl (preferably, hydrogen or halogen; more preferably, hydrogen);
R³ is hydrogen, deuterium, halogen, —C₁₋₃alkyl or -haloC₁₋₃alkyl;
R⁴ is —C₁₋₁₀alkyl, —C₁₋₁₀alkoxy, C₃₋₁₀cycloalkyl, 6-membered aryl, 5- to 6-membered heteroaryl containing 1, 2 or 3 heteroatoms independently selected from nitrogen, oxygen and optionally oxidized sulfur, or 4- to 8-membered heterocyclyl containing 1, 2 or 3 heteroatoms independently selected from nitrogen, oxygen and optionally oxidized sulfur;
   wherein said —C₁₋₁₀alkyl is unsubstituted or substituted with 1, 2, or 3 substituents independently selected from halogen, —OC₁₋₁₀alkyl, cycloalkyl, cycloalkenyl, cycloalkynyl, heterocyclyl, aryl, heteroaryl, —CN, —NO₂, —CO₂C₁₋₁₀alkyl, —N(C₁₋₁₀alkyl)₂, —NH(C₁₋₁₀alkyl), and —NH₂;
wherein each of said C₃₋₁₀cycloalkyl, 6-membered aryl, 5- to 6-membered heteroaryl, or 4- to 8-membered heterocyclyl is unsubstituted or substituted with 1, 2, or 3 R^{1d};
R^{1d} is selected from halogen, —C₁₋₁₀alkyl, —C₁₋₁₀alkoxy, and -haloC₁₋₁₀alkyl;
wherein said —C₁₋₁₀alkyl is unsubstituted or substituted with 1, 2, or 3 substituents independently selected from —CN, —OR¹h, and —NR^{1h}R¹ⁱ;
each R^{1h} and R¹ⁱ is independently selected from hydrogen, methyl, ethyl, 1-propyl, and 2-propyl;
   X¹ is N or CR⁶¹; if present R⁶¹ is hydrogen, or —C₁₋₁₀alkoxy;
R⁶² is hydrogen, halogen, deuterium, —C₁₋₁₀alkyl, —SC₁₋₁₀alkyl, —OC₁₋₁₀deuteroalkyl, —C₂₋₁₀alkenyl, —C₂₋₁₀alkynyl, —C₁₋₁₀alkoxy, C₃₋₁₀ monocyclic cycloalkyl, C₃₋₁₀ monocyclic cycloalkenyl, C₃₋₁₀ monocyclic cycloalkynyl, 4- to 10-membered monocyclic heterocyclyl containing 1, 2 or 3 heteroatoms independently selected from nitrogen, oxygen and optionally oxidized sulfur, 6-membered monocyclic aryl, 5- or 6-membered monocyclic heteroaryl, —CN, or —NR^{3a}R^{3b}; wherein each of said —C₁₋₁₀alkyl, —C₂₋₁₀alkenyl, —C₂₋₁₀alkynyl, —C₁₋₁₀alkoxy, C₁₋₁₀ monocyclic cycloalkyl, C₁₋₁₀ monocyclic cycloalkenyl, C₁₋₁₀ monocyclic cycloalkynyl, 4- to 10-membered monocyclic heterocyclyl containing 1, 2 or 3 heteroatoms independently selected from nitrogen, oxygen and optionally oxidized sulfur, 6-membered monocyclic aryl, or 5- or 6-membered monocyclic heteroaryl is unsubstituted or substituted with 1, 2 or 3 R^{3d};
R^{3a} and R^{3b} are each independently hydrogen, —C₁₋₅alkyl, —C₂₋₅alkenyl, —C₂₋₅alkynyl, or C₃₋₆ monocyclic cycloalkyl;
each R^{3d} is independently hydrogen, halogen, —C₁₋₁₀alkyl, —C₂₋₁₀alkenyl, —C₁₋₁₀alkoxy, —C₂₋₁₀alkynyl or C₃₋₆ monocyclic cycloalkyl.
   R⁶³ is hydrogen or deuterium;
   R⁶⁴ is hydrogen, deuterium, halogen, —CN, —C₁₋₁₀alkyl, or —C₁₋₁₀alkoxy.

In some embodiments of the compound of formula (I) or formula (III-1), A⁵ is N or CR⁵; if present, R⁵ is hydrogen, halogen, —C₁₋₃alkyl or -haloC₁₋₃alkyl;
R¹ is —OR^{1a}; and R^{1a} is hydrogen, —C₁₋₁₀deuteroalkyl(preferably, —C₁₋₅deuteroalkyl), —C₁₋₁₀alkyl(preferably, —C₁₋₅alkyl) or -haloC₁₋₁₀alkyl(preferably, -haloC₁₋₅alkyl);
R² is hydrogen, deuterium, halogen, —C₁₋₃alkyl or -haloC₁₋₃alkyl;
R³ is hydrogen, halogen, —C₁₋₃alkyl or -haloC₁₋₃alkyl;
R⁴ is methyl, ethyl, 1-propyl, 2-propyl, 1-butyl, 2-methyl-1-propyl, 1-methylpropyl, 1,1-dimethylethyl, methoxy, cyclopropyl, cyclobutyl, cyclopentyl, triazolyl, morpholinyl, piperidinyl, tetrahydropyranyl, oxetanyl, tetrahydrofuranyl, dioxanyl, oxazolyl, 8-oxa-3-azabicyclo[3.2.1]octanyl, 2-oxaspiro[3.3]heptanyl, or pyrazolyl;
wherein each of said methyl, ethyl, 1-propyl, 2-propyl, 1-butyl, 2-methyl-1-propyl, 1-methylpropyl, 1,1-dimethylethyl, is unsubstituted or substituted with 1, 2, or 3 substituents independently selected from halogen, —C₁₋₁₀alkyl, -haloC₁₋₁₀alkyl, —OC₁₋₁₀alkyl, cycloalkyl, cycloalkenyl, cycloalkynyl, heterocyclyl, aryl, heteroaryl, —CN, —NO₂, —CO₂C₁₋₁₀alkyl, —N(C₁₋₁₀alkyl)₂, —NH(C₁₋₁₀alkyl), and —NH₂; and
wherein each of said cyclopropyl, cyclobutyl, cyclopentyl, triazolyl, morpholinyl, piperidinyl, tetrahydropyranyl, oxetanyl, tetrahydrofuranyl, dioxanyl, oxazolyl, or pyrazolyl is unsubstituted or substituted with 1, 2, or 3
R^{1d}; and
R^{1d} is selected from halogen, —C₁₋₁₀alkyl, —C₁₋₁₀alkoxy, and -haloC₁₋₁₀alkyl;
wherein said —C₁₋₁₀alkyl is unsubstituted or substituted with 1, 2, or 3 substituents independently selected from —CN, hydroxy, methoxy, —NH₂, and —N(CH₃)₂.

In some embodiments of the compound of formula (I) or formula (III-1), R⁴ is 2-oxabicyclo[2.1.1]hexanyl, or bicyclo[1.1.1]pentanyl.

In some embodiments of the compound of formula (I) or formula (III-1), A⁵ is N or CR⁵; if present, R⁵ is hydrogen, halogen, —C₁₋₃alkyl or -haloC₁₋₃alkyl;
R¹ is —OR^{1a}; and R^{1a} is hydrogen, —C₁₋₁₀deuteroalkyl(preferably, —C₁₋₅deuteroalkyl), —C₁₋₁₀alkyl(preferably, —C₁₋₅alkyl) or -haloC₁₋₁₀alkyl(preferably, -haloC₁₋₅alkyl);
R² is hydrogen, deuterium, halogen, —C₁₋₃alkyl or -haloC₁₋₃alkyl;
R³ is hydrogen, deuterium, halogen, —C₁₋₃alkyl or -haloC₁₋₃alkyl;

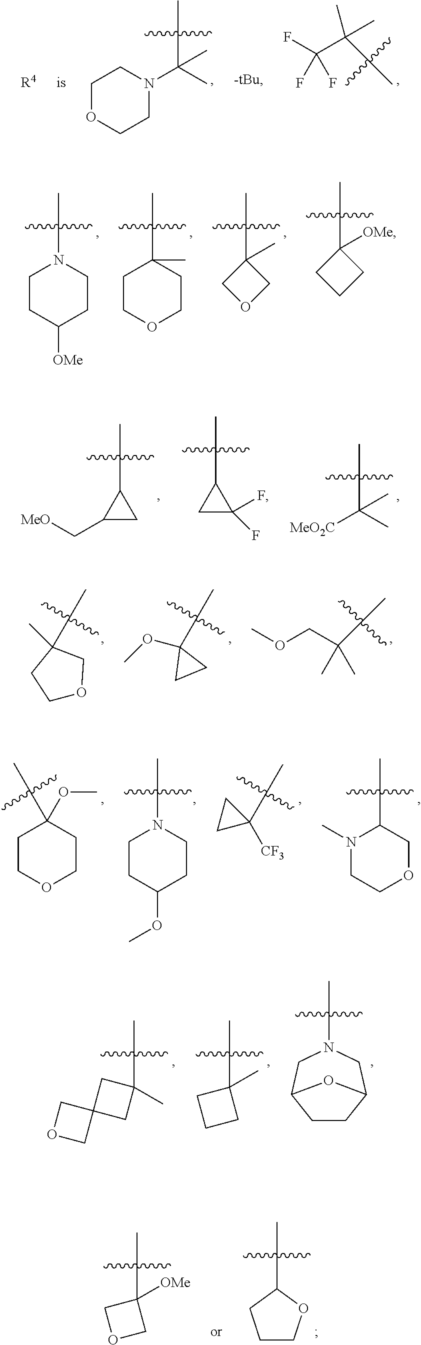

X¹ is N or CR⁶¹,
if R⁶¹ is present, R⁶¹, R⁶³, and R⁶⁴ are hydrogen;
R⁶² is hydrogen, methyl, —OCD₃, ethyl, n-propyl, isopropyl, ethenyl, prop-1-enyl, prop-2-enyl, ethynyl, 1-propynyl, cyclopropyl, F, Cl, Br, I, —N(CH₃)₂, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂ or —CH₂OCH₃.

In some embodiments of the compound of formula (I) or formula (III-1), R⁴ is

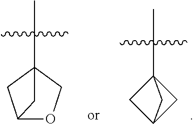 [SMILES: *C12COC(C1)C2 ; *C12CC(C1)C2]

In some embodiments of the compound of formula (I) or formula (III-1), A⁵ is N or CR⁵; if present, R⁵ is hydrogen, halogen, —C₁₋₃alkyl or -haloC₁₋₃alkyl;
R¹ is —OR^{1a}; and R^{1a} is hydrogen, —C₁₋₁₀deuteroalkyl (preferably, —C₁₋₅deuteroalkyl), —C₁₋₁₀alkyl (preferably, —C₁₋₅alkyl) or -haloC₁₋₁₀alkyl (preferably, -haloC₁₋₅alkyl);
R² is hydrogen, halogen, —C₁₋₃alkyl or -haloC₁₋₃alkyl;
R³ is hydrogen, halogen, —C₁₋₃alkyl or -haloC₁₋₃alkyl;
R⁴ is -tBu,

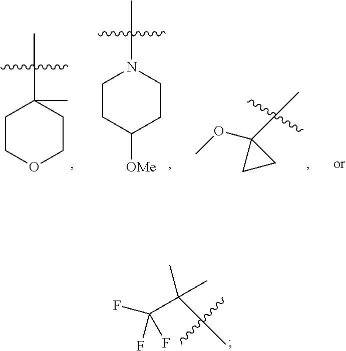

R⁶¹, R⁶³, and R⁶⁴ are hydrogen;
R⁶² is hydrogen, methyl, —OCD₃, ethyl, n-propyl, isopropyl, ethenyl, prop-1-enyl, prop-2-enyl, ethynyl, 1-propynyl, cyclopropyl, F, Cl, Br, I, —N(CH₃)₂, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂ or —CH₂OCH₃.

In some embodiments of the compound of formula (I) or formula (III-1), A⁵ is N or CR⁵; R⁵ is hydrogen,
R¹ is —OR^{1a}; and R^{1a} is hydrogen, —C₁₋₁₀deuteroalkyl(preferably, —C₁₋₅deuteroalkyl), —C₁₋₁₀alkyl(-C₁₋₅alkyl) or -haloC₁₋₁₀alkyl(-haloC₁₋₅alkyl);
R² is hydrogen;
R³ is hydrogen;
R⁴ is —C₁₋₅alkyl or -haloC₁₋₅alkyl;
X¹ is N or CR⁶¹,
if R⁶¹ is present, R⁶¹, R⁶³ and R⁶⁴ are each independently hydrogen;
R⁶² is each independently —C₁₋₅alkyl, —C₂₋₅alkenyl, C₃₋₆ monocyclic cycloalkyl, or —C₁₋₅alkoxy.

In some embodiments of the compound of formula (I) or formula (III-1), A⁵ is N or CR⁵; R⁵ is hydrogen,
R¹ is —OR^{1a}; and R^{1a} is hydrogen, —C₁₋₁₀deuteroalkyl, —C₁₋₁₀alkyl or -haloC₁₋₁₀alkyl;
R² is hydrogen;
R³ is hydrogen;
R⁴ is —C₁₋₅alkyl or -haloC₁₋₅alkyl;
X¹ is N or CR⁶¹,
if R⁶¹ is present, R⁶¹, R⁶³ and R⁶⁴ are each independently hydrogen;
R⁶² is each independently —OC₁₋₁₀deuteroalkyl.

In some embodiments of the compound of formula (I) or formula (III-1), A⁵ is N or CR⁵; R⁵ is hydrogen,
R¹ is —OR^{1a}; and R^{1a} is hydrogen, —C₁₋₁₀deuteroalkyl(preferably, —C₁₋₅deuteroalkyl), —C₁₋₁₀alkyl(-C₁₋₅alkyl) or -haloC₁₋₁₀alkyl(-haloC₁₋₅alkyl);
R² is hydrogen;
R³ is hydrogen;
R⁴ is 1,1-dimethylethyl, or 1,1,1-trifluoro-2-methylpropanyl;
X¹ is N or CR⁶¹,
if R⁶¹ is present, R⁶¹, R⁶³ and R⁶⁴ are each independently hydrogen;
R⁶² is each independently methyl, ethyl, cyclopropyl, ethenyl, —OCD₃, or methoxy.

In some embodiments of the compound of formula (I) or formula (III-1), A⁵ is N or CR⁵; R⁵ is hydrogen,
R¹ is —OR^{1a}; and R^{1a} is hydrogen, —C₁₋₅deuteroalkyl, —C₁₋₅alkyl or -haloC₁₋₅alkyl;
R² is hydrogen;
R³ is hydrogen;
R⁴ is 1,1-dimethylethyl, or 1,1,1-trifluoro-2-methylpropanyl;
X¹ is N or CR⁶¹,
if R⁶¹ is present, R⁶¹, R⁶³ and R⁶⁴ are each independently hydrogen;
R⁶² is each independently-OCD₃.

In some embodiments of Aspect 1, the compound is of Formula (VI):

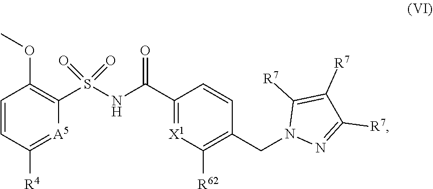

or a pharmaceutically acceptable salt, deuterated analog, N-oxide, or tautomer thereof, wherein:
X¹ is N or C(H);
R⁶² is ethyl, ethenyl, cyclopropyl, —OCD₃, or methoxy;
R⁴ is

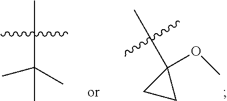

A⁵ is CR⁵ or N; if present, R⁵ is hydrogen; and
R^{4c} is methyl or —CF₃; or
R⁵ and R^{4c}, together with the atoms to which they are attached and intervening atoms, form a dihydrofuranyl or dihydropyranyl ring which is unsubstituted or substituted with 1 or 2 R^{1d};
   R^{1d} is methyl.

In some embodiments of Aspect 7, the compound of formula (I) is a compound of formula (III-2):

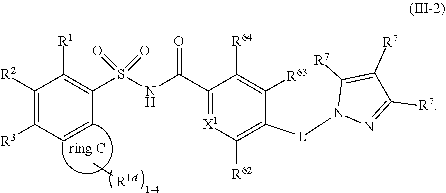

In some embodiments of the compound of formula (III-2),
R¹ is —OR^{1a}; and R^{1a} is hydrogen, —C₁₋₁₀deuteroalkyl, —C₁₋₁₀alkyl or -haloC₁₋₁₀alkyl;
R² is hydrogen, halogen, —C₁₋₃alkyl or -haloC₁₋₃alkyl;
R³ is hydrogen, halogen, —C₁₋₃alkyl or -haloC₁₋₃alkyl;
ring C is 5- to 7-membered heterocyclyl containing 1, 2 or 3 heteroatoms independently selected from nitrogen, oxygen and optionally oxidized sulfur;
   each R^{1d} is independently hydrogen, halogen, —C₁₋₅alkyl, or -haloC₁₋₅alkyl; or two R^{1d} substituted at same or different atom(s), together with the atom(s) to which they are attached and any intervening atoms, form C₃₋₁₀ monocyclic cycloalkyl, or 4- to 10-membered monocyclic heterocyclyl containing 1, 2 or 3 heteroatoms independently selected from nitrogen, oxygen and optionally oxidized sulfur;
   X¹ is N or CR⁶¹; if present R⁶¹ is hydrogen, or —C₁₋₁₀alkoxy;
R⁶² is hydrogen, halogen, —C₁₋₁₀alkyl, —OC₁₋₁₀deuteroalkyl, —C₂₋₁₀alkenyl, —C₂₋₁₀alkynyl, —C₁₋₁₀alkoxy, C₃₋₁₀ monocyclic cycloalkyl, C₃₋₁₀ monocyclic cycloalkenyl, C₃₋₁₀ monocyclic cycloalkynyl, 4- to 10-membered monocyclic heterocyclyl containing 1, 2 or 3 heteroatoms independently selected from nitrogen, oxygen and optionally oxidized sulfur, 6-membered monocyclic aryl, 5- or 6-membered monocyclic heteroaryl, —CN, or —NR^{3a}R^{3b}; wherein each of said —C₁₋₁₀alkyl, —C₂₋₁₀alkenyl, —C₂₋₁₀alkynyl, —C₁₋₁₀alkoxy, C₁₋₁₀ monocyclic cycloalkyl, C₁₋₁₀ monocyclic cycloalkenyl, C₁₋₁₀ monocyclic cycloalkynyl, 4- to 10-membered monocyclic heterocyclyl containing 1, 2 or 3 heteroatoms independently selected from nitrogen, oxygen and optionally oxidized sulfur, 6-membered monocyclic aryl, or 5- or 6-membered monocyclic heteroaryl is unsubstituted or substituted with 1, 2 or 3 R^{3a};
R^{3a} and R^{3b} are each independently hydrogen, —C₁₋₅alkyl, —C₂₋₅alkenyl, —C₂₋₅alkynyl, or C₃₋₆ monocyclic cycloalkyl;
each R^{3d} is independently hydrogen, halogen, —C₁₋₁₀alkyl, —C₂₋₁₀alkenyl, —C₁₋₁₀alkoxy, —C₂₋₁₀alkynyl or C₃₋₆ monocyclic cycloalkyl.
   R⁶³ is hydrogen;
R⁶⁴ is hydrogen, halogen, —CN, —C₁₋₁₀alkyl, or —C₁₋₁₀alkoxy.

In some embodiments of the compound of formula (III-2), ring C is dihydrofuranyl or dihydro-pyranyl;
each R^{1d} is independently hydrogen, halogen, —C₁₋₄alkyl, -haloC₁₋₄alkyl, or —C₁₋₄alkoxy; or two R^{1d} substituted at same or different atom(s), together with the atom(s) to which they are attached and any intervening atoms, form cyclobutyl ring, or tetrahydropyranyl.

In some embodiments of the compound of formula (III-2), the compound is of formula (IV-1):

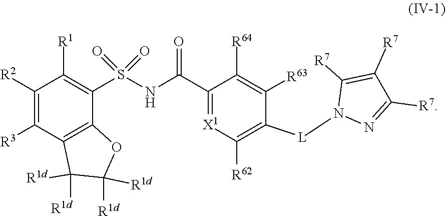

In some embodiments of the compound of formula (IV-1), R¹ is —OR^{1a}; and R^{1a} is hydrogen, —C₁₋₁₀deuteroalkyl(preferably, —C₁₋₅deuteroalkyl), —C₁₋₁₀alkyl(preferably, —C₁₋₅alkyl) or -haloC₁₋₁₀alkyl(-haloC₁₋₅alkyl);
R² is hydrogen, halogen, —C₁₋₃alkyl or -haloC₁₋₃alkyl;
R³ is hydrogen, halogen, —C₁₋₃alkyl or -haloC₁₋₃alkyl;
   each R^{1d} is independently hydrogen, F, methyl, ethyl, 1-propyl, 2-propyl, 1-butyl, 2-methyl-1-propyl, 1-methylpropyl, or 1,1-dimethylethyl; or
two R^{1d} substituted at same or different atom(s), together with the atom(s) to which they are attached and any intervening atoms, form cyclobutyl ring, or tetrahydropyranyl;
X¹ is N or CR⁶¹;
if present R⁶¹ is hydrogen;
R⁶³ and R⁶⁴ are hydrogen;
R⁶² is hydrogen, —OCD₃, methyl, ethyl, n-propyl, isopropyl, ethenyl, prop-1-enyl, prop-2-enyl, ethynyl, 1-propynyl, cyclopropyl, F, Cl, Br, I, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂, or —CH₂OCH₃.

In some embodiments of the compound of formula (III-2), the compound is of formula (V-1):

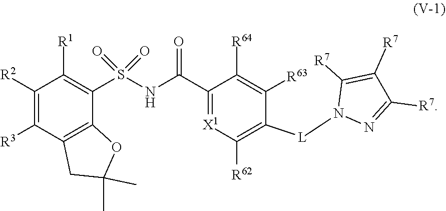

In some embodiments of the compound of formula (III-2), the compound is of formula (V-2):

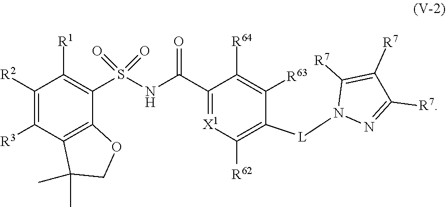

In some embodiments of the compound of formula (III-2), the compound is of formula (V-3):

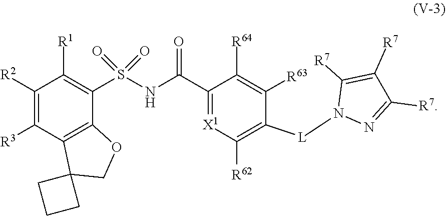

In some embodiments of the compound of formula (V-1), formula (V-2), or formula (V-3),
R¹ is —OR^{1a}; and R^{1a} is hydrogen, —C₁₋₁₀deuteroalkyl(preferably, —C₁₋₅deuteroalkyl), —C₁₋₁₀alkyl(preferably, —C₁₋₅alkyl) or -haloC₁₋₁₀alkyl(preferably, -haloC₁₋₅alkyl);
R² is hydrogen, halogen, —C₁₋₃alkyl or -haloC₁₋₃alkyl;
R³ is hydrogen, halogen, —C₁₋₃alkyl or -haloC₁₋₃alkyl;
   X¹ is N or CR⁶¹;
   if present R⁶¹ is hydrogen;
each R^{1d} is independently hydrogen, F, methyl, ethyl, 1-propyl, 2-propyl, 1-butyl, 2-methyl-1-propyl, 1-methylpropyl, or 1,1-dimethylethyl; or two R^{1d} substituted at same or different atom(s), together with the atom(s) to which they are attached and any intervening atoms, form cyclobutyl ring, or tetrahydropyranyl;
X¹ is N or CR⁶¹;
if present R⁶¹ is hydrogen;
R⁶³ and R⁶⁴ are hydrogen;
R⁶² is hydrogen, methyl, —OCD₃, ethyl, n-propyl, isopropyl, ethenyl, prop-1-enyl, prop-2-enyl, ethynyl, 1-propynyl, cyclopropyl, F, Cl, Br, I, —N(CH₃)₂, methoxy, ethoxy, propoxy, pyrrolidinyl, furanyl, pyrazolyl, oxazolyl, oxetanyl, pyrrolidinyl, methoxycyclopentyl, —CH₂F, —CH₂CHF₂, or —CH₂OCH₃.

In some embodiments of the compound of formula (III-2), the compound is of formula (IV-4):

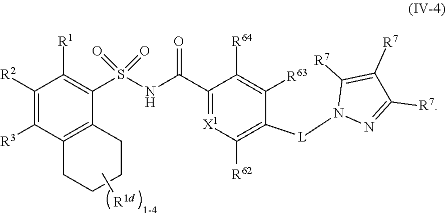

In some embodiments of the compound of formula (V-4),
R¹ is —OR^{1a}; and R^{1a} is hydrogen, —C₁₋₁₀deuteroalkyl(preferably, —C₁₋₅deuteroalkyl), —C₁₋₁₀alkyl(-C₁₋₅alkyl) or -haloC₁₋₁₀alkyl(-haloC₁₋₅alkyl);
R² is hydrogen, halogen, —C₁₋₃alkyl or -haloC₁₋₃alkyl;
R³ is hydrogen, halogen, —C₁₋₃alkyl or -haloC₁₋₃alkyl;
R^{1d} is each independently hydrogen, hydrogen, F, methyl, ethyl, 1-propyl, 2-propyl, 1-butyl, 2-methyl-1-propyl, 1-methylpropyl, or 1,1-dimethylethyl;
   X¹ is N or CR⁶¹;
   if present R⁶¹ is hydrogen;
R⁶³, and R⁶⁴ are hydrogen;
R⁶² is hydrogen, methyl, —OCD₃, ethyl, n-propyl, isopropyl, ethenyl, prop-1-enyl, prop-2-enyl, ethynyl, 1-propynyl, cyclopropyl, F, Cl, Br, I, —N(CH₃)₂, methoxy, ethoxy, propoxy, pyrrolidinyl, furanyl, pyrazolyl, oxazolyl, oxetanyl, methoxycyclopentyl, —CH₂F, —CH₂CHF₂ or —CH₂OCH₃.

In some embodiments of the compound (III-2), the compound is of formula (V-4):

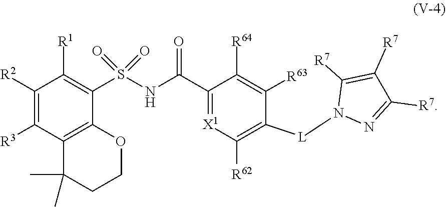

In some embodiments of the compound (III-2), the compound is of formula (V-5):

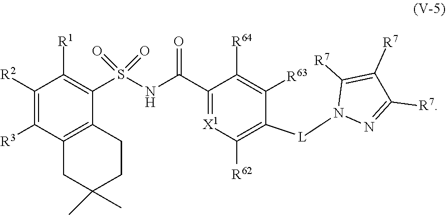

In some embodiments of the compound of formula (V-4), or formula (V-5),
X¹ is N or CR⁶¹;
if present R⁶¹ is hydrogen;
R⁶³, and R⁶⁴ are hydrogen;
R⁶² is hydrogen, methyl, ethyl, n-propyl, isopropyl, ethenyl, prop-1-enyl, prop-2-enyl, ethynyl, 1-propynyl, cyclopropyl, F, Cl, Br, I, —N(CH₃)₂, methoxy, —OCD₃, ethoxy, propoxy, pyrrolidinyl, furanyl, pyrazolyl, oxazolyl, oxetanyl, methoxycyclopentyl, —CH₂F, —CH₂CHF₂ or —CH₂OCH₃.

In some embodiments of the compound of formula (III-2), formula (IV-1), formula (IV-4), formula (V-1), formula (V-2), formula (V-4), or formula (V-5),
R¹ is —OR^{1a}; and R^{1a} is methyl;
R² is hydrogen;
R³ is hydrogen;
R⁶³ and R⁶⁴ are hydrogen;
R⁶² is methyl, ethyl, cyclopropyl, methoxy, or ethoxy.

In some embodiments of the compound of formula (III-2), formula (IV-1), formula (IV-4), formula (V-1), formula (V-2), formula (V-4), or formula (V-5),
R¹ is —OR^{1a}; and R^{1a} is methyl;
R² is hydrogen;
R³ is hydrogen;
R⁶³ and R⁶⁴ are hydrogen;
   R⁶² is methyl, ethyl, cyclopropyl, methoxy, —OCD₃, or ethoxy.

In some embodiments of the compound of formula (III-2), formula (IV-1), formula (IV-4), formula (V-1), formula (V-2), formula (V-4), or formula (V-5), R¹ is —OR^{1a}; and R^{1a} is methyl;
R² is hydrogen;
R³ is hydrogen;
R⁶¹, R⁶³ and R⁶⁴ are hydrogen;
R⁶² is methyl, ethenyl, cyclopropyl, methoxy, or ethoxy.

In some embodiments of the compound of formula (III-2), formula (IV-1), formula (IV-4), formula (V-1), formula (V-2), formula (V-4), or formula (V-5),
R¹ is —OR^{1a}; and R^{1a} is methyl;
R² is hydrogen;
R³ is hydrogen;
R⁶¹, R⁶³ and R⁶⁴ are hydrogen;
R⁶² is methoxy, —OCD₃, or ethoxy.

In a first embodiment of Aspect 7, R¹ is —OR^{1a}; and R^{1a} is hydrogen, —CH₂F, —CHF₂, —CF₃, methyl, —CD₃, ethyl, 1-propyl, 2-propyl, 1-butyl, 2-methyl-1-propyl, 1-methylpropyl, or 1,1-dimethylethyl.

In a second embodiment of Aspect 7, R¹ is —OR^{1a}; and R^{1a} is methyl, ethyl, 1-propyl, or 2-propyl.

In a third embodiment of Aspect 7, R¹ is —OR^{1a}; and R^{1a} is methyl.

In a fourth embodiment of Aspect 7, R¹ is —OR^{1a}; and R^{1a} is —CD₃.

Aspect 8, The compound of any one of Aspects 1-7, R² is hydrogen.

Aspect 9, The compound of any one of Aspects 1-8, A⁵ is CR⁵; A⁴ is CR⁴; and A³ is CR³.

Aspect 10, The compound of any one of Aspects 1-9, R³ and R⁵ are hydrogen; and R⁴ is —C₁₋₁₀alkyl, —C₁₋₁₀alkoxy, C₃₋₁₀cycloalkyl, 6-membered aryl, 5- to 6-membered heteroaryl containing 1, 2 or 3 heteroatoms independently selected from nitrogen, oxygen and optionally oxidized sulfur, or 4- to 6-membered heterocyclyl containing 1, 2 or 3 heteroatoms independently selected from nitrogen, oxygen and optionally oxidized sulfur;
wherein said —C₁₋₁₀alkyl is unsubstituted or substituted with 1, 2, or 3 substituents independently selected from halogen, —OC₁₋₁₀alkyl, cycloalkyl, cycloalkenyl, cycloalkynyl, heterocyclyl, aryl, heteroaryl, —CN, —NO₂, —CO₂C₁₋₁₀alkyl, —N(C₁₋₁₀alkyl)₂, —NH(C₁₋₁₀alkyl), and —NH₂;
wherein each of said C₃₋₁₀cycloalkyl, 6-membered aryl, 5- to 6-membered heteroaryl, or 4- to 6-membered heterocyclyl is unsubstituted or substituted with 1, 2, or 3 substituents independently selected from halogen, —C₁₋₁₀alkyl, —C₁₋₁₀alkoxy, and -haloC₁₋₁₀alkyl.

In a first embodiment of Aspect 10, wherein R³ and R⁵ are hydrogen; R⁴ is methyl, ethyl, 1-propyl, 2-propyl, 1-butyl, 2-methyl-1-propyl, 1-methylpropyl, 1,1-dimethylethyl, methoxy, cyclopropyl, cyclobutyl, cyclopentyl, triazolyl, morpholinyl, piperidinyl, tetrahydropyranyl, oxetanyl, tetrahydrofuranyl, dioxanyl, oxazolyl, or pyrazolyl;
wherein each of said methyl, ethyl, 1-propyl, 2-propyl, 1-butyl, 2-methyl-1-propyl, 1-methylpropyl, 1,1-dimethylethyl, is unsubstituted or substituted with 1, 2, or 3 substituents independently selected from halogen, —OC₁₋₁₀alkyl, cycloalkyl, cycloalkenyl, cycloalkynyl, heterocyclyl, aryl, heteroaryl, —CN, —NO₂, —CO₂C₁₋₁₀alkyl, —N(C₁₋₁₀alkyl)₂, —NH(C₁₋₁₀alkyl), and —NH₂; and
   wherein each of said cyclopropyl, cyclobutyl, cyclopentyl, triazolyl, morpholinyl, piperidinyl, tetrahydropyranyl, oxetanyl, tetrahydrofuranyl, dioxanyl, oxazolyl, or pyrazolyl is unsubstituted or substituted with 1, 2, or 3 substituents independently selected from halogen, —C₁₋₁₀alkyl, —OC₁₋₁₀alkyl, and haloC₁₋₁₀alkyl.

In a second embodiment of Aspect 10, R³ and R⁵ are hydrogen; R⁴ is

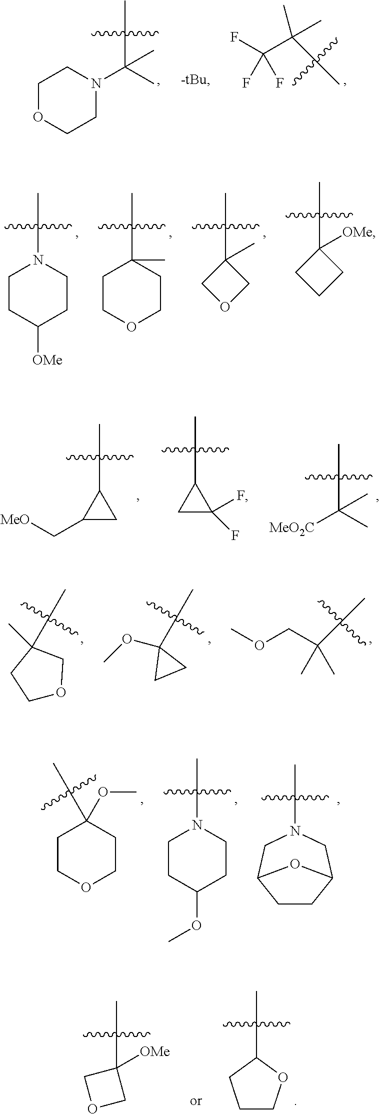

In a third embodiment of Aspect 10, R³ and R⁵ are hydrogen; R⁴ is -tBu,

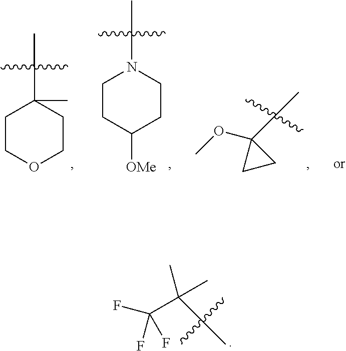

In a fourth embodiment of Aspect 10, R³ and R⁵ are hydrogen; R⁴ is -tBu.

Aspect 11, The compound of any one of Aspects 1-8, R³ is hydrogen; and (R⁴ and R), together with the atoms to which they are attached, form a 5- to 6-membered heterocyclyl ring containing 1, 2 or 3 heteroatoms independently selected from nitrogen, oxygen and optionally oxidized sulfur, wherein said 5- to 6-membered heterocyclyl ring is unsubstituted or substituted with 1, 2, 3, or 4 R^{1d}; and
each R^{1d} is independently hydrogen, halogen, —C₁₋₁₀alkyl, —C₁₋₁₀alkoxy, 4- to 6-membered 4- to 6-membered heterocyclyl, or -haloC₁₋₁₀alkyl.

In a first embodiment of Aspect 11, R³ is hydrogen; and (R⁴ and R), together with the atoms to which they are attached, form dihydrofuranyl or dihydro-pyranyl;
wherein each of said dihydrofuranyl, or dihydro-pyranyl is unsubstituted or substituted with 1, 2, 3, or 4 R^{1d}; and
each R^{1d} is independently hydrogen, halogen, —C₁₋₄alkyl, -haloC₁₋₄alkyl, or —C₁₋₄alkoxy.

In a second embodiment of Aspect 11, R³ is hydrogen; (R⁴ and R⁵), together with the atoms to which they are attached, form

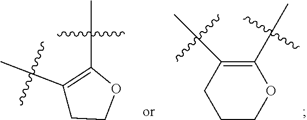 [SMILES: *C1=C(*)OCC1 ; *C1=C(*)OCCC1]

which is unsubstituted or substituted with 1, 2, 3, or 4 R^{1d}; and
each R^{1d} is independently hydrogen, F, methyl, ethyl, 1-propyl, 2-propyl, 1-butyl, 2-methyl-1-propyl, 1-methylpropyl, or 1,1-dimethylethyl.

In a third embodiment of Aspect 11, R³ is hydrogen; and (R⁴ and R⁵), together with the atoms to which they are attached, form

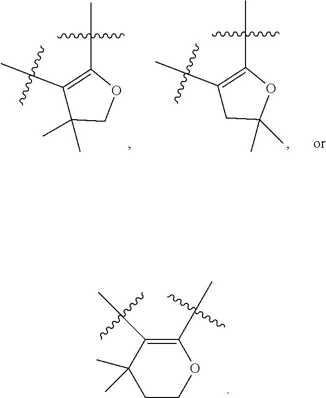

In a fourth embodiment of Aspect 11, R⁵ is hydrogen; and (R³ and R⁴), together with the atoms to which they are attached, form a 5- to 6-membered cycloalkyl ring, 5- to 6-membered heterocyclyl ring containing 1, 2 or 3 heteroatoms independently selected from nitrogen, oxygen and optionally oxidized sulfur, wherein said 5- to 6-membered heterocyclyl ring is unsubstituted or substituted with 1, 2, 3, or 4 R^{1d}; and
each R^{1d} is independently hydrogen, halogen, —C₁₋₁₀alkyl, —C₁₋₁₀alkoxy, or -haloC₁₋₁₀alkyl.

In a fifth embodiment of Aspect 11, R⁵ is hydrogen; and (R³ and R⁴), together with the atoms to which they are attached, form cyclopentyl or cyclopropanyl;
wherein each of said dihydro-pyrrolyl or pyrrolidinyl is unsubstituted or substituted with 1, 2, 3, or 4 R^{1d}; and
each R^{1d} is independently hydrogen, F, methyl, ethyl, 1-propyl, 2-propyl, 1-butyl, 2-methyl-1-propyl, 1-methylpropyl, or 1,1-dimethylethyl.

In a sixth embodiment of Aspect 11, R³ is hydrogen; and (R⁴ and R⁵), together with the atoms to which they are attached, form

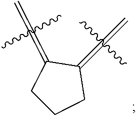 [SMILES: *=C1CCCC1=*]

which is further unsubstituted or substituted with 1 or 2R^{d}; and
each R^{1d} is independently hydrogen, F, methyl, ethyl, 1-propyl, 2-propyl, 1-butyl, 2-methyl-1-propyl, 1-methylpropyl, or 1,1-dimethylethyl.

Aspect 12, The compound of any one of Aspects 1-8, A⁵ is N; A⁴ is CR⁴; A³ is CR³; R³ is H; R⁴ is —C₁₋₁₀alkyl, C₃₋₁₀cycloalkyl, or 4- to 6-membered heterocyclyl containing 1, 2 or 3 heteroatoms independently selected from nitrogen, oxygen and optionally oxidized sulfur;
wherein each of said —C₁₋₁₀alkyl is unsubstituted or substituted with 1, 2, or 3 substituents independently selected from halogen, C₁₋₁₀alkoxy, cycloalkyl, cycloalkenyl, cycloalkynyl, heterocyclyl, aryl, heteroaryl, —CN, —NO₂, —CO₂C₁₋₁₀alkyl, —N(C₁₋₁₀alkyl)₂, —NH(C₁₋₁₀alkyl), or —NH₂; wherein each of said C₃₋₁₀cycloalkyl or 4- to 6-membered heterocyclyl is unsubstituted or substituted with 1, 2, or 3 substituents independently selected from halogen, —C₁₋₁₀alkyl, —C₁₋₁₀alkoxy, and -haloC₁₋₁₀alkyl.

In a first embodiment of Aspect 12, A⁵ is N; A⁴ is —CR⁴; A³ is —CR³; R³ is H; R⁴ is methyl, ethyl, 1-propyl, 2-propyl, 1-butyl, 2-methyl-1-propyl, 1-methylpropyl, 1,1-dimethylethyl, cyclopropyl, cyclobutyl, or cyclopentyl;
wherein each of said methyl, ethyl, 1-propyl, 2-propyl, 1-butyl, 2-methyl-1-propyl, 1-methylpropyl, 1,1-dimethylethyl, cyclopropyl, cyclobutyl, or cyclopentyl is unsubstituted or substituted with 1, 2 or 3 substituents independently selected from methoxy, ethoxy, propoxy, isopropoxy, or butoxy.

In a second embodiment of Aspect 12, A⁵ is N; A⁴ is CR⁴; A³ is CR³; R³ is H; and R⁴ is -tBu, or

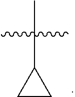 [SMILES: *C1CC1]

In a third embodiment of Aspect 12, A⁵ is N; A⁴ is CR⁴; A³ is CR³; (R³ and R⁴), together with the atoms to which they are attached, form a 5- to 6-membered heterocyclyl ring containing 1, 2 or 3 heteroatoms independently selected from nitrogen, oxygen and optionally oxidized sulfur, wherein said 5- to 6-membered heterocyclyl ring is unsubstituted or substituted with 1, 2 or 3 R^{1d}; and
each R^{1d} is independently hydrogen, or —C₁₋₁₀alkyl.

In a fourth embodiment of Aspect 12, A⁵ is N; A⁴ is CR⁴; A³ is CR³; and (R³ and R⁴), together with the atoms to which they are attached, form dihydro-pyrrolyl or pyrrolidinyl;
wherein each of said dihydro-pyrrolyl or pyrrolidinyl is unsubstituted or substituted with 1, 2 or 3 R^{1d}; and
each R^{1d} is independently hydrogen, methyl, ethyl, 1-propyl, 2-propyl, 1-butyl, 2-methyl-1-propyl, 1-methylpropyl, or 1,1-dimethylethyl.

In a fifth embodiment of Aspect 12, A⁵ is N; A⁴ is CR⁴; A³ is CR³; and (R³ and R⁴), together with the atoms to which they are attached, form

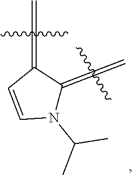 [SMILES: *=c1ccn(C(C)C)c1=*]

which is further unsubstituted or substituted with 1, 2 or 3 R^{1d}; and
each R^{1d} is independently hydrogen, methyl, ethyl, 1-propyl, 2-propyl, 1-butyl, 2-methyl-1-propyl, 1-methylpropyl, or 1,1-dimethylethyl.

In a sixth embodiment of Aspect 12, A³ is N; A⁴ is CR⁴; A⁵ is CR⁵; R⁴ is H; and R⁵ is —C₁₋₁₀alkoxy.

In a seventh embodiment of Aspect 12, A³ is N; A⁴ is CR⁴; A⁵ is CR⁵; R⁴ is H; and R⁵ is methoxy, ethoxy, propoxy, isopropoxy, or butoxy.

In a eighth embodiment of Aspect 12, A⁴ is N; A³ is CR³; A⁵ is CR⁵; R³ is H; and R⁵ is —C₁₋₁₀alkoxy.

In a ninth embodiment of Aspect 12, A⁴ is N; A³ is CR³; A⁵ is CR⁵; R³ is H; and R⁵ is methoxy, ethoxy, propoxy, isopropoxy, or butoxy.

In some embodiments of any one of Aspects 1-12, each R⁷ is hydrogen.

Aspect 13. A compound of Formula (I), wherein the compound is of Formula (VI):

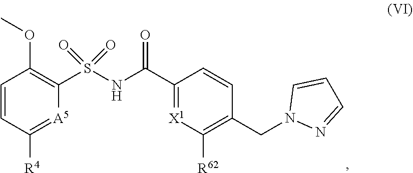

or a pharmaceutically acceptable salt, deuterated analog, N-oxide, or tautomer thereof, wherein:
X¹ is N or CH;
R⁶² is ethyl, ethenyl, cyclopropyl, or methoxy;
A⁵ is N or CR⁵; if present, R⁵ is hydrogen;
R⁴ is

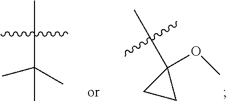

and
A⁵ is CR⁵ or N; if present, R⁵ is hydrogen.

Aspect 14, A compound selected from

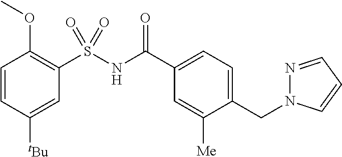 [SMILES: COc1ccc(C(C)(C)C)cc1S(=O)(=O)NC(=O)c1ccc(Cn2cccn2)c(C)c1]

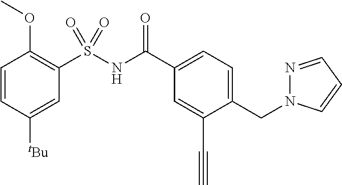 [SMILES: C#Cc1cc(C(=O)NS(=O)(=O)c2cc(C(C)(C)C)ccc2OC)ccc1Cn1cccn1]

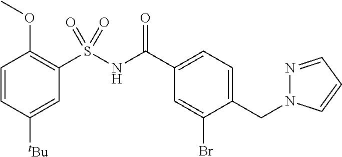 [SMILES: COc1ccc(C(C)(C)C)cc1S(=O)(=O)NC(=O)c1ccc(Cn2cccn2)c(Br)c1]

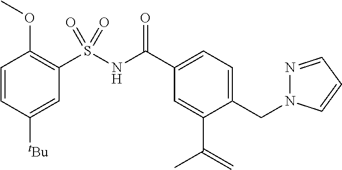 [SMILES: C=C(C)c1cc(C(=O)NS(=O)(=O)c2cc(C(C)(C)C)ccc2OC)ccc1Cn1cccn1]

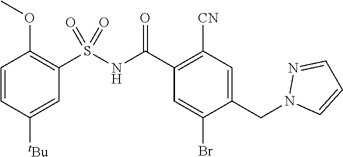 [SMILES: COc1ccc(C(C)(C)C)cc1S(=O)(=O)NC(=O)c1cc(Br)c(Cn2cccn2)cc1C#N]

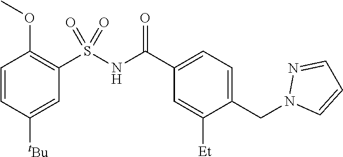 [SMILES: CCc1cc(C(=O)NS(=O)(=O)c2cc(C(C)(C)C)ccc2OC)ccc1Cn1cccn1]

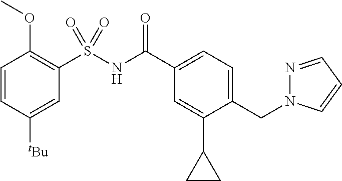 [SMILES: COc1ccc(C(C)(C)C)cc1S(=O)(=O)NC(=O)c1ccc(Cn2cccn2)c(C2CC2)c1]

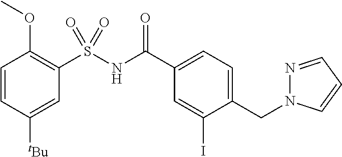 [SMILES: COc1ccc(C(C)(C)C)cc1S(=O)(=O)NC(=O)c1ccc(Cn2cccn2)c(I)c1]

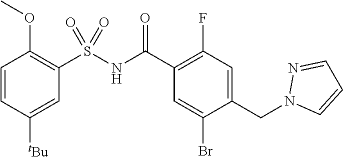 [SMILES: COc1ccc(C(C)(C)C)cc1S(=O)(=O)NC(=O)c1cc(Br)c(Cn2cccn2)cc1F]

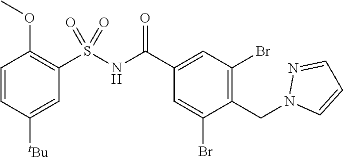 [SMILES: COc1ccc(C(C)(C)C)cc1S(=O)(=O)NC(=O)c1cc(Br)c(Cn2cccn2)c(Br)c1]

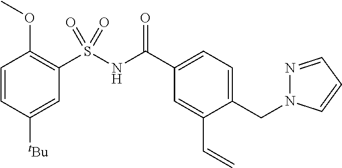 [SMILES: C=Cc1cc(C(=O)NS(=O)(=O)c2cc(C(C)(C)C)ccc2OC)ccc1Cn1cccn1]

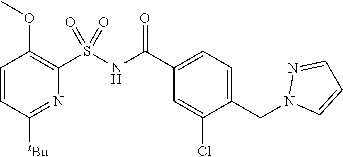 [SMILES: COc1ccc(C(C)(C)C)nc1S(=O)(=O)NC(=O)c1ccc(Cn2cccn2)c(Cl)c1]

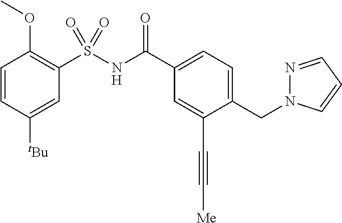 [SMILES: CC#Cc1cc(C(=O)NS(=O)(=O)c2cc(C(C)(C)C)ccc2OC)ccc1Cn1cccn1]

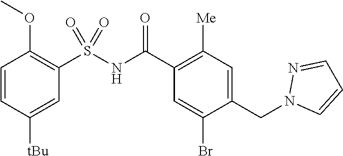 [SMILES: COc1ccc(C(C)(C)C)cc1S(=O)(=O)NC(=O)c1cc(Br)c(Cn2cccn2)cc1C]

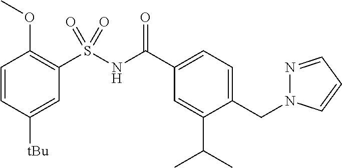 [SMILES: COc1ccc(C(C)(C)C)cc1S(=O)(=O)NC(=O)c1ccc(Cn2cccn2)c(C(C)C)c1]

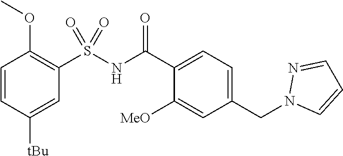 [SMILES: COc1cc(Cn2cccn2)ccc1C(=O)NS(=O)(=O)c1cc(C(C)(C)C)ccc1OC]

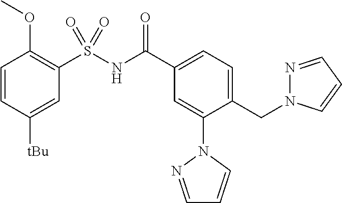 [SMILES: COc1ccc(C(C)(C)C)cc1S(=O)(=O)NC(=O)c1ccc(Cn2cccn2)c(-n2cccn2)c1]

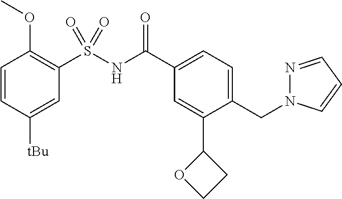 [SMILES: COc1ccc(C(C)(C)C)cc1S(=O)(=O)NC(=O)c1ccc(Cn2cccn2)c(C2CCO2)c1]

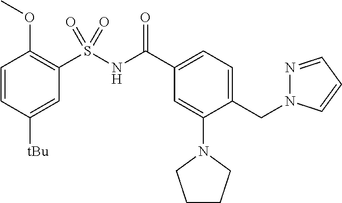 [SMILES: COc1ccc(C(C)(C)C)cc1S(=O)(=O)NC(=O)c1ccc(Cn2cccn2)c(N2CCCC2)c1]

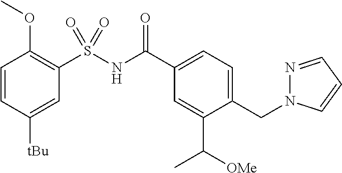 [SMILES: COc1ccc(C(C)(C)C)cc1S(=O)(=O)NC(=O)c1ccc(Cn2cccn2)c(C(C)OC)c1]

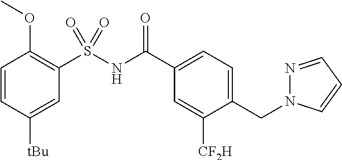 [SMILES: COc1ccc(C(C)(C)C)cc1S(=O)(=O)NC(=O)c1ccc(Cn2cccn2)c(C(F)F)c1]

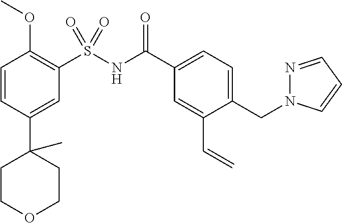 [SMILES: C=Cc1cc(C(=O)NS(=O)(=O)c2cc(C3(C)CCOCC3)ccc2OC)ccc1Cn1cccn1]

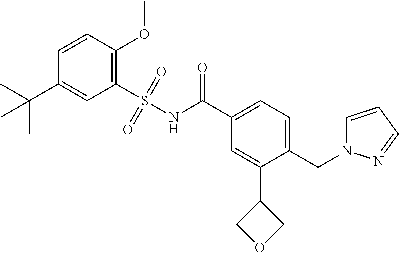 [SMILES: COc1ccc(C(C)(C)C)cc1S(=O)(=O)NC(=O)c1ccc(Cn2cccn2)c(C2COC2)c1]

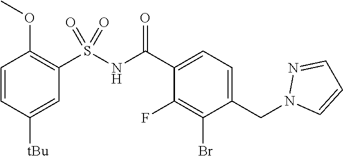 [SMILES: COc1ccc(C(C)(C)C)cc1S(=O)(=O)NC(=O)c1ccc(Cn2cccn2)c(Br)c1F]

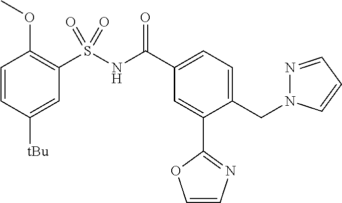 [SMILES: COc1ccc(C(C)(C)C)cc1S(=O)(=O)NC(=O)c1ccc(Cn2cccn2)c(-c2ncco2)c1]

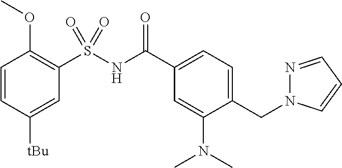 [SMILES: COc1ccc(C(C)(C)C)cc1S(=O)(=O)NC(=O)c1ccc(Cn2cccn2)c(N(C)C)c1]

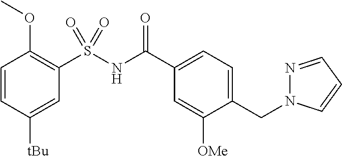 [SMILES: COc1cc(C(=O)NS(=O)(=O)c2cc(C(C)(C)C)ccc2OC)ccc1Cn1cccn1]

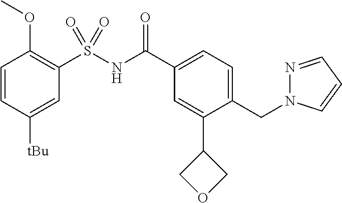 [SMILES: COc1ccc(C(C)(C)C)cc1S(=O)(=O)NC(=O)c1ccc(Cn2cccn2)c(C2COC2)c1]

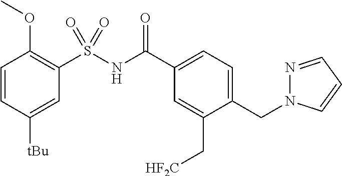

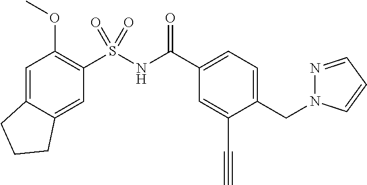 [SMILES: C#Cc1cc(C(=O)NS(=O)(=O)c2cc3c(cc2OC)CCC3)ccc1Cn1cccn1]

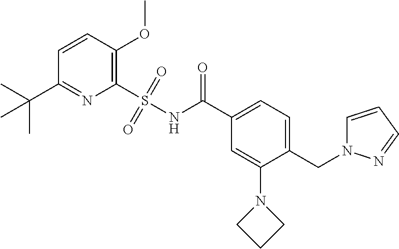 [SMILES: COc1ccc(C(C)(C)C)nc1S(=O)(=O)NC(=O)c1ccc(Cn2cccn2)c(N2CCC2)c1]

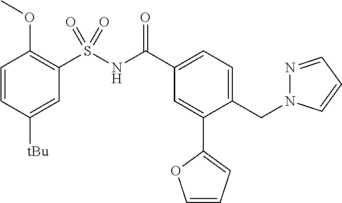 [SMILES: COc1ccc(C(C)(C)C)cc1S(=O)(=O)NC(=O)c1ccc(Cn2cccn2)c(-c2ccco2)c1]

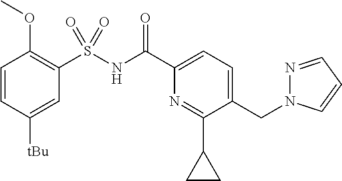 [SMILES: COc1ccc(C(C)(C)C)cc1S(=O)(=O)NC(=O)c1ccc(Cn2cccn2)c(C2CC2)n1]

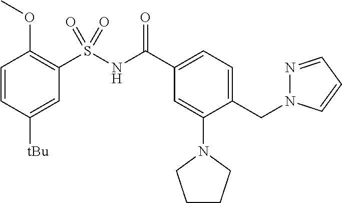 [SMILES: COc1ccc(C(C)(C)C)cc1S(=O)(=O)NC(=O)c1ccc(Cn2cccn2)c(N2CCCC2)c1]

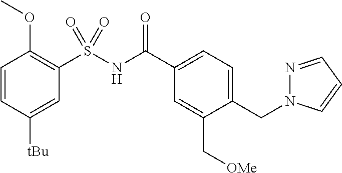 [SMILES: COCc1cc(C(=O)NS(=O)(=O)c2cc(C(C)(C)C)ccc2OC)ccc1Cn1cccn1]

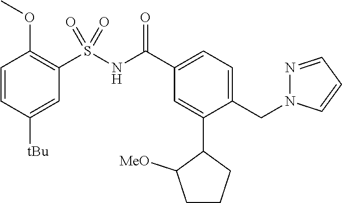 [SMILES: COc1ccc(C(C)(C)C)cc1S(=O)(=O)NC(=O)c1ccc(Cn2cccn2)c(C2CCCC2OC)c1]

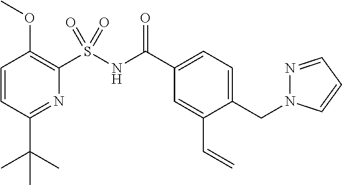 [SMILES: C=Cc1cc(C(=O)NS(=O)(=O)c2nc(C(C)(C)C)ccc2OC)ccc1Cn1cccn1]

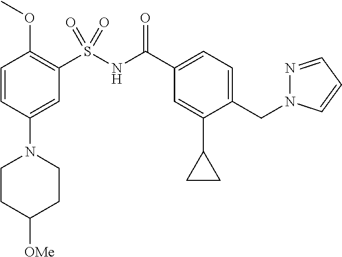 [SMILES: COc1ccc(N2CCC(OC)CC2)cc1S(=O)(=O)NC(=O)c1ccc(Cn2cccn2)c(C2CC2)c1]

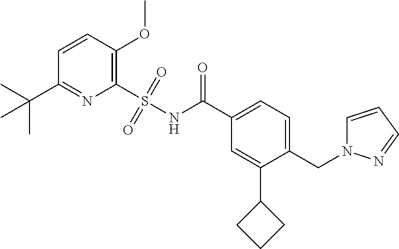 [SMILES: COc1ccc(C(C)(C)C)nc1S(=O)(=O)NC(=O)c1ccc(Cn2cccn2)c(C2CCC2)c1]

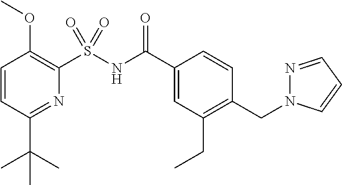 [SMILES: CCc1cc(C(=O)NS(=O)(=O)c2nc(C(C)(C)C)ccc2OC)ccc1Cn1cccn1]

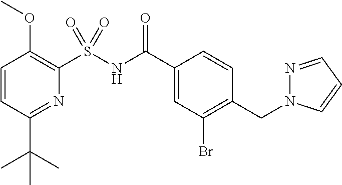 [SMILES: COc1ccc(C(C)(C)C)nc1S(=O)(=O)NC(=O)c1ccc(Cn2cccn2)c(Br)c1]

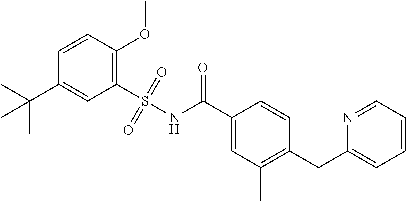 [SMILES: COc1ccc(C(C)(C)C)cc1S(=O)(=O)NC(=O)c1ccc(Cc2ccccn2)c(C)c1]

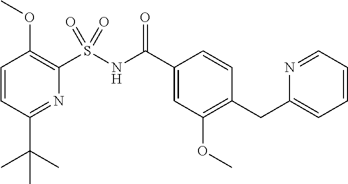 [SMILES: COc1cc(C(=O)NS(=O)(=O)c2nc(C(C)(C)C)ccc2OC)ccc1Cc1ccccn1]

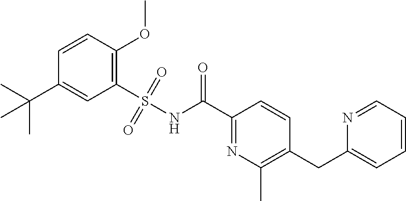 [SMILES: COc1ccc(C(C)(C)C)cc1S(=O)(=O)NC(=O)c1ccc(Cc2ccccn2)c(C)n1]

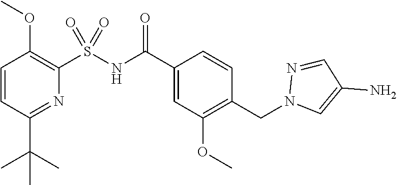 [SMILES: COc1cc(C(=O)NS(=O)(=O)c2nc(C(C)(C)C)ccc2OC)ccc1Cn1cc(N)cn1]

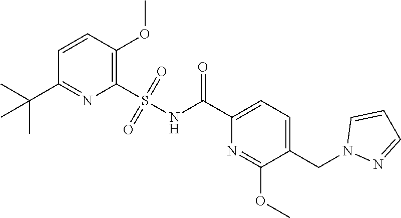 [SMILES: COc1ccc(C(C)(C)C)nc1S(=O)(=O)NC(=O)c1ccc(Cn2cccn2)c(OC)n1]

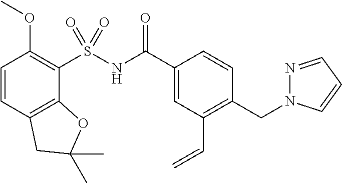 [SMILES: C=Cc1cc(C(=O)NS(=O)(=O)c2c(OC)ccc3c2OC(C)(C)C3)ccc1Cn1cccn1]

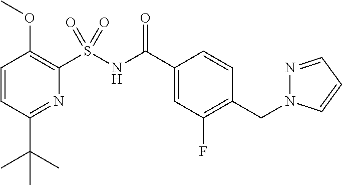 [SMILES: COc1ccc(C(C)(C)C)nc1S(=O)(=O)NC(=O)c1ccc(Cn2cccn2)c(F)c1]

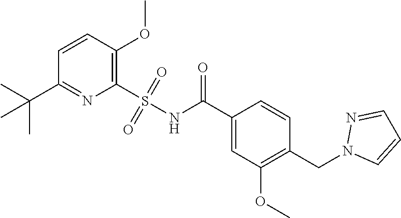 [SMILES: COc1cc(C(=O)NS(=O)(=O)c2nc(C(C)(C)C)ccc2OC)ccc1Cn1cccn1]

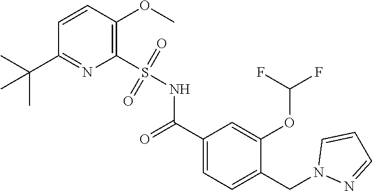 [SMILES: COc1ccc(C(C)(C)C)nc1S(=O)(=O)NC(=O)c1ccc(Cn2cccn2)c(OC(F)F)c1]

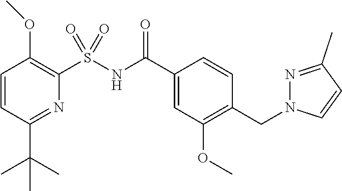 [SMILES: COc1cc(C(=O)NS(=O)(=O)c2nc(C(C)(C)C)ccc2OC)ccc1Cn1ccc(C)n1]

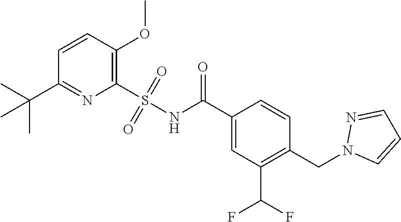 [SMILES: COc1ccc(C(C)(C)C)nc1S(=O)(=O)NC(=O)c1ccc(Cn2cccn2)c(C(F)F)c1]

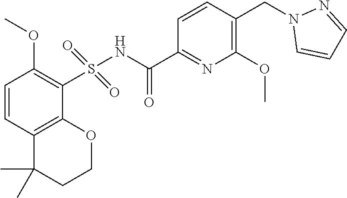 [SMILES: COc1ccc2c(c1S(=O)(=O)NC(=O)c1ccc(Cn3cccn3)c(OC)n1)OCCC2(C)C]

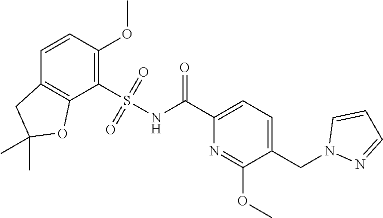 [SMILES: COc1ccc2c(c1S(=O)(=O)NC(=O)c1ccc(Cn3cccn3)c(OC)n1)OC(C)(C)C2]

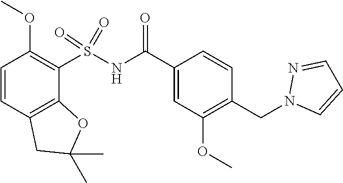 [SMILES: COc1cc(C(=O)NS(=O)(=O)c2c(OC)ccc3c2OC(C)(C)C3)ccc1Cn1cccn1]

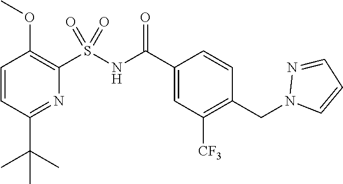 [SMILES: COc1ccc(C(C)(C)C)nc1S(=O)(=O)NC(=O)c1ccc(Cn2cccn2)c(C(F)(F)F)c1]

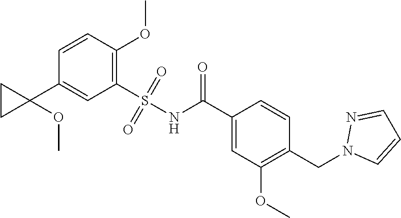 [SMILES: COc1cc(C(=O)NS(=O)(=O)c2cc(C3(OC)CC3)ccc2OC)ccc1Cn1cccn1]

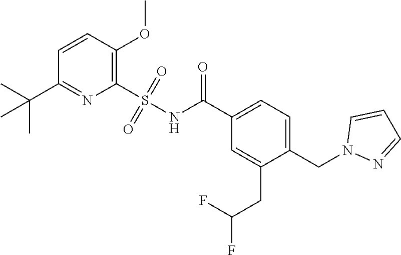 [SMILES: COc1ccc(C(C)(C)C)nc1S(=O)(=O)NC(=O)c1ccc(Cn2cccn2)c(CC(F)F)c1]

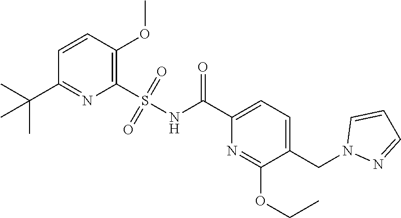 [SMILES: CCOc1nc(C(=O)NS(=O)(=O)c2nc(C(C)(C)C)ccc2OC)ccc1Cn1cccn1]

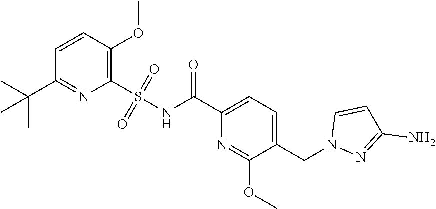 [SMILES: COc1ccc(C(C)(C)C)nc1S(=O)(=O)NC(=O)c1ccc(Cn2ccc(N)n2)c(OC)n1]

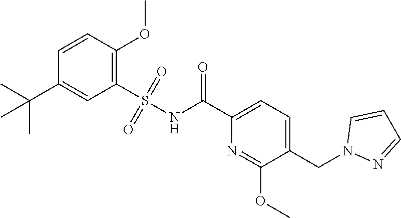 [SMILES: COc1ccc(C(C)(C)C)cc1S(=O)(=O)NC(=O)c1ccc(Cn2cccn2)c(OC)n1]

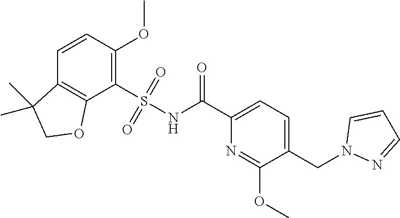 [SMILES: COc1ccc2c(c1S(=O)(=O)NC(=O)c1ccc(Cn3cccn3)c(OC)n1)OCC2(C)C]

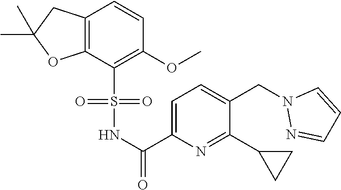 [SMILES: COc1ccc2c(c1S(=O)(=O)NC(=O)c1ccc(Cn3cccn3)c(C3CC3)n1)OC(C)(C)C2]

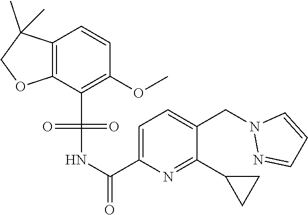

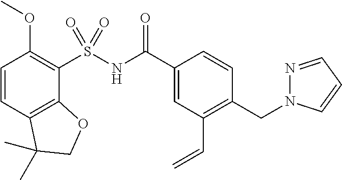 [SMILES: C=Cc1cc(C(=O)NS(=O)(=O)c2c(OC)ccc3c2OCC3(C)C)ccc1Cn1cccn1]

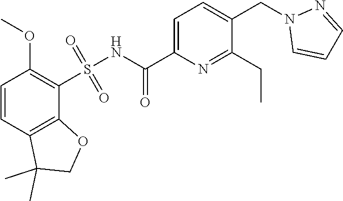 [SMILES: CCc1nc(C(=O)NS(=O)(=O)c2c(OC)ccc3c2OCC3(C)C)ccc1Cn1cccn1]

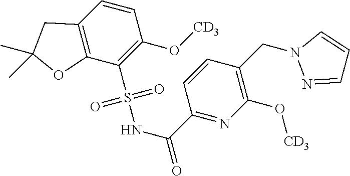 [SMILES: [2H]C([2H])([2H])Oc1ccc2c(c1S(=O)(=O)NC(=O)c1ccc(Cn3cccn3)c(OC([2H])([2H])[2H])n1)OC(C)(C)C2]

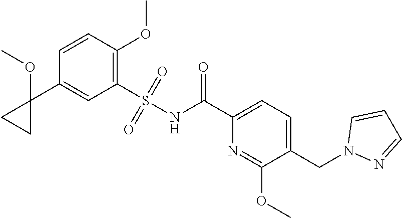 [SMILES: COc1ccc(C2(OC)CC2)cc1S(=O)(=O)NC(=O)c1ccc(Cn2cccn2)c(OC)n1]

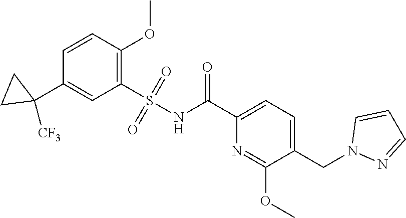 [SMILES: COc1ccc(C2(C(F)(F)F)CC2)cc1S(=O)(=O)NC(=O)c1ccc(Cn2cccn2)c(OC)n1]

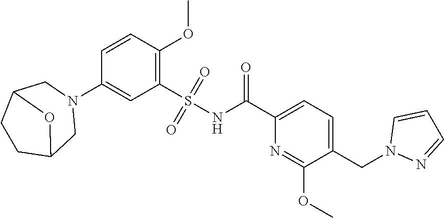 [SMILES: COc1ccc(N2CC3CCC(C2)O3)cc1S(=O)(=O)NC(=O)c1ccc(Cn2cccn2)c(OC)n1]

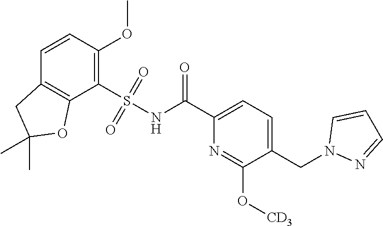 [SMILES: [2H]C([2H])([2H])Oc1nc(C(=O)NS(=O)(=O)c2c(OC)ccc3c2OC(C)(C)C3)ccc1Cn1cccn1]

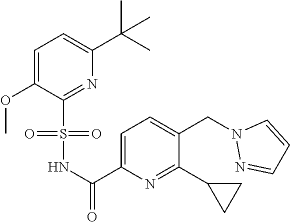 [SMILES: COc1ccc(C(C)(C)C)nc1S(=O)(=O)NC(=O)c1ccc(Cn2cccn2)c(C2CC2)n1]

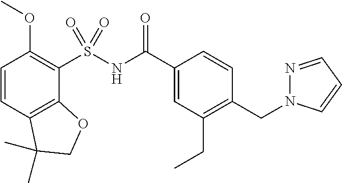 [SMILES: CCc1cc(C(=O)NS(=O)(=O)c2c(OC)ccc3c2OCC3(C)C)ccc1Cn1cccn1]

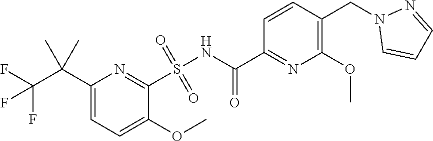 [SMILES: COc1ccc(C(C)(C)C(F)(F)F)nc1S(=O)(=O)NC(=O)c1ccc(Cn2cccn2)c(OC)n1]

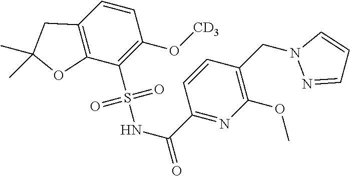 [SMILES: [2H]C([2H])([2H])Oc1ccc2c(c1S(=O)(=O)NC(=O)c1ccc(Cn3cccn3)c(OC)n1)OC(C)(C)C2]

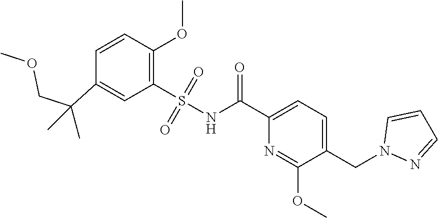 [SMILES: COCC(C)(C)c1ccc(OC)c(S(=O)(=O)NC(=O)c2ccc(Cn3cccn3)c(OC)n2)c1]

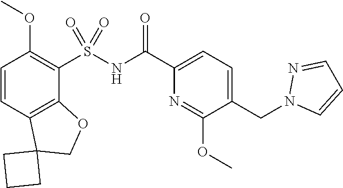 [SMILES: COc1ccc2c(c1S(=O)(=O)NC(=O)c1ccc(Cn3cccn3)c(OC)n1)OCC21CCC1]

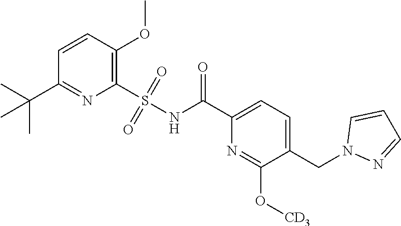 [SMILES: [2H]C([2H])([2H])Oc1nc(C(=O)NS(=O)(=O)c2nc(C(C)(C)C)ccc2OC)ccc1Cn1cccn1]

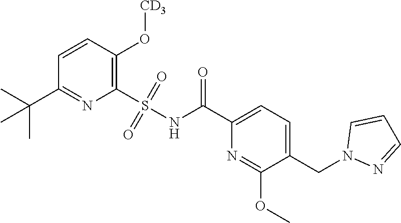 [SMILES: [2H]C([2H])([2H])Oc1ccc(C(C)(C)C)nc1S(=O)(=O)NC(=O)c1ccc(Cn2cccn2)c(OC)n1]

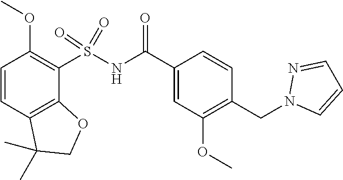 [SMILES: COc1cc(C(=O)NS(=O)(=O)c2c(OC)ccc3c2OCC3(C)C)ccc1Cn1cccn1]

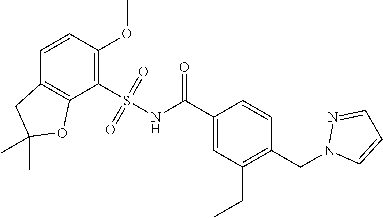 [SMILES: CCc1cc(C(=O)NS(=O)(=O)c2c(OC)ccc3c2OC(C)(C)C3)ccc1Cn1cccn1]

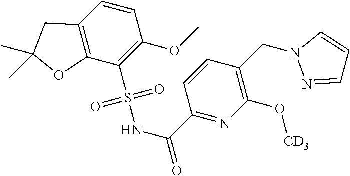 [SMILES: [2H]C([2H])([2H])Oc1nc(C(=O)NS(=O)(=O)c2c(OC)ccc3c2OC(C)(C)C3)ccc1Cn1cccn1]

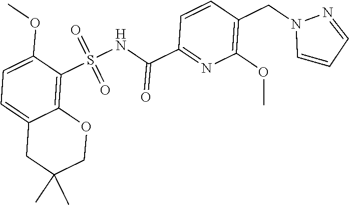 [SMILES: COc1ccc2c(c1S(=O)(=O)NC(=O)c1ccc(Cn3cccn3)c(OC)n1)OCC(C)(C)C2]

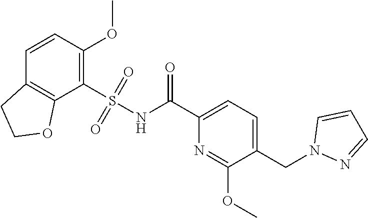 [SMILES: COc1ccc2c(c1S(=O)(=O)NC(=O)c1ccc(Cn3cccn3)c(OC)n1)OCC2]

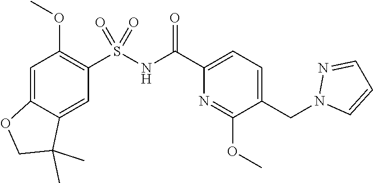 [SMILES: COc1cc2c(cc1S(=O)(=O)NC(=O)c1ccc(Cn3cccn3)c(OC)n1)C(C)(C)CO2]

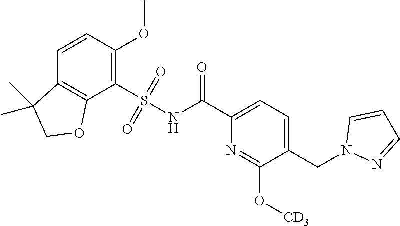 [SMILES: [2H]C([2H])([2H])Oc1nc(C(=O)NS(=O)(=O)c2c(OC)ccc3c2OCC3(C)C)ccc1Cn1cccn1]

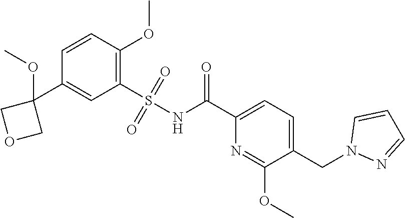 [SMILES: COc1ccc(C2(OC)COC2)cc1S(=O)(=O)NC(=O)c1ccc(Cn2cccn2)c(OC)n1]

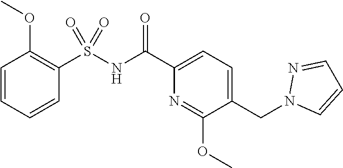 [SMILES: COc1ccccc1S(=O)(=O)NC(=O)c1ccc(Cn2cccn2)c(OC)n1]

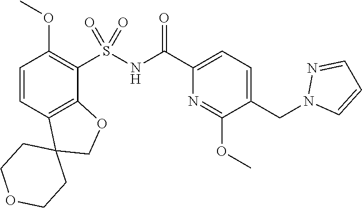 [SMILES: COc1ccc2c(c1S(=O)(=O)NC(=O)c1ccc(Cn3cccn3)c(OC)n1)OCC21CCOCC1]

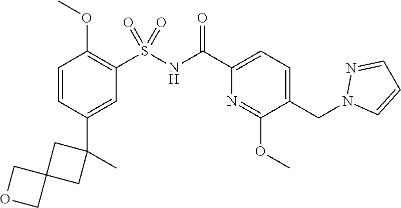 [SMILES: COc1ccc(C2(C)CC3(COC3)C2)cc1S(=O)(=O)NC(=O)c1ccc(Cn2cccn2)c(OC)n1]

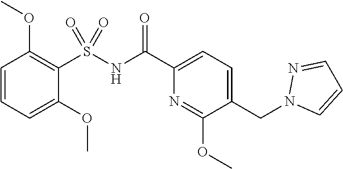 [SMILES: COc1cccc(OC)c1S(=O)(=O)NC(=O)c1ccc(Cn2cccn2)c(OC)n1]

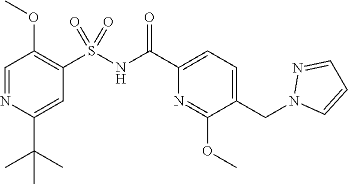 [SMILES: COc1cnc(C(C)(C)C)cc1S(=O)(=O)NC(=O)c1ccc(Cn2cccn2)c(OC)n1]

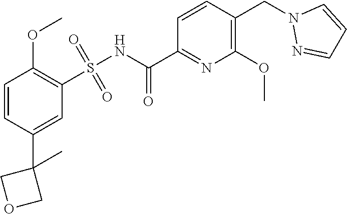 [SMILES: COc1ccc(C2(C)COC2)cc1S(=O)(=O)NC(=O)c1ccc(Cn2cccn2)c(OC)n1]

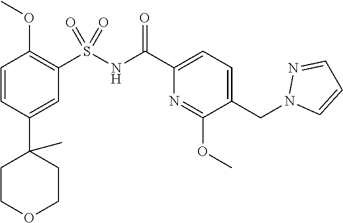 [SMILES: COc1ccc(C2(C)CCOCC2)cc1S(=O)(=O)NC(=O)c1ccc(Cn2cccn2)c(OC)n1]

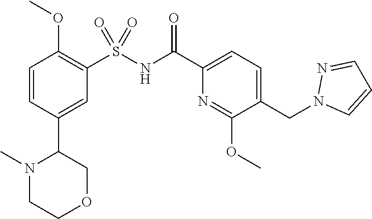 [SMILES: COc1ccc(C2COCCN2C)cc1S(=O)(=O)NC(=O)c1ccc(Cn2cccn2)c(OC)n1]

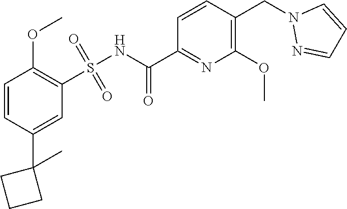 [SMILES: COc1ccc(C2(C)CCC2)cc1S(=O)(=O)NC(=O)c1ccc(Cn2cccn2)c(OC)n1]

or a pharmaceutically acceptable salt thereof, or a deuterated analog thereof, or an N-oxide thereof, or a tautomer thereof.

Aspect 14′ A compound elected from

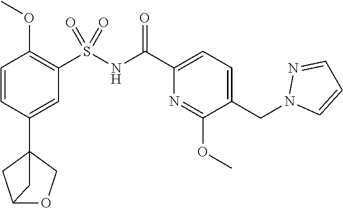 [SMILES: COc1ccc(C23COC(C2)C3)cc1S(=O)(=O)NC(=O)c1ccc(Cn2cccn2)c(OC)n1]

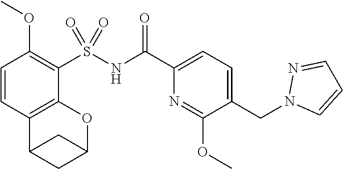 [SMILES: COc1ccc2c(c1S(=O)(=O)NC(=O)c1ccc(Cn3cccn3)c(OC)n1)OC1CC2C1]

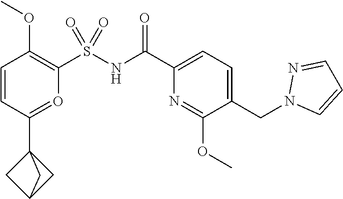

or a pharmaceutically acceptable salt thereof, or a deuterated analog thereof, or an N-oxide thereof, or a tautomer thereof.

Aspect 15, A pharmaceutical composition comprising a compound of any one of Aspects 1-14, or a pharmaceutically acceptable salt thereof, or a deuterated analog thereof, or an N-oxide thereof, or a tautomer thereof, and at least one pharmaceutically acceptable carrier or excipient.

Aspect 16, A method of treating breast cancer, comprising administering to a subject in need thereof a compound of any one of Aspects 1-14, or a pharmaceutically acceptable salt, or a deuterated analog thereof, or an N-oxide thereof, or a tautomer thereof.

Aspect 17. Use of a compound of Formula (I), or a pharmaceutically acceptable salt, or a deuterated analog thereof, or an N-oxide thereof, or a tautomer thereof for the treatment of breast cancer in a subject in need thereof, comprising administering to the subject a therapeutically effective amount of the compound.

Aspect 18. A compound of Formula (I), or a pharmaceutically acceptable salt, or a deuterated analog thereof, or an N-oxide thereof, or a tautomer thereof for use in the treatment of breast cancer in a subject in need thereof, the treatment comprising administering to the subject a therapeutically effective amount of the compound.

Aspect 19. A compound of Formula (I), or a pharmaceutically acceptable salt, or a deuterated analog thereof, or an N-oxide thereof, or a tautomer thereof for the manufacture of a medicament useful in the treatment of breast cancer in a subject in need thereof.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise.

Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

1H NMR spectra were recorded on a Varian instrument operating at 400 MHz. 1H-NMR spectra were obtained using CDCl3, CD2Cl2, CD3OD, D2O, d6-DMSO, d6-acetone or (CD3)2CO as solvent and tetramethylsilane (0.00 ppm) or residual solvent (CDCl3: 7.25 ppm; CD3OD: 3.31 ppm; D2O: 4.79 ppm; d6-DMSO: 2.50 ppm; d6-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz). Compound names except the reagents were generated by ChemDraw version 12.0.

### Abbreviations:

EDCI 1-ethyl-(3-dimethylaminopropyl)carbonyldiimide hydrochloride Acetic acid
AcOH acetonitrile
ACN
Aq Aqueous
AIBN 2,2′-Azobis(2-methylpropionitrile)
Brine Saturated aqueous sodium chloride solution
Bn Benzyl
BnBr Benzyl Bromide
(Boc)₂O di-tert-butyl decarbonate
Cy₃P tricyclohexylphosphine
DMF N,N-Dimethylformamide
BPO Dibenzoyl peroxide
Dppf 1,1″-bis(diphenylphosphino)ferrocene
DBU 1,8-diazabicyclo[5.4.0]undec-7-ene
DIEA or DIPEA N-ethyl-N-isopropylpropan-2-amine
DMAP 4-N,N-dimethylaminopyridine
DMAC Dimethylacetamide
DMF N,N-dimethylformamide
DMSO Dimethyl sulfoxide
DME 1,2-dimethoxyethane
Dtbpy 4,4-Di-tert-butyl bipyridine
DCM Dichloromethane
DIC N,N′-Diisopropylcarbodiimide
DABCO 1,4-Diazabicyclo[2.2.2]octane
EA Ethyl acetate
EtOH Ethanol
Et₂O or ether Diethyl ether
Et₃N Triethyl amine
HATU O-(7-Azabenzotriazol-1-yl)-N,N,N′,N′-tetramethyluronium hexafluorophosphate
HPLC High-performance liquid chromatography
IPA 2-propanol
LDA lithium diisopropylamide
i-PrOH Isopropyl alcohol
ms or MS Mass spectrum
MTBE 2-methoxy-2-methylpropane
NaHMDS Sodium Hexamethylenedisilazane
NBS N-Bromosuccinimide
PE petroleum ether
PPA Polyphosphoric acid
p-TSA p-Tolunesulfonic acid
Rt Retention time
rt Room temperature
TBAF Tetra-butyl ammonium fluoride
TBSCl tert-Butyldimethylsilyl chloride
TFA Trifluoroacetic acid
TEA Triethyl amine
THF tetrahydrofuran
TLC thin layer chromatography
Pre-TLC preparation thin layer chromatography
hr/hrs hour

### Example A

### Example A1: Synthesis of 4-((1H-pyrazol-1-yl)methyl)-3-iodobenzoic acid

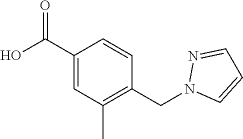 [SMILES: Cc1cc(C(=O)O)ccc1Cn1cccn1]

### Step 1: methyl 4-(bromomethyl)-3-iodobenzoate

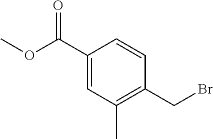

To a stirred solution of methyl 3-iodo-4-methylbenzoate (4 g, 14.49 mmol) and NBS (2.58 g, 14.49 mmol) in CCl4 (50 mL) was added AIBN (0.24 g, 1.45 mmol) at rt. The resulting mixture was stirred at 70° C. for 6 h. After cooled to rt, the resulting mixture was concentrated under reduced pressure. The residue was purified by silica gel column chromatography (PE/EA=5:1) to afford desired product (3.6 g, 70%). 1H NMR (300 MHz, DMSO-d6) δ 8.36 (s, 1H), 7.95 (dd, J=8.0, 1.8 Hz, 1H), 7.75 (d, J=8.0 Hz, 1H), 4.76 (s, 2H), 3.86 (s, 3H).

### Step 2: methyl 4-((1H-pyrazol-1-yl)methyl)-3-iodobenzoate

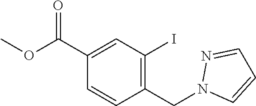 [SMILES: COC(=O)c1ccc(Cn2cccn2)c(I)c1]

To a stirred solution of methyl 4-(bromomethyl)-3-iodobenzoate (3.6 g, 10.14 mmol) and 1H-pyrazole (0.69 g, 10.14 mmol) in ACN (30 mL) was added K2CO3 (2.80 g, 20.28 mmol) at rt. The resulting mixture was stirred at 70° C. overnight. After cooled to rt, the resulting mixture was concentrated under reduced pressure. The residue was purified by silica gel column chromatography (PE/EA=3:1) to afford product (2.5 g, 72%). MS (ESI) m/e [M+H]+=343.

### Step 3: 4-((1H-pyrazol-1-yl)methyl)-3-iodobenzoic acid

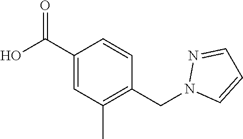 [SMILES: Cc1cc(C(=O)O)ccc1Cn1cccn1]

To a solution of methyl 4-((1H-pyrazol-1-yl)methyl)-3-iodobenzoate (800 mg, 1.46 mmol) in MeOH (5 mL) and THF (5 mL) was added NaOH (2N, 5 mL) dropwise. The resulting mixture was stirred at rt for overnight. Upon completion of the reaction, the mixture was concentrated and acidified by HCl (3N) to pH ˜4. The precipitated solid was collected by filtration to give the title compound (730 mg, 95%). MS (ESI) m/e [M+H]+=329.

### Example A2: Synthesis of 4-((1H-pyrazol-1-yl)methyl)-3-ethynylbenzoic acid

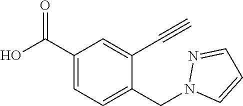 [SMILES: C#Cc1cc(C(=O)O)ccc1Cn1cccn1]

### Step 1: Methyl 4-((1H-pyrazol-1-yl)methyl)-3-((trimethylsilyl)ethynyl)benzoate

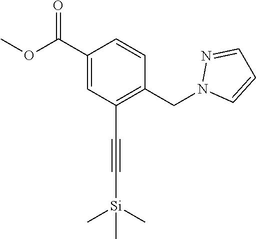 [SMILES: COC(=O)c1ccc(Cn2cccn2)c(C#C[Si](C)(C)C)c1]

A solution of methyl 4-((1H-pyrazol-1-yl)methyl)-3-iodobenzoate (300 mg, 0.88 mmol), trimethylsilylacetylene (129 mg, 1.31 mmol), Pd(dppf)Cl2 (64 mg, 0.09 mmol), CuI (167 mg, 0.88 mmol) and TEA (366 uL, 2.63 mmol) in ACN (5 mL) was stirred at 80° C. under nitrogen atmosphere overnight. After cooled to rt, the resulting mixture was concentrated under reduced pressure. The residue was purified by silica gel column chromatography, eluted with PE/EA (10:1) to afford the product (260 mg, 95%). MS (ESI) m/e [M+H]+=313.1.

### Step 2: 4-((1H-pyrazol-1-yl)methyl)-3-ethynylbenzoic acid

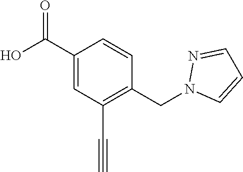 [SMILES: C#Cc1cc(C(=O)O)ccc1Cn1cccn1]

To a stirred solution of methyl 4-((1H-pyrazol-1-yl)methyl)-3-((trimethylsilyl)ethynyl)benzoate (260 mg, 0.83 mmol) in THF (6 mL) and H₂O (2 mL) was added LiOH (40 mg, 1.67 mmol) in portions at 0° C. The resulting mixture was stirred at 70° C. for 1h. After cooled to rt, the mixture was acidified to pH=2 with HCl (1N). The resulting mixture was concentrated under reduced pressure. The residue was purified by reversed-phase flash chromatography (C18; mobile phase, ACN in Water (0.1% FA), 10% to 50% gradient in 20 min). The product containing fractions were combined and evaporated to give the product (99.5 mg, 53%). ¹H NMR (300 MHz, DMSO-d₆) δ 8.00 (s, 1H), 7.93-7.81 (m, 2H), 7.51 (s, 1H), 6.92 (d, J=8.1 Hz, 1H), 6.31 (s, 1H), 5.54 (s, 2H), 4.61 (s, 1H). MS (ESI) m/e [M+H]⁺=227.0.

### Example A3: Synthesis of 4-((1H-pyrazol-1-yl)methyl)-3-bromobenzoic acid

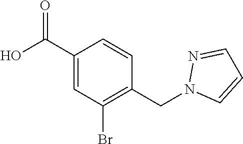 [SMILES: O=C(O)c1ccc(Cn2cccn2)c(Br)c1]

### Step 1: Methyl 3-bromo-4-(bromomethyl)benzoate

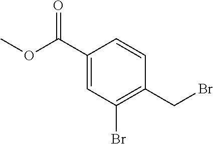

To a solution of methyl 3-bromo-4-methylbenzoate (5.0 g, 21.9 mmol), NBS (4.3 g, 24.1 mmol) and AIBN (360 mg, 2.2 mmol) in CCl₄ (50 mL) was stirred at 95° C. for 3 h. After cooled to rt, the mixture was quenched by water. The aqueous layer was extracted with DCM. The organic layer was concentrated, and the crude was purified by silica gel column chromatography (PE/EA=20:1) to give the product (4.9 g, 73%). MS (ESI) m/e [M+H]⁺=307, 309.

### Step 2: Methyl 4-((1H-pyrazol-1-yl)methyl)-3-bromobenzoate

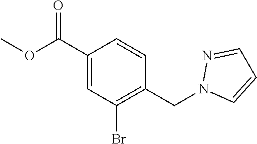 [SMILES: COC(=O)c1ccc(Cn2cccn2)c(Br)c1]

A solution of methyl 3-bromo-4-(bromomethyl)benzoate (4.7 g, 15.4 mmol), 1H-pyrazole (1.26 g, 18.4 mmol) and K2CO3 (4.25 g, 30.8 mmol) in DMF (100 mL) was stirred at 60° C. for 2 h. After cooled to rt, the mixture was quenched by water. The aqueous layer was extracted with DCM. The organic layer was concentrated, and the crude was purified by silica gel column chromatography (PE/EA=4:1) to give the product (3.7 g, 82%). MS (ESI) m/e [M+H]+=295, 297.

### Step 3: 4-((1H-pyrazol-1-yl)methyl)-3-bromobenzoic acid

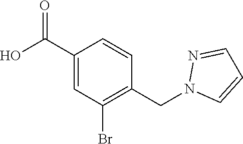 [SMILES: O=C(O)c1ccc(Cn2cccn2)c(Br)c1]

To a solution of methyl 4-((1H-pyrazol-1-yl)methyl)-3-bromobenzoate (1.0 g, 3.4 mmol) in MeOH (5 mL) and THF (5 mL) was added NaOH (2N, 5 mL). The resulting mixture was stirred at rt overnight. Upon completion of the reaction, the mixture was concentrated in vacuo. The pH of the reaction was adjusted to 3 by HCl (3N). The precipitated solid was collected by filtration and dried to give the title compound (800 mg, 84%). MS (ESI) m/e [M+H]+=281, 283.

### Example A4: Synthesis of 4-((1H-pyrazol-1-yl)methyl)-3-vinylbenzoic acid

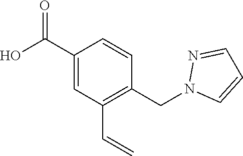 [SMILES: C=Cc1cc(C(=O)O)ccc1Cn1cccn1]

### Step 1: Methyl 4-((1H-pyrazol-1-yl)methyl)-3-vinylbenzoate

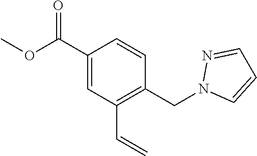 [SMILES: C=Cc1cc(C(=O)OC)ccc1Cn1cccn1]

A solution of methyl 4-((1H-pyrazol-1-yl)methyl)-3-bromobenzoate (300 mg, 1 mmol), tributyl(vinyl)stannane (477 mg, 1.5 mmol) and Pd(PPh₃)₄ (58 mg, 0.05 mmol) in THF (10 mL) was stirred at 80° C. for 4 h. After cooled to rt, the reaction was concentrated. Water was added, and the mixture was extracted with EA. The organic layer was concentrated, and the crude was purified by silica gel column chromatography (PE/EA=4:1) to give the product (240 mg, 97%). MS (ESI) m/e [M+H]⁺=243.

### Step 2: 4-((1H-pyrazol-1-yl)methyl)-3-vinylbenzoic acid

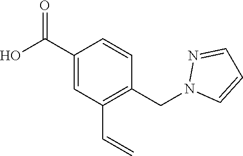 [SMILES: C=Cc1cc(C(=O)O)ccc1Cn1cccn1]

To a solution of methyl 4-((1H-pyrazol-1-yl)methyl)-3-vinylbenzoate (240 mg, 1 mmol) in MeOH (5 mL) and THF (5 mL) was added NaOH (2N, 5 mL). The resulting mixture was stirred at rt for overnight. Upon completion of the reaction, the mixture was concentrated. The pH of the reaction was adjusted to 3 by HCl (3N). The precipitated solid was collected by filtration to give the title compound (180 mg, 80%). MS (ESI) m/e [M+H]⁺=229.

### Example A5: Synthesis of 4-((1H-pyrazol-1-yl)methyl)-3-cyclopropylbenzoic acid

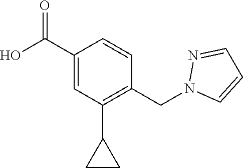 [SMILES: O=C(O)c1ccc(Cn2cccn2)c(C2CC2)c1]

### Step 1: Methyl 4-((1H-pyrazol-1-yl)methyl)-3-cyclopropylbenzoate

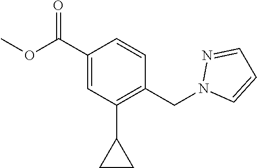 [SMILES: COC(=O)c1ccc(Cn2cccn2)c(C2CC2)c1]

A solution of methyl 4-((1H-pyrazol-1-yl)methyl)-3-bromobenzoate (200 mg, 0.68 mmol), cyclopropylboronic acid (117 mg, 1.36 mmol), Pd(OAc)₂ (16 mg, 0.07 mmol), Cy₃P (38 mg, 0.14 mmol) and K₃PO₄ (289 mg, 1.36 mmol) in DMAc (5 mL) was stirred at 140° C. under N₂ for 4h in a microwave reactor. After cooled to rt, the mixture was quenched by water. The aqueous layer was extracted with EA. The organic layer was concentrated, and the crude was purified by Prep-TLC (PE/EA=1:3) to give the product (20 mg, 11.5%). MS (ESI) m/e [M+H]⁺=257.

### Step 2: 4-((1H-pyrazol-1-yl)methyl)-3-cyclopropylbenzoic acid

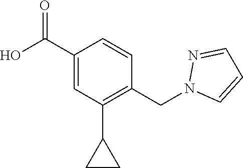 [SMILES: O=C(O)c1ccc(Cn2cccn2)c(C2CC2)c1]

To a solution of methyl 4-((1H-pyrazol-1-yl)methyl)-3-cyclopropylbenzoate (20 mg, 0.08 mmol) in MeOH (2 mL) and THF (2 mL) was added NaOH (2N, 2 mL). The resulting mixture was stirred at rt for overnight. Upon completion of the reaction, the mixture was concentrated. The pH of the reaction was adjusted to 3 by HCl (3N). The precipitated solid was collected by filtration to give the title compound (15 mg, 79%). MS (ESI) m/e [M+H]⁺=243.

### Example A6: Synthesis of 4-((1H-pyrazol-1-yl)methyl)-3-(methoxymethyl)benzoic acid

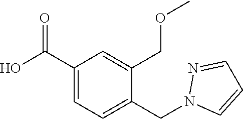 [SMILES: COCc1cc(C(=O)O)ccc1Cn1cccn1]

### Step 1: Methyl 4-((1H-pyrazol-1-yl)methyl)-3-(methoxymethyl)benzoate

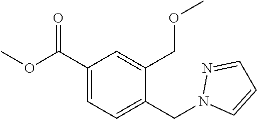 [SMILES: COCc1cc(C(=O)OC)ccc1Cn1cccn1]

A mixture of methyl 4-((1H-pyrazol-1-yl)methyl)-3-bromobenzoate (100 mg, 0.34 mmol), potassium methoxy-methyltrifluoroborate (62 mg, 0.41 mmol), Ir[dF(CF3)ppy]2(dtbbpy)PF6 (8 mg, 0.007 mmol), NiCl2·glyme (8 mg, 0.04 mmol), 4,4′-di-tert-butyl-2,2′-bipyridine (10 mg, 0.04 mmol) and K2HPO4 (174 mg, 1 mmol) in DMAC (2 mL) was bubbled with N2 for 2 min and then stirred at rt under blue LED for 6h. Upon completion of the reaction, the mixture was diluted with EA. The organic layer was washed with water, brine, dried over with Na2SO4, filtered and concentrated. The residue was purified by silica gel column chromatography with PE/EA (eluted from PE to EA) to give the title product (25 mg, 26%). MS (ESI) m/e [M+H]+=261.2.

### Step 2: 4-((1H-pyrazol-1-yl)methyl)-3-(methoxymethyl)benzoic acid

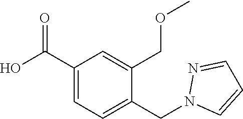 [SMILES: COCc1cc(C(=O)O)ccc1Cn1cccn1]

To a solution of methyl 4-((1H-pyrazol-1-yl)methyl)-3-(methoxymethyl)benzoate (25 mg, 0.1 mmol) in THF (1 mL), MeOH (1 mL) and water (1 mL) was added NaOH (0.2 mL, 1N, 0.2 mmol) dropwise. The solution was stirred at rt for 2 h. Upon completion of the reaction, the mixture was diluted with EA and citric acid solution. The organic layer was washed with brine, dried over with Na₂SO₄, filtered and concentrated to give the title product (23 mg, crude). MS (ESI) m/e [M+H]⁺=247.1.

### Example 7: Synthesis of 4-((1H-pyrazol-1-yl)methyl)-3-(dimethylamino)benzoic acid

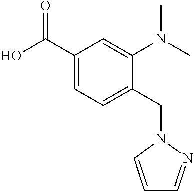 [SMILES: CN(C)c1cc(C(=O)O)ccc1Cn1cccn1]

### Step 1: Methyl 4-((1H-pyrazol-1-yl)methyl)-3-(dimethylamino)benzoate

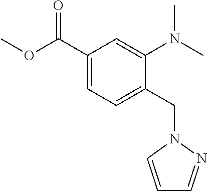

A solution of methyl 4-((1H-pyrazol-1-yl)methyl)-3-bromobenzoate (150 mg, 0.5 mmol), dimethylamine hydrogen chloride salt (61 mg, 0.75 mmol), Ir[dF(CF₃)ppy]₂(dtbbpy)PF₆ (11 mg, 0.01 mmol), NiCl₂·glyme (11 mg, 0.05 mmol) and DABCO (196 mg, 1.75 mmol) in DMAC (5 mL) was bubbled with N₂ for 2 min and then stirred at rt under blue LED for overnight. Upon completion of the reaction, the mixture was diluted with EA. The organic layer was washed with water, brine, dried over with Na₂SO₄, filtered and concentrated. The residue was purified by silica gel column chromatography (PE:EA=2:1) to give the title product (27 mg, 21%). MS (ESI) m/e [M+H]⁺=260.1.

### Step 2: 4-((1H-pyrazol-1-yl)methyl)-3-(dimethylamino)benzoic acid

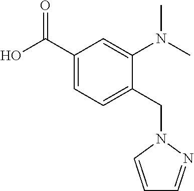 [SMILES: CN(C)c1cc(C(=O)O)ccc1Cn1cccn1]

To a solution of methyl 4-((1H-pyrazol-1-yl)methyl)-3-(dimethylamino)benzoate (27 mg, 0.1 mmol) in THF (1 mL), MeOH (1 mL) and water (1 mL) was added NaOH (0.2 mL, 1N, 0.2 mmol) dropwise. The solution was stirred at rt for 2 h. Upon completion of the reaction, the mixture was acidified by HCl (1N) to pH value to 4 and concentrated. The residue was suspended in DCM/MeOH (10:1, 5 mL) for 5 min and filtered. The filtrate was concentrated, and the residue was directly used in the next step. (25 mg, crude). MS (ESI) m/e [M+H]+=246.3.

### Example A8: Synthesis of 4-((1H-pyrazol-1-yl)methyl)-3-methoxybenzoic acid

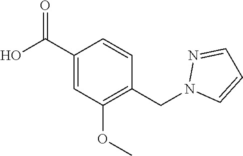 [SMILES: COc1cc(C(=O)O)ccc1Cn1cccn1]

### Step 1: Methyl 4-((1H-pyrazol-1-yl)methyl)-3-methoxybenzoate

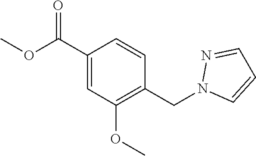 [SMILES: COC(=O)c1ccc(Cn2cccn2)c(OC)c1]

A solution of methyl 4-(bromomethyl)-3-methoxybenzoate (1.0 g, 3.88 mmol), 1H-pyrazole (290 mg, 4.26 mmol) and K2CO3 (1.07 g, 7.76 mmol) in DMF (100 mL) was stirred at 60° C. overnight. After cooled to rt, the mixture was quenched by water. The aqueous layer was extracted with EA. The organic layer was concentrated, and the crude was purified by silica gel column chromatography (PE/EA=3:7) to give the product (576 mg, 60.4%). MS (ESI) m/e [M+H]+=247.

### Step 2: 4-((1H-pyrazol-1-yl)methyl)-3-methoxybenzoic acid

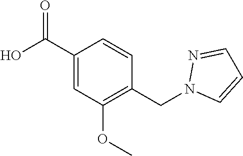 [SMILES: COc1cc(C(=O)O)ccc1Cn1cccn1]

To a solution of methyl 4-((1H-pyrazol-1-yl)methyl)-3-methoxybenzoate (576 mg, 2.34 mmol) in MeOH (10 mL) and THF (10 mL) was added NaOH (2N, 10 mL). The resulting mixture was stirred at rt overnight. Upon completion of the reaction, the mixture was concentrated. The PH of the reaction was adjusted to 3 by HCl (3N). The precipitated solid was collected by filtration to give the title compound (460 mg, 84.7%). MS (ESI) m/e [M+H]+=233.

### Example A9: Synthesis of 4-((1H-pyrazol-1-yl)methyl)-5-bromo-2-fluorobenzoic acid

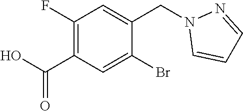 [SMILES: O=C(O)c1cc(Br)c(Cn2cccn2)cc1F]

### Step 1: Methyl 5-bromo-2-fluoro-4-methylbenzoate

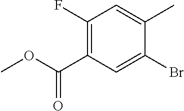

To a solution of 5-bromo-2-fluoro-4-methylbenzoic acid (1 g, 4.5 mmol) in MeOH (10 mL) was added conc. H2SO4 (5 drops) and the solution was stirred at 80° C. for 16 h. After cooled to rt, the solvent was evaporated and the residue was dissolved in EA. The organic layer was washed with NaHCO₃ solution, brine, dried over with Na2SO4, filtered and concentrated. The residue was purified by silica gel column chromatography (PE:EA=1:1) to give the title product (1 g, 95%). MS (ESI) m/e [M+H]+=247, 249.

### Step 2: Methyl 5-bromo-4-(bromomethyl)-2-fluorobenzoate

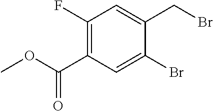

A solution of methyl 5-bromo-2-fluoro-4-methylbenzoate (1 g, 4.1 mmol), NBS (796 mg, 4.4 mmol) and BPO (48 mg, 0.2 mmol) in CCl4 (20 mL) was stirred at 80° C. for 16 h. After cooled to rt, the solid was filtered out. The filtrate was washed with NaHCO₃ solution, brine, dried over with Na2SO4, filtered and concentrated. The residue was purified by silica gel column chromatography (PE:EA=1:1) to give the title product (1.2 g, 90%). MS (ESI) m/e [M+H]+=325, 327.

### Step 3: Methyl 4-((1H-pyrazol-1-yl)methyl)-5-bromo-2-fluorobenzoate

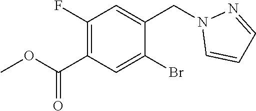 [SMILES: COC(=O)c1cc(Br)c(Cn2cccn2)cc1F]

To a solution of methyl 5-bromo-4-(bromomethyl)-2-fluorobenzoate (324 mg, 1 mmol) and 1H-pyrazole (102 mg, 1.5 mmol) in THF (10 mL) was added tBuOK (134 mg, 1.2 mmol) portionwise. The reaction mixture was stirred at rt for 2h. Upon completion of the reaction, the mixture was diluted with EA. The organic layer was washed with water, brine, dried over with Na2SO4, filtered and concentrated. The residue was purified by silica gel column chromatography with PE/EA (eluted from PE to EA) to give the title product (330 mg, 100%). MS (ESI) m/e [M+H]+=313, 315.

### Step 4: 4-((1H-pyrazol-1-yl)methyl)-5-bromo-2-fluorobenzoic acid

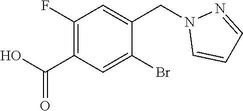 [SMILES: O=C(O)c1cc(Br)c(Cn2cccn2)cc1F]

To a solution of methyl 4-((1H-pyrazol-1-yl)methyl)-5-bromo-2-fluorobenzoate (330 mg, 1 mmol) in THF (2 mL), MeOH (2 mL) and water (2 mL) was added NaOH (0.34 mL, 6N, 2 mmol) dropwise. The solution was stirred at rt for 16h. Upon completion of the reaction, the mixture was diluted with EA and citric acid solution. The organic layer was washed with brine, dried over with Na2SO4, filtered and concentrated to give the title product (300 mg, 99%). MS (ESI) m/e [M+H]+=299, 301.

### Example A10: Synthesis of 4-((1H-pyrazol-1-yl)methyl)-3-bromo-2-fluorobenzoic acid

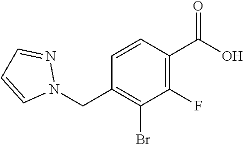 [SMILES: O=C(O)c1ccc(Cn2cccn2)c(Br)c1F]

### Step 1: 3-bromo-2-fluoro-4-methylbenzoic acid

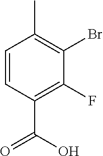

To a solution of 2-bromo-1-fluoro-3-methylbenzene (1.89 g, 10 mmol) in THF (30 mL) was added LDA (5.25 mL, 10.5 mmol, 2M in THF) dropwise below −65° C. and the solution was stirred at this temperature for 30 min. Crushed dry ice was added to the solution and the solution was stirred at −65° C. for 1 h. Then the mixture was allowed to warm to rt and stirred overnight. The reaction mixture was diluted with EA, washed with citric acid solution, brine, dried over with Na2SO4, filtered and concentrated to give the title product (2.1 g, crude). MS (ESI) m/e [M+H]+=233, 235.

### Step 2: Methyl 3-bromo-2-fluoro-4-methylbenzoate

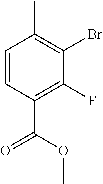

To a solution of 3-bromo-2-fluoro-4-methylbenzoic acid (2.1 g, 10 mmol) in MeOH (10 mL) was added dropwise SOCl2 (2 mL) carefully and the solution was stirred at 80° C. for 3 h. After cooled to rt, the solvent was evaporated and the residue was purified by silica gel column chromatography (PE:EA=10:1) to give the title product (1.4 g, 57% for two steps). MS (ESI) m/e [M+H]+=247, 249.

### Step 3: Methyl 3-bromo-4-(bromomethyl)-2-fluorobenzoate

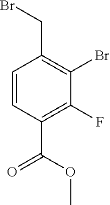

A mixture of methyl 3-bromo-2-fluoro-4-methylbenzoate (246 mg, 1 mmol), NBS (187 mg, 1.05 mmol) and BPO (12 mg, 0.05 mmol) in CCl4 (10 mL) was stirred at 80° C. for 16 h. After cooled to rt, the solid was filtered out. The filtrate was concentrated and purified by silica gel column chromatography (PE:EA=5:1) to give the title product (140 mg, 90%). MS (ESI) m/e [M+H]+=325, 327.

### Step 4: Methyl 4-((1H-pyrazol-1-yl)methyl)-3-bromo-2-fluorobenzoate

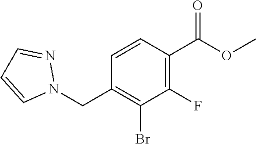 [SMILES: COC(=O)c1ccc(Cn2cccn2)c(Br)c1F]

To a mixture of methyl 3-bromo-4-(bromomethyl)-2-fluorobenzoate (140 mg, 0.43 mmol) and 1H-pyrazole (44 mg, 0.65 mmol) in THF (10 mL) was added tBuOK (58 mg, 0.51 mmol) portion wise. The reaction mixture was stirred at rt for 1h. Upon completion of the reaction, the mixture was concentrated. The residue was purified by silica gel column chromatography with PE/EA (eluted from PE to EA) to give the title product (80 mg, 60%). MS (ESI) m/e [M+H]+=313, 315.

### Step 5: 4-((1H-pyrazol-1-yl)methyl)-3-bromo-2-fluorobenzoic acid

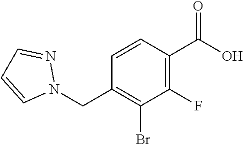 [SMILES: O=C(O)c1ccc(Cn2cccn2)c(Br)c1F]

To a solution of methyl 4-((1H-pyrazol-1-yl)methyl)-3-bromo-2-fluorobenzoate (80 mg, 0.26 mmol) in THF (1 mL), MeOH (1 mL) and water (1 mL) was added NaOH (0.1 mL, 6N, 0.6 mmol) dropwise. The solution was stirred at rt for 2h. Upon completion of the reaction, the mixture was diluted with EA and citric acid solution, washed with brine, dried over with Na2SO4, filtered and concentrated to give the title product (60 mg, 77%). MS (ESI) m/e [M+H]+=299, 301.

### Example A11: 5-((1H-pyrazol-1-yl)methyl)-6-cyclopropylpicolinic acid

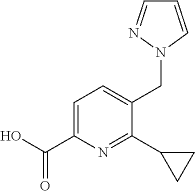 [SMILES: O=C(O)c1ccc(Cn2cccn2)c(C2CC2)n1]

### Step 1: 2-bromo-3-(chloromethyl)pyridine

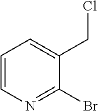

To a solution of (2-bromopyridin-3-yl)methanol (1.87 g, 10 mmol) in DCM (10 mL) was added SOCl2 (2.38 g, 20 mmol) dropwise at 0° C. The mixture was stirred at rt overnight. Upon completion of the reaction, the mixture was diluted with water and extracted with DCM. The organic layer was concentrated to give the product (2.00 g, 97%). MS (ESI) m/e [M+H]+=206, 208.

### Step 2: 3-((1H-pyrazol-1-yl)methyl)-2-bromopyridine

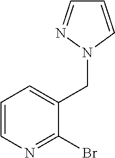 [SMILES: Brc1ncccc1Cn1cccn1]

A solution of 2-bromo-3-(chloromethyl)pyridine (2.00 g, 9.76 mmol), 1H-pyrazole (680 mg, 10 mmol) and Cs2CO3 (6.52 g, 10 mmol) in DMF (10 mL) was stirred at 80° C. overnight. Upon completion of the reaction, the solvent was removed in vacuo. The crude was purified by silica gel column chromatography (PE/EA=3:1) to give the product (2 g, 86%). MS (ESI) m/e [M+H]+=238, 240.

### Step 3: 3-((1H-pyrazol-1-yl)methyl)-2-cyclopropylpyridine

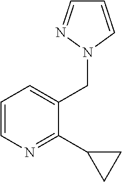 [SMILES: c1cnc(C2CC2)c(Cn2cccn2)c1]

A solution of 3-((1H-pyrazol-1-yl)methyl)-2-bromopyridine (2.37 g, 10 mmol), cyclopropylboronic acid (1.70 g, 20 mmol), Pd(PPh3)4 (1.15 g, 0.1 mmol) and K3PO4 (4.24 g, 20 mmol) in dioxane/water (5:1)(10 mL) was stirred at 140° C. under N2 overnight. Upon completion of the reaction, the mixture was concentrated. The crude was purified by silica gel column chromatography (PE/EA=3:1) to give the product (1 g, 50%). MS (ESI) m/e [M+H]+=200.2.

### Step 4: 3-((1H-pyrazol-1-yl)methyl)-2-cyclopropylpyridine 1-oxide

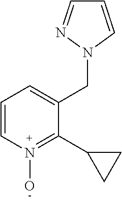

A solution of 3-((1H-pyrazol-1-yl)methyl)-2-cyclopropylpyridine (1.30 g, 6.53 mmol) and m-CPBA (1.4 g, 8.13 mmol) in DCM (10 mL) was stirred at rt overnight. Upon completion of the reaction, the mixture was concentrated. The residue was purified by silica gel column chromatography (PE/EA=2:1) to give the product (1 g, 71%). MS (ESI) m/e [M+H]+=216.2.

### Step 5: 3-((1H-pyrazol-1-yl)methyl)-6-chloro-2-cyclopropylpyridine

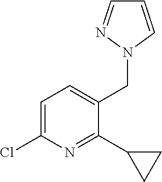 [SMILES: Clc1ccc(Cn2cccn2)c(C2CC2)n1]

A solution 3-((1H-pyrazol-1-yl)methyl)-2-cyclopropylpyridine 1-oxide (1.00 g, 4.65 mmol) and POCl3 (2.13 g, 13.95 mmol) in DCE (10 mL) was stirred at 90° C. overnight. Upon completion of the reaction, the solution was diluted with water and extracted with EA. The organic layer was concentrated, and the crude was purified by C18 column (0.1% FA in water/ACN) to give the product (300 mg, 28%). 1H NMR (400 MHz, DMSO-d6) δ 7.61-7.56 (m, 1H), 7.39-7.34 (m, 1H), 7.12 (d, J=8.0 Hz, 1H), 7.02 (d, J=8.0 Hz, 1H), 6.36-6.30 (m, 1H), 5.49 (s, 2H), 2.05-1.96 (m, 1H), 1.19-1.11 (m, 2H), 1.02-0.95 (m, 2H). MS (ESI) m/e [M+H]+=234.1.

### Step 6: Methyl 5-((1H-pyrazol-1-yl)methyl)-6-cyclopropylpicolinate

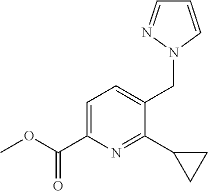 [SMILES: COC(=O)c1ccc(Cn2cccn2)c(C2CC2)n1]

A solution of 3-((1H-pyrazol-1-yl)methyl)-6-chloro-2-cyclopropylpyridine (300 mg, 1.28 mmol), Pd(dppf)2Cl2 (73 mg, 0.1 mmol) and TEA (387 mg, 3.83 mmol) in MeOH (10 mL) was stirred at 110° C. under CO (g) (15 atm) for overnight. After cooled to rt, the solution was concentrated. The residue was purified by silica gel column chromatography (PE/EA=2:1) to give the product (200 mg, 61%). MS (ESI) m/e [M+H]+=258.2.

### Step 7: 5-((1H-pyrazol-1-yl)methyl)-6-cyclopropylpicolinic acid

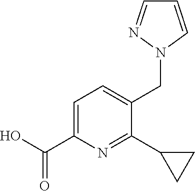 [SMILES: O=C(O)c1ccc(Cn2cccn2)c(C2CC2)n1]

A solution of methyl 5-((1H-pyrazol-1-yl)methyl)-6-cyclopropylpicolinate (200 mg, 0.78 mmol) and LiOH·H2O (100 mg, 2.38 mmol) in THF/H2O (1:1) (10 mL) was stirred at 50° C. overnight. After cooled to rt, the solution was concentrated in vacuo. The crude was purified by C18 column (0.1% FA in water/ACN) to give the product (150 mg, 79%). MS (ESI) m/e [M+H]+=244.1.

### Example A12: Synthesis of 4-((1H-pyrazol-1-yl)methyl)-3-(oxetan-2-yl)benzoic acid

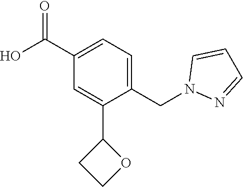 [SMILES: O=C(O)c1ccc(Cn2cccn2)c(C2CCO2)c1]

### Step 1: methyl 4-((1H-pyrazol-1-yl)methyl)-3-(oxetan-2-yl)benzoate

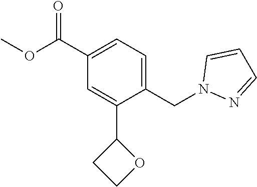 [SMILES: COC(=O)c1ccc(Cn2cccn2)c(C2CCO2)c1]

A mixture of methyl 4-((1H-pyrazol-1-yl)methyl)-3-bromobenzoate (294 mg, 1 mmol), oxetane-2-carboxylic acid (204 mg, 4 mmol), Ir[dF(CF3)ppy]2(dtbbpy)PF6 (22 mg, 0.02 mmol), NiCl2·glyme (22 mg, 0.2 mmol), 4,4′-di-tert-butyl-2,2′-bipyridine (80 mg, 0.3 mmol) and 2-(tert-butyl)-1,1,3,3-tetramethylguanidine (BTMG, 342 mg, 2 mmol) in DMAC (10 mL) was bubbled with N2 for 2 min and then stirred at rt under blue LED for overnight. Upon completion of the reaction, the mixture was diluted with EA, washed with water, brine, dried over with Na2SO4, filtered and concentrated. The residue was purified by silica gel column with PE/EA (eluted from PE to EA) to give the title product (140 mg, 51%). MS (ESI) m/e [M+H]+=273.2.

### Step 2: 4-((1H-pyrazol-1-yl)methyl)-3-(oxetan-2-yl)benzoic acid

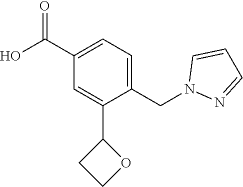 [SMILES: O=C(O)c1ccc(Cn2cccn2)c(C2CCO2)c1]

To a solution of methyl 4-((1H-pyrazol-1-yl)methyl)-3-(oxetan-2-yl)benzoate (140 mg, 0.51 mmol) in THF (5 mL), MeOH (5 mL) and water (5 mL) was added NaOH (1 mL, 1N, 1 mmol) and the solution was stirred at rt for 2 h. Upon completion of the reaction, the reaction mixture was added 1N HCl to pH value to 4 and extracted with EA, washed with brine, dried, concentrated and concentrated to give the product (80 mg, 61%). MS (ESI) m/e [M+H]+=259.0.

### Example A13: Synthesis of 4-((1H-pyrazol-1-yl)methyl)-3-(oxetan-3-yl)benzoic acid

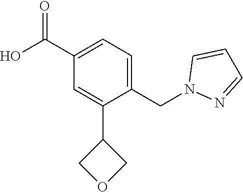 [SMILES: O=C(O)c1ccc(Cn2cccn2)c(C2COC2)c1]

### Step 1: methyl 4-((1H-pyrazol-1-yl)methyl)-3-(oxetan-3-yl)benzoate

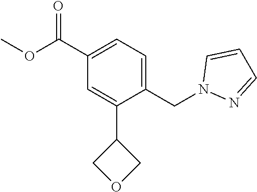 [SMILES: COC(=O)c1ccc(Cn2cccn2)c(C2COC2)c1]

To a mixture of oxetan-3-ol (111 mg, 1.5 mmol) and 5,7-di-tert-butyl-3-phenylbenzo[d]oxazol-3-ium tetrafluoroborate (NHC, 553 mg, 1.4 mmol) in 2-methoxy-2-methylpropane (MTBE, 5 mL) was added pyridine (110 mg, 1.4 mmol) and the mixture was stirred at rt under N2 for 20 min. In another flask, a mixture of methyl 4-((1H-pyrazol-1-yl)methyl)-3-bromobenzoate (294 mg, 1 mmol), NiBr2·DME (22 mg, 0.1 mmol), Ir(ppy)2(dtbbpy)PF6 (27 mg, 0.03 mmol), quinuclidine (122 mg, 1.1 mmol) and LiBr (87 mg, 1 mmol) in DMAC (5 mL) was bubbled with N2 for 2 min. The MTBE reaction mixture was filtered and the filtrate was added to the DMAC solution and the resultant mixture was bubbled N2 for another 2 min and stirred at rt under blue LED for overnight. Upon completion of the reaction, the mixture was diluted with EA, washed with water, brine, dried over with Na2SO4, filtered and concentrated. The residue was purified by silica gel column with PE/EA (eluted from PE to PE/EA=1:2) to give the title product (130 mg, 48%). MS (ESI) m/e [M+H]+=273.1.

### Step 2: 4-((1H-pyrazol-1-yl)methyl)-3-(oxetan-3-yl)benzoic acid

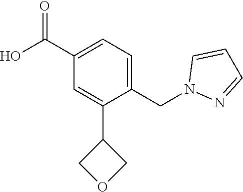 [SMILES: O=C(O)c1ccc(Cn2cccn2)c(C2COC2)c1]

To a solution of methyl 4-((1H-pyrazol-1-yl)methyl)-3-(oxetan-3-yl)benzoate (130 mg, 0.48 mmol) in THF (3 mL), MeOH (3 mL) and water (3 mL) was added NaOH (0.96 mL, 1N, 0.96 mmol) and the solution was stirred at rt for 2 h. Upon completion of the reaction, the reaction mixture was diluted with EA and citric acid solution, washed with brine, dried over with Na2SO4, filtered and concentrated to give the title product (90 mg, crude). MS (ESI) m/e [M+H]+=259.1.

### Example A14: Synthesis of 4-((1H-pyrazol-1-yl)methyl)-3-(azetidin-1-yl)benzoic acid

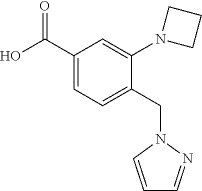 [SMILES: O=C(O)c1ccc(Cn2cccn2)c(N2CCC2)c1]

### Step 1: methyl 4-((1H-pyrazol-1-yl)methyl)-3-(azetidin-1-yl)benzoate

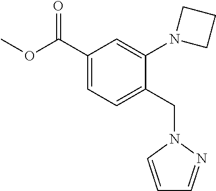 [SMILES: COC(=O)c1ccc(Cn2cccn2)c(N2CCC2)c1]

A mixture of methyl 4-((1H-pyrazol-1-yl)methyl)-3-iodobenzoate (342 mg, 1 mmol), azetidine (114 mg, 2 mmol), Ir[dF(CF3)ppy]2(dtbbpy)PF6 (22 mg, 0.02 mmol), NiCl2·glyme (22 mg, 0.1 mmol) and DABCO (392 mg, 3.5 mmol) in DMAC (10 mL) was bubbled with N2 for 2 min and then stirred at rt under blue LED for overnight. Upon completion of the reaction, the mixture was diluted with EA, washed with water, brine, dried over with Na2SO4, filtered and concentrated. The residue was purified by silica gel column with PE/EA (eluted from PE to EA) to give the title product (200 mg, 74%). MS (ESI) m/e [M+H]+=272.2.

### Step 2: 4-((1H-pyrazol-1-yl)methyl)-3-(azetidin-1-yl)benzoic acid

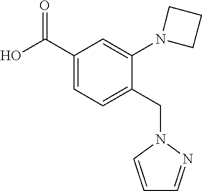 [SMILES: O=C(O)c1ccc(Cn2cccn2)c(N2CCC2)c1]

To a solution of methyl 4-((1H-pyrazol-1-yl)methyl)-3-(azetidin-1-yl)benzoate (200 mg, 0.74 mmol) in THF (5 mL), MeOH (5 mL) and water (5 mL) was added NaOH (1.5 mL, 1N, 1.48 mmol) and the solution was stirred at rt for 2 h. Upon completion of the reaction, the reaction mixture was added citric acid solution to pH value to 4 and diluted with EA and washed with brine, dried and concentrated to give the product (120 mg, 63%). MS (ESI) m/e [M+H]+=258.1.

### Example A15: Synthesis of 4-((1H-pyrazol-1-yl)methyl)-3-cyclobutylbenzoic acid

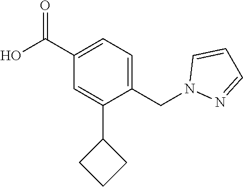 [SMILES: O=C(O)c1ccc(Cn2cccn2)c(C2CCC2)c1]

### Step 1: methyl 4-((1H-pyrazol-1-yl)methyl)-3-cyclobutylbenzoate

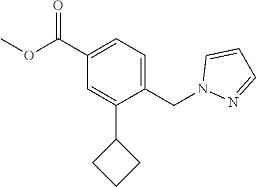 [SMILES: COC(=O)c1ccc(Cn2cccn2)c(C2CCC2)c1]

To a mixture of cyclobutanol (108 mg, 1.5 mmol) and 5,7-Di-tert-butyl-3-phenylbenzo[d]oxazol-3-ium tetrafluoroborate (NHC, 553 mg, 1.4 mmol) in 2-methoxy-2-methylpropane (MTBE, 5 mL) was added pyridine (110 mg, 1.4 mmol) and the mixture was stirred at rt under N2 for 20 min. In another flask, a mixture of methyl 4-((1H-pyrazol-1-yl)methyl)-3-bromobenzoate (294 mg, 1 mmol), NiBr2·DME (22 mg, 0.1 mmol), Ir(ppy)2(dtbbpy)PF6 (27 mg, 0.03 mmol), quinuclidine (122 mg, 1.1 mmol) and LiBr (87 mg, 1 mmol) in DMAC (5 mL) was bubbled with N2 for 2 min. The MTBE reaction mixture was filtered and the filtrate was added to the DMAC solution and the resultant mixture was bubbled N2 for another 2 min and stirred at rt under blue LED for overnight. Upon completion of the reaction, the mixture was diluted with EA, washed with water, brine, dried over with Na2SO4, filtered and concentrated. The residue was purified by silica gel column with PE/EA (eluted from PE to PE/EA=1:1) to give the title product (220 mg, 81%). MS (ESI) m/e [M+H]+=271.1.

### Step 2: 4-((1H-pyrazol-1-yl)methyl)-3-cyclobutylbenzoic acid

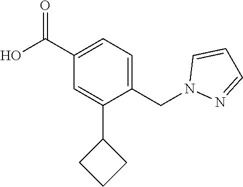 [SMILES: O=C(O)c1ccc(Cn2cccn2)c(C2CCC2)c1]

To a solution of methyl 4-((1H-pyrazol-1-yl)methyl)-3-cyclobutylbenzoate (220 mg, 0.81 mmol) in THF (5 mL), MeOH (5 mL) and water (5 mL) was added NaOH (0.3 mL, 6N, 1.8 mmol) and the solution was stirred at rt for 3 h. Upon completion of the reaction, the reaction mixture was diluted with EA and washed with citric acid solution, brine, dried over with Na2SO4, filtered and concentrated to give the title product (150 mg, 72%). MS (ESI) m/e [M+H]+=257.1.

### Example A16: Synthesis of 4-((1H-pyrazol-1-yl)methyl)-3-chlorobenzoic acid

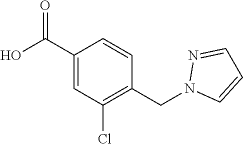 [SMILES: O=C(O)c1ccc(Cn2cccn2)c(Cl)c1]

### Step 1: methyl 4-((1H-pyrazol-1-yl)methyl)-3-chlorobenzoate

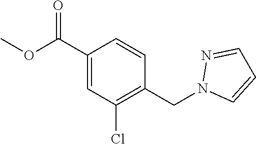 [SMILES: COC(=O)c1ccc(Cn2cccn2)c(Cl)c1]

A mixture of methyl 4-(bromomethyl)-3-chlorobenzoate (900 mg, 3.44 mmol), 1H-pyrazole (351 mg, 5.15 mmol) and Cs2CO3 (2.25 g, 6.88 mmol) in DMF (20 mL) was stirred at 80° C. for 1 h. After cooled to rt, the mixture was quenched by water. The aqueous layer was extracted with DCM. The organic layer was concentrated, and the crude was purified by silica gel column (PE/EA=7:3) to give the product (800 mg, 93%). MS (ESI) m/e [M+H]+=251.

### Step 2: 4-((1H-pyrazol-1-yl)methyl)-3-chlorobenzoic acid

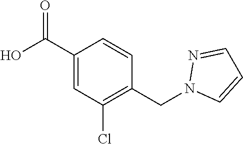 [SMILES: O=C(O)c1ccc(Cn2cccn2)c(Cl)c1]

To a solution of methyl 4-((1H-pyrazol-1-yl)methyl)-3-bromobenzoate (800 mg, 3.39 mmol) in MeOH (5 mL) and THF (5 mL) was added NaOH (2N, 5 mL), the resulting mixture was stirred at rt for overnight. Upon completion of the reaction, the reaction was concentrated, the pH was adjusted to 7 with HCl, the precipitated solid was collected by filtration to give the title compound (700 mg, 93%). MS (ESI) m/e [M+H]+=237.

### Example A17: Synthesis of 5-((1H-pyrazol-1-yl)methyl)-6-methoxypicolinic acid

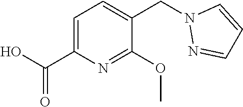 [SMILES: COc1nc(C(=O)O)ccc1Cn1cccn1]

### Step 1: methyl 6-methoxy-5-methylpicolinate

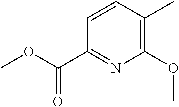

To a solution of methyl 6-chloro-5-methylpicolinate (500 mg, 2.69 mmol) in MeOH (2 mL) was added NaOMe/MeOH solution (2 mL, 30% wt/wt) and the solution was stirred at 85° C. for 3 h. After cooling to rt, the solution was diluted with EA, washed with citric acid solution, brine, dried over with Na2SO4, filtered and concentrated to give the title product (350 mg, 72%). MS (ESI) m/e [M+H]+=182.1.

### Step 2: methyl 5-(bromomethyl)-6-methoxypicolinate

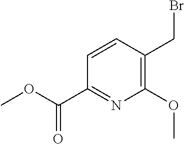

A mixture of methyl 6-methoxy-5-methylpicolinate (326 mg, 1.8 mmol), NBS (325 mg, 1.89 mmol) and BPO (22 mg, 0.09 mmol) in CCl4 (10 mL) was stirred at 80° C. for 4 h. After cooling to rt, the solid was filtered out and the filtrate was washed with NaHCO₃ solution, brine, dried over with Na2SO4, filtered and concentrated. The residue was dissolved in THF (10 mL) and diethyl phosphite (496 mg, 3.6 mmol) followed by TEA (363 mg, 3.6 mmol). The resultant solution was stirred at rt for 1 h and diluted with EA. The solution was washed with citric acid, brine, dried over with Na2SO4, filtered and concentrated. The residue was purified by silica gel column with PE/EA (eluted from PE to PE:EA=2:1) to give the title product (400 mg, 86%). MS (ESI) m/e [M+H]+=260, 262.

### Step 3: methyl 5-((1H-pyrazol-1-yl)methyl)-6-methoxypicolinate

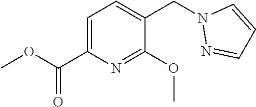 [SMILES: COC(=O)c1ccc(Cn2cccn2)c(OC)n1]

To a mixture of methyl 5-(bromomethyl)-6-methoxypicolinate (400 mg, 1.54 mmol) and 1H-pyrazole (315 mg, 4.63 mmol) in THF (10 mL) was added tBuOK (519 mg, 4.63 mmol) and the reaction mixture was stirred at rt for 1 h. Upon completion of the reaction, the reaction mixture was diluted with EA, washed with water, brine, dried over with Na2SO4, filtered and concentrated. The residue was purified by silica gel column with PE/EA (eluted from PE to PE:EA=1:4) to give the title product (180 mg, 47%). MS (ESI) m/e [M+H]+=248.1.

### Step 4: 5-((1H-pyrazol-1-yl)methyl)-6-methoxypicolinic acid

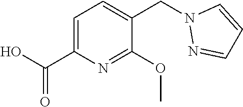 [SMILES: COc1nc(C(=O)O)ccc1Cn1cccn1]

To a solution of methyl 5-((1H-pyrazol-1-yl)methyl)-6-methoxypicolinate (170 mg, 0.69 mmol) in THF (5 mL), MeOH (5 mL) and water (5 mL) was added NaOH (0.22 mL, 6N, 1.32 mmol) and the solution was stirred at rt for overnight. Upon completion of the reaction, the reaction mixture was diluted with EA and citric acid solution, washed with brine, dried over with Na2SO4, filtered and concentrated to give the title product (100 mg, 63%). 1H NMR (400 MHz, DMSO-d6) δ 13.04 (s, 1H), 7.79 (d, J=2.0 Hz, 1H), 7.58 (d, J=7.5 Hz, 1H), 7.46 (d, J=2.0 Hz, 1H) 7.12 (d, J=7.5 Hz, 1H), 6.26 (t, J=2.0 Hz, 1H), 5.30 (s, 2H), 3.92 (s, 3H). MS (ESI) m/e [M+H]+=234.0.

### Example A18: Synthesis of 4-((1H-pyrazol-1-yl)methyl)-3-fluorobenzoic acid

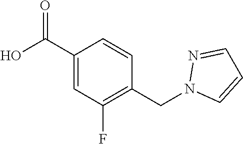 [SMILES: O=C(O)c1ccc(Cn2cccn2)c(F)c1]

### Step 1: methyl 4-((1H-pyrazol-1-yl)methyl)-3-fluorobenzoate

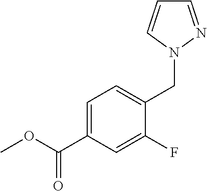 [SMILES: COC(=O)c1ccc(Cn2cccn2)c(F)c1]

A mixture of methyl 4-(bromomethyl)-3-fluorobenzoate (500 mg, 2.02 mmol), 1H-pyrazole (275 mg, 4.05 mmol) and Cs2CO3 (2.00 g, 6.07 mmol) in DMF (10 mL) was stirred at 80° C. for 2 h. After cooled to room temperature, the resulting mixture was concentrated under vacuum. The residue was purified by flash chromatography on silica gel column (eluted with 10%-50% EA in PE) to give the desired product (400 mg, 84%). MS (ESI) m/e [M+1]+=235.

### Step 2: 4-((1H-pyrazol-1-yl)methyl)-3-fluorobenzoic acid

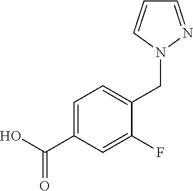 [SMILES: O=C(O)c1ccc(Cn2cccn2)c(F)c1]

A mixture of methyl 4-((1H-pyrazol-1-yl)methyl)-3-fluorobenzoate (400 mg, 1.70 mmol) and NaOH (340 mg, 8.51 mmol) in MeOH (5 mL) and H2O (5 mL) was stirred at rt for 16 h. Upon completion of the reaction, the resulting mixture was adjusted pH value to 4-5 by HCl (1N). The resulting solution was extracted with DCM. The organic layer was concentrated. The residue was purified by reverse flash chromatography (column, C18 reversed phase; mobile phase, MeCN in water (0.1% FA), 10% to 100% gradient in 30 min; detector, UV 254 nm) to give the desired product (200 mg, 53%). MS (ESI) m/e [M+1]+=221.

### Example A19: Synthesis of 4-((1H-pyrazol-1-yl)methyl)-3-(trifluoromethyl)benzoic acid

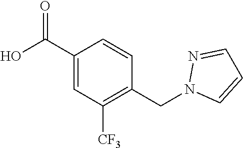 [SMILES: O=C(O)c1ccc(Cn2cccn2)c(C(F)(F)F)c1]

### Step 1: methyl 4-((1H-pyrazol-1-yl)methyl)-3-(trifluoromethyl)benzoate

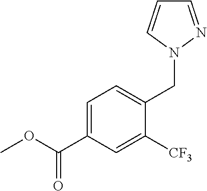 [SMILES: COC(=O)c1ccc(Cn2cccn2)c(C(F)(F)F)c1]

A mixture of methyl 4-(bromomethyl)-3-(trifluoromethyl)benzoate (80 mg, 0.27 mmol), 1H-pyrazole (55 mg, 0.81 mmol) and Cs2CO3 (263 mg, 0.81 mmol) in DMF (5 mL) was stirred at 80° C. for 1 h. After cooled to room temperature, the resulting mixture was concentrated under vacuum. The residue was purified by flash chromatography on silica gel column (eluted with 10%-50% EA in PE) to give the desired product (70 mg, 91%). MS (ESI) m/e [M+1]+=285.

### Step 2: 4-((1H-pyrazol-1-yl)methyl)-3-(trifluoromethyl)benzoic acid

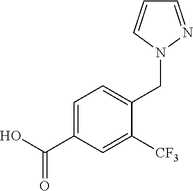 [SMILES: O=C(O)c1ccc(Cn2cccn2)c(C(F)(F)F)c1]

A mixture of methyl 4-((1H-pyrazol-1-yl)methyl)-3-(trifluoromethyl)benzoate (70 mg, 0.25 mmol) and NaOH (49 mg, 1.23 mmol) in MeOH (3 mL) and H2O (3 mL) was stirred at rt for 16 h. Upon completion of the reaction, the resulting mixture was adjusted pH value to 4-5 by HCl (1N). The resulting solution was extracted with DCM. The organic layer was concentrated. The residue was purified by reverse flash chromatography (column, C18 reversed phase; mobile phase, MeCN in water (0.1% FA), 10% to 100% gradient in 30 min; detector, UV 254 nm) to give the desired product (40 mg, 60%). MS (ESI) m/e [M+1]+=271.

### Example A20: Synthesis of 3-methyl-4-(pyridin-2-ylmethyl)benzoic acid

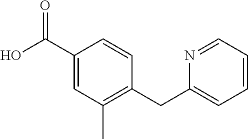 [SMILES: Cc1cc(C(=O)O)ccc1Cc1ccccn1]

### Step 1: methyl 3-methyl-4-(pyridin-2-ylmethyl)benzoate

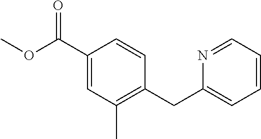 [SMILES: COC(=O)c1ccc(Cc2ccccn2)c(C)c1]

A mixture of methyl 4-bromo-3-methylbenzoate (350 mg, 1.5 mmol), potassium picolinate (340 mg, 1.8 mmol), Pd2dba3 (20 mmol, 0.02 mmol) and XantPhos (36 mg, 0.06 mmol) in diglyme (3 mL) was stirred at 150° C. for 16 h. After cooled to rt, the reaction mixture was diluted with EA and filtered through celite, washed by water and brine. The organic phase was dried over anhydrous Na2SO4, filtered and concentrated. The residue was purified by silica gel column (PE/EA=2:1) to give the desired product (230 mg, 64%). MS (ESI) m/e [M+H]+=242.

### Step 2: 3-methyl-4-(pyridin-2-ylmethyl)benzoic acid

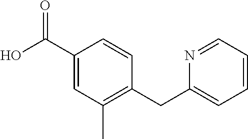 [SMILES: Cc1cc(C(=O)O)ccc1Cc1ccccn1]

A mixture of methyl 3-methyl-4-(pyridin-2-ylmethyl)benzoate (230 mg, 0.95 mmol) and LiOH—H2O (210 mg, 5 mmol) in THF (5 mL)/H2O (5 mL) was stirred at rt for 24 h. Upon completion of the reaction, the reaction mixture was adjusted to pH=6 by 1M aq. HCl, and extracted by EA. The organic phase was dried over anhydrous Na2SO4, filtered and concentrated to give the title compound (120 mg, 55%). MS (ESI) m/e [M+H]+=228.

### Example A21: Synthesis of 4-((1H-pyrazol-1-yl)methyl)-3-(difluoromethoxy)benzoic acid

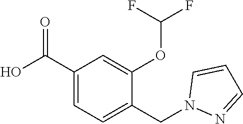 [SMILES: O=C(O)c1ccc(Cn2cccn2)c(OC(F)F)c1]

### Step 1: methyl 3-(difluoromethoxy)-4-methylbenzoate

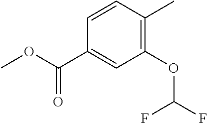

To a solution of methyl 3-hydroxy-4-methylbenzoate (1.66 g, 10 mmol) in ACN (20 mL) was added KOH solution (10 mL, 5N, 50 mmol) and the solution was stirred at 0° C. for 5 min. Diethyl (bromodifluoromethyl)phosphonate (5.34 g, 20 mmol) was added to solution and the reaction mixture was stirred at rt for 3h. Upon completion of the reaction, the solution was diluted with EA, washed with citric acid solution, brine, dried over with Na2SO4, filtered and concentrated. The residue was purified by silica gel column with PE/EA (eluted from PE to PE:EA=4:1) to give the title product (1.5 g, 69%). MS (ESI) m/e [M+H]+=216.9.

### Step 2: methyl 4-(bromomethyl)-3-(difluoromethoxy)benzoate

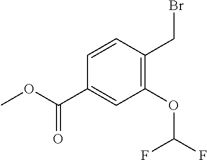 [SMILES: COC(=O)c1ccc(CBr)c(OC(F)F)c1]

A mixture of methyl 3-(difluoromethoxy)-4-methylbenzoate (1.5 g, 6.9 mmol), NBS (1.36 g, 7.6 mmol) and BPO (167 mg, 0.69 mmol) in CCl4 (20 mL) was stirred at 80° C. for overnight. After cooling to rt, the solid was filtered out and the filtrate was washed with NaHCO3 solution, brine, dried over with Na2SO4, filtered and concentrated. The residue was dissolved in THF (20 mL) and diethyl phosphite (1.9 g, 13.8 mmol) followed by TEA (1.39 g, 13.8 mmol). The resultant solution was stirred at rt for 1h and diluted with EA. The solution was washed with citric acid, brine, dried over with Na2SO4, filtered and concentrated. The residue was purified by silica gel column with PE/EA (eluted from PE to PE:EA=4:1) to give the title product (1 g, 49%). MS (ESI) m/e [M+H]+=295, 297.

### Step 3: methyl 4-((1H-pyrazol-1-yl)methyl)-3-(difluoromethoxy)benzoate

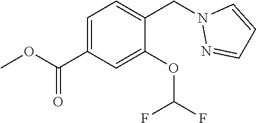 [SMILES: COC(=O)c1ccc(Cn2cccn2)c(OC(F)F)c1]

To a mixture of methyl 4-(bromomethyl)-3-(difluoromethoxy)benzoate (1 g, 3.9 mmol) and 1H-pyrazole (462 mg, 6.8 mmol) in THF (20 mL) was added tBuOK (571 mg, 5.1 mmol) and the reaction mixture was stirred at rt for overnight. Upon completion of the reaction, the reaction mixture was diluted with EA, washed with water, brine, dried over with Na2SO4, filtered and concentrated. The residue was purified by silica gel column with PE/EA (eluted from PE to PE:EA=1:2) to give the title product (800 mg, 73%). MS (ESI) m/e [M+H]+=283.0.

### Step 4: 4-((1H-pyrazol-1-yl)methyl)-3-(difluoromethoxy)benzoic acid

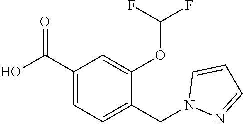 [SMILES: O=C(O)c1ccc(Cn2cccn2)c(OC(F)F)c1]

To a solution of methyl 4-((1H-pyrazol-1-yl)methyl)-3-(difluoromethoxy)benzoate (800 mg, 2.84 mmol) in THF (5 mL), MeOH (5 mL) and water (5 mL) was added NaOH (0.9 mL, 6N, 5.4 mmol) and the solution was stirred at rt for 2h. Upon completion of the reaction, the reaction mixture was diluted with EA and citric acid solution, washed with brine, dried over with Na2SO4, filtered and concentrated to give the title product (600 mg, 79%). MS (ESI) m/e [M+H]+=268.9.

### Example A22: Synthesis of 4-((1H-pyrazol-1-yl)methyl)-3-(2,2-difluoroethyl)benzoic acid

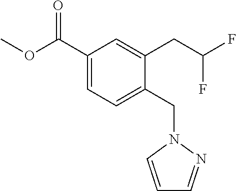 [SMILES: COC(=O)c1ccc(Cn2cccn2)c(CC(F)F)c1]

### Step 1: methyl 4-((1H-pyrazol-1-yl)methyl)-3-vinylbenzoate

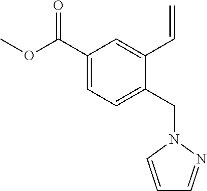 [SMILES: C=Cc1cc(C(=O)OC)ccc1Cn1cccn1]

A mixture of methyl 4-((1H-pyrazol-1-yl)methyl)-3-iodobenzoate (1.03 g, 3 mmol), tributyl(vinyl)stannane (999 mg, 3.15 mmol), Pd(PPh3)4 (173 mg, 0.15 mmol) and LiCl (126 mg, 3 mmol) in 1,4-dioxane (20 mL) was stirred at 70° C. for 16 h. After cooling to rt, the solution was diluted with EA, washed with brine, dried over with Na2SO4, filtered and concentrated. The residue was purified by silica gel column with PE/EA (eluted from PE to PE:EA=1:1) to give the title product (600 mg, 83%). MS (ESI) m/e [M+H]+=242.9.

### Step 2: methyl 4-((1H-pyrazol-1-yl)methyl)-3-formylbenzoate

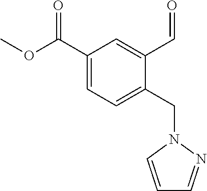 [SMILES: COC(=O)c1ccc(Cn2cccn2)c(C=O)c1]

To a solution of methyl 4-((1H-pyrazol-1-yl)methyl)-3-vinylbenzoate (600 mg, 2.48 mmol) in THF (20 mL) and water (10 mL) was added K2OsO42H2O (46 mg, 0.12 mmol) and the solution was stirred at rt for 10 min. NaIO4 (1.6 g, 7.44 mmol) was added and the mixture was stirred at rt for 16h. Upon completion of the reaction, the mixture was diluted with EA, washed with water, Na2S2O3 solution, brine, dried over with Na2SO4, filtered and concentrated. The residue was purified by silica gel column with PE/EA (eluted from PE to PE:EA=1:4) to give the title product (500 mg, 82%). MS (ESI) m/e [M+H]+=245.1.

### Step 3: methyl 4-((1H-pyrazol-1-yl)methyl)-3-(hydroxymethyl)benzoate

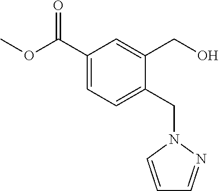 [SMILES: COC(=O)c1ccc(Cn2cccn2)c(CO)c1]

To a solution of methyl 4-((1H-pyrazol-1-yl)methyl)-3-formylbenzoate (300 mg, 1.23 mmol) in MeOH (10 mL) was added NaBH4 (70 mg, 1.84 mmol) and the solution was stirred at rt for 2h. Upon completion of the reaction, the mixture was diluted with EA, washed with water, brine, dried over with Na2SO4, filtered and concentrated to give the title product (280 mg, 92%). MS (ESI) m/e [M+H]+=247.0.

### Step 4: methyl 4-((1H-pyrazol-1-yl)methyl)-3-(2,2-difluoroethyl)benzoate

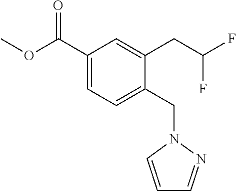 [SMILES: COC(=O)c1ccc(Cn2cccn2)c(CC(F)F)c1]

To a mixture of methyl 4-((1H-pyrazol-1-yl)methyl)-3-(hydroxymethyl)benzoate (246 mg, 1 mmol) and 5,7-Di-tert-butyl-3-phenylbenzo[d]oxazol-3-ium tetrafluoroborate (NHC, 474 mg, 1.2 mmol) in 2-methoxy-2-methylpropane (MTBE, 4 mL) was added pyridine (118 mg, 1.5 mmol) and the mixture was stirred at rt under N2 for 30 min. In another flask, a mixture of 2-((difluoromethyl)sulfonyl)benzo[d]thiazole (274 mg, 1.1 mmol), Cu(TMHD)2 (43 mg, 0.1 mmol), Ir(ppy)2(dtbbpy)PF6 (46 mg, 0.05 mmol), t-Bu-terpy (44 mg, 0.11 mmol) and tetrabutylammonium benzoate (437 mg, 1.2 mmol) in DMSO (20 mL) was bubbled with N2 for 2 min. The MTBE reaction mixture was filtered and the filtrate was added to the DMSO solution and the resultant mixture was bubbled N2 for another 2 min and stirred at rt under blue LED for overnight. Upon completion of the reaction, the mixture was diluted with EA, washed with water, brine, dried over with Na2SO4, filtered and concentrated. The residue was purified by silica gel column with PE/EA (eluted from PE to EA) to give the title product (200 mg, 71%). MS (ESI) m/e [M+H]+=281.0.

### Step 5: 4-((1H-pyrazol-1-yl)methyl)-3-(2,2-difluoroethyl)benzoic acid

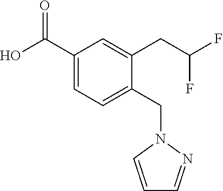 [SMILES: O=C(O)c1ccc(Cn2cccn2)c(CC(F)F)c1]

To a solution of methyl 4-((1H-pyrazol-1-yl)methyl)-3-(2,2-difluoroethyl)benzoate (200 mg, 0.71 mmol) in THF (3 mL), MeOH (3 mL) and water (3 mL) was added NaOH (0.24 mL, 6N, 1.44 mmol) and the solution was stirred at rt for 16h. Upon completion of the reaction, the reaction mixture was adjusted pH to 5 with citric acid solution and then diluted with EA, the mixture was washed with brine, dried over with Na2SO4, filtered and concentrated to give the title product (170 mg, 90%). MS (ESI) m/e [M+H]+=267.1.

### Example A23: Synthesis of 3-methoxy-4-(pyridin-2-ylmethyl)benzoic acid

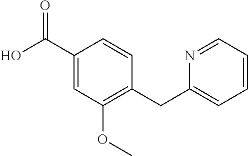 [SMILES: COc1cc(C(=O)O)ccc1Cc1ccccn1]

### Step 1: methyl 3-methoxy-4-(pyridin-2-ylmethyl)benzoate

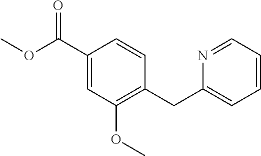 [SMILES: COC(=O)c1ccc(Cc2ccccn2)c(OC)c1]

A mixture of methyl 4-bromo-3-methoxybenzoate (100 mg, 0.4 mmol), potassium picolinate (80 mg, 0.48 mmol), Pd2dba3 (20 mmol, 0.02 mmol) and XantPhos (24 mg, 0.04 mmol) in diglyme (5 mL) was stirred at 150° C. for 16 h. After cooled to rt, the mixture was quenched by water. The aqueous layer was extracted with DCM. The organic layer was concentrated, and the crude was purified by silica gel column (PE/EA=1:1) to give the product (65 mg, 62%). MS (ESI) m/e [M+H]+=258.

### Step 2: 3-methoxy-4-(pyridin-2-ylmethyl)benzoic acid

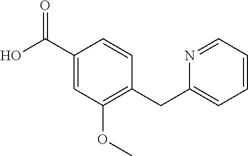 [SMILES: COc1cc(C(=O)O)ccc1Cc1ccccn1]

To a solution of methyl 3-methoxy-4-(pyridin-2-ylmethyl)benzoate (65 mg, 0.25 mmol) in MeOH (5 mL) and THF (5 mL) was added NaOH (2N, 5 mL), the resulting mixture was stirred at rt for overnight. Upon completion of the reaction, the reaction was concentrated, the pH value was adjusted to 7 with HCl, the precipitated solid was collected by filtration to give the title compound (55 mg, 89%). MS (ESI) m/e [M+H]+=244.

### Example A24: Synthesis of 3-methoxy-4-((3-methyl-1H-pyrazol-1-yl)methyl)benzoic acid

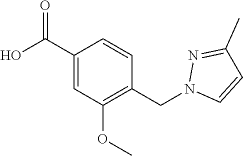 [SMILES: COc1cc(C(=O)O)ccc1Cn1ccc(C)n1]

### Step 1: methyl 4-(bromomethyl)-3-methoxybenzoate

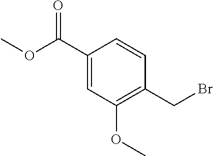

A mixture of methyl 3-methoxy-4-methylbenzoate (1.0 g, 5.56 mmol), NBS (1.09 g, 6.11 mmol) and AIBN (183 mg, 1.11 mmol) in CCl4 (20 mL) was stirred at 90° C. for 2 h. After cooled to rt, the mixture was quenched by water. The aqueous layer was extracted with DCM. The organic layer was concentrated, and the crude was purified by silica gel column chromatography (PE/EA=10:1) to give the product (1.0 g, 70%). MS (ESI) m/e [M+H]+=259, 261.

### Step 2: methyl 3-methoxy-4-((3-methyl-1H-pyrazol-1-yl)methyl)benzoate

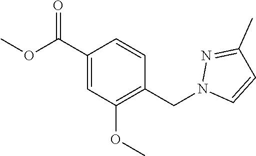 [SMILES: COC(=O)c1ccc(Cn2ccc(C)n2)c(OC)c1]

To a solution of 3-methyl-1H-pyrazole (38 mg, 0.47 mmol) in THF (3 mL) was added NaH (60% in mineral oil, 22 mg, 0.55 mmol), the resulting mixture was stirred at rt for 30 min. Methyl 4-(bromomethyl)-3-methoxybenzoate (100 mg, 0.39 mmol) was added, the resulting mixture was stirred at rt for 30 min. Upon completion of the reaction, the mixture was quenched by water. The aqueous layer was extracted with DCM. The organic layer was concentrated, and the crude was purified by silica gel column (PE/EA=1:1) to give the product (70 mg, 69%). MS (ESI) m/e [M+H]+=261.

### Step 3: 3-methoxy-4-((3-methyl-1H-pyrazol-1-yl)methyl)benzoic acid

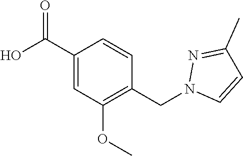 [SMILES: COc1cc(C(=O)O)ccc1Cn1ccc(C)n1]

To a solution of methyl 3-methoxy-4-((3-methyl-1H-pyrazol-1-yl)methyl)benzoate (70 mg, 0.27 mmol) in MeOH (5 mL) and THF (5 mL) was added NaOH (2N, 0.5 mL), the resulting mixture was stirred at rt for overnight. Upon completion of the reaction, the reaction was concentrated, the pH value was adjusted to 7 with HCl, the precipitated solid was collected by filtration to give the title compound (60 mg, 91%). MS (ESI) m/e [M+H]+=247.

### Example A25: Synthesis of 5-((1H-pyrazol-1-yl)methyl)-6-ethoxypicolinic acid

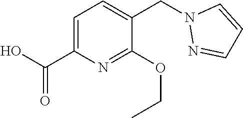 [SMILES: CCOc1nc(C(=O)O)ccc1Cn1cccn1]

### Step 1: methyl 6-ethoxy-5-methylpicolinate

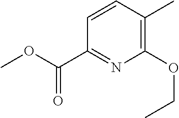

To a solution of methyl 6-chloro-5-methylpicolinate (500 mg, 2.69 mmol) in toluene (10 mL) was added NaOEt/EtOH solution (2.75 g, 8.1 mmol, 20% wt/wt) and the solution was stirred at 100° C. for 16 h. After cooling to rt, the solution was diluted with EA, washed with citric acid solution, brine, dried over with Na2SO4, filtered and concentrated, the residue was purified by silica gel column with PE/EA (eluted from PE to PE:EA=1:1) to give the title product (450 mg, 80%). MS (ESI) m/e [M+H]+=196.0.

### Step 2: methyl 5-(bromomethyl)-6-ethoxypicolinate

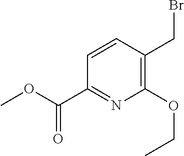

A mixture of methyl 6-ethoxy-5-methylpicolinate (450 mg, 2.29 mmol), NBS (421 mg, 2.37 mmol) and BPO (51 mg, 0.21 mmol) in CCl4 (10 mL) was stirred at 80° C. for 3 h. After cooling to rt, the solid was filtered out and the filtrate was washed with NaHCO₃ solution, brine, dried over with Na2SO4, filtered and concentrated. The residue was dissolved in THF (10 mL) and diethyl phosphite (581 mg, 2.29 mmol) followed by TEA (233 mg, 2.29 mmol). The resultant solution was stirred at rt for 2 h and diluted with EA. The solution was washed with citric acid, brine, dried over with Na2SO4, filtered and concentrated. The residue was purified by silica gel column with PE/EA (eluted from PE to EA) to give the title product (350 mg, 56%). MS (ESI) m/e [M+H]+=273.9.

### Step 3: 5-((1H-pyrazol-1-yl)methyl)-6-ethoxypicolinic acid

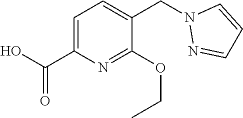 [SMILES: CCOc1nc(C(=O)O)ccc1Cn1cccn1]

To a mixture of methyl 5-(bromomethyl)-6-ethoxypicolinate (350 mg, 1.28 mmol) and 1H-pyrazole (166 mg, 2.44 mmol) in THF (20 mL) was added tBuOK (273 mg, 2.44 mmol) and the reaction mixture was stirred at rt for 16 h. Upon completion of the reaction, NaOH solution (1 mL, 6N, 6 mmol) was added to the reaction mixture and stirred for further 2 h. The mixture was adjusted pH to 5 with citric acid solution and diluted with EA, washed with water, brine, dried over Na2SO4, filtered and concentrated. The residue was purified by C18 column with 0.1% FA in water/ACN (eluted from 10% of 0.1% FA in water to ACN) to give the title product (100 mg, 31%). MS (ESI) m/e [M+H]+=248.0.

### Example A26: Synthesis of 6-methyl-5-(pyridin-2-ylmethyl)picolinic acid

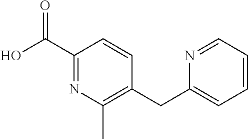 [SMILES: Cc1nc(C(=O)O)ccc1Cc1ccccn1]

### Step 1: methyl 6-methyl-5-(pyridin-2-ylmethyl)picolinate

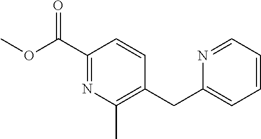 [SMILES: COC(=O)c1ccc(Cc2ccccn2)c(C)n1]

A mixture of methyl 5-bromo-6-methylpicolinate (350 mg, 1.5 mmol), potassium picolinate (340 mg, 1.8 mmol), Pd2dba3 (20 mmol, 0.02 mmol) and XantPhos (36 mg, 0.06 mmol) in diglyme (3 mL) was stirred at 150° C. for 16 h. After cooled to rt, the reaction mixture was diluted with EA and filtered through celite, washed by water and brine. The organic phase was dried over anhydrous Na2SO4, filtered and concentrated. The residue was purified by silica gel column (PE/EA=1:1 to 1:3) to give the desired product (160 mg, 44%). MS (ESI) m/e [M+H]+=243.

### Step 2: 6-methyl-5-(pyridin-2-ylmethyl)picolinic acid

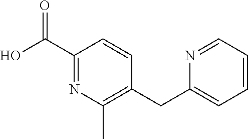 [SMILES: Cc1nc(C(=O)O)ccc1Cc1ccccn1]

A mixture of methyl 6-methyl-5-(pyridin-2-ylmethyl)picolinate (120 mg, 0.5 mmol) and LiOH—H2O (210 mg, 5 mmol) in THF (5 mL)/H2O (5 mL) was stirred at rt for 24 h. Upon completion of the reaction, the reaction mixture was adjusted to pH=6 by 1M aq. HCl, and extracted by EA. The organic phase was dried over anhydrous Na2SO4, filtered and concentrated to give the title compound (60 mg, 52%). MS (ESI) m/e [M+H]+=229.

### Example A27: Synthesis of 4-((1H-pyrazol-1-yl)methyl)-3-(difluoromethyl)benzoic acid

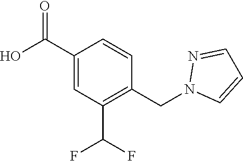 [SMILES: O=C(O)c1ccc(Cn2cccn2)c(C(F)F)c1]

### Step 1: methyl 3-(difluoromethyl)-4-methylbenzoate

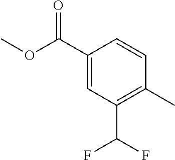

To a solution of methyl 3-formyl-4-methylbenzoate (300 mg, 1.69 mmol) in DCM (5 mL) was added DAST (2.7 g, 16.9 mmol), the resulting mixture was stirred at 50° C. for 1h. After cooled to rt, the mixture was poured into ice water and extracted with EA. The organic layer was washed with sat.NaHCO₃, brine and concentrated to give the product (300 mg, 89%). MS (ESI) m/e [M+H]+=201.

### Step 2: methyl 4-(bromomethyl)-3-(difluoromethyl)benzoate

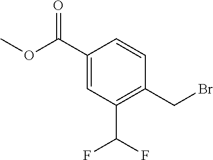

A mixture of methyl 3-(difluoromethyl)-4-methylbenzoate (280 mg, 1.4 mmol), NBS (300 mg, 1.68 mmol) and AIBN (23 mmol, 0.14 mmol) in CCl4 (5 mL) was stirred at 90° C. for 2 h. After cooled to rt, the mixture was quenched by water. The aqueous layer was extracted with DCM. The organic layer was concentrated, and the crude was purified by silica gel column (PE/EA=10:1) to give the product (380 mg, 98%). MS (ESI) m/e [M+H]+=279, 281.

### Step 3: methyl 4-((1H-pyrazol-1-yl)methyl)-3-(difluoromethyl)benzoate

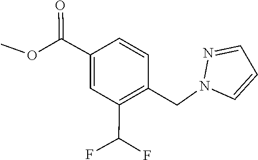 [SMILES: COC(=O)c1ccc(Cn2cccn2)c(C(F)F)c1]

To a solution of 1H-pyrazole (91 mg, 1.34 mmol) in THF (10 mL) was added NaH (60% in mineral oil, 68 mg, 0.55 mmol) at 0° C., the resulting mixture was stirred at rt for 30 min. Methyl 4-(bromomethyl)-3-(difluoromethyl)benzoate (310 mg, 1.12 mmol) was added, the resulting mixture was stirred at rt for 1h. Upon completion of the reaction, the mixture was quenched by water. The aqueous layer was extracted with DCM. The organic layer was concentrated, and the crude was purified by silica gel column (PE/EA=7:3) to give the product (50 mg, 17%). MS (ESI) m/e [M+H]+=267.

### Step 4: 4-((1H-pyrazol-1-yl)methyl)-3-(difluoromethyl)benzoic acid

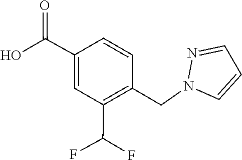 [SMILES: O=C(O)c1ccc(Cn2cccn2)c(C(F)F)c1]

To a solution of methyl 4-((1H-pyrazol-1-yl)methyl)-3-(difluoromethyl)benzoate (45 mg, 0.17 mmol) in MeOH (5 mL) and THF (5 mL) was added NaOH (2N, 0.5 mL), the resulting mixture was stirred at rt for overnight. Upon completion of the reaction, the reaction was concentrated, the pH value was adjusted to 7 with HCl, the precipitated solid was collected by filtration to give the title compound (40 mg, 94%). MS (ESI) m/e [M+H]+=253.

### Example A28: Synthesis of 5-((3-((tert-butoxycarbonyl)amino)-1H-pyrazol-1-yl)methyl)-6-methoxypicolinic acid

### Step 1: methyl 5-((3-((tert-butoxycarbonyl)amino)-1H-pyrazol-1-yl)methyl)-6-methoxypicolinate

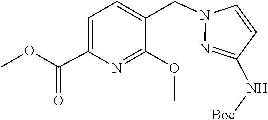 [SMILES: COC(=O)c1ccc(Cn2ccc(NC(=O)OC(C)(C)C)n2)c(OC)n1]

To a mixture of tert-butyl (1H-pyrazol-3-yl)carbamate (100 mg, 0.55 mmol) in THF (10 mL) was added tBuOK (62 mg, 0.55 mmol) and the mixture was stirred at 0° C. for 10 min. Methyl 5-(bromomethyl)-6-methoxypicolinate (130 mg, 0.5 mmol) was added and the reaction mixture was stirred at rt for overnight. Upon completion of the reaction, the reaction mixture was diluted with EA, washed with citric acid, water, brine, dried over with Na2SO4, filtered and concentrated to give the crude product (240 mg, crude). MS (ESI) m/e [M+H]+=363.0.

### Step 2: 5-((3-((tert-butoxycarbonyl)amino)-1H-pyrazol-1-yl)methyl)-6-methoxypicolinic acid

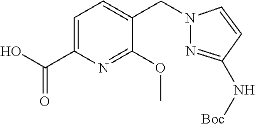 [SMILES: COc1nc(C(=O)O)ccc1Cn1ccc(NC(=O)OC(C)(C)C)n1]

To a solution of methyl 5-((3-((tert-butoxycarbonyl)amino)-1H-pyrazol-1-yl)methyl)-6-methoxypicolinate (240 mg, 0.5 mmol) in THF (5 mL), MeOH (5 mL) and water (5 mL) was added NaOH (0.17 mL, 6N, 1.02 mmol) and the solution was stirred at rt for 2h. Upon completion of the reaction, the reaction mixture was diluted with EA and citric acid solution, washed with brine, dried over with Na2SO4, filtered and concentrated. The residue was purified by silica gel column with DCM/MeOH (eluted from DCM to DCM:MeOH=5:1) to give the title product (80 mg, 46% for two steps). MS (ESI) m/e [M+H]+=349.0.

### Example A29: Synthesis of 4-((4-((tert-butoxycarbonyl)amino)-1H-pyrazol-1-yl)methyl)-3-methoxybenzoic acid

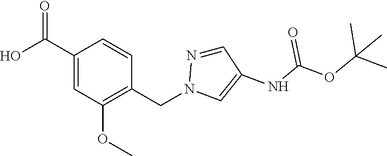 [SMILES: COc1cc(C(=O)O)ccc1Cn1cc(NC(=O)OC(C)(C)C)cn1]

### Step 1: methyl 4-((4-((tert-butoxycarbonyl)amino)-1H-pyrazol-1-yl)methyl)-3-methoxybenzoate

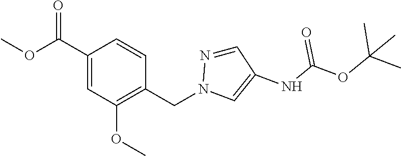 [SMILES: COC(=O)c1ccc(Cn2cc(NC(=O)OC(C)(C)C)cn2)c(OC)c1]

A mixture of methyl 4-(bromomethyl)-3-methoxybenzoate (300 mg, 1.16 mmol), tert-butyl (1H-pyrazol-4-yl)carbamate (256 mg, 1.40 mmol) and Cs2CO3 (757 mg, 2.32 mmol) in DMF (10 mL) was stirred at 70° C. for 1h. After cooled to rt, the mixture was quenched by water. The aqueous layer was extracted with DCM. The organic layer was concentrated, and the crude was purified by silica gel column (PE/EA=3:2) to give the product (400 mg, 95%). MS (ESI) m/e [M+H]+=362.

### Step 2: 4-((4-((tert-butoxycarbonyl)amino)-1H-pyrazol-1-yl)methyl)-3-methoxybenzoic acid

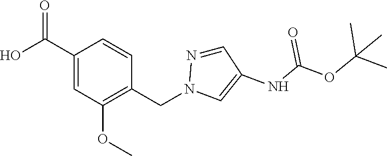 [SMILES: COc1cc(C(=O)O)ccc1Cn1cc(NC(=O)OC(C)(C)C)cn1]

To a solution of methyl 4-((4-((tert-butoxycarbonyl)amino)-1H-pyrazol-1-yl)methyl)-3-methoxybenzoate (400 mg, 1.11 mmol) in MeOH (5 mL) and THF (5 mL) was added NaOH (2N, 1 mL), the resulting mixture was stirred at rt for overnight. Upon completion of the reaction, the reaction was concentrated, the pH value was adjusted to 7 with HCl, the precipitated solid was collected by filtration to give the title compound (330 mg, 86%). MS (ESI) m/e [M+H]+=348.

### Example A30: Synthesis of 5-((1H-pyrazol-1-yl)methyl)-6-ethylpicolinic acid

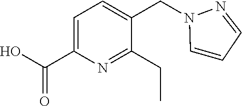 [SMILES: CCc1nc(C(=O)O)ccc1Cn1cccn1]

### Step 1: 2-bromo-3-(chloromethyl)pyridine

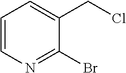

To a solution of (2-bromopyridin-3-yl)methanol (1.87 g, 10 mmol) in DCM (10 mL) was added SOCl2 (2.38 g, 20 mmol) at 0° C. and stirred at r.t overnight. Upon completion of the reaction, the mixture was diluted with water, extracted with EA. The organic layer was concentrated to give the crude product (2.00 g, 97%). MS (ESI) m/e [M+H]+=206, 208.

### Step 2: 3-((1H-pyrazol-1-yl)methyl)-2-bromopyridine

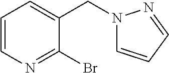 [SMILES: Brc1ncccc1Cn1cccn1]

A solution of 2-bromo-3-(chloromethyl)pyridine (2.00 g, 9.76 mmol), 1H-imidazo[1,2-a]imidazole (680 mg, 10.00 mmol) and Cs2CO3 (6.52 g, 10 mmol) in DMF (10 mL) was stirred at 80° C. for overnight. Upon completion of the reaction, the solution was concentrated and purified by silica gel column chromatograph (PE/EA=3:1) to give the product (2.00 g, 86%). MS (ESI) m/e [M+H]+=238, 240.

### Step 3: 3-((1H-pyrazol-1-yl)methyl)-2-vinylpyridine

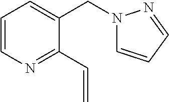

A solution of 3-((1H-pyrazol-1-yl)methyl)-2-bromopyridine (2.00 g, 8.43 mmol), vinylboronic acid trifluoroborate potassium salt (2.26 g, 16.86 mmol), Pd(PPh3)4 (924 mg, 0.8 mmol) and K2CO3 (2.33 g, 16.86 mmol) in dioxane/water (10 mL, v/v=5:1) was stirred at 100° C. under N2 overnight. Upon completion of the reaction, the mixture was concentrated and purified by silica gel column chromatography (PE/EA=3:1) to give the product (1 g, 64%). MS (ESI) m/e [M+H]+=186.2.

### Step 4: 3-((1H-pyrazol-1-yl)methyl)-2-ethylpyridine

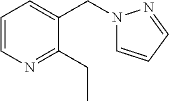

A solution of 3-((1H-pyrazol-1-yl)methyl)-2-vinylpyridine (2.00 g, 10.81 mmol) and Pd/C (10% on carbon, wet) (200 mg) in MeOH (10 mL) was stirred at r.t under H2 (2 atm) for 4 hr. Upon completion of the reaction, the mixture was filtered. The filtrate was concentrated to give the product (2 g, 99%). MS (ESI) m/e [M+H]+=188.3.

### Step 5: 3-((1H-pyrazol-1-yl)methyl)-2-ethylpyridine 1-oxide

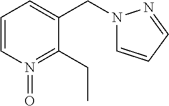

A solution of 3-((1H-pyrazol-1-yl)methyl)-2-ethylpyridine (1.30 g, 6.53 mmol) and m-CPBA (3.70 g, 21.40 mmol) in DCM (20 mL) was stirred at r.t overnight. Upon completion of the reaction, the mixture was concentrated. The crude was purified by silica gel column chromatography (PE/EA=2:1) to give the product (500 mg, 38%). MS (ESI) m/e [M+H]+=204.2.

### Step 6: 3-((1H-pyrazol-1-yl)methyl)-6-chloro-2-ethylpyridine

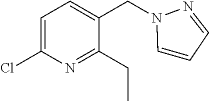

A solution of 3-((1H-pyrazol-1-yl)methyl)-2-ethylpyridine 1-oxide (406 mg, 2 mmol) and POCl3 (918 mg, 6.00 mmol) in DCE (10 mL) was stirred at 100° C. overnight. Upon completion of the reaction, the solution was diluted with water and extracted with EA. The organic layer was concentrated and purified by Combi-Flash (Column=C18 spherical 20-35 um; mobile phase: [water (0.1% FA)-ACN], B %=5%-80%; 7.0 min) to give the product (200 mg, 45%). 1H NMR (400 MHz, DMSO-d6) δ 7.86 (d, J=2.0 Hz, 1H), 7.51 (d, J=2.3 Hz, 1H), 7.42 (d, J=8.3 Hz, 1H), 7.34 (d, J=8.2 Hz, 1H), 6.56-6.52 (m, 1H), 5.60 (s, 2H), 2.94-2.84 (m, 2H), 1.22 (t, J=7.6 Hz, 3H). MS (ESI) m/e [M+H]+=222.2.

### Step 7: Methyl 5-((1H-pyrazol-1-yl)methyl)-6-ethylpicolinate

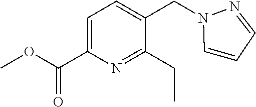 [SMILES: CCc1nc(C(=O)OC)ccc1Cn1cccn1]

A solution of 3-((1H-pyrazol-1-yl)methyl)-6-chloro-2-ethylpyridine (200 mg, 0.90 mmol), Pd(dppf)2Cl2 (66 mg, 0.09 mmol) and TEA (182 mg, 1.8 mmol) in MeOH (10 mL) was stirred at 100° C. under CO (15 atm) overnight. After cooled to r.t, the solution was concentrated. The crude was purified by silica gel column chromatography (PE/EA=2:1) to give the product (200 mg, 91%). MS (ESI) m/e [M+H]+=246.2.

### Step 8: 5-((1H-pyrazol-1-yl)methyl)-6-ethylpicolinic acid

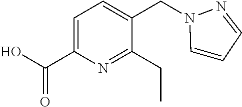 [SMILES: CCc1nc(C(=O)O)ccc1Cn1cccn1]

A solution of methyl 5-((1H-pyrazol-1-yl)methyl)-6-ethylpicolinate (100 mg, 0.81 mmol) and LiOH·H2O (100 mg, 2.38 mmol) in THF/H2O (10 mL, v/v=1/1) was stirred at 50° C. for 2 hr. Upon completion of the reaction, the solution was concentrated. The crude was purified by Combi-Flash (Column=C18 spherical 20-35 um; mobile phase: [water (0.1% FA)-ACN], B %=5%-80%; 5.0 min) to give the product (100 mg, 53%). MS (ESI) m/e [M+H]+=232.2.

### Example A31: Synthesis of 5-((1H-pyrazol-1-yl)methyl)-6-(methoxy-d3)picolinic acid

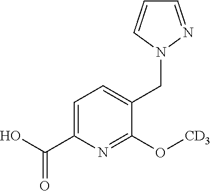 [SMILES: [2H]C([2H])([2H])Oc1nc(C(=O)O)ccc1Cn1cccn1]

### Step 1: methyl-d3 6-chloro-5-methylpicolinate

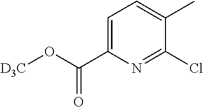

To a solution of 6-chloro-5-methylpicolinic acid (5 g, 29.2 mmol) in DMF (50 mL) was added K2CO3 (4.43 g, 32.1 mmol) followed by CD3I (4.66 g, 32.1 mmol) and the mixture was stirred at rt for overnight. Upon completion of the reaction, the reaction mixture was poured into ice-water (200 mL) and extract with EA for twice. The extracts were washed with citric acid solution, brine, dried over with Na2SO4, filtered and concentrated to give the title product (4.8 g, 87%). MS (ESI) m/e [M+H]+=189.1.

### Step 2: methyl 6-(methoxy-d3)-5-methylpicolinate

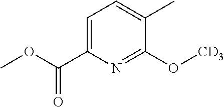 [SMILES: [2H]C([2H])([2H])Oc1nc(C(=O)OC)ccc1C]

To a solution of CD3OD (4.79 g, 106 mmol) (4 g, 21.3 mmol) in THF (100 mL) was added NaH (60% in mineral oil, 2.13 g, 53 mmol) at 0° C. and stirred at 0° C. for 30 min. Dmethyl-d3 6-chloro-5-methylpicolinate (4 g, 21.3 mmol) was added and the solution was stirred at 50° C. for 3 h. After cooling to rt, the reaction mixture was poured into ice-water and citric acid solution (500 mL) and extracted with EA for twice. The extracts were washed with brine, dried over with Na2SO4, filtered and concentrated and the residue was purified by silica gel column chromatography (PE:EA=1:0 to 1:1) to give the title product (2.3 g, 58%). MS (ESI) m/e [M+H]+=185.2.

### Step 3: methyl 5-(bromomethyl)-6-(methoxy-d3)picolinate

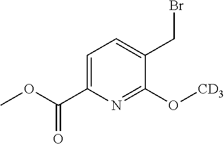 [SMILES: [2H]C([2H])([2H])Oc1nc(C(=O)OC)ccc1CBr]

A mixture of methyl 6-(methoxy-d3)-5-methylpicolinate (2.3 g, 12.4 mmol), NBS (2.43 g, 13.7 mmol) and BPO (150 mg, 0.62 mmol) in CCl4 (50 mL) was stirred at 80° C. for 4 h. After cooling to room temperature, the solid was filtered out and the filtrate was washed with NaHCO3 solution, brine, dried over with Na2SO4, filtered and concentrated. The residue was dissolved in THF (50 mL) and diethyl phosphite (1.67 g, 12.4 mmol) followed by TEA (1.25 g, 12.4 mmol). The resultant solution was stirred at rt for 4 h and diluted with EA. The solution was washed with water, brine, dried over with Na2SO4, filtered and concentrated to give the title product (3 g, crude). MS (ESI) m/e [M+H]+=263, 265.

### Step 4: methyl 5-((1H-pyrazol-1-yl)methyl)-6-(methoxy-d3)picolinate

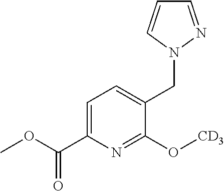 [SMILES: [2H]C([2H])([2H])Oc1nc(C(=O)OC)ccc1Cn1cccn1]

To a mixture of methyl 5-(bromomethyl)-6-(methoxy-d3)picolinate (3 g, 11.45 mmol) and 1H-pyrazole (1.24 g, 18.32 mmol) in DMF (50 mL) was added Cs2CO3 (3.92 g, 12 mmol) and the reaction mixture was stirred at 80° C. for 4 h. After cooling to rt, the reaction mixture was poured into ice-water and citric acid solution and extracted with EA for twice. The combined organic layers were washed with water, brine, dried over with Na2SO4, filtered and concentrated. The residue was purified by silica gel column with PE/EA (eluted from PE to EA) to give the title product (1.1 g, 38%). MS (ESI) m/e [M+H]+=251.2.

### Step 5: 5-((1H-pyrazol-1-yl)methyl)-6-(methoxy-d3)picolinic acid

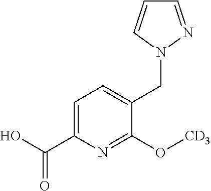 [SMILES: [2H]C([2H])([2H])Oc1nc(C(=O)O)ccc1Cn1cccn1]

To a solution of methyl 5-((1H-pyrazol-1-yl)methyl)-6-(methoxy-d3)picolinate (1.1 g, 4.4 mmol) in THF (10 mL), MeOH (10 mL) and water (10 mL) was added NaOH (1.1 mL, 6N, 6.6 mmol) and the solution was stirred at rt for 2h. Upon completion of the reaction, the reaction mixture was diluted with water and adjusted pH to 5 with citric acid solution and extracted with EA for twice. The extracts were washed with water, brine, dried and concentrated to give the product. (1 g, 96%). 1H NMR (400 MHz, DMSO-d6) δ 13.03 (s, 1H), 7.86 (d, J=1.9 Hz, 1H), 7.65 (d, J=7.5 Hz, 1H), 7.54 (d, J=1.1 Hz, 1H), 7.26-7.18 (m, 1H), 6.34 (t, J=2.0 Hz, 1H), 5.37 (s, 2H). MS (ESI) m/e [M+H]+=237.1.

### Example B

### Example B1: Synthesis of 5-(tert-butyl)-2-methoxybenzenesulfonamide

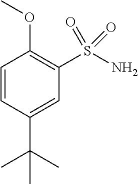 [SMILES: COc1ccc(C(C)(C)C)cc1S(N)(=O)=O]

To a solution of 1-(tert-butyl)-4-methoxybenzene (82 g, 0.5 mol) in DCM (200 mL) was added sulfurochloridic acid (115 mL, 1.75 mol) in DCM (120 mL) dropwise at 0° C. The mixture was stirred at 0° C. for 1 h and rt for 1 hr. The mixture was poured into ice water and extracted with DCM. The organic layer was dried over with Na2SO4 and filtered. The filtrate was concentrated, and the crude was redissolved in CH3CN (250 mL) and then NH3·H2O (80 mL) was added dropwise at 0° C. The mixture was stirred at rt for 10 hrs. The solvent was removed, and the crude was purified by silica gel chromatography column to give the desired product (70 g, 57%). 1H NMR (399 MHz, CDCl3) δ 7.93-7.89 (m, 1H), 7.55 (d, J=8.7, 1H), 6.98 (d, J=8.7 Hz, 1H), 5.08 (s, 2H), 3.99 (s, 3H), 1.40-1.23 (m, 9H). MS (ESI) m/e [M+H]+=244.1.

### Example B2: Synthesis of 6-(tert-butyl)-3-methoxypyridine-2-sulfonamide

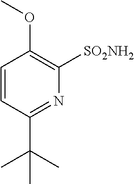 [SMILES: COc1ccc(C(C)(C)C)nc1S(N)(=O)=O]

### Step 1: 6-(tert-butyl)-2-fluoro-3-methoxypyridine

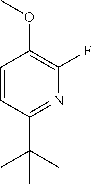

To a mixture of CuI (18.5 g, 97 mmol) and tert-butylmagnesium bromide (1 M in THF, 97 mL) was added a solution of 6-bromo-2-fluoro-3-methoxypyridine (5 g, 24 mmol) in THF (3 mL) dropwise at 0° C. The reaction mixture was allowed to warm up and stirred at 20° C. for 16 h. The reaction was quenched by saturated aq. NH4Cl (150 ml) and extracted with EA (100 ml×3). The combined organic layers were washed with brine, dried over anhydrous Na2SO4, filtered and concentrated in vacuum. The residue was purified by silica gel column chromatography (PE:EA=100/1 to 0/1) to give the title compound (3.4 g, 77% yield). MS(ESI) m/e [M+H]+=184.4.

### Step 2: 2-(benzylthio)-6-(tert-butyl)-3-methoxypyridine

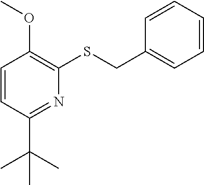 [SMILES: COc1ccc(C(C)(C)C)nc1SCc1ccccc1]

To a solution of phenylmethanethiol (3.5 g, 28 mmol) in THF (70 mL) was added NaH (60% in mineral oil, 1.12 g, 28 mmol) slowly and stirred for 30 min at 0° C. 6-(tert-butyl)-2-fluoro-3-methoxypyridine (3.4 g, 18.7 mmol) was added at 0° C. The reaction was stirred at 70° C. for 16 hr. The reaction mixture was poured into saturated aq.NH4Cl (100 ml), extracted by EA (300 ml×3). The combined organic layers were washed with brine, dried over anhydrous Na2SO4, filtered and concentrated in vacuum. The residue was purified by silica gel column chromatography (PE:EA=100/1 to 10/1) to give the title compound (4.05 g, 75% yield). 1H NMR: (400 MHz, CDCl3) δ 7.50-7.40 (m, 2H), 7.35-7.18 (m, 3H), 7.01-6.96 (m, 1H), 6.95-6.89 (m, 1H), 4.51 (s, 2H), 3.85 (s, 3H), 1.34 (s, 9H). MS(ESI) m/e [M+H]+=288.3.

### Step 3: 6-(tert-butyl)-3-methoxypyridine-2-sulfonamide

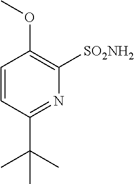 [SMILES: COc1ccc(C(C)(C)C)nc1S(N)(=O)=O]

To a solution of 2-(benzylthio)-6-(tert-butyl)-3-methoxypyridine (1.95 g, 6.8 mmol) in AcOH (18 mL) and H2O (6 mL) was added NCS (5.44 g, 40.7 mmol) at 20° C. The reaction was stirred at 20° C. for 1 hr. After addition of 7M NH3 in MeOH (9.7 mL), the reaction was stirred at 20° C. for another 1 hr. The reaction mixture was and then diluted with H2O 10 mL and extracted by EA (30 mL×3). The combined organic layers were washed with brine, dried over anhydrous Na2SO4, filtered and concentrated in vacuum. The combined organic layers were filtered and concentrated under reduced pressure. The residue was purified by silica gel column chromatography (PE/THF=100/1 to 0/1) to give the title compound (1 g, 62% yield). 1H NMR: (400 MHz, DMSO-d6) δ 7.65-7.55 (m, 2H), 7.16 (br s, 2H), 3.89 (s, 3H), 1.31 (s, 9H). MS(ESI) m/e [M+H]+=245.4.

### Example B3: Synthesis of 2-methoxy-5-(4-methoxypiperidin-1-yl)benzenesulfonamide

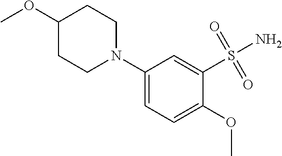 [SMILES: COc1ccc(N2CCC(OC)CC2)cc1S(N)(=O)=O]

A mixture of 5-bromo-2-methoxybenzenesulfonamide (250 mg, 0.94 mmol), 4-methoxypiperidine (218 mg, 1.88 mmol), Xphos-Pd-G4 (81 mg, 0.09 mmol) and t-BuONa (276 mg, 2.81 mmol) in dioxane (10 mL) was stirred at 120° C. for 2 h under nitrogen atmosphere. After cooled to room temperature, the resulting mixture was concentrated under vacuum. The residue was purified by flash chromatography on silica gel eluted with 5%-10% MeOH in DCM to give the desired product (200 mg, 71%). MS (ESI) m/e [M+H]+=301.

### Example B4: Synthesis of 6-methoxy-2,3-dihydro-1H-indene-5-sulfonamide

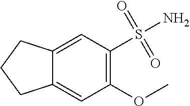

To a solution of methyl 5-methoxy-2,3-dihydro-1H-indene (148 mg, 1 mmol) in 10 ml DCM was added sulfuryl chloride (232 mg, 2 mmol) at 0° C. and stirred for 2 h at rt. The reaction mixture was washed by water. The organic phase was concentrated. The residue was dissolved in acetonitrile (5 mL), added 25% aq. NH3 (1 ml) and stirred at r.t for another 1 h. Upon completion of the reaction, the mixture was diluted with water, extracted by EA. The combined organic layers were washed with brine, dried over anhydrous Na2SO4, filtered, and concentrated to give the title compound (50 mg, 22% yield). MS (ESI) m/e [M+H]+=227.9.

### Example B5: Synthesis of 2-methoxy-5-(1-methoxycyclopropyl)benzenesulfonamide

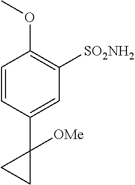 [SMILES: COc1ccc(C2(OC)CC2)cc1S(N)(=O)=O]

### Step 1: 1-(3-bromo-4-methoxyphenyl)cyclopropan-1-ol

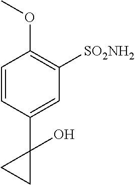 [SMILES: COc1ccc(C2(O)CC2)cc1S(N)(=O)=O]

A mixture of methyl 3-bromo-4-methoxy-benzoate (4 g, 16.3 mmol) and tetraisopropoxytitanium (6.5 g, 23 mmol) in THF (40 mL) was stirred for 30 min at 25° C. under N2, added bromo(ethyl)magnesium (1 M in THF, 45.7 mL) dropwise at 0° C. The reaction was stirred at 25° C. for 12 hr. The reaction mixture was quenched by H2O (40 mL), filtered and extracted with EA (40 mL×3). The combined organic layers were washed with brine, dried over anhydrous Na2SO4, filtered and concentrated under reduced. The residue was purified by silica gel column chromatography (PE:EA=1/0 to 3/1) to give the product (2.23 g, 56% yield). 1H NMR (400 MHz, CDCl3) δ 7.52 (d, J=2.4 Hz, 1H), 7.24 (dd, J=2.4, 8.8 Hz, 1H), 6.87 (d, J=8.8 Hz, 1H), 3.90 (s, 3H), 3.50 (s, 1H), 1.25-1.22 (m, 2H), 1.01-0.97 (m, 2H).

### Step 2: Synthesis of -methoxy-5-(1-methoxycyclopropyl)benzenesulfonamide

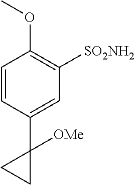 [SMILES: COc1ccc(C2(OC)CC2)cc1S(N)(=O)=O]

To a solution of 1-(3-bromo-4-methoxy-phenyl)cyclopropanol (2.58 g, 10.6 mmol) in THF (26 mL) was added NaH (60% in mineral oil, 424 mg, 10.6 mmol) and stirred for 30 min at 0° C. Mel (1.66 g, 11.7 mmol) was added dropwise. The reaction was stirred at 25° C. for 1 hr. The reaction mixture was quenched by H2O (20 mL). The aqueous phase was extracted with EA (20 mL×3). The combined organic phases were washed by brine, dried over anhydrous Na2SO4, filtered and concentrated in vacuum. The residue was purified by silica gel column chromatography (PE:EA=3:1) to give 2-bromo-1-methoxy-4-(1-methoxycyclopropyl)benzene (1 g). To a solution of 2-bromo-1-methoxy-4-(1-methoxycyclopropyl)benzene (500 mg, 1.94 mmol) in THF (5 mL) was added n-BuLi (1.6 M in Hexane, 1.2 mL) dropwise, and stirred at −60° C. for 30 min. The reaction was added sulfuryl chloride (262 mg, 1.94 mmol) at −60° C., stirred at 25° C. for 30 min, added 7M NH3 in MeOH (2.8 mL). The reaction was stirred at 25° C. for 2 hr. The reaction mixture was quenched by H2O, extracted with EA (5 mL×3). The combined organic layers were washed with brine, dried over anhydrous Na2SO4, filtered and concentrated under reduced pressure. The residue was purified by prep-HPLC (column: Waters Xbridge Prep OBD C18; mobile phase: [H2O (10 mM NH4HCO3)-ACN]; gradient: 15%-55% B over 8.0 min) to give the title compound (152 mg, 30% yield). 1H NMR (400 MHz, DMSO-d6) δ 7.67 (d, J=2.4 Hz, 1H), 7.45 (dd, J=2.4, 8.8 Hz, 1H), 7.18 (d, J=8.8 Hz, 1H), 7.05 (s, 2H), 3.89 (s, 3H), 3.10 (s, 3H), 1.15-1.10 (m, 2H), 0.92-0.88 (m, 2H). MS(ESI) m/e [M−H]−=225.9.

### Example B6: Synthesis of 6-methoxy-3,3-dimethyl-2,3-dihydrobenzofuran-7-sulfonamide

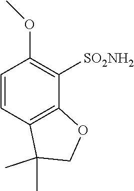 [SMILES: COc1ccc2c(c1S(N)(=O)=O)OCC2(C)C]

### Step 1: 1-bromo-4-methoxy-2-((2-methylallyl)oxy)benzene

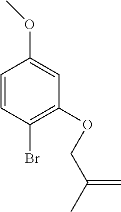

To a solution of 2-bromo-5-methoxy-phenol (5 g, 24.6 mmol) and K2CO3 (8.5 g, 61.6 mmol,) in ACN (50 mL) was added 3-bromo-2-methyl-prop-1-ene (3.66 g, 27.1 mmol) at 25° C., then the solution was stirred at 85° C. for 12 hr. The mixture was filtered and concentrated under reduced pressure to give a residue. The residue was purified by silica gel column chromatography (PE:EA=10/1) to give the title compound (6.3 g, 99% yield). 1H NMR: (400 MHz, CDCl3) δ 7.42 (d, J=8.8 Hz, 1H), 6.48 (d, J=2.8 Hz, 1H), 6.41 (dd, J=2.8, 8.8 Hz, 1H), 5.17 (s, 1H), 5.03 (s, 1H), 4.48 (s, 2H), 3.79 (s, 3H), 1.86 (s, 3H).

### Step 2: 6-methoxy-3,3-dimethyl-2,3-dihydrobenzofuran

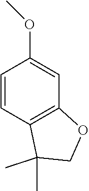

To a solution of 1-bromo-4-methoxy-2-(2-methylallyloxy)benzene (3 g, 11.6 mmol) in toluene (30 mL) was added sequentially Bu3SnH (5.1 g, 17.50 mmol) and AIBN (250 mg, 1.5 mmol) at 25° C., then the solution was stirred at 110° C. for 12 hr. The reaction mixture was cooled down to room temperature and quenched by aq. KF, extracted with DCM (30 mL×3). The combined organic layers were washed with brine, dried over anhydrous Na2SO4, filtered, and concentrated under reduced pressure. The residue was purified by silica gel column chromatography (PE:EA=1/0 to 3/1) to give the title compound (1.78 g, 86% yield). 1H NMR: (400 MHz, CDCl3) δ 6.98 (d, J=8.4 Hz, 1H), 6.44 (dd, J=2.0, 8.4 Hz, 1H), 6.40 (d, J=2.0 Hz, 1H), 4.25 (s, 2H), 3.78 (s, 3H), 1.33 (s, 6H).

### Step 3: 7-bromo-6-methoxy-3,3-dimethyl-2,3-dihydrobenzofuran

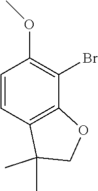

To a solution of 6-methoxy-3,3-dimethyl-2H-benzofuran (1.43 g, 8 mmol) in THF (28 mL) was added n-BuLi (1.6 M in Hexane, 6.5 mL) at 0° C. The reaction mixture was refluxed at 45° C. for 90 minutes. Subsequently, the mixture was cooled to 0° C. and a solution of 1,2-dibromo-1,1,2,2-tetrachloro-ethane (5.23 g, 16 mmol) was added. The resulting mixture was stirred for 12 hr. The solution was quenched by H2O (30 mL), extracted with EA (30 mL×3). The combined organic layers were washed with brine, dried over Na2SO4, filtered and concentrated under reduced pressure. The residue was purified by silica gel column chromatography (PE:EA=1/0 to 0/1) to give the title compound (1.2 g, 58% yield). 1H NMR: (400 MHz, CDCl3) δ 6.95 (d, J=8.4 Hz, 1H), 6.44 (d, J=8.4 Hz, 1H), 4.36 (s, 2H), 3.88 (s, 3H), 1.35 (s, 6H).

### Step 4: 6-methoxy-3,3-dimethyl-2,3-dihydrobenzofuran-7-sulfonamide

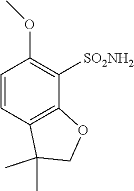 [SMILES: COc1ccc2c(c1S(N)(=O)=O)OCC2(C)C]

To a solution of 7-bromo-6-methoxy-3,3-dimethyl-2H-benzofuran (600 mg, 2.3 mmol) in THF (6 mL) under N2, the mixture was cooled to −60° C., followed by the addition of a solution of n-BuLi (1.6 M in n-hexane, 1.46 mL) dropwise. The mixture was stirred at −60° C. for 30 min. To the reaction mixture was added sulfuryl chloride (315 mg, 2.3 mmol) at −60° C. and the solution was stirred at 25° C. for 30 min. The reaction mixture was added 7 M NH3 in MeOH (3.33 mL) and stirred at 25° C. for 1 hr. The reaction was quenched by H2O, extracted with EA (5 mL×3). The combined organic layers were washed by brine, dried over Na2SO4, filtered and concentrated under reduced pressure. The residue was purified by prep-HPLC (column: Waters Xbridge Prep OBD C18 150×40 mm×10 um; mobile phase: [H2O (10 mM NH4HCO3)-ACN]; gradient: 15%-55% B over 8.0 min) to give the title compound (100 mg, 16% yield). 1H NMR: (400 MHz, DMSO-d6) δ 7.29 (d, J=8.4 Hz, 1H), 6.98 (s, 2H), 6.61 (d, J=8.4 Hz, 1H), 4.27 (s, 2H), 3.82 (s, 3H), 1.27 (s, 6H). MS(ESI) m/e [M−H]−=256.0.

### Example B7: synthesis of 6-methoxy-2,2-dimethyl-2,3-dihydrobenzofuran-7-sulfonamide

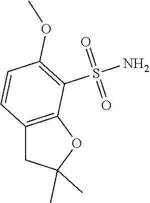 [SMILES: COc1ccc2c(c1S(N)(=O)=O)OC(C)(C)C2]

### Step 1: methyl 2-(3-methoxyphenoxy)acetate

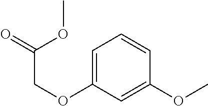

A solution of 3-methoxyphenol (3.00 g, 24.19 mmol), methyl 2-bromoacetate (4.41 g, 29.03 mmol), Cs2CO3 (15.77 g, 48.38 mmol) in 10 ml DMF was stirred at rt for overnight, Upon completion of the reaction, the mixture was concentrated and purified by silica gel column chromatography (PE/EA=5:1) to give the product (3.80 g, 80%). MS (ESI) m/e [M+H]+=197.2.

### Step 2: 1-(3-methoxyphenoxy)-2-methylpropan-2-ol

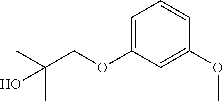

To a solution of methyl 2-(3-methoxyphenoxy)acetate (3.80 g, 19.39 mmol) in 20 ml THF was added CH3MgBr (3M, 12.93 ml, 38.78 mmol) at 0° C. and stirred for 2 h, Upon completion of the reaction, the solution was diluted with water and extracted with EA. The combined organic layers were washed with brine, dried over Na2SO4, filtered and concentrated. The residue was purified by silica gel column chromatography (PE/EA=5:1) to give the title compound (3.80 g, 100%). MS (ESI) m/e [M+H]+=197.1.

### Step 3: 6-methoxy-2,2-dimethyl-2,3-dihydrobenzofuran

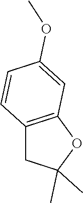

To a solution of 1-(3-methoxyphenoxy)-2-methylpropan-2-ol (3.80 g, 19.38 mmol) in 10 ml methanesulfonic acid was added P2O5 (8.66 g, 61.00 mmol) at rt and stirred for 2 hr, Upon completion of the reaction, the solution was diluted with water and extracted with EA. The combined organic layers were washed with brine, dried over Na2SO4, filtered and concentrated. The residue was purified by silica gel column chromatography (PE/EA=10:1) to give the title compound (1 g, 29%). 1H NMR (400 MHz, CDCl3) δ 7.00 (d, J=8.0 Hz, 1H), 6.38 (d, J=8.0 Hz, 1H), 6.34 (s, 1H), 3.76 (s, 3H), 2.94 (s, 2H). MS (ESI) m/e [M+H]+=179.1.

### Step 4: 6-methoxy-2,2-dimethyl-2,3-dihydrobenzofuran-7-sulfinic acid

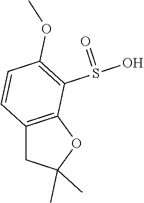 [SMILES: COc1ccc2c(c1S(=O)O)OC(C)(C)C2]

To a solution of 6-methoxy-2,2-dimethyl-2,3-dihydrobenzofuran (1.78 g, 3.00 mmol), TMEDA (1.16 g, 10 mmol) in 30 ml hexane was added n-BuLi (12 ml, 30 mmol) at 0° C. and stirred for 10 min, the mixture was cooled to −78° C. and SO2 was bobbled for 5 min. Upon completion of the reaction, the mixture was quenched by water. The solution was concentrated and purified by reversed phase column chromatography (Column=C18 spherical 20-35 um; mobile phase: [water (0.1% FA)-ACN], B %=5%-80%) to give the title compound (1.80 g, 74%). MS (ESI) m/e [M+Na]+=243.2.

### Step 5: 6-methoxy-2,2-dimethyl-2,3-dihydrobenzofuran-7-sulfonyl chloride

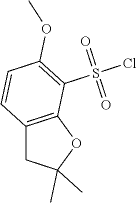 [SMILES: COc1ccc2c(c1S(=O)(=O)Cl)OC(C)(C)C2]

To a solution of 6-methoxy-2,2-dimethyl-2,3-dihydrobenzofuran (1.80 g, 7.4 mmol) in 10 ml DCM was added sulfuryl dichloride (992 mg, 7.4 mmol) dropwise at 0° C. and stirred for 1 hr. Upon completion of the reaction, the mixture was poured into ice water and extracted with DCM. The organic layer was concentrated to give the title compound (1.80 g, 88%). MS (ESI) m/e [M+Na]+=276.1.

### Step 6: 6-methoxy-2,2-dimethyl-2,3-dihydrobenzofuran-7-sulfonamide

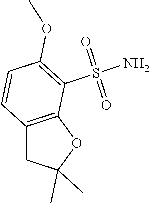 [SMILES: COc1ccc2c(c1S(N)(=O)=O)OC(C)(C)C2]

To a solution of 6-methoxy-2,2-dimethyl-2,3-dihydrobenzofuran-7-sulfonyl chloride (200 mg, 0.72 mmol) in 1 ml acetonitrile was added NH3·H2O (1 mL) dropwise at 0° C. The reaction mixture was stirred at rt for 1 h. Upon completion of the reaction. The reaction mixture was added H2O and extracted with EA. The combined organic layers were washed with brine, dried over Na2SO4, filtered and concentrated. The residue was purified by silica gel column chromatography (PE/EA=1:1) to give the title compound (100 mg, 54%). 1H NMR (400 MHz, CDCl3) δ 7.17 (d, J=8.2 Hz, 1H), 6.43 (d, J=8.2 Hz, 1H), 5.24 (s, 2H), 3.93 (s, 3H), 2.95 (s, 2H), 1.52 (s, 6H). MS (ESI) m/e [M+H]+=258.1.

### Example B8: Synthesis of 7-methoxy-4,4-dimethylchromane-8-sulfonamide

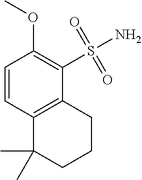 [SMILES: COc1ccc2c(c1S(N)(=O)=O)CCCC2(C)C]

### Step 1: 1-bromo-4-methoxy-2-((3-methylbut-2-en-1-yl)oxy)benzene

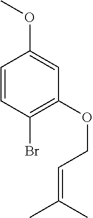

A mixture of 2-bromo-5-methoxyphenol (3.00 g, 14.78 mmol), 3-methylbut-2-en-1-ol (1.50 g, 17.73 mmol), PPh3 (5.80 g, 22.17 mmol) and di-tert-butyl-diazene-1,2-dicarboxylate (5.10 g, 22.17 mmol) in THF (30 mL) was stirred at 70° C. for 16 h under nitrogen atmosphere. After cooled to room temperature, the resulting mixture was concentrated under vacuum. The residue was purified by flash chromatography on silica gel eluted with 10%-40% EA in PE to give the desired product (2.00 g, 50%). MS (ESI) m/e [M+H]+=271.

### Step 2: 7-methoxy-4,4-dimethylchromane

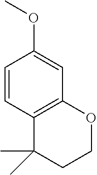

A mixture of 1-bromo-4-methoxy-2-((3-methylbut-2-en-1-yl)oxy)benzene (800 mg, 2.96 mmol), NaOAc (728 mg, 8.89 mmol), Sodium formate (304 mg, 4.44 mmol), tetraethylammonium chloride (732 mg, 4.44 mmol) and Pd(OAc)2 (331 mg, 1.48 mmol) in DMF (30 mL) was stirred at 90° C. for 16 h under nitrogen atmosphere. After cooled to room temperature, the resulting mixture was concentrated under vacuum. The residue was purified by flash chromatography on silica gel eluted with 5%-20% EA in PE to give the desired product (280 mg, 49%). MS (ESI) m/e [M+H]+=193.

### Step 3: 7-methoxy-4,4-dimethylchromane-8-sulfinic acid

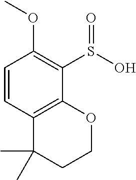 [SMILES: COc1ccc2c(c1S(=O)O)OCCC2(C)C]

To a stirred mixture of 7-methoxy-4,4-dimethylchromane (280 mg, 1.46 mmol) and TMEDA (169 mg, 1.46 mmol) in n-hexane (15 mL) was added n-BuLi (2.5M in n-hexane) (1.75 ml, 4.38 mmol) dropwise at 0° C. under nitrogen atmosphere. The resulting mixture was stirred at 0° C. for 30 min. The mixture was cooled to −78° C. and SO2 was bubbled for 5 min. Upon completion of the reaction, the mixture was quenched by water and extracted with DCM. The combined organic layers were washed with brine, dried over Na2SO4, filtered, and concentrated to give the desired product (200 mg, 53%). MS (ESI) m/e [M+H]+=257.

### Step 4: 7-methoxy-4,4-dimethylchromane-8-sulfonyl chloride

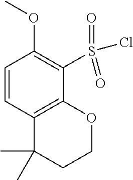 [SMILES: COc1ccc2c(c1S(=O)(=O)Cl)OCCC2(C)C]

To a stirred mixture of 7-methoxy-4,4-dimethylchromane-8-sulfinic acid (200 mg, 0.78 mmol) in DCM (10 mL) was added SO2Cl2 (105 mg, 0.78 mmol) dropwise at 0° C. The resulting mixture was stirred at 0° C. for 30 min. Upon completion of the reaction, the reaction mixture was added H2O and extracted with EA. The combined organic layers were washed with brine, dried over Na2SO4, filtered, and concentrated to give the desired product (200 mg, 88%). MS (ESI) m/e [M+H]+=291.

### Step 5: 7-methoxy-4,4-dimethylchromane-8-sulfonamide

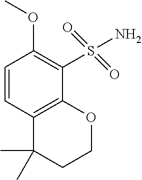 [SMILES: COc1ccc2c(c1S(N)(=O)=O)OCCC2(C)C]

To a solution of 7-methoxy-4,4-dimethylchromane-8-sulfonyl chloride (200 mg, 0.69 mmol) in THF (5 mL) was added NH3·H2O (2 mL) dropwise at 0° C. The reaction mixture was stirred at room temperature for 1h. Upon completion of the reaction, the reaction mixture was added H2O and extracted with EA. The combined organic layers were washed with brine, dried over Na2SO4, filtered and concentrated. The residue was purified by silica gel column chromatography (PE/EA=1:1) to give the desired product (100 mg, 53%). 1H NMR (400 MHz, DMSO-d6) δ 7.39 (d, J=8.0 Hz, 1H), 6.82 (brs, 2H), 6.61 (d, J=8.0 Hz, 1H), 4.25-4.06 (m, 2H), 3.71 (s, 3H), 1.78-1.62 (m, 2H), 1.20 (s, 6H). MS (ESI) m/e [M+H]+=272.

### Example B9: Synthesis of 3-methoxy-6-(1,1,1-trifluoro-2-methylpropan-2-yl)pyridine-2-sulfonamide

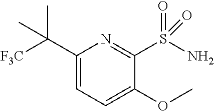 [SMILES: COc1ccc(C(C)(C)C(F)(F)F)nc1S(N)(=O)=O]

### Step 1: 1-(6-bromo-5-methoxypyridin-2-yl)ethan-1-one

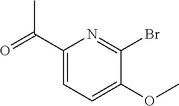

To a solution of 2-bromo-6-iodo-3-methoxypyridine (30 g, 95.5 mmol) and N-methoxy-N-methylacetamide (10.8 g, 105 mmol) in THF (300 mL) was added iPrMgCl (48 mL, 96 mmol) at −40° C. dropwise under N2. The mixture was stirred at −40° C. for 2h. Upon completion of the reaction, the mixture was quenched by H2O. The mixture was extracted with EA. The organic layer was concentrated, and the crude was purified by silica gel column chromatography (PE/EA=5:1) to give the title compound (21.1 g, 96%). MS (ESI) m/e [M+H]+=230.

### Step 2: 2-bromo-3-methoxy-6-(1,1,1-trifluoro-2-((trimethylsilyl)oxy)propan-2-yl)pyridine

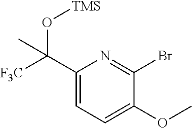 [SMILES: COc1ccc(C(C)(O[Si](C)(C)C)C(F)(F)F)nc1Br]

To a solution of 1-(6-bromo-5-methoxypyridin-2-yl)ethan-1-one (21 g, 91.3 mmol) and KOAc (8.9 g, 91.3 mmol) in DMSO (100 mL) was added TMSCF3 (26 g, 183 mmol) dropwise at 0° C. The mixture was stirred at rt for 5h. Upon completion of the reaction, the mixture was quenched by H2O, extracted with EA. The organic layer was dried and concentrated to give the crude. The crude was used in next step without purification (34 g, crude). MS (ESI) m/e [M+H]+=374.

### Step 3: 2-(6-bromo-5-methoxypyridin-2-yl)-1,1,1-trifluoropropan-2-ol

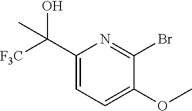 [SMILES: COc1ccc(C(C)(O)C(F)(F)F)nc1Br]

A solution of 2-bromo-3-methoxy-6-(1,1,1-trifluoro-2-((trimethylsilyl)oxy)propan-2-yl)pyridine (34 g, 91.3 mmol) and K2CO3 (25.3 g, 183 mmol) in MeOH (200 mL) was stirred at rt for 2h. Upon completion of the reaction, the mixture was filtered and concentrated. The crude was purified by silica gel column chromatography (PE/EA=3:1) to give the title compound (22 g, 80.3%). MS (ESI) m/e [M+H]+=300.

### Step 4: 2-(6-bromo-5-methoxypyridin-2-yl)-1,1,1-trifluoropropan-2-yl methanesulfonate

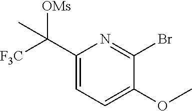 [SMILES: COc1ccc(C(C)(OS(C)(=O)=O)C(F)(F)F)nc1Br]

To a solution of 2-(6-bromo-5-methoxypyridin-2-yl)-1,1,1-trifluoropropan-2-ol (22 g, 73.3 mmol) in DCM (200 mL) was added MsCl (11 g, 95.3 mmol) dropwise at 0° C., following by TEA (14.8 g, 147 mmol). The mixture was stirred at 0° C. for 2h. Upon completion of the reaction, the mixture was washed with H2O. The organic layer was dried and concentrated. The crude was used in next step without purification (27 g, crude). MS (ESI) m/e [M+H]+=378.

### Step 5: 2-bromo-3-methoxy-6-(1,1,1-trifluoro-2-methylpropan-2-yl)pyridine

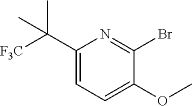 [SMILES: COc1ccc(C(C)(C)C(F)(F)F)nc1Br]

To a solution of 2-(6-bromo-5-methoxypyridin-2-yl)-1,1,1-trifluoropropan-2-yl methanesulfonate (27 g, crude) in DCM (200 mL) was added AlMe3 (73.3 mL, 146.6 mmol) at 0° C. under N2. The mixture was stirred at 0° C. for 3h. Upon completion of the reaction, the mixture was quenched by H2O. The organic layer was dried and concentrated. The crude was purified by silica gel column chromatography (PE/EA=10:1) to give the title compound (9 g, 41.2%). MS (ESI) m/e [M+H]+=298.

### Step 6: 3-methoxy-6-(1,1,1-trifluoro-2-methylpropan-2-yl)pyridine-2-sulfonamide

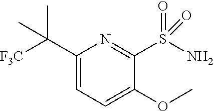 [SMILES: COc1ccc(C(C)(C)C(F)(F)F)nc1S(N)(=O)=O]

To a solution of 2-bromo-3-methoxy-6-(1,1,1-trifluoro-2-methylpropan-2-yl)pyridine (9 g, 30.2 mmol) in THF (200 mL) was added n-BuLi (13.3 mL, 33.2 mmol) at −78° C. under N2. The mixture was stirred at −78° C. for 0.5h. Then SO2 (gas) was bubbled into the reaction below −40° C. Upon completion of the reaction, the mixture was quenched by H2O (20 mL). The mixture was concentrated to dry. The crude was redissolved in NH3·H2O (12.5%) (70 mL) and THF (70 mL), 12 (7.5 g, 60 mmol) was added at r.t portion wise. The mixture was stirred at rt for 1 h. Then the mixture was diluted by EA. The organic layer was washed with H2O, dried and concentrated. The crude was purified by silica gel column chromatography (PE/EA=2:1) to give the title compound (4.9 g, 54.4%). 1H NMR (400 MHz, CDCl3) δ 7.64 (d, J=8.7 Hz, 1H), 7.40 (d, J=8.8 Hz, 1H), 5.14 (s, 2H), 3.99 (s, 3H), 1.57 (s, 6H). MS (ESI) m/e [M+H]+=299.

### Example B10: Synthesis of 2-methoxy-5-(1-(trifluoromethyl)cyclopropyl) benzenesulfonamide

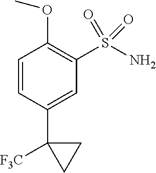 [SMILES: COc1ccc(C2(C(F)(F)F)CC2)cc1S(N)(=O)=O]

1-Methoxy-4-[1-(trifluoromethyl)cyclopropyl]benzene (5 g, 23 mmol) was added to chlorosulfonic acid (13.5 g, 115 mmol) at 0° C. dropwise under N2 atmosphere. The reaction was stirred at this temperature for 1 hr. The reaction mixture was added into ice-water dropwise. The precipitate was filtered and purified by silica gel column chromatography (PE:EA=3:1) to give the 2-methoxy-5-(1-(trifluoromethyl)cyclopropyl) benzenesulfonyl chloride (3.0 g, 41%). To a solution of 2-methoxy-5-(1-(trifluoromethyl) cyclopropyl) benzenesulfonyl chloride (2.0 g, 6.4 mmol) in THF (20 mL) was added NH3·H2O (10.0 mL) dropwise at 0° C. The reaction mixture was stirred at room temperature for 1 hr. The reaction mixture was added water and extracted with EA. The combined organic layers were washed with brine, dried over Na2SO4, filtered and concentrated. The residue was purified by silica gel column chromatography (PE:EA=1:1) to give the title compound (1 g, 53%). 1H NMR (400 MHz, DMSO-d6): δ 7.78 (d, J=2.4 Hz, 1H), 7.65 (d, J=8.4, 1H), 7.22 (d, J=8.8 Hz, 1H), 7.15 (s, 2H), 3.91 (s, 3H), 1.43-1.30 (m, 2H), 1.12-1.08 (m, 2H). MS (ESI) m/e [M+H]+=296.1.

### Example B11: Synthesis of 2-methoxy-5-(1-methylcyclobutyl)benzenesulfonamide

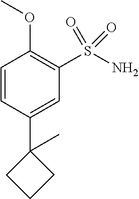 [SMILES: COc1ccc(C2(C)CCC2)cc1S(N)(=O)=O]

### Step 1: 1-(4-methoxyphenyl)cyclobutan-1-ol

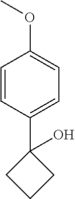

To a solution of 1-bromo-4-methoxybenzene (3.0 g, 16.1 mmol) in THF (50 mL) was added n-BuLi (2.5M, 7.1 mL, 17.7 mmol) dropwised at −70° C., the mixture was stirred at −70° C. for 30 mins. cyclobutanone (1.36 g, 19.4 mmol) in THF (5 mL) was added at −70° C., the resulting mixture was stirred at −70° C. for 1h. After warmed to rt, the mixture was quenched by sat.NH4Cl and extracted with EA, the organic layer was washed with brine, dried over Na2SO4 and concentrated. The crude was purified by silica gel column chromatography (PE:EA=1:3) to give the desired product (2.57 g, 90%).

### Step 2: 1-methoxy-4-(1-methylcyclobutyl)benzene

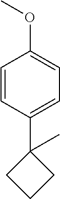

To a solution of 1-(4-methoxyphenyl)cyclobutan-1-ol (2.5 g, 14.0 mmol) in DCM (100 mL) was added TiCl4 (5.34 g, 28.1 mmol) at −70° C., the mixture was stirred at −70° C. for 1h. ZnMe2 (LOM, 42.1 mL, 42.1 mmol) was added dropwised at −70° C., the resulting mixture was stirred at −70° C. for 2 h. After warmed to rt, the mixture was quenched by brine and extracted with EA, the organic layer was dried over Na2SO4 and concentrated to give the desired product (2.4 g, 97%).

### Step 3: 2-methoxy-5-(1-methylcyclobutyl)benzenesulfonic acid

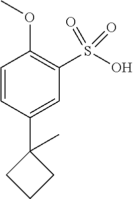 [SMILES: COc1ccc(C2(C)CCC2)cc1S(=O)(=O)O]

To a solution of 1-methoxy-4-(1-methylcyclobutyl)benzene (2.2 g, 12.5 mmol) in DCM (50 mL) was added ClSO3H (1.8 g, 15.0 mmol) in DCM (10 mL) at −10° C., the resulting mixture was stirred at −10° C. for 1h. The mixture was quenched by ice water and concentrated. The crude was purified by silica C18 (ACN/H2O=15:85) to give the desired product (3.2 g, 100%). MS (ESI) m/e [M−H]−=255.

### Step 4: 2-methoxy-5-(1-methylcyclobutyl)benzenesulfonyl chloride

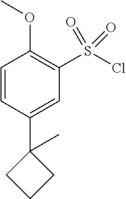 [SMILES: COc1ccc(C2(C)CCC2)cc1S(=O)(=O)Cl]

A solution of 2-methoxy-5-(1-methylcyclobutyl)benzenesulfonic acid (3.1 g, 12.1 mmol) in SOCl2 (40 mL) was stirred at 85° C. for 2 h. The mixture was concentrated to give the crude product (3.2 g).

### Step 5: 2-methoxy-5-(1-methylcyclobutyl)benzenesulfonamide

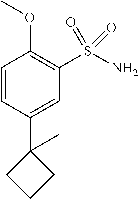 [SMILES: COc1ccc(C2(C)CCC2)cc1S(N)(=O)=O]

To a solution of 2-methoxy-5-(1-methylcyclobutyl)benzenesulfonyl chloride (3.2 g, 11.7 mmol) in THF (50 mL) was added NH3·H2O (20 mL), the resulting mixture was stirred at rt for 30 mins. The reaction was extracted with EA, the organic layer was washed with brine, dried over Na2SO4 and concentrated. the crude was purified by silica gel column chromatography (PE:EA=7:3) to give the desired product (950 mg, 32%). MS (ESI) m/e [M+H]+=256.

### Example B12: Synthesis of 2-methoxy-5-(3-methyloxetan-3-yl)benzenesulfonamide

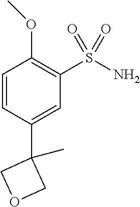 [SMILES: COc1ccc(C2(C)COC2)cc1S(N)(=O)=O]

### Step 1: 1,3-dioxoisoindolin-2-yl 3-methyloxetane-3-carboxylate

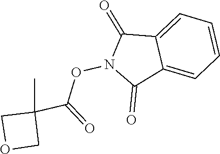 [SMILES: CC1(C(=O)ON2C(=O)c3ccccc3C2=O)COC1]

To a solution of 3-methyloxetane-3-carboxylic acid (1 g, 8.6 mmol) and 2-hydroxyisoindoline-1,3-dione (1.4 g, 8.6 mmol) in DCM (80 mL) was added DMAP (105 mg, 861 μmol) and DIC (1.2 g, 9.4 mmol). The reaction was stirred at 25° C. for 12 h. The reaction mixture was concentrated in vacuum. The residue was purified by silica gel column chromatography (PE:EA=1:1) to give a crude residue. The residue was triturated with MeOH (8 mL) to give the title compound (1.3 g, 58% yield). 1H NMR: (400 MHz, CDCl3) δ 7.94-7.89 (m, 2H), 7.84-7.80 (m, 2H), 5.19 (d, J=6.4 Hz, 2H), 4.56 (d, J=6.4 Hz, 2H), 1.86 (s, 3H).

### Step 2: 3-(4-methoxyphenyl)-3-methyloxetane

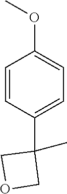

A solution of NiBr2·glyme (83 mg, 268 μmol) and 4,4-di-tert-butyl-N-cyano-[2,2-bipyridine]-6-carboximidamide (90 mg, 268 μmol) in THF (5 mL) was stirred at 25° C. for 0.5 h used glovebox, resulting in a homogeneous solution. 1,3-dioxoisoindolin-2-yl 3-methyloxetane-3-carboxylate (1 g, 3.8 mmol), 1-iodo-4-methoxybenzene (895.9 mg, 3.8 mmol) and Zn (0.9 g, 13.7 mmol, 3.6 eq) in the other flask was added 5 mL of the prepared catalyst solution. The reaction was stirred at 35° C. for 16 h. The reaction mixture was diluted with DCM (10 mL), filtered, and concentrated in vacuum. The residue was purified by silica gel column chromatography (PE:EA=1:0 to 3:1) to give the title compound (390 mg, 57% yield). 1H NMR: (400 MHz, CDCl3) δ 7.18-7.14 (m, 2H), 6.92-6.89 (m, 2H), 4.95 (d, J=5.6 Hz, 2H), 4.63 (d, J=5.6 Hz, 2H), 3.82 (s, 3H), 1.72 (s, 3H).

### Step 3: 3-(3-bromo-4-methoxyphenyl)-3-methyloxetane

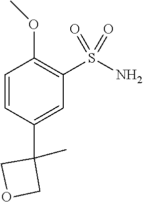 [SMILES: COc1ccc(C2(C)COC2)cc1S(N)(=O)=O]

To a solution of 3-(4-methoxyphenyl)-3-methyloxetane (0.3 g, 1.6 mmol, 1 eq) in ACN (3 mL) was added NBS (299.6 mg, 1.7 mmol, 1.05 eq) at 25° C. under N2. The mixture was stirred at 25° C. for 12 h. The mixture was concentrated in vacuum. The residue was purified by silica gel column chromatography (PE:EA=1:0 to 3:1) to give 3-(3-bromo-4-methoxyphenyl)-3-methyloxetane (0.37 g). To a solution of 3-(3-bromo-4-methoxyphenyl)-3-methyloxetane (0.3 g, 1.2 mmol) in THF (3 mL) was added dropwise n-BuLi (2.5 M in hexane, 467 L) at −60° C. under N2. The reaction was stirred at −60° C. for 30 min. To the reaction mixture was added sulfuryl chloride (157.4 mg, 1.2 mmol) at −45° C. and the reaction was stirred at −45° C. for 30 min. 7M NH3 in MeOH (1.7 mL) was added. The reaction was stirred at 25° C. for 10 min. The reaction mixture was concentrated in vacuum. The residue was purified by prep-HPLC (Column=Waters Xbridge Prep OBD C18 150×40 mm×10 um; mobile phase: [water (NH4HCO3)-MeCN], B %=2%-40%; 8.0 min) to give the title compound (61 mg, 19% yield). 1H NMR: (400 MHz, DMSO-d6) δ 7.58 (d, J=2.4 Hz, 1H), 7.47 (dd, J=2.4, 8.8 Hz, 1H), 7.20 (d, J=8.8 Hz, 1H), 6.03 (br s, 2H), 4.74 (d, J=5.6 Hz, 2H), 4.55 (d, J=5.6 Hz, 2H), 3.89 (s, 3H), 1.62 (s, 3H). MS(ESI) m/e [M−H]−=256.1.

### Example B13: Synthesis of 2-methoxy-5-(3-methoxyoxetan-3-yl)benzenesulfonamide

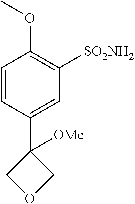 [SMILES: COc1ccc(C2(OC)COC2)cc1S(N)(=O)=O]

### Step 1: 3-(3-bromo-4-methoxyphenyl)oxetan-3-ol

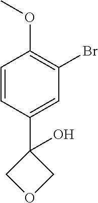

To a solution of 2-bromo-4-iodo-1-methoxy-benzene (10 g, 32 mmol), oxetan-3-one (3.45 g, 48 mmol) in THF (100 mL) was added dropwise n-BuLi (2.5 M in hexane, 15.3 mL, 38 mmol) at −65° C. The resulting mixture was stirred at −65° C. for 3 hr. Upon completion of the reaction, the mixture was quenched with saturated aqueous ammonium chloride (200 mL) and extracted with EA. The organic phase was washed with brine, dried over Na2SO4, filtered and concentrated in vacuum. The residue was purified by silica gel chromatography (PE:EA=50:0 to 10:1) to give the title compound (3.5 g, 30% yield). 1H NMR (400 MHz, DMSO-d6) δ=7.72 (d, J=2.3 Hz, 1H), 7.58 (dd, J=2.3, 8.5 Hz, 1H), 7.15 (d, J=8.5 Hz, 1H), 4.74 (d, J=6.8 Hz, 2H), 4.68-4.59 (d, J=6.8 Hz, 2H), 3.86 (s, 3H). MS(ESI) m/e [M−H]−=257, 259.

### Step 2: 3-(3-bromo-4-methoxyphenyl)-3-methoxyoxetane

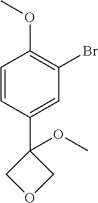

To a solution of 3-(3-bromo-4-methoxy-phenyl)oxetan-3-ol (2.4 g, 9.3 mmol) in THF (30 mL) was added portionwise NaH (60% in mineral oil, 560 mg, 14 mmol) at 0° C. After addition, the mixture was stirred at 0° C. for 30 min, and then Mel (2 g, 14 mmol) was added dropwise at 0° C. The resulting mixture was stirred at 20° C. for 4 hr. Upon completion of the reaction, mixture was poured into aq.NH4Cl (5 ml), and extracted with EA (3 mL×3). The combined organic layers were concentrated. The crude product was purified by silica gel chromatography (PE:EA=50:1 to 3:1) to give the title compound (2.85 g, crude). 1H NMR (400 MHz, DMSO-d6) δ=7.59 (d, J=2.1 Hz, 1H), 7.42 (dd, J=2.2, 8.6 Hz, 1H), 7.16 (d, J=8.5 Hz, 1H), 4.73 (s, 4H), 3.87 (s, 3H), 2.99 (s, 3H). MS(ESI) m/e [M-OMe]+=241, 243.

### Step 3: 3-(3-(benzylthio)-4-methoxyphenyl)-3-methoxyoxetane

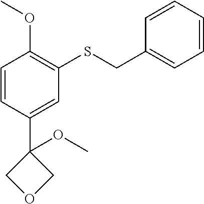 [SMILES: COc1ccc(C2(OC)COC2)cc1SCc1ccccc1]

A mixture of 3-(3-bromo-4-methoxy-phenyl)-3-methoxy-oxetane (1 g, 3.7 mmol), phenylmethanethiol (682 mg, 5.5 mmol), DIEA (946 mg, 7.3 mmol), Xantphos (424 mg, 732 μmol) and Pd2(dba)3 (335 mg, 366 μmol) in dioxane (15 mL) was degassed and purged with N2 for 3 times. The reaction was stirred at 110° C. for 16 hr under N2 atmosphere. Upon completion of the reaction, the mixture was quenched by H2O (30 ml) and extracted with EA. The combined organic layers were concentrated. The crude product was purified by silica gel chromatography (PE:EA=50:1 to 3:1) to give the title compound (1 g, 80% yield). 1H NMR (400 MHz, DMSO-d6) δ=7.36-7.31 (m, 2H), 7.30-7.23 (m, 2H), 7.23-7.17 (m, 2H), 7.16-7.14 (m, 1H), 7.03-6.97 (m, 1H), 4.71-4.63 (m, 4H), 4.18 (s, 2H), 3.84 (s, 3H), 2.86 (s, 3H). MS(ESI) m/e [M-OMe]+=285.3.

### Step 4: 2-methoxy-5-(3-methoxyoxetan-3-yl)benzenesulfonamide

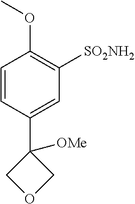 [SMILES: COc1ccc(C2(OC)COC2)cc1S(N)(=O)=O]

To a solution of 3-(3-benzylsulfanyl-4-methoxy-phenyl)-3-methoxy-oxetane (800 mg, 2.53 mmol) in DCM (15 mL), H2O (5 mL) was added TCCA (1.18 g, 5.06 mmol). The mixture was stirred at 0-15° C. for 16 hrs. Then the NH3/MeOH (7 M, 4.00 mL, 11.07 eq) was added. The mixture was stirred for 1 h. Upon completion of the reaction, the mixture was concentrated. The crude product was purified by silica gel chromatography (PE:EA=50:1 to 3:1) to give the title compound (276 mg, 39% yield). 1H NMR (400 MHz, DMSO-d6) δ=7.74 (d, J=2.3 Hz, 1H), 7.66 (dd, J=2.3, 8.6 Hz, 1H), 7.27 (d, J=8.6 Hz, 1H), 7.14 (s, 2H), 4.82-4.75 (m, 2H), 4.74-4.68 (m, 2H), 3.93 (s, 3H), 3.02 (s, 3H). MS(ESI) m/e [M−H]+=272.4.

### Example B14: Synthesis of 2-methoxy-5-(6-methyl-2-oxaspiro[3.3]heptan-6-yl)benzenesulfonamide

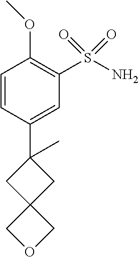 [SMILES: COc1ccc(C2(C)CC3(COC3)C2)cc1S(N)(=O)=O]

### Step 1: 6-(4-methoxyphenyl)-2-oxaspiro[3.3]heptan-6-ol

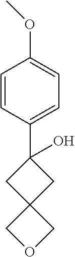 [SMILES: COc1ccc(C2(O)CC3(COC3)C2)cc1]

To a solution of 1-bromo-4-methoxybenzene (2.0 g, 10.8 mmol) in THF (30 mL) was added n-BuLi (2.5M, 4.5 mL, 11.3 mmol) dropwised at −70° C., the mixture was stirred at −70° C. for 30 mins. 2-oxaspiro[3.3]heptan-6-one (1.33 g, 11.9 mmol) in THF (5 mL) was added at −70° C., the resulting mixture was stirred at −70° C. for 1h. After warmed to rt, the mixture was quenched by sat.NH4Cl and extracted with EA, the organic layer was washed with brine, dried over Na2SO4 and concentrated. The crude was purified by silica gel column chromatography (PE:EA=1:1) to give the title compound (1.7 g, 72%).

### Step 2: 6-(4-methoxyphenyl)-6-methyl-2-oxaspiro[3.3]heptane

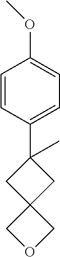 [SMILES: COc1ccc(C2(C)CC3(COC3)C2)cc1]

To a solution of 6-(4-methoxyphenyl)-2-oxaspiro[3.3]heptan-6-ol (800 mg, 3.64 mmol) in DCM (40 mL) was added TiCl4 (1.38 g, 7.27 mmol) at −70° C., the mixture was stirred at −70° C. for 1h. ZnMe2 (1.0M in toluene, 11 mL, 10.92 mmol) was added dropwise at −70° C., the resulting mixture was stirred at −70° C. for 2 h. After warmed to rt, the mixture was quenched by brine and extracted with EA, the organic layer was dried over Na2SO4 and concentrated to give the title compound (800 mg).

### Step 3: 6-(3-bromo-4-methoxyphenyl)-6-methyl-2-oxaspiro[3.3]heptane

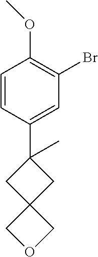 [SMILES: COc1ccc(C2(C)CC3(COC3)C2)cc1Br]

To a solution of 6-(4-methoxyphenyl)-6-methyl-2-oxaspiro[3.3]heptane (580 mg, 2.66 mmol) in ACN (20 mL) was added NBS (427 mg, 2.39 mmol), the resulting mixture was stirred at rt for overnight. The mixture was quenched by water and extracted with EA, the organic layer was washed with brine, dried over Na2SO4 and concentrated. The crude was purified by silica gel column chromatography (PE:EA=4:1) to give the title compound (460 mg, 58%).

### Step 4: 6-(3-(benzylthio)-4-methoxyphenyl)-6-methyl-2-oxaspiro[3.3]heptane

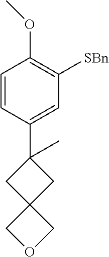

A solution of 6-(3-bromo-4-methoxyphenyl)-6-methyl-2-oxaspiro[3.3]heptane (360 mg, 1.22 mmol), phenylmethanethiol (166 mg, 1.34 mmol), Pd2dba3 (112 mg, 0.12 mmol), XantPhos (71 mg, 0.12 mmol) and TEA (247 mg, 2.44 mmol) in 1,4-Dioxane (10 mL) was stirred at 100° C. for overnight. After cooling to rt. the mixture was quenched by brine and extracted with EA, the organic layer was dried over Na2SO4 and concentrated, the crude was purified by silica gel column chromatography (PE:EA=3:1) to give the title compound (341 mg, 82%). MS (ESI) m/e [M+H]+=341.

### Step 5: 2-methoxy-5-(6-methyl-2-oxaspiro[3.3]heptan-6-yl)benzenesulfonamide

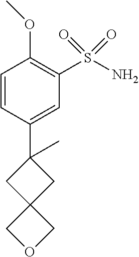 [SMILES: COc1ccc(C2(C)CC3(COC3)C2)cc1S(N)(=O)=O]

To a solution of 6-(3-(benzylthio)-4-methoxyphenyl)-6-methyl-2-oxaspiro[3.3]heptane (386 mg, 1.44 mmol) in ACN (8 mL) and water (2 mL) was added NCS (533 mg, 3.97 mmol), the resulting mixture was stirred at rt for 1 h. The mixture was added to THF (20 mL) and NH3·H2O (15 mL) at 0° C., the resulting mixture was stirred at rt for overnight. The reaction was extracted with EA, the organic layer was washed with brine, dried over Na2SO4 and concentrated. the crude was purified by silica gel column chromatography (PE:EA=7:3) to give the title compound (235 mg, 70%). MS (ESI) m/e [M+H]+=298.

### Example B15: Synthesis of 2-methoxy-5-(4-methyltetrahydro-2H-pyran-4-yl)benzenesulfonamide

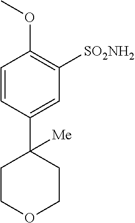 [SMILES: COc1ccc(C2(C)CCOCC2)cc1S(N)(=O)=O]

### Step 1: 4-(4-methoxyphenyl)tetrahydropyran-4-ol

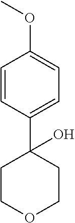

To a solution of 1-bromo-4-methoxybenzene (3 g, 16 mmol) in THF (30 mL) at −78° C. was added dropwise n-BuLi (2.5 M in Hexane, 7.7 mL) under N2 atmosphere. The resulting mixture was stirred at −78° C. for 1 h. Then tetrahydro-4H-pyran-4-one (1.93 g, 19.3 mmol) was added. The mixture was stirred at −78° C. for 2 hrs. The reaction mixture was quenched by addition of saturated aq. NH4Cl (50 mL) at 25° C. The aqueous phase was extracted with EA. The combined organic phase was washed with brine, dried over anhydrous Na2SO4, filtered and concentrated in vacuum. The residue was purified by silica gel column (PE:EA=1:0 to 0:1) to give the title compound (2.3 g, 69% yield). 1H NMR: (400 MHz, DMSO-d6) δ 7.39 (d, J=8.8 Hz, 2H), 6.86 (d, J=8.8 Hz, 2H), 4.90 (br s, 1H), 3.83-3.64 (m, 7H), 2.00-1.83 (m, 2H), 1.51 (d, J=12.8 Hz, 2H).

### Step 2: 4-(4-methoxyphenyl)-4-methyltetrahydro-2H-pyran

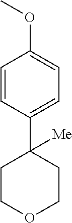

To a solution of 4-(4-methoxyphenyl)tetrahydro-2H-pyran-4-ol (2 g, 9.6 mmol) in DCM (50 mL) was added dropwise TiCl4 (3.64 g, 19.2 mmol) at −78° C. under N2 atmosphere. After addition, the mixture was stirred at −78° C. for 1 h, and then dimethylzinc (1 M in toluene, 38.4 mL) was added dropwise at −78° C. The resulting mixture was stirred at −78° C. for 2 hrs. The reaction mixture was added H2O (50 mL) and extracted with H2O (35 mL×3). The combined organic phases were washed with brine, dried over anhydrous Na2SO4, filtered and concentrated in vacuum. The residue was purified by silica gel column chromatography (PE:EA=1:0 to 0:1) to give the title compound (600 mg, 29% yield). 1H NMR: (400 MHz, DMSO-d6) δ 7.26 (d, J=8.8 Hz, 2H), 6.88 (d, J=8.8 Hz, 2H), 3.72 (s, 3H), 3.64-3.61 (m, 2H), 3.54-3.46 (m, 2H), 1.98-1.91 (m, 2H), 1.68-1.62 (m, 2H), 1.18 (s, 3H).

### Step 3: 2-methoxy-5-(4-methyltetrahydro-2H-pyran-4-yl)benzenesulfonamide

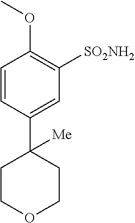 [SMILES: COc1ccc(C2(C)CCOCC2)cc1S(N)(=O)=O]

To a solution of 4-(4-methoxyphenyl)-4-methyltetrahydro-2H-pyran (500 mg, 2.4 mmol) in DCM (10 mL) was added sulfurochloridic acid (847 mg, 7.2 mmol) at 0° C. under N2 atmosphere. The mixture was stirred at 0° C. for 2 hrs. The reaction mixture was added H2O (10 mL) and extracted with DCM (7 mL×2). The combined organic phase was washed with brine (7 mL), dried over anhydrous Na2SO4, filtered, and concentrated in vacuum to give 2-methoxy-5-(4-methyltetrahydro-2H-pyran-4-yl)benzenesulfonic acid (150 mg, 22% yield). To a solution of 2-methoxy-5-(4-methyltetrahydro-2H-pyran-4-yl)benzenesulfonic acid (150 mg, 0.5 mmol) in DCM (2 mL) was added DMF (383 g) and (COCl)2 (100 mg, 0.75 mmol) at 0° C. under N2. The mixture was stirred at 20° C. for 2 h. The reaction mixture was concentrated under reduced pressure to give 2-methoxy-5-(4-methyltetrahydro-2H-pyran-4-yl)benzenesulfonyl chloride (150 mg, crude). A solution of crude 2-methoxy-5-(4-methyltetrahydro-2H-pyran-4-yl)benzenesulfonyl chloride (150 mg, 0.5 mmol) in 7M NH3 in MeOH (1 mL) was stirred at 20° C. for 10 min. DCM (10 mL) was added in the reaction mixture. The combined organic phases were washed with brine (2 mL), dried over anhydrous Na2SO4, filtered and concentrated in vacuum. The residue was purified by prep-HPLC (Column=Waters Xbridge Prep OBD C18 150×40 mm×10 um; mobile phase: [water (NH4HCO3)-ACN], B %=15%-50%; 8.0 min) to give the title compound (36 mg, 26% yield). MS(ESI) m/e [M-1]284.1. 1H NMR: (400 MHz, DMSO-d6) δ 7.67 (s, 1H), 7.56 (d, J=6.4 Hz, 1H), 7.16 (d, J=8.4 Hz, 1H), 7.05 (br s, 2H), 3.88 (s, 3H), 3.68-3.64 (m, 2H), 3.55-3.51 (m, 2H), 1.95-1.90 (m, 2H), 1.72-1.67 (m, 2H), 1.22 (s, 3H).

### Example B16: Synthesis of 2-methoxy-5-(4-methylmorpholin-3-yl)benzenesulfonamide

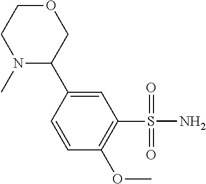 [SMILES: COc1ccc(C2COCCN2C)cc1S(N)(=O)=O]

### Step 1: 2-methoxy-5-(4-methylmorpholin-3-yl)benzenesulfonic acid

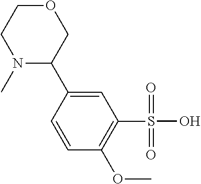 [SMILES: COc1ccc(C2COCCN2C)cc1S(=O)(=O)O]

To a solution of 3-(4-methoxyphenyl)-4-methyl-morpholine (1 g, 4.8 mmol) in DCM (20 mL) was added sulfurochloridic acid (1.7 g, 14.5 mmol) under N2 atmosphere at 0° C. The mixture was stirred at RT for 2 hr. Upon completion of the reaction, the mixture was quenched by MeOH (2 mL), and concentrated. The residue was purified by prep-HPLC (column: Phenomenex luna C18 (250*70 mm, 15 um); mobile phase: [H2O(0.04% HCl)-ACN]; B %: 0%, isocratic elution mode) to give the title compound (700 mg, 50% yield). MS(ESI) m/e [M+H]+=288.1.

### Step 2: 2-methoxy-5-(4-methylmorpholin-3-yl)benzenesulfonamide

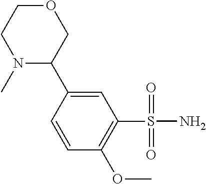 [SMILES: COc1ccc(C2COCCN2C)cc1S(N)(=O)=O]

To a solution of 2-methoxy-5-(4-methylmorpholin-3-yl)benzenesulfonic acid (600 mg, 2.1 mmol) in DCM (6 mL) was added DMF (15 mg, 210 μmol) and (COCl)2 (1.06 g, 8.4 mmol) at 0° C. The mixture was stirred at RT for 12 h, added 7 M NH3 in MeOH (3 mL), and stirred for another 0.5 h. Upon completion of the reaction the mixture was concentrated. The residue was purified by Prep-HPLC (column: WePure Biotech XP tC18; mobile phase: [H2O (10 mM NH4HCO3)-ACN]; gradient: 1%-30% B over 8.0 min) to give the title compound (242 mg, 40% yield). 1H NMR (400 MHz, DMSO-d6) δ=7.71 (s, 1H), 7.52 (d, J=8.0 Hz, 1H), 7.17 (d, J=8.0 Hz, 1H), 6.05 (br s, 2H), 3.89 (s, 3H), 3.82 (d, J=10.8 Hz, 1H), 3.66-3.53 (m, 2H), 3.16 (br t, J=10.8 Hz, 1H), 3.04 (br dd, J=2.4, 10.4 Hz, 1H), 2.82 (br d, J=12.0 Hz, 1H), 2.33-2.22 (m, 1H), 1.95 (s, 3H). MS(ESI) m/e [M−H]−=285.4.

### Example B17: Synthesis of Synthesis of 5-(8-oxa-3-azabicyclo[3.2.1]octan-3-yl)-2-methoxybenzenesulfonamide

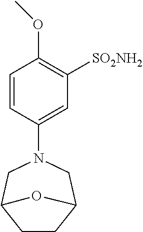 [SMILES: COc1ccc(N2CC3CCC(C2)O3)cc1S(N)(=O)=O]

To a mixture of 5-bromo-2-methoxybenzenesulfonamide (200 mg, 0.8 mmol) and 8-oxa-3-azabicyclo[3.2.1]octane (110 mg, 1.0 mmol) in dioxane (10 mL) was added Pd-PEPPSI-IPent (60 mg, 0.1 mmol) and t-BuOK (110 mg, 1.1 mmol). The resulting mixture was degassed with nitrogen and heated to 120° C. with stirring for 1 hr. The resulting reaction was cooled to room temperature. Solids were filtered out. The filtration was concentrated. The residue was applied onto a silica gel column chromatography (EA:PE to 12:1) to give the desired product (100 mg, 44%). MS (ESI) m/e [M+H]+=299.

### Example B18: Synthesis of 6-methoxy-2,3-dihydrobenzofuran-7-sulfonamide

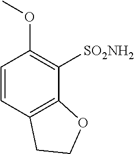

### Step 1: 1-bromo-2-(2-bromoethoxy)-4-methoxybenzene

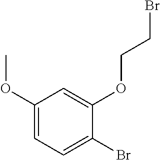

To a suspension of 2-bromo-5-methoxyphenol (2.03 g, 10 mmol) and K2CO3 (2.1 g, 15 mmol,) in CH3CN (50 mL) was added 1,2-dibromoethane (9.4 g, 50 mmol) at rt and the reaction mixture was stirred at 80° C. for 12 hr. The mixture was filtered and the filtrate was diluted with EA and washed with citric acid, brine, dried, concentrated and the residue was purified by silica gel column chromatography (PE/EA=10/1-3/1) to give the title compound (2.1 g, 68% yield). MS(ESI) m/e [M+H] 308.9.

### Step 2: 6-methoxy-2,3-dihydrobenzofuran

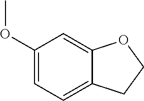

To a solution of 1-bromo-2-(2-bromoethoxy)-4-methoxybenzene (1.9 g, 6.2 mmol) in THF (40 mL) was added dropwise n-BuLi (2.7 mL, 6.8 mmol, 2.5M in hexane) below −65° C. and then the resultant solution was stirred at −65° C. for 30 min and allowed to warm to rt and stirred for 16 h. The solution was quenched by adding water, extracted with EA for twice. The combined organic layers were washed with water, brine, dried over anhydrous Na2SO4, filtered, and concentrated under reduced pressure. The residue was purified by silica gel column chromatography (PE/EA=4/1) to give the title compound (550 mg, 59% yield). MS(ESI) m/e [M+H]+=151.0.

### Step 3: 6-methoxy-2,3-dihydrobenzofuran-7-sulfinic acid

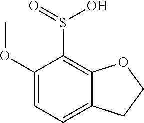

To a solution of 6-methoxy-2,3-dihydrobenzofuran (300 mg, 2 mmol) and TMEDA (464 mg, 4 mmol) in n-hexane (10 mL) was added n-BuLi (2.5 M in Hexane, 2.4 mL, 6 mmol) at 0° C. The reaction mixture was stirred at 0° C. for 1h and then cooled to −70° C. and SO2 (g) was bubbled into the solution for 1 min and then stirred at this temperature for 30 min. The reaction mixture was quenched by adding water (4 mL) and concentrated. The residue was purified by C18 column [(water (0.1% HCOOH)], B %=0%-70%) to give the title compound (360 mg, 72% yield). MS(ESI) m/e [M-OH]−=197.0.

### Step 4: 6-methoxy-2,3-dihydrobenzofuran-7-sulfonamide

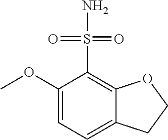

To a solution of 6-methoxy-2,3-dihydrobenzofuran-7-sulfinic acid (360 mg, 1.83 mmol) in DCM (10 mL) was added dropwise SO2Cl2 (247 mg, 1.83 mmol) at 0° C. and the reaction mixture was stirred at 0° C. for 30 min. The solution was diluted with DCM and washed with water, dried with Na2SO4, filtered, concentrated and the residue was dissolved in THF (5 mL) and ammonia water (1 mL) and stirred at rt for 1h. The solution was concentrated and the residue was purified by C18 column with 0.1% FA in water/ACN (eluted from 10% to 100%) to give the title product (100 mg, 24%). 1H NMR (400 MHz, DMSO-d6) δ 7.31 (d, J=8.2 Hz, 1H), 6.97 (s, 2H), 6.56 (d, J=8.2 Hz, 1H), 4.59 (t, J=8.7 Hz, 2H), 3.81 (s, 3H), 3.11 (t, J=8.7 Hz, 2H). MS (ESI) m/e [M+H]+=229.9.

### Example B19: Synthesis of 6-methoxy-2H-spiro[benzofuran-3,1′-cyclobutane]-7-sulfonamide

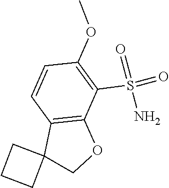 [SMILES: COc1ccc2c(c1S(N)(=O)=O)OCC21CCC1]

### Step 1: 1-(2-fluoro-4-methoxyphenyl)cyclobutane-1-carbonitrile

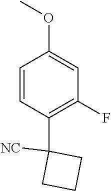

To a solution of 2-(2-fluoro-4-methoxyphenyl)acetonitrile (6.8 g, 41 mmol) and 1,3-dibromopropane (10 g, 49 mmol) in DMF (70 mL) was added NaH (60% in mineral oil, 3.3 g, 82 mmol,) at 0° C. The mixture was stirred at 15° C. for 2 hr. Upon completion of the reaction, the reaction mixture was quenched by aq. NH4Cl (100 mL) and extracted with EA. The combined organic layers were washed with brine, dried over Na2SO4, filtered and concentrated under reduced pressure. The residue was purified by silica gel column chromatography (PE:EA=30:1 to 5:1) to give the title compound (4.4 g, 52%). 1H NMR (400 MHz, DMSO-d6) δ=7.31 (t, J=9.2 Hz, 1H), 6.92 (dd, J=2.4, 9.2 Hz, 1H), 6.82 (dd, J=2.4, 9.2 Hz, 1H), 3.78 (s, 3H), 2.76-2.66 (m, 2H), 2.65-2.55 (m, 2H), 2.36-2.22 (m, 1H), 2.01-1.90 (m, 1H).

### Step 2: 1-(2-fluoro-4-methoxyphenyl)cyclobutane-1-carbaldehyde

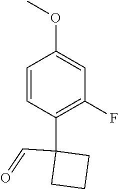

To a solution of 1-(2-fluoro-4-methoxyphenyl)cyclobutane-1-carbonitrile (4.4 g, 21 mmol) in THF (90 mL) was added DIBAL-H (1 M in THF, 43 mL) at −70° C. The mixture was stirred at 15° C. for 16 hr. Upon completion of the reaction, the reaction mixture was quenched by addition 1 M HCl (45 mL) at −30° C. then warmed to rt and stirred for 0.5 h, the mixture was filtered through celite and the filtrate was extracted with EA and washed with water and brine, dried over Na2SO4, filtered and concentrated in vacuum. The residue was purified by silica gel column chromatography (PE:EA=30:1 to 5:1) to give the title compound (2.5 g, 56%). 1H NMR (400 MHz, DMSO-d6) δ=9.62 (s, 1H), 7.16 (t, J=9.2 Hz, 1H), 6.85-6.81 (m, 1H), 6.80 (s, 1H), 3.76 (s, 3H), 2.66-2.56 (m, 2H), 2.42-2.32 (m, 2H), 2.05-1.83 (m, 2H).

### Step 3: (1-(2-fluoro-4-methoxyphenyl)cyclobutyl)methanol

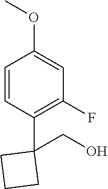

To a solution of 1-(2-fluoro-4-methoxyphenyl)cyclobutane-1-carbaldehyde (2.5 g, 12 mmol) in MeOH (25 mL) was added NaBH4 (477 mg, 12.6 mmol) at 0° C. The mixture was stirred at 0° C. for 1 hr. Upon completion of the reaction, the reaction mixture was quenched by addition aq.NH4Cl and extracted with EA. The combined organic layers were washed with brine, dried over Na2SO4, filtered and concentrated under reduced pressure. The residue was purified by silica gel column chromatography (PE:EA=20:1 to 5:1) to give the desired product (2 g, 79%). 1H NMR (400 MHz, DMSO-d6) δ=7.01-6.94 (m, 1H), 6.72-6.65 (m, 2H), 4.72 (t, J=5.6 Hz, 1H), 3.73 (s, 3H), 3.52 (d, J=5.6 Hz, 2H), 2.25-2.16 (m, 4H), 2.05-1.92 (m, 1H), 1.83-1.70 (m, 1H).

### Step 4: 6-methoxy-2H-spiro[benzofuran-3,1′-cyclobutane]

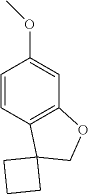

To a solution of (1-(2-fluoro-4-methoxyphenyl)cyclobutyl)methanol (2 g, 9.5 mmol) in THF (20 mL) was added t-BuOK (1 M in THF, 14.3 mL). The mixture was stirred at 70° C. for 12 hr. Upon completion of the reaction, the reaction was cooled to rt, the reaction mixture was quenched by water and extracted with EA. The combined organic layers were washed with brine, dried over Na2SO4, filtered and concentrated under reduced pressure. The residue was purified by silica gel column chromatography (PE:EA=1:0 to 40:1) to give the desired product (1 g, 55%). 1H NMR (400 MHz, DMSO-d6) δ=7.31 (d, J=8.0 Hz, 1H), 6.45 (dd, J=2.4, 8.0 Hz, 1H), 6.33 (d, J=2.4 Hz, 1H), 4.52 (s, 2H), 3.69 (s, 3H), 2.34-2.14 (m, 4H), 2.04-1.80 (m, 2H). MS(ESI) m/e [M+H]+=191.5.

### Step 5: lithium 6-methoxy-2H-spiro[benzofuran-3,1′-cyclobutane]-7-sulfinate

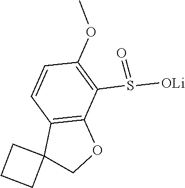

To a solution of 6-methoxy-2H-spiro[benzofuran-3,1′-cyclobutane](1000 mg, 5.26 mmol), TMEDA (610 mg, 5.26 mmol) in 20 ml hexane was added n-BuLi (2.1 ml, 5.26 mmol) at 0° C. and stirred for 10 min, the mixture was cooled to −78° C. and SO2 was bobbled for 5 min. Upon completion of the reaction, the mixture was filtered, the cake was collected to give the product (1.34 g, 100%). MS (ESI) m/e [M+H]+=255.3.

### Step 6: 6-methoxy-2H-spiro[benzofuran-3,1′-cyclobutane]-7-sulfonyl chloride

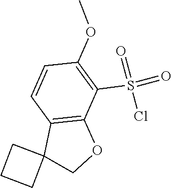 [SMILES: COc1ccc2c(c1S(=O)(=O)Cl)OCC21CCC1]

To a solution of lithium 6-methoxy-2H-spiro[benzofuran-3,1′-cyclobutane]-7-sulfinate (1.34 g, 5.3 mmol) in 10 ml AcOH/water (3/1) was added NCS (1.75 g, 13.2 mmol) at 0° C. and stirred for 10 min. Upon completion of the reaction, the solution was diluted with water, the mixture was filtered. The cake was collected to give the desired product (1.51 g, 100%). MS (ESI) m/e [M+H]+=288.2.

### Step 7: 6-methoxy-2H-spiro[benzofuran-3,1′-cyclobutane]-7-sulfonamide

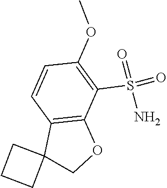 [SMILES: COc1ccc2c(c1S(N)(=O)=O)OCC21CCC1]

To a solution of 6-methoxy-2H-spiro[benzofuran-3,1′-cyclobutane]-7-sulfonyl chloride (1.51 g, 5.26 mmol) in THF (10 ml) was added NH3·H2O (1 mL) dropwise at 0° C. The reaction mixture was stirred at room temperature for 1 h. Upon completion of the reaction. The reaction mixture was added H2O and extracted with EA. The combined organic layers were washed with brine, dried over Na2SO4, filtered and concentrated, the residue was diluted with EA (10 ml) and stirred for 1 h, the mixture was filtered and the filtered cake was collected to give the desired product (1 g, 71%). MS (ESI) m/e [M+H]+=270.3.

### Example B20: Synthesis of 6-methoxy-2′,3′,5′,6′-tetrahydro-2H-spiro[benzofuran-3,4′-pyran]-7-sulfonamide

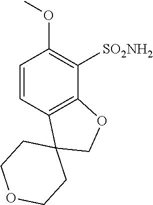 [SMILES: COc1ccc2c(c1S(N)(=O)=O)OCC21CCOCC1]

### Step 1: methyl 4-(2-fluoro-4-methoxyphenyl)tetrahydro-2H-pyran-4-carboxylate

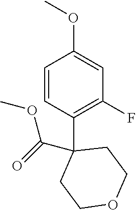 [SMILES: COC(=O)C1(c2ccc(OC)cc2F)CCOCC1]

To a solution of methyl 2-(2-fluoro-4-methoxy-phenyl)acetate (6 g, 30 mmol) and 1-bromo-2-(2-bromoethoxy)ethane (35 g, 151 mmol) in DMF (60 mL) was added t-BuOK (1 M in THF, 121 mL) dropwise at 0° C. The mixture was stirred at 15° C. for 1h. Upon completion of the reaction. The reaction mixture was quenched by addition of water, and then extracted with EA. The combined organic layers were washed with brine, dried over Na2SO4, filtered and concentrated under reduced pressure to give a residue. The residue was purified by silica gel column chromatography (PE:EA=3:1) to give the title compound (7.5 g, 92% yield). 1H NMR (400 MHz, CDCl3) δ=7.23 (t, J=8.8 Hz, 1H), 6.70 (dd, J=2.6, 8.8 Hz, 1H), 6.60 (dd, J=2.6, 13.8 Hz, 1H), 3.86 (td, J=4.0, 12.0 Hz, 2H), 3.80 (s, 3H), 3.78-3.71 (m, 2H), 3.70 (s, 3H), 2.41 (br d, J=13.6 Hz, 2H), 2.14-2.05 (m, 2H).

### Step 2: (4-(2-fluoro-4-methoxyphenyl)tetrahydro-2H-pyran-4-yl)methanol

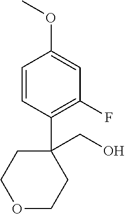 [SMILES: COc1ccc(C2(CO)CCOCC2)c(F)c1]

To a solution of methyl 4-(2-fluoro-4-methoxy-phenyl)tetrahydropyran-4-carboxylate (7.5 g, 28 mmol) in THF (120 mL) was added LiBH4 (2 M in THF, 28 mL, 56 mmol) at 0° C. The mixture was stirred at 40° C. for 16 hr. Upon completion of the reaction, the mixture was cooled to rt, quenched by addition of water and extracted with EA. The combined organic layers were washed with brine, dried over Na2SO4, filtered and concentrated under reduced pressure to give the crude product (6.6 g) without further purification. 1H NMR (400 MHz, DMSO-d6) δ=7.27-7.16 (m, 1H), 6.76-6.69 (m, 2H), 4.62 (t, J=5.6 Hz, 1H), 3.75 (s, 3H), 3.73-3.65 (m, 2H), 3.48 (d, J=5.6 Hz, 2H), 3.42-3.33 (m, 2H), 2.04 (br d, J=14.4 Hz, 2H), 1.86-1.79 (m, 2H).

### Step 3: 6-methoxy-2′,3′,5′,6′-tetrahydro-2H-spiro[benzofuran-3,4′-pyran]

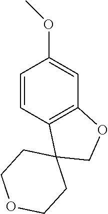 [SMILES: COc1ccc2c(c1)OCC21CCOCC1]

To a solution of [4-(2-fluoro-4-methoxy-phenyl)tetrahydropyran-4-yl]methanol (5.6 g, 23 mmol) in THF (60 mL) was added t-BuOK (1 M in THF, 35 mL, 35 mmol) at 15° C. The mixture was stirred at 70° C. for 3 hr. Upon completion of the reaction, the mixture was cooled to rt, the reaction mixture was quenched by addition of water and extracted with EA. The combined organic layers were washed with brine, dried over Na2SO4, filtered and concentrated under reduced pressure to give the crude product. The crude product was triturated with MTBE and filtered. The filter-cake was dried in vacuum to give the desired product (4.2 g, 69%). 1H NMR (400 MHz, DMSO-d6) δ=7.11 (d, J=8.4 Hz, 1H), 6.42 (dd, J=2.4, 8.4 Hz, 1H), 6.38 (d, J=2.4 Hz, 1H), 4.45 (s, 2H), 3.88-3.79 (m, 2H), 3.69 (s, 3H), 3.40 (dt, J=2.0, 12.0 Hz, 2H), 1.84 (dt, J=3.6, 12.0 Hz, 2H), 1.59-1.47 (m, 2H). MS(ESI) m/e [M+H]+=221.5.

### Step 4: 6-methoxy-2′,3′,5′,6′-tetrahydro-2H-spiro[benzofuran-3,4′-pyran]-7-sulfonamide

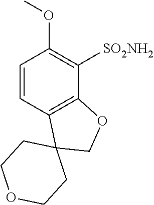 [SMILES: COc1ccc2c(c1S(N)(=O)=O)OCC21CCOCC1]

A mixture of 6-methoxy-2′,3′,5′,6′-tetrahydro-2H-spiro[benzofuran-3,4′-pyran](1 g, 4.5 mmol) and TMEDA (580 mg, 5 mmol) in THF (10 mL) was added n-BuLi (2.5 M in n-hexane, 2 mL, 5 mmol) at 0° C. The mixture was stirred at 0° C. for 0.5 hr and cooled to −60° C. The sulfuryl chloride (920 mg, 6.8 mmol) was added to the mixture at −60° C. The mixture was stirred at 15° C. for 12 hr. Upon completion of the reaction, the reaction was poured into water and extracted with EA. The combined organic layers were washed with brine, dried over Na2SO4, filtered and concentrated to give the crude sulfonyl chloride (1.4 g). To a solution of the crude sulfonyl chloride (1.4 g, 4.4 mmol) in MeOH (10 mL) was added NH3/MeOH (7 M, 5 mL). The mixture was stirred at 15° C. for 0.5 hr. Upon completion of the reaction, the mixture was filtered. The filtrate was dried in vacuum to give the crude product, which was purified by prep-HPLC (column: Waters Xbridge Prep OBD C18 150×40 mm×1Oum; mobile phase: [H2O (10 mM NH4HCO3)-ACN]; gradient: 1%-40% B over 8.0 min) to give the title compound (135.5 mg, 10%). 1H NMR (400 MHz, DMSO-d6) δ=7.35 (d, J=8.4 Hz, 1H), 6.99 (s, 2H), 6.61 (d, J=8.4 Hz, 1H), 4.55 (s, 2H), 3.86 (br d, J=2.4 Hz, 2H), 3.83 (s, 3H), 3.46-3.36 (m, 2H), 1.87 (dt, J=4.0, 12.4 Hz, 2H), 1.54 (br d, J=12.4 Hz, 2H). MS(ESI) m/e [M−H]−=298.3

### Example B21: Synthesis of 7-methoxy-3,3-dimethylchromane-8-sulfonamide

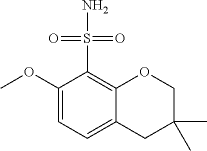

### Step 1: 7-hydroxy-3,3-dimethylchroman-4-one

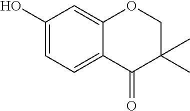

To a suspension of tBuOK (6.72 g, 60 mmol) in THF (70 mL) was added dropwise 7-hydroxychroman-4-one (1.64 g, 10 mmol) and CH3I (7.1 g, 50 mmol) in THF (30 mL) at −70° C. and the solution was stirred at this temperature for 4 h and allowed to warm to rt and stirred for 1 h. The solution was diluted with water, citric acid, extracted with EA. The extract layer was washed with brine, dried over with Na2SO4, filtered and concentrated to give the title product (3 g, crude). MS (ESI) m/e [M+H]+=193.1.

### Step 2: 7-methoxy-3,3-dimethylchroman-4-one

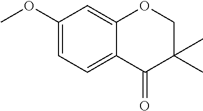

To a suspension of 7-hydroxy-3,3-dimethylchroman-4-one (3 g, 10 mmol) and K2CO3 (2.76 g, 20 mmol) in DMF (40 mL) was added CH3I (2.84 g, 20 mmol) and the mixture was stirred at 90° C. for overnight. After cooling to rt, the solution was diluted with EA, washed with water, brine, dried over with Na2SO4, filtered and concentrated. The residue was purified by silica gel column chromatography (PE:EA=1:0 to 4:1) to give the title product (1.6 g, 78% for two steps). MS (ESI) m/e [M+H]+=207.0.

### Step 3: 7-methoxy-3,3-dimethylchromane

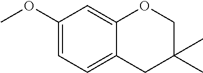

To a suspension of 7-methoxy-3,3-dimethylchroman-4-one (1.6 g, 7.76 mmol) and Zn powder (4 g, 621 mmol) in MeOH (30 mL) and conc.HCl (10 mL) and the mixture was stirred at rt for 4h. Upon completion of the reaction, the mixture was diluted with EA, washed with water, brine, dried over with Na2SO4, filtered and concentrated. The residue was purified by silica gel column chromatography (PE:EA=2:1) to give the title product (1.1 g, 74%). MS (ESI) m/e [M+H]+=193.1.

### Step 4: 7-methoxy-3,3-dimethylchromane-8-sulfinic acid

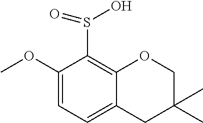

To a solution of 7-methoxy-3,3-dimethylchromane (576 mg, 3 mmol) and TMEDA (348 mg, 9 mmol) in n-hexane (20 mL) was added dropwise n-BuLi (3.6 mL, 9 mmol, 2.5M in n-hexane) below 10° C. and the reaction mixture was stirred at 10° C. for 30 min. The solution was cooled to −60° C. and the SO2 (g) was bubbled into the solution for 1 min and stirred at −60° C. for 30 min. Upon completion of the reaction, 1N HCl (5 mL) was added to the reaction mixture and the solution was diluted with water, extracted with EA, the extract layer was washed with brine, dried over with Na2SO4, filtered and concentrated to give the title product (600 mg, 78%). MS (ESI) m/e [M+H]+=257.0.

### Step 5: 7-methoxy-3,3-dimethylchromane-8-sulfonamide

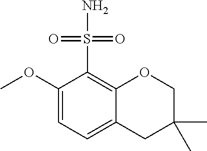

To a solution of 7-methoxy-3,3-dimethylchromane-8-sulfinic acid (600 mg, 2.34 mmol) in AcOH (10 mL) and H2O (3 mL) was added NCS (939 mg, 7 mmol) at rt and the reaction mixture was stirred at rt for 2 h. The solution was diluted with water and extracted with EA. The extracted layer was washed with brine, dried with Na2SO4, filtered and concentrated. The residue was dissolved in THF (10 mL) and NH40H solution (2 mL). After stirred at rt for 1 h, the solution was concentrated and the residue was purified by C18 column with 0.1% FA in water/ACN (eluted from 10% to 100%) to give the title product (200 mg, 31%). MS (ESI) m/e [M+H]+=272.1.

### Example B22: Synthesis of 2-methoxy-5-(1-methoxy-2-methylpropan-2-yl)benzenesulfonamide

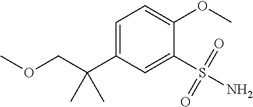 [SMILES: COCC(C)(C)c1ccc(OC)c(S(N)(=O)=O)c1]

### Step 1: 2-[3-[bis[(2,4-dimethoxyphenyl)methyl]sulfamoyl]-4-methoxy-phenyl]-2-methyl-propanoate

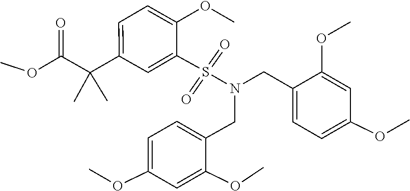

To a solution of methyl 2-[3-[bis[(2,4-dimethoxyphenyl)methyl]sulfamoyl]-4-methoxy-phenyl]acetate (15 g, 26.8 mmol) in THF (150 mL) was added tBuOK (9 g, 80.4 mmol) and Mel (11.4 g, 80.4 mmol) slowly at 0° C. The reaction was stirred at rt for 3 hrs. Upon completion of the reaction, the reaction mixture was quenched by H2O, and extracted with EA. The combined organic layers were concentrated. The residue was purified by silica gel column chromatography (PE/EA=10:1 to 1:1) to give the title compound (12 g, 76% yield). 1H NMR (400 MHz, DMSO-d6) δ=7.62-7.55 (m, 1H), 7.53-7.45 (m, 1H), 7.14-7.06 (m, 1H), 6.96-6.88 (m, 2H), 6.43-6.31 (m, 4H), 4.36-4.23 (m, 4H), 3.78 (s, 3H), 3.71 (s, 6H), 3.58 (s, 6H), 1.48 (s, 6H).

### Step 2: N,N-bis(2,4-dimethoxybenzyl)-5-(1-hydroxy-2-methylpropan-2-yl)-2-methoxybenzenesulfonamide

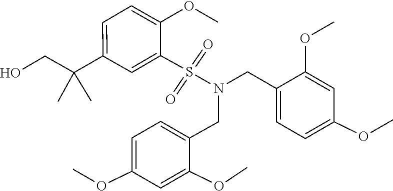 [SMILES: COc1ccc(CN(Cc2ccc(OC)cc2OC)S(=O)(=O)c2cc(C(C)(C)CO)ccc2OC)c(OC)c1]

To a solution of methyl 2-[3-[bis[(2,4-dimethoxyphenyl)methyl]sulfamoyl]-4-methoxy-phenyl]-2-methyl-propanoate (1.5 g, 2.5 mmol) in THF (20 mL) was added LiBH4 (2 M in THF, 2.55 mL) at 0° C. The reaction was stirred at 70° C. for 16 hr. The reaction mixture was quenched by saturated aq. NH4Cl (30 mL) and extracted with EA. The combined organic layers were washed with brine, dried over Na2SO4, filtered and concentrated under reduced pressure to give a residue. The residue was purified by silica gel column chromatography (PE/EA=5:1 to 1:1) to give the title compound (1.1 g, 77% yield). 1H NMR (400 MHz, DMSO-d6) δ 7.62 (br d, J=1.6 Hz, 1H), 7.54 (br d, J=8.4 Hz, 1H), 7.06 (br d, J=8.4 Hz, 1H), 6.93 (br d, J=8.0 Hz, 2H), 6.45-6.31 (m, 4H), 4.72 (br t, J=4.8 Hz, 1H), 4.29 (s, 4H), 3.77 (s, 3H), 3.70 (s, 6H), 3.58 (s, 6H), 3.36 (br d, J=4.8 Hz, 2H), 1.19 (s, 6H).

### Step 3: N,N-bis(2,4-dimethoxybenzyl)-2-methoxy-5-(1-methoxy-2-methylpropan-2-yl)benzenesulfonamide

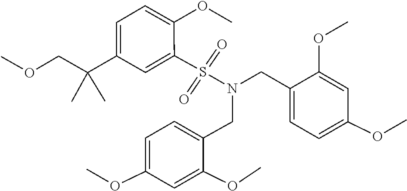

To a solution of N,N-bis[(2,4-dimethoxyphenyl)methyl]-5-(2-hydroxy-1,1-dimethyl-ethyl)-2-methoxy-benzenesulfonamide (1.1 g, 2 mmol) in THF (15 mL) was added NaH (60% in mineral oil, 120 mg, 3 mmol) at 0° C. The mixture was stirred at 0° C. for 0.5 hr. Then Mel (418 mg, 3 mmol) was added to the mixture at 0° C. The reaction was stirred at 70° C. for 12 hr. After cooling down to rt, the reaction mixture was quenched by aq. NH4Cl (20 mL) and extracted with EA. The combined organic layers were washed with brine, dried over Na2SO4, filtered and concentrated under reduced pressure. The residue was purified by silica gel column chromatography (PE/EA=10:1 to 4:1) to give the title compound (400 mg, 35% yield). 1H NMR (400 MHz, DMSO-d6) δ 7.58 (d, J=2.4 Hz, 1H), 7.54 (d, J=8.8 Hz, 1H), 7.07 (d, J=8.8 Hz, 1H), 6.93 (d, J=8.0 Hz, 2H), 6.43-6.30 (m, 4H), 4.29 (s, 4H), 3.78 (s, 3H), 3.70 (s, 6H), 3.58 (s, 6H), 3.29 (s, 2H), 3.20 (s, 3H), 1.21 (s, 6H).

### Step 4: 2-methoxy-5-(1-methoxy-2-methylpropan-2-yl)benzenesulfonamide

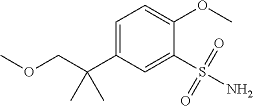

To a solution of N,N-bis[(2,4-dimethoxyphenyl)methyl]-2-methoxy-5-(2-methoxy-1,1-dimethyl-ethyl)benzenesulfonamide (350 mg, 0.6 mmol) in DCM (5 mL) was added TFA (1 mL). The reaction mixture was stirred at rt for 1 hr. The reaction mixture was concentrated in vacuum. The residue was purified by prep-HPLC (column: Phenomenex luna C18; mobile phase: [H2O (0.1% TFA)-ACN]; gradient: 15%-45% B over 8.0 min) to give the title compound (72 mg, 43% yield). 1H NMR (400 MHz, DMSO-d6) δ 7.70 (d, J=2.4 Hz, 1H), 7.56 (d, J=8.8 Hz, 1H), 7.12 (d, J=8.8 Hz, 1H), 7.01 (s, 2H), 3.87 (s, 3H), 3.33 (br s, 2H), 3.21 (s, 3H), 1.24 (s, 6H).

### Example B23: Synthesis of 2-(tert-butyl)-5-methoxypyridine-4-sulfonamide

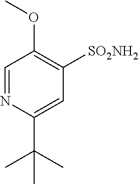 [SMILES: COc1cnc(C(C)(C)C)cc1S(N)(=O)=O]

To a solution of 2-tert-butyl-5-methoxy-pyridine (180 mg, 1.1 mmol), TMEDA (152 mg, 1.3 mmol) in THF (3 mL) was added n-BuLi (2.5 M in n-hexane, 480 L) dropwise at −60° C. The mixture was stirred at −60° C. for 1 hr, and N-sulfinyl-O-(tert-butyl)-hydroxylamine (221 mg, 1.6 mmol) in THF (0.5 mL) was added dropwise. The reaction was stirred at 15° C. for 16 h. The reaction was quenched by saturated aq. NH4Cl (5 ml), and extracted by EA. The combined organic layers were washed by brine, dried over Na2SO4, filtered and concentrated under reduced pressure. The residue was purified by prep-HPLC (column: Waters Xbridge Prep OBD C18 150×40 mm×10 um, mobile phase: [H2O (10 mM NH4HCO3)-ACN]; gradient: 10%-40% B over 8.0 min) to give the title compound (8.5 mg, 3% yield). MS(ESI) m/e [M+1]245.4. 1H NMR: (400 MHz, DMSO-d6) δ 8.55 (s, 1H), 7.61 (s, 1H), 6.55 (br s, 2H), 3.99 (s, 3H), 1.31 (s, 9H).

### Example B24: Synthesis of 6-(tert-butyl)-3-(methoxy-d3)pyridine-2-sulfonamide

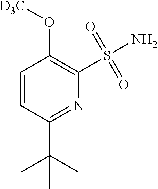 [SMILES: [2H]C([2H])([2H])Oc1ccc(C(C)(C)C)nc1S(N)(=O)=O]

### Step 1: 2-fluoro-6-iodopyridin-3-ol

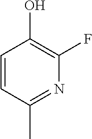

To a solution of 2-fluoropyridin-3-ol (5 g, 44.2 mmol) and K2CO3 (12.2 g, 88.4 mmol) in water (200 mL) was added I2 (12.4 g, 48.7 mmol) and the mixture was stirred at rt for 3 h. The reaction mixture was adjusted pH to 6 with citric acid and extracted with EA for twice. The combined organic layers were washed with Na2S2O3 solution, brine, dried over anhydrous Na2SO4, filtered and concentrated to give the product (11 g, crude). MS(ESI) m/e [M+H]+=239.9.

### Step 2: 2-fluoro-6-iodo-3-(methoxy-d3)pyridine

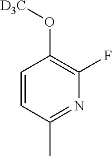

To a solution of 2-fluoro-6-iodopyridin-3-ol (11 g, 44.2 mmol) in DMF (110 L) was added NaH (60% in mineral oil, 1.96 g, 48.6 mmol) and the mixture was stirred at rt for 30 min. CD3I (7 g, 48.5 mmol) was added and the mixture was stirred at rt for 3 h. The reaction was diluted with EA, washed with water, brine, dried over anhydrous Na2SO4, filtered and concentrated and the residue was purified by silica gel column chromatography (PE to PE/EA=1:1) to give the product (6 g, 53%). MS(ESI) m/e [M+H]+=257.0.

### Step 3: 2-(benzylthio)-6-iodo-3-(methoxy-d3)pyridine

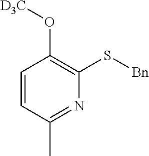 [SMILES: [2H]C([2H])([2H])Oc1ccc(C)nc1SCc1ccccc1]

To a solution of phenylmethanethiol (2.79 g, 22.5 mmol) in THF (100 mL) was added NaH (60% in mineral oil, 900 mg, 22.5 mmol) slowly and stirred at 0° C. for 30 min. 2-fluoro-6-iodo-3-(methoxy-d3)pyridine (4.8 g, 18.8 mmol) was added and the mixture was stirred at 70° C. for 4 h. The reaction mixture was poured into ice-water and extracted with EA for twice. The combined organic layers were washed with brine, dried over anhydrous Na2SO4, filtered and concentrated in vacuum. The residue was purified by silica gel column chromatography (PE to PE/EA=3:1) to give the compound (5.1 g, 75% yield). MS(ESI) m/e [M+H]+=361.1.

### Step 4: 2-(benzylthio)-6-(tert-butyl)-3-(methoxy-d3)pyridine

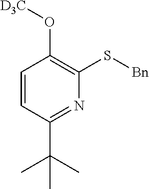 [SMILES: [2H]C([2H])([2H])Oc1ccc(C(C)(C)C)nc1SCc1ccccc1]

To a mixture of CuCN (1.24 g, 13.9 mmol) in THF (20 mL) was added dropwise tert-butylmagnesium chloride (27.8 mL, 1M in THF, 27.8 mmol) below −65° C. and stirred at −65° C. for 1h. 2-(benzylthio)-6-iodo-3-(methoxy-d3)pyridine (2 g, 5.55 mmol) in THF (5 mL) was added dropwise below −65° C. and stirred at −65° C. for 1h and allowed to warm to rt and stirred for 3 h. The reaction mixture was poured into saturated aq. NH4Cl (100 ml) and extracted with EtOAc (100 ml×2). The combined organic layers were washed with saturated aq. NH4Cl, brine, dried over anhydrous Na2SO4, filtered and concentrated in vacuum. The residue was purified by silica gel column chromatography (PE to PE:EA=4:1) to give the title compound (1 g, 62% yield). MS(ESI) m/e [M+H]+=291.2.

### Step 5: 6-(tert-butyl)-3-(methoxy-d3)pyridine-2-sulfonamide

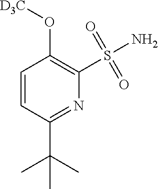 [SMILES: [2H]C([2H])([2H])Oc1ccc(C(C)(C)C)nc1S(N)(=O)=O]

To a solution of 2-(benzylthio)-6-(tert-butyl)-3-(methoxy-d3)pyridine (1 g, 93.45 mmol) in AcOH (20 mL) and H2O (5 mL) was added NCS (1.38 g, 10.34 mmol) and the mixture was stirred at rt for 1 hr. Upon completion of the reaction, the solution was poured into ice-water and extracted with EA for twice. The extracts were washed with water, brine, dried, concentrated and the residue was dissolved in THF (10 mL) and NH40H (25%, 2 mL) was added and the reaction was stirred at rt for 1 h. The solution was concentrated and the residue was purified by C18 column with 0.1% FA in water/ACN (eluted from 10% to 60%) to give the title product (500 mg, 59%). 1H NMR (400 MHz, DMSO-d6) δ 7.62-7.61 (m, 2H), 7.15 (s, 2H), 1.31 (s, 9H). MS (ESI) m/e [M+H]+=248.1.

### Example B25: Synthesis of 6-methoxy-3,3-dimethyl-2,3-dihydrobenzofuran-5-sulfonamide

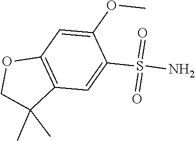 [SMILES: COc1cc2c(cc1S(N)(=O)=O)C(C)(C)CO2]

### Step 1: 6-methoxy-3,3-dimethyl-2,3-dihydrobenzofuran-5-sulfonyl chloride

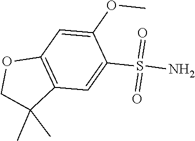 [SMILES: COc1cc2c(cc1S(N)(=O)=O)C(C)(C)CO2]

To a solution of 6-methoxy-3,3-dimethyl-2,3-dihydrobenzofuran (1 g, 5.6 mmol) in DCM (10 ml) was added sulfurochloridic acid (651 mg, 5.6 mol) dropwise at 0° C. and stirred for 1 hr. Upon completion of the reaction, the mixture was poured into ice water and extracted with DCM. The organic layer was concentrated to give the title product (1 g, 64%). MS (ESI) m/e [M+Na]+=277.2.

### Step 2: 6-methoxy-3,3-dimethyl-2,3-dihydrobenzofuran-5-sulfonamide

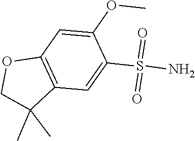 [SMILES: COc1cc2c(cc1S(N)(=O)=O)C(C)(C)CO2]

To a solution of 6-methoxy-3,3-dimethyl-2,3-dihydrobenzofuran-5-sulfonyl chloride (1.00 g, 3.60 mmol) in THF (10 ml) was added NH3·H2O (5 mL) dropwise at 0° C. The reaction mixture was stirred at room temperature for 1 h. Upon completion of the reaction. The reaction mixture was added H2O and extracted with EA. The combined organic layers were washed with brine, dried over Na2SO4, filtered and concentrated. The residue was diluted with EA (10 ml) and stirred for 1 h. The mixture was filtered and the filter cake was collected to give the desired product (50 mg, 5%). 1H NMR (400 MHz, CDCl3) δ 7.64 (s, 1H), 6.48 (s, 1H), 4.93 (s, 2H), 4.32 (s, 2H), 3.96 (s, 3H), 1.33 (s, 6H). MS (ESI) m/e [M+H]+=257.3.

### Example B26: Synthesis of 5-(2-oxabicyclo[2.1.1]hexan-4-yl)-2-methoxybenzenesulfonamide

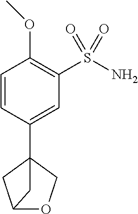 [SMILES: COc1ccc(C23COC(C2)C3)cc1S(N)(=O)=O]

### Step 1: 4-(4-methoxyphenyl)-2-oxabicyclo[2.1.1]hexane

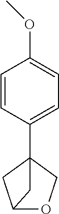

To a solution of 3-(hydroxymethyl)-3-(4-methoxyphenyl)cyclobutan-1-ol (1 g, 4.8 mmol) in pyridine (3.2 mL) and DCM (8 mL) was added 4-toluenesulfonyl chloride (1.37 g, 7.2 mmol, 1.5 eq) at 0° C. under N2. The reaction was stirred at 25° C. for 2 h. Upon completion of the reaction, the mixture was diluted with DCM, washed with water, 1 N aq. HCl and brine, dried over anhydrous Na2SO4, filtered and concentrated in vacuo to give crude (3-hydroxy-1-(4-methoxyphenyl)cyclobutyl)methyl 4-methylbenzenesulfonate.

To a solution of crude (3-hydroxy-1-(4-methoxyphenyl)cyclobutyl)methyl 4-methylbenzenesulfonate in THF (80 mL) was added NaH (60% in mineral oil, 320 mg, 8 mmol) at 0° C. under N2. The reaction was stirred at 0° C. for 30 min, then warmed to 80° C. and stirred at this temperature for 12 h. After being cooled to rt, MeOH (10 mL) was added. The mixture was concentrated in vacuo. The residue was purified by silica gel column chromatography (PE:EA=1:0 to 1:1) to give the title compound (0.2 g, 19% yield). 1H NMR (400 MHz, DMSO-d6) δ=7.24-7.19 (m, 2H), 6.92-6.88 (m, 2H), 4.55 (s, 1H), 3.73 (s, 3H), 3.71 (s, 2H), 2.00-1.94 (m, 2H), 1.78-1.72 (m, 2H).

### Step 2: 4-(3-bromo-4-methoxyphenyl)-2-oxabicyclo[2.1.1]hexane

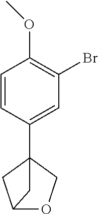

To a solution of 4-(4-methoxyphenyl)-2-oxabicyclo[2.1.1]hexane (0.2 g, 1.0 mmol) in ACN (3 mL) was added NBS (187.1 mg, 1.0 mmol, 1 eq). The reaction was stirred at 20° C. for 12 h. Upon completion of the reaction, the mixture was concentrated in vacuo. The residue was purified by silica gel column chromatography (PE:EA=1:0 to 1:1) to give the title compound (0.2 g, 71% yield). 1H NMR (400 MHz, CDCl3) δ=7.43 (d, J=2.0 Hz, 1H), 7.17-7.14 (m, 1H), 6.88 (d, J=8.4 Hz, 1H), 4.65 (s, 1H), 3.90 (s, 3H), 3.82 (s, 2H), 2.03-1.97 (m, 2H), 1.96-1.89 (m, 2H)

### Step 3: 4-(3-(benzylthio)-4-methoxyphenyl)-2-oxabicyclo[2.1.1]hexane

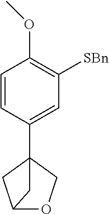

To a solution of 4-(3-bromo-4-methoxyphenyl)-2-oxabicyclo[2.1.1]hexane (150 mg, 557 mol) in dioxane (2 mL) was added Pd2(dba)3 (51 mg, 56 μmol) and Xantphos (65 mg, 112 μmol). The mixture was stirred at rt for 30 min under N2. Then a solution of phenylmethanethiol (139 mg, 1.1 mmol) and DIEA (144 mg, 1.1 mmol) in dioxane (2 mL) was added into the reaction mixture. The reaction was stirred at 110° C. for 12 h. After being cooled to rt, the reaction mixture was concentrated in vacuo. The residue was purified by silica gel column chromatography (PE:EA=1:0 to 3:1) to give the title compound (140 mg, 80% yield). 1H NMR (400 MHz, CDCl3) δ 7.30-7.27 (m, 3H), 7.25-7.20 (m, 2H), 7.07-7.03 (m, 2H), 6.82 (d, J=8.0 Hz, 1H), 4.61 (s, 1H), 4.09 (s, 2H), 3.90 (s, 3H), 3.72 (s, 2H), 1.93-1.83 (m, 4H).

### Step 4: 5-(2-oxabicyclo[2.1.1]hexan-4-yl)-2-methoxybenzenesulfonamide

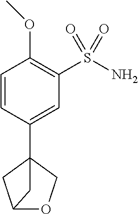 [SMILES: COc1ccc(C23COC(C2)C3)cc1S(N)(=O)=O]

To a solution of 4-(3-(benzylthio)-4-methoxyphenyl)-2-oxabicyclo[2.1.1]hexane (140 mg, 448 μmol) in AcOH (2.4 mL), ACN (0.6 mL) and H2O (0.06 mL) was added sulfuryl chloride (181 mg, 1.3 mmol) at 0° C. The reaction was stirred at 0° C. for 1 h. Upon completion of the reaction, the mixture was concentrated in vacuum to give crude 5-(2-oxabicyclo[2.1.1]hexan-4-yl)-2-methoxybenzenesulfonyl chloride, which was dissolved in

7M NH3 in MeOH (2.6 mL). The reaction was stirred at rt for 10 min. Upon completion of the reaction, the mixture was filtered through celite and concentrated in vacuo. The residue was purified by prep-HPLC (column: Phenomenex Gemini C18 75×40 mm×3 um; mobile phase: [H2O(NH4HCO3) ACN]; gradient: 5%-30% B over 8.0 min) to give the title compound (85.3 mg, 70% yield). 1H NMR (400 MHz, DMSO-d6) δ 7.61 (d, J=2.0 Hz, 1H), 7.51-7.48 (m, 1H), 7.19 (d, J=8.4 Hz, 1H), 6.04 (br s, 1H), 4.57 (s, 1H), 3.89 (s, 3H), 3.73 (s, 2H), 2.03-1.97 (m, 2H), 1.84-1.77 (m, 2H). MS(ESI) m/e [M+H]+=270.1.

### Example B27: synthesis of 7-methoxy-3,4-dihydro-2H-2,4-methanochromane-8-sulfonamide

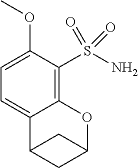 [SMILES: COc1ccc2c(c1S(N)(=O)=O)OC1CC2C1]

### Step 1: 2-(3-(benzyloxy)cyclobutyl)-5-methoxyphenol

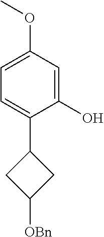 [SMILES: COc1ccc(C2CC(OCc3ccccc3)C2)c(O)c1]

To a solution of 2-bromo-5-methoxyphenol (1.1 g, 5.4 mmol) in DME (330 mL) was added ((3-bromocyclobutoxy)methyl)benzene (1.3 g, 5.4 mmol), tris(trimethylsilyl)silane (1.6 g, 6.5 mmol), 2,6-dimethylpyridine (1.2 g, 10.8 mmol), Ir[dF(CF3)ppy)]2(dtbbpy)PF6 (60.8 mg, 54 μmol) and (dtbbpy)NiCl2 (22 mg, 54 μmol) at 25° C. under N2. The solution was injected into a photo-flow reactor {FEP, Coils reactor, 3.175 (⅛″) mm, 55 mL, 395 nm, 200 W*2, 40° C.} with a flow rate of 40 mL/min. 3 reactions were set up in parallel. Upon completion of the reaction, the combined mixture of 3 reactions was poured into H2O, extracted with EA. The combined organic layers were washed with brine, dried over anhydrous Na2SO4, filtered and concentrated in vacuo. The residue was purified by prep-TLC (silica gel, PE:EA=10:1 to 3:1) to give the title compound (0.9 g, 20% yield). MS(ESI) m/e [M+H]+=285.1.

### Step 2: 2-(3-hydroxycyclobutyl)-5-methoxy-phenol

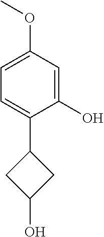

To a mixture of 2-(3-benzyloxycyclobutyl)-5-methoxyphenol (0.9 g, 3.2 mmol) and Pd/C (0.4 g, 10% wt/wt) in MeOH (12 mL) and EA (48 mL) was added conc. HCl (0.3 mL). The suspension was degassed and purged with H2 for 3 times, stirred under H2 (15 psi) at 25° C. for 16 h. Upon completion of the reaction, the mixture was filtered through celite. The filtrate was concentrated in vacuo. The residue was purified by prep-TLC (silica gel, PE:EA=1:1) to give the title compound (0.45 g, 72% yield). MS(ESI) m/e [M-OH]+=177.1.

### Step 3: 7-methoxy-3,4-dihydro-2H-2,4-methanochromane

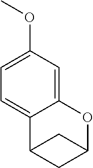

A mixture of 2-(3-hydroxycyclobutyl)-5-methoxyphenol (250 mg, 1.3 mmol) and 2-(Tributylphosphoranylidene)acetonitrile (Tsunoda reagent, 940 mg, 3.9 mmol) in dioxane (7.5 mL) was stirred at 150° C. under microwave for 1 h. After being cooled to rt, the solution was diluted with EA and washed with brine, dried over with Na2SO4, filtered and concentrated in vacuo. The residue was purified by silica gel column chromatography (PE:EA=2:1) to give the title compound (80 mg, 35%). 1H NMR (400 MHz, DMSO-d6) δ 6.94 (d, J=8.1 Hz, 1H), 6.39 (d, J=2.4 Hz, 1H), 6.34 (dd, J=2.4, 8.1 Hz, 1H), 5.04 (td, J=3.7, 6.3 Hz, 1H), 3.69 (s, 3H), 3.32-3.27 (m, 1H), 2.43-2.36 (m, 2H), 1.44-1.38 (m, 2H). MS(ESI) m/e [M+H]+=177.1.

### Step 4: 7-methoxy-3,4-dihydro-2H-2,4-methanochromane-8-sulfonamide

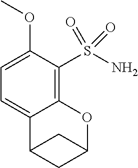 [SMILES: COc1ccc2c(c1S(N)(=O)=O)OC1CC2C1]

To a mixture of 7-methoxy-3,4-dihydro-2H-2,4-methanochromane (80 mg, 0.46 mmol) in n-hexane (10 mL) was added TMEDA (106 mg, 0.92 mmol) and n-BuLi (1.6 M in n-hexane, 0.40 mL, 0.56 mmol) at 0° C., stirred at 0° C. for 30 min. The solution was cooled to −70° C., and bubbled with SO2 (gas) for 1 min. The reaction was further stirred at −70° C. for 30 min. Upon completion of the reaction, the mixture was concentrated in vacuo to give the crude 7-methoxy-3,4-dihydro-2H-2,4-methanochromane-8-sulfinic acid.

To a mixture of crude 7-methoxy-3,4-dihydro-2H-2,4-methanochromane-8-sulfinic acid in acetic acid (12 mL) and water (4 mL) was added NCS (190 mg, 0.84 mmol). The reaction was stirred at rt for 2 h. Upon completion of the reaction, the reaction mixture was concentrated to remove acetic acid. The residual was extracted with EA. The combined organic phases were washed with brine, dried over anhydrous Na2SO4, concentrated in vacuo to give the crude 7-methoxy-3,4-dihydro-2H-2,4-methanochromane-8-sulfonyl chloride.

To a mixture of crude 7-methoxy-3,4-dihydro-2H-2,4-methanochromane-8-sulfonyl chloride in THF (6 mL) was added NH3·H2O (0.5 mL). The reaction was stirred at rt for 2 h. Upon completion of the reaction, the mixture was concentrated in vacuo. The residue was purified by silica gel column chromatography (PE:EA=1:1) to give the title compound (60 mg, 51% yield). MS(ESI) m/z [M+H]+=256.0.

### Example B28: Synthesis of 6-(bicyclo[1.1.1]pentan-1-yl)-3-methoxypyridine-2-sulfonamide

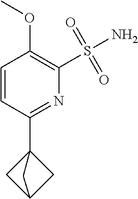 [SMILES: COc1ccc(C23CC(C2)C3)nc1S(N)(=O)=O]

### Step 1: 6-(bicyclo[1.1.1]pentan-1-yl)-2-fluoro-3-methoxypyridine

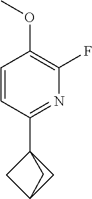

To a mixture of 6-bromo-2-fluoro-3-methoxypyridine (412 mg, 2 mmol) in DMA (10 mL) was added bicyclo[1.1.1]pentane-1-carboxylic acid (336 mg, 3 mmol), (dtbbpy)NiCl2 (80 mg, 0.2 mmol), Ir[dF(CF3)ppy]2(dtbbpy)PF6 (224 mg, 0.2 mmol), 2-(tert-butyl)-1,1,3,3-tetramethylguanidine (513 mg, 3 mmol) and Phthalimide (294 mg, 2 mmol). The mixture was exchanged with N2 for three times, stirred at rt under the blue light (440 nm) for 16 h. Upon completion of the reaction, the mixture was concentrated in vacuo. The residue was purified by silica gel column chromatography (PE:EA=2:1) to give the title compound (70 mg, 18%). MS(ESI) m/z [M+H]+=194.2.

### Step 2: 2-(benzylthio)-6-(bicyclo[1.1.1]pentan-1-yl)-3-methoxypyridine

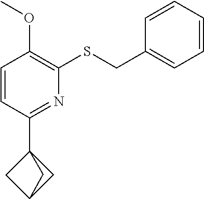 [SMILES: COc1ccc(C23CC(C2)C3)nc1SCc1ccccc1]

To a mixture of phenylmethanethiol (35 mg, 0.26 mmol) in THF (10 mL) was added NaH (60% in mineral oil, 11.2 mg, 0.26 mmol), and stirred at 0° C. for about 30 min. 6-(bicyclo[1.1.1]pentan-1-yl)-2-fluoro-3-methoxypyridine (50 mg, 0.26 mmol) in THF (2 mL) was added to the above mixture at 0° C. The reaction was warmed to 60° C. and stirred at this temperature for 3 h. Upon completion of the reaction, the mixture was cooled to r.t, quenched with H2O, and extracted with EA. The combined organic phases were washed with brine, dried over Na2SO4, and concentrated. The residue was purified silica gel column chromatography (PE:EA=3:1) to give the title compound (60 mg, 78%). MS(ESI) m/z [M+H]+=298.2.

### Step 3: 6-(bicyclo[1.1.1]pentan-1-yl)-3-methoxypyridine-2-sulfonamide

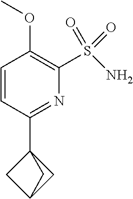 [SMILES: COc1ccc(C23CC(C2)C3)nc1S(N)(=O)=O]

To a mixture of 2-(benzylthio)-6-(bicyclo[1.1.1]pentan-1-yl)-3-methoxypyridine (60 mg, 0.20 mmol) in acetic acid (6 mL) and water (2 mL) was added NCS (180 mg, 0.8 mmol). The reaction was stirred at rt for 2 h. Upon completion of the reaction, the mixture was concentrated in vacuo to remove acetic acid. The residue was diluted with H2O and extracted with EA. The combined organic phases were washed brine, dried over anhydrous Na2SO4, concentrated to give the crude 6-(bicyclo[1.1.1]pentan-1-yl)-3-methoxypyridine-2-sulfonyl chloride.

To a mixture of crude 6-(bicyclo[1.1.1]pentan-1-yl)-3-methoxypyridine-2-sulfonyl chloride in THF (5 mL) was added NH3·H2O (1 mL). The reaction was stirred at rt for 2 h. Upon completion of the reaction, the reaction mixture was concentrated in vacuo. The residue was diluted with DCM and water. The aqueous phase was adjusted to pH=2-3 with 1 N HCl (aq.), extracted with DCM. The combined organic phases were washed by brine, dried over anhydrous Na2SO4, concentrated to give the title compound without further purification (50 mg, 99%). MS(ESI) m/z [M+H]+=255.1.

### Example C

### Example C1: Synthesis of 4-((1H-pyrazol-1-yl)methyl)-N-((5-(tert-butyl)-2-methoxyphenyl)sulfonyl)-3-iodobenzamide

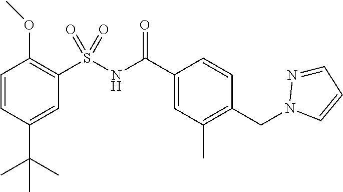 [SMILES: COc1ccc(C(C)(C)C)cc1S(=O)(=O)NC(=O)c1ccc(Cn2cccn2)c(C)c1]

A solution of 4-((1H-pyrazol-1-yl)methyl)-3-iodobenzoic acid (730 mg, 2.23 mmol), 5-(tert-butyl)-2-methoxybenzenesulfonamide (541 mg, 2.23 mmol), EDCI (852 mg, 4.46 mmol) and DMAP (817 mg, 6.69 mmol) in DCM (20 mL) was stirred at rt for overnight. Upon completion of the reaction, the mixture was quenched by water. The aqueous layer was extracted with DCM. The organic layer was concentrated, and the residue was purified by silica gel column chromatography (MeOH/DCM=1:20) to give the title compound (1.1 g, 89%). 1H NMR (400 MHz, DMSO-d6) δ 12.57 (s, 1H), 8.37 (s, 1H), 7.84 (d, J=12.0 Hz, 2H), 7.78 (d, J=8.0 Hz, 1H), 7.67 (d, J=7.1 Hz, 1H), 7.52 (s, 1H), 7.13 (d, J=8.3 Hz, 1H), 6.65 (d, J=8.0 Hz, 1H), 6.32 (s, 1H), 5.37 (s, 2H), 3.81 (s, 3H), 1.28 (s, 9H). MS (ESI) m/e [M+H]+=554.

### Example C2: Synthesis of 4-((1H-pyrazol-1-yl)methyl)-N-((5-(tert-butyl)-2-methoxyphenyl)sulfonyl)-3-cyclopropylbenzamide

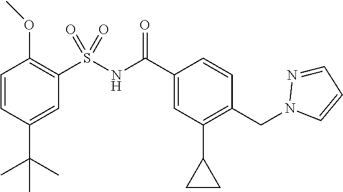 [SMILES: COc1ccc(C(C)(C)C)cc1S(=O)(=O)NC(=O)c1ccc(Cn2cccn2)c(C2CC2)c1]

A solution of 4-((1H-pyrazol-1-yl)methyl)-N-((5-(tert-butyl)-2-methoxyphenyl)sulfonyl)-3-iodobenzamide (50 mg, 0.09 mmol), cyclopropyl boronic acid (16 mg, 0.18 mmol), Pd(OAc)2 (2 mg, 0.009 mmol), Cy3P (5 mg, 0.018 mmol) and K3PO4 (39 mg, 0.18 mmol) in 1,4-dioxane (4 mL)/water (0.4 mL) was stirred at 140° C. for 4 h under microwave irritation. After cooled to rt, the reaction was concentrated, water was added, the mixture was extracted with MeOH/DCM, the organic layer was concentrated, and the residue was purified by Prep-HPLC (Waters Sunfire C18, 55%-70% ACN in H2O/0.1% FA) to give the product (1 mg, 2.4%). 1H NMR (400 MHz, DMSO-d6) δ 13.00-12.15 (m, 1H), 8.12 (s, 1H), 7.84-7.73 (m, 2H), 7.59 (d, J=7.6 Hz, 1H), 7.56-7.51 (m, 1H), 7.46 (s, 1H), 6.96-6.83 (m, 1H), 6.74 (d, J=8.2 Hz, 1H), 6.27 (s, 1H), 5.51 (s, 2H), 3.65 (s, 3H), 2.04-1.98 (m, 1H), 1.25 (s, 9H), 0.95-0.90 (m, 2H), 0.65-0.54 (m, 2H). MS (ESI) m/e [M+H]+=468.

Examples C3 below (Table 1) were synthesized starting from the corresponding starting materials according to the similar procedures described as those of Example C2.

TABLE 1

| | | | ¹H NMR data LC/MS m/z |
|---|---|---|---|
| Example | Compound | Chemical Name | (M + 1) |
| C3 | 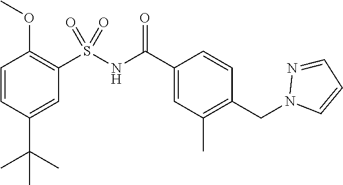 [SMILES: COc1ccc(C(C)(C)C)cc1S(=O)(=O)NC(=O)c1ccc(Cn2cccn2)c(C)c1] | 4-((1H-pyrazol-1- yl)methyl)-N-((5-(tert- butyl)-2- methoxyphenyl) sulfonyl)- 3-methylbenzamide | ¹H NMR (400 MHz, DMSO-d₆) δ 12.34 (s, 1H), 7.85-7.74 (m, 2H), 7.73- 7.69 (m, 1H), 7.69-7.54 (m, 2H), 7.49-7.45 (m, 1H), 7.17-7.00 (m, 1H), 6.80 (d, J = 8.0 Hz, 1H), 6.28 (s, 1H), 5.38 (s, 2H), 3.77 (s, 3H), 2.30 (s, 3H), 1.27 (s, 9H). MS (ESI) m/e [M + H]⁺ = 442. |

### Example C4: Synthesis of 4-((1H-pyrazol-1-yl)methyl)-N-((5-(tert-butyl)-2-methoxyphenyl)sulfonyl)-3-vinylbenzamide

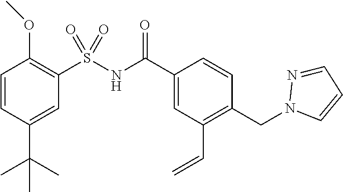 [SMILES: C=Cc1cc(C(=O)NS(=O)(=O)c2cc(C(C)(C)C)ccc2OC)ccc1Cn1cccn1]

A solution of 4-((1H-pyrazol-1-yl)methyl)-N-((5-(tert-butyl)-2-methoxyphenyl)sulfonyl)-3-iodobenzamide (150 mg, 0.27 mmol), tributyl(vinyl)stannane (172 mg, 0.54 mmol) and Pd(PPh₃)₄ (31 mg, 0.027 mmol) in THF (10 mL) was stirred at 80° C. for overnight. After cooled to rt, the reaction was concentrated, water was added, the mixture was extracted with MeOH/DCM, the organic layer was concentrated, and the crude was purified by silica gel column chromatography (MeOH/DCM=1:20) to give the product (100 mg, 81%). ¹H NMR (400 MHz, DMSO-d₆) δ 12.52 (s, 1H), 8.12 (s, 1H), 7.84 (d, J=2.3 Hz, 1H), 7.78 (d, J=1.5 Hz, 1H), 7.71-7.64 (m, 2H), 7.46 (d, J=1.1 Hz, 1H), 7.13 (d, J=17.5 Hz, 2H), 6.93 (d, J=8.1 Hz, 1H), 6.29-6.25 (m, 1H), 5.93 (d, J=17.2 Hz, 1H), 5.50-5.41 (m, 3H), 3.80 (s, 3H), 1.28 (s, 9H). MS (ESI) m/e [M+H]⁺=454.

Examples C5 below (Table 2) were synthesized starting from the corresponding starting materials according to the similar procedures described as those of Example C4.

TABLE 2

| | | | ¹H NMR data LC/MS m/z |
|---|---|---|---|
| Example | Compound | Chemical Name | (M + 1) |
| C5 | 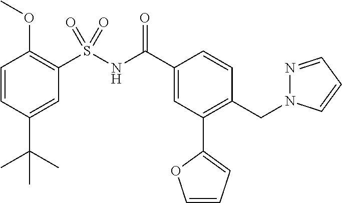 [SMILES: COc1ccc(C(C)(C)C)cc1S(=O)(=O)NC(=O)c1ccc(Cn2cccn2)c(-c2ccco2)c1] | 4-((1H-pyrazol-1- yl)methyl)-N-((5-(tert- butyl)-2- methoxyphenyl)sulfonyl)- 3-(furan-2- yl)benzamide | ¹H NMR (400 MHz, DMSO-d₆) δ 12.56 (s, 1H), 8.22 (s, 1H), 7.91-7.82 (m, 2H), 7.81-7.76 (m, 1H), 7.74-7.62 (m, 2H), 7.54- 7.49 (m, 1H), 7.12 (d, J = 7.9 Hz, 1H), 7.00-6.93 (m, 1H), 6.73-6.65 (m, 2H), 6.31 (s, 1H), 5.62 (s, 2H), 3.81 (s, 3H), 1.28 (s, 9H). MS (ESI) m/e [M + H]⁺ = 494. |

### Example C6: Synthesis of 4-((1H-pyrazol-1-yl)methyl)-N-((5-(tert-butyl)-2-methoxyphenyl)sulfonyl)-3-ethylbenzamide

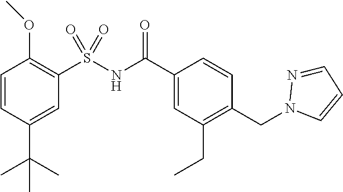 [SMILES: CCc1cc(C(=O)NS(=O)(=O)c2cc(C(C)(C)C)ccc2OC)ccc1Cn1cccn1]

A solution of 4-((1H-pyrazol-1-yl)methyl)-N-((5-(tert-butyl)-2-methoxyphenyl)sulfonyl)-3-vinylbenzamide (50 mg, 0.11 mmol) and Pd/C (10 mg, 10%) in MeOH (5 mL) was introduced H₂ (1 atm), the resulting mixture was stirred at 30° C. for 4 h. After cooled to rt, the reaction was filtered out and the filtrate was concentrated, the residue was purified by Prep-HPLC (Waters Sunfire C18, 50%-70% ACN in H₂O/0.1% FA) to give the product (5.52 mg, 11.0%). ¹H NMR (400 MHz, DMSO-d₆) δ 12.40 (s, 1H), 7.84 (d, J=2.2 Hz, 1H), 7.81-7.72 (m, 2H), 7.70-7.59 (m, 2H), 7.47 (s, 1H), 7.12 (d, J=8.7 Hz, 1H), 6.86 (d, J=8.1 Hz, 1H), 6.28 (s, 1H), 5.41 (s, 2H), 3.80 (s, 3H), 2.70 (q, J=7.4 Hz, 2H), 1.28 (s, 9H), 1.13 (t, J=7.5 Hz, 3H). MS (ESI) m/e [M+H]⁺=456.

### Example C7: Synthesis of 4-((1H-pyrazol-1-yl)methyl)-N-((5-(tert-butyl)-2-methoxyphenyl)sulfonyl)-3-methoxybenzamide

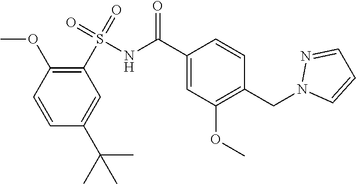 [SMILES: COc1cc(C(=O)NS(=O)(=O)c2cc(C(C)(C)C)ccc2OC)ccc1Cn1cccn1]

A solution of 4-((1H-pyrazol-1-yl)methyl)-3-methoxybenzoic acid (25 mg, 0.11 mmol), 5-(tert-butyl)-2-methoxybenzenesulfonamide (27 mg, 0.11 mmol), EDCI (42 mg, 0.22 mmol) and DMAP (41 mg, 0.33 mmol) in DCM (50 mL) was stirred at rt for overnight. Upon completion of the reaction, the mixture was quenched by water. The aqueous layer was extracted with DCM. The organic layer was concentrated, and the residue was purified by Prep-HPLC (Waters Sunfire C18, 50%-70% ACN in H₂O/0.1% FA) to give the product (21 mg, 42MH). ¹H NMR (400 MHz, DMSO-d₆) δ 12.46 (s, 1H), 7.84 (d, J=2.3 Hz, 1H), 7.77 (d, J=1.5 Hz, 1H), 7.68 (d, J=7.7 Hz, 1H), 7.52 (s, 1H), 7.48-7.43 (m, 1H), 7.39 (d, J=7.8 Hz, 1H), 7.13 (d, J=8.7 Hz, 1H), 6.74 (d, J=7.9 Hz, 1H), 6.27 (s, 1H), 5.31 (s, 2H), 3.89 (s, 3H), 3.81 (s, 3H), 1.28 (s, 9H). MS (ESI) m/e [M+H]⁺=458.

Examples C8-C17 below (Table 3) were synthesized starting from the corresponding starting materials according to the similar procedures described as those of Example C7.

TABLE 3

| | | Chemical | |
|---|---|---|---|
| Example | Structure | Name | ¹H NMR data LC/MS m/z (M + H) |
| C8 | 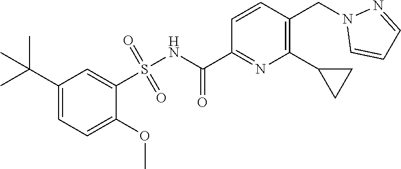 [SMILES: COc1ccc(C(C)(C)C)cc1S(=O)(=O)NC(=O)c1ccc(Cn2cccn2)c(C2CC2)n1] | 5-((1H- pyrazol-1- yl)methyl)-N- ((5-(tert- butyl)-2- methoxyphenyl) sulfonyl)-6- cyclopropyl- picolinamide | ¹H NMR (400 MHz, DMSO-d₆) δ 11.40 (s, 1H), 7.86 (d, J = 1.6 Hz, 1H), 7.83 (d, J = 2.5 Hz, 1H), 7.72-7.66 (m, 1H), 7.64 (d, J = 7.9 Hz, 1H), 7.50 (d, J = 1.6 Hz, 1H), 7.25 (d, J = 7.9 Hz, 1H), 7.14 (d, J = 8.8 Hz, 1H), 6.32- 6.29 (m, 1H), 5.63 (s, 2H), 3.80 (s, 3H), 2.42-2.34 (m, 1H), 1.27 (s, 9H), 1.24-1.18 (m, 2H), 1.06-0.97 (m, 2H).MS (ESI) m/e [M + 1]⁺ = 469.3. |
| C9 | 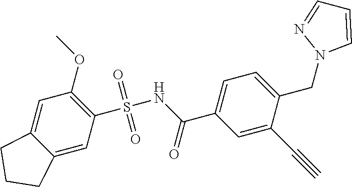 [SMILES: C#Cc1cc(C(=O)NS(=O)(=O)c2cc3c(cc2OC)CCC3)ccc1Cn1cccn1] | 4-((1H- pyrazol-1- yl)methyl)-3- ethynyl-N-((6- methoxy-2,3- dihydro-1H- inden-5- yl)sulfonyl) benzamide | ¹H NMR (400 MHz, DMSO-d₆)δ 12.46 (s, 1H), 7.99 (s, 1H), 7.86-7.74 (m, 2H), 7.70-7.55 (m, 1H), 7.53-7.45 (m, 1H), 7.15-7.69 (m, 1H), 6.90-6.75 (m, 1H), 6.30-6.26 (s, 1H), 5.48 (s, 2H), 4.70-4.40 (m, 1H), 3.85-3.55 (m, 3H), 2.95-2.75 (m, 4H), 2.06-1.94 (m, 2H). MS (ESI) m/e [M + 1]⁺ = 436.3. |
| C10 | 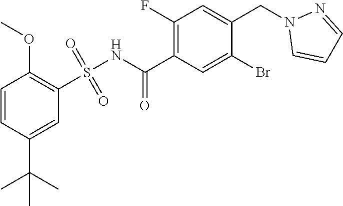 [SMILES: COc1ccc(C(C)(C)C)cc1S(=O)(=O)NC(=O)c1cc(Br)c(Cn2cccn2)cc1F] | 4-((1H- pyrazol-1- yl)methyl)-5- bromo-N-((5- (tert-butyl)-2- methoxyphenyl) sulfonyl)-2- fluorobenzamide | ¹H NMR (400 MHz, DMSO-d₆) δ 12.73 (s, 1H), 7.90 (d, J = 1.5 Hz, 1H), 7.84 − 7.80 (m, 2H), 7.75 − 7.67 (m, 1H), 7.55 (s, 1H), 7.22 − 7.15 (m, 1H), 6.57 (d, J = 10.7 Hz, 1H), 6.37 − 6.33 (m, 1H), 5.44 (s, 2H), 3.86 (s, 3H), 1.29 (s, 9H). MS (ESI) m/e [M + H]⁺ = 524.2. |
| C11 | 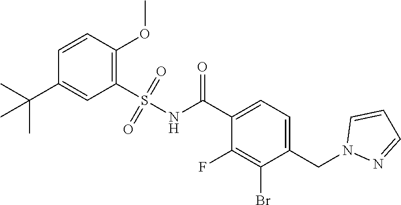 [SMILES: COc1ccc(C(C)(C)C)cc1S(=O)(=O)NC(=O)c1ccc(Cn2cccn2)c(Br)c1F] | 4-((1H- pyrazol-1- yl)methyl)-3- bromo-N-((5- (tert-butyl)-2- methoxyphenyl) sulfonyl)-2- fluorobenzamide | ¹H NMR (400 MHz, DMSO-d₆) δ 12.74 (s, 1H), 7.89 (d, J = 1.5 Hz, 1H), 7.82 (d, J = 2.3 Hz, 1H), 7.75 − 7.68 (m, 1H), 7.54 (s, 1H), 7.48 − 7.42 (m, 1H), 7.19 (d, J = 8.4 Hz, 1H), 6.60 (d, J = 7.9 Hz, 1H), 6.36 − 6.32 (m, 1H), 5.49 (s, 2H), 3.86 (s, 3H), 1.29 (s, 9H). MS (ESI) m/e [M + H]⁺ = 524.2. |
| C12 | 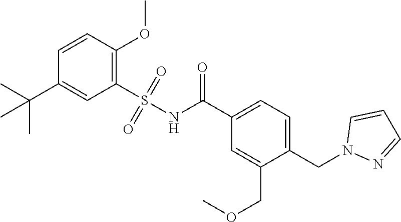 [SMILES: COCc1cc(C(=O)NS(=O)(=O)c2cc(C(C)(C)C)ccc2OC)ccc1Cn1cccn1] | 4-((1H- pyrazol-1- yl)methyl)-N- ((5-(tert- butyl)-2- methoxyphenyl) sulfonyl)-3- (methoxymethyl) benzamide | ¹H NMR (400 MHz, DMSO-d₆) δ 12.51 (s, 1H), 7.91 − 7.88 (m, 1H), 7.85 − 7.82 (m, 1H), 7.81 (s, 1H), 7.74 (d, J = 8.1 Hz, 1H), 7.64 − 7.54 (m, 1H), 7.49 (s, 1H), 7.11 − 7.00 (m, 1H), 6.82 (d, J = 7.8 Hz, 1H), 6.32 − 6.27 (m, 1H), 5.43 (s, 2H), 4.54 (s, 2H), 3.75 (s, 3H), 3.33 (s, 3H), 1.28 (s, 9H). MS (ESI) m/e [M + H]⁺ = 472.4. |
| C13 | 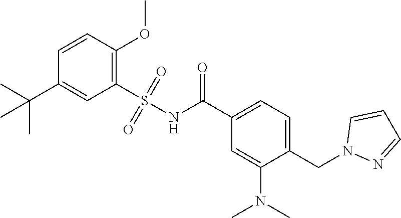 [SMILES: COc1ccc(C(C)(C)C)cc1S(=O)(=O)NC(=O)c1ccc(Cn2cccn2)c(N(C)C)c1] | 4-((1H- pyrazol-1- yl)methyl)-N- ((5-(tert- butyl)-2- methoxyphenyl) sulfonyl)-3- (dimethylamino) benzamide | ¹H NMR (400 MHz, DMSO-d₆) δ 12.50 (s, 1H), 7.87 − 7.83 (m, 2H), 7.72 − 7.65 (m, 2H), 7.49 (s, 1H), 7.46 − 7.41 (m, 1H), 7.15 (d, J = 8.6 Hz, 1H), 6.62 (d, J = 7.9 Hz, 1H), 6.32 − 6.29 (m, 1H), 5.45 (s, 2H), 3.83 (s, 3H), 2.70 (s, 6H), 1.29 (s, 9H). MS (ESI) m/e [M + H]⁺ = 471.4. |
| C14 | 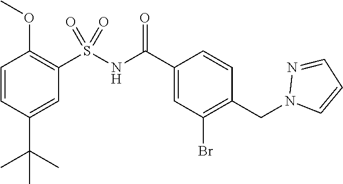 [SMILES: COc1ccc(C(C)(C)C)cc1S(=O)(=O)NC(=O)c1ccc(Cn2cccn2)c(Br)c1] | 4-((1H- pyrazol-1- yl)methyl)-3- bromo-N-((5- (tert-butyl)-2- methoxyphenyl) sulfonyl) benzamide | ¹H NMR (400 MHz, DMSO-d₆) δ 12.61 (s, 1H), 8.17-8.13 (m, 1H), 7.89- 7.86 (m, 1H), 7.85-7.81 (m, 1H), 7.78 (d, J = 8.1 Hz, 1H), 7.68 (d, J = 7.8 Hz, 1H), 7.52 (s, 1H), 7.13 (d, J = 8.7 Hz, 1H), 6.77 (d, J = 8.1 Hz, 1H), 6.32 (s, 1H), 5.45 (s, 2H), 3.81 (s, 3H), 1.28 (s, 9H). MS (ESI) m/e [M + H]⁺ = 506. |
| C15 | 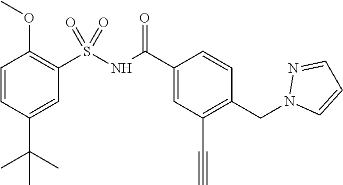 [SMILES: C#Cc1cc(C(=O)NS(=O)(=O)c2cc(C(C)(C)C)ccc2OC)ccc1Cn1cccn1] | 4-((1H- pyrazol-1- yl)methyl)-N- ((5-(tert- butyl)-2- methoxyphenyl) sulfonyl)-3- ethynylbenzamide | ¹H NMR (400 MHz, DMSO-d₆) δ 12.55 (s, 1H), 8.01 (s, 1H), 7.85 −7.81 (m, 2H), 7.79 (br d, J = 7.8 Hz, 1H), 7.68 (br s, 1H), 7.48 (s, 1H), 7.13 (br d, J = 7.8 Hz, 1H), 6.85 (br d, J = 8.8 Hz, 1H), 6.29 (br s, 1H), 5.50 (s, 2H), 4.64 (s, 1H), 3.79 (s, 3H), 1.27 (s, 9H). MS (ESI) m/e [M + H]⁺ = 452.4. |
| C16 | 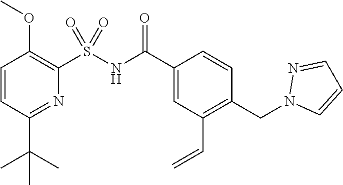 [SMILES: C=Cc1cc(C(=O)NS(=O)(=O)c2nc(C(C)(C)C)ccc2OC)ccc1Cn1cccn1] | 4-((1H- pyrazol-1- yl)methyl)-N- ((6-(tert- butyl)-3- methoxypyridin-2- yl)sulfonyl)-3- vinylbenzamide | ¹H NMR (400 MHz, DMSO-d₆) δ 12.37 (s, 1H), 8.18-8.13 (m, 1H), 7.83- 7.78 (m, 1H), 7.75-7.68 (m, 2H), 7.65 (d, J = 8.6 Hz, 1H), 7.51-7.46 (m, 1H), 7.16 (dd, J = 17.2, 11.0 Hz, 1H), 6.93 (d, J = 8.1 Hz, 1H), 6.29 (s, 1H), 5.89 (d, J = 17.3 Hz, 1H), 5.50 (s, 2H), 5.45 (d, J = 11.1 Hz, 1H), 3.90 (s, 3H), 1.13 (s, 9H). MS (ESI) m/e [M + H]⁺ = 455. |
| C17 | 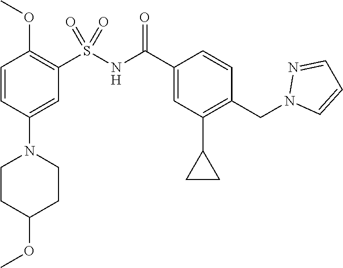 [SMILES: COc1ccc(N2CCC(OC)CC2)cc1S(=O)(=O)NC(=O)c1ccc(Cn2cccn2)c(C2CC2)c1] | 4-((1H- pyrazol-1- yl)methyl)-3- cyclopropyl- N-((2- methoxy-5-(4- methoxypiperidin- 1- yl)phenyl) sulfonyl) benzamide | ¹H NMR (400 MHz, DMSO-d₆) δ 12.43 (s, 1H), 7.80 (s, 1H), 7.60-7.51 (m, 2H), 7.50-7.45 (m, 1H), 7.41-7.35 (m, 1H), 7.31-7.16 (m, 1H), 7.13-6.98 (m, 1H), 6.75 (d, J = 8.0 Hz, 1H), 6.29 (s, 1H), 5.55 (s, 2H), 3.73 (s, 3H), 3.25 (s, 3H), 2.87-2.76 (m, 2H), 2.49 (s, 3H), 2.09-2.01 (m, 1H), 1.98-1.89 (m, 2H), 1.59-1.48 (m, 2H), 0.94 (d, J = 7.4 Hz, 2H), 0.83-0.66 (m, 2H). MS (ESI) m/e [M + H]⁺ = 525. |
| C18 | 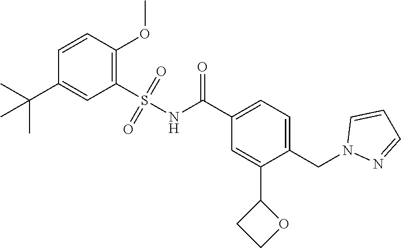 [SMILES: COc1ccc(C(C)(C)C)cc1S(=O)(=O)NC(=O)c1ccc(Cn2cccn2)c(C2CCO2)c1] | 4-((1H- pyrazol-1- yl)methyl)-N- ((5-(tert- butyl)-2- methoxyphenyl) sulfonyl)-3- (oxetan-2- yl)benzamide | ¹H NMR (400 MHz, DMSO-d₆) δ 12.62 (s, 1H), 8.18 − 8.16 (m, 1H), 7.88 − 7.85 (m, 1H), 7.80 (s, 1H), 7.74 (d, J = 8.0 Hz, 1H), 7.72 − 7.67 (m, 1H), 7.48 (s, 1H), 7.16 (s, 1H), 6.91 (d, J = 7.6 Hz, 1H), 6.31 − 6.29 (m, 1H), 6.09 − 6.03 (m, 1H), 5.31 (s, 2H), 4.74 − 4.68 (m, J = 6.4 Hz, 1H), 4.60 − 4.53 (m, 1H), 3.83 (s, 3H), 2.96 − 2.87 (m, 1H), 2.46 − 2.38 (m, 1H), 1.30 (s, 9H). MS (ESI) m/e [M + H]⁺ = 484.4. |
| C19 | 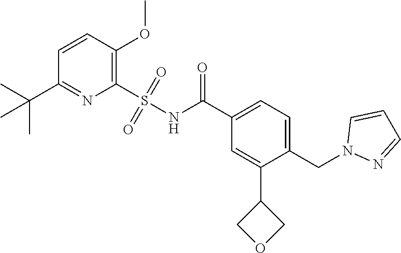 [SMILES: COc1ccc(C(C)(C)C)nc1S(=O)(=O)NC(=O)c1ccc(Cn2cccn2)c(C2COC2)c1] | 4-((1H- pyrazol-1- yl)methyl)-N- ((6-(tert- butyl)-3- methoxypyridin- 2- yl)sulfonyl)-3- (oxetan-3- yl)benzamid | ¹H NMR (400 MHz, DMSO-d₆) δ 12.48 (s, 1H), 8.22 (s, 1H), 7.80 − 7.76 (m, 1H), 7.75 − 7.64 (m, 3H), 7.53 − 7.49 (m, 1H), 6.93 (d, J = 8.1 Hz, 1H), 6.32 − 6.29 (m, 1H), 5.34 (s, 2H), 4.91 − 4.84 (m, 2H), 4.75 − 4.63 (m, 3H), 3.93 (s, 3H), 1.16 (s, 9H). MS (ESI) m/e [M + H]⁺ = 485.4. |
| C20 | 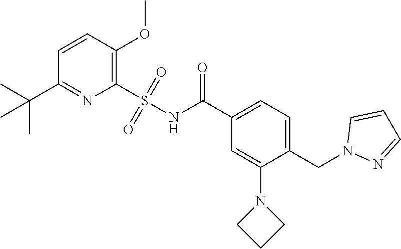 [SMILES: COc1ccc(C(C)(C)C)nc1S(=O)(=O)NC(=O)c1ccc(Cn2cccn2)c(N2CCC2)c1] | 4-((1H- pyrazol-1- yl)methyl)-3- (azetidin-1- yl)-N-((6- (tert-butyl)-3- methoxypyridin- 2- yl)sulfonyl) benzamide | ¹H NMR (400 MHz, DMSO-d₆) δ 12.26 (s, 1H), 7.78 − 7.64 (m, 3H), 7.53 (s, 1H), 7.21 (d, J = 8.1 Hz, 1H), 7.07 − 7.04 (m, 1H), 6.46 (d, J = 7.9 Hz, 1H), 6.34 − 6.31 (m, 1H), 5.31 (s, 2H), 3.98 (t, J = 6.9 Hz, 4H), 3.92 (s, 3H), 2.29 − 2.21 (m, 2H), 1.17 (s, 9H). MS (ESI) m/e [M + H]⁺ = 484.4. |
| C21 | 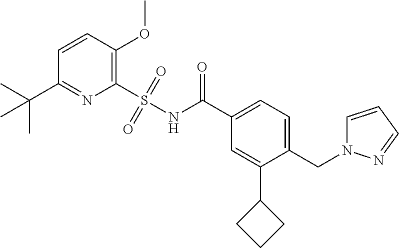 [SMILES: COc1ccc(C(C)(C)C)nc1S(=O)(=O)NC(=O)c1ccc(Cn2cccn2)c(C2CCC2)c1] | 4-((1H- pyrazol-1- yl)methyl)-N- ((6-(tert- butyl)-3- methoxypyridin-2- yl)sulfonyl)-3- cyclobutyl- benzamide | ¹H NMR (400 MHz, DMSO-d₆) δ 12.39 (s, 1H), 7.93 (s, 1H), 7.83 − 7.77 (m, 1H), 7.76 − 7.60 (m, 3H), 7.51 (s, 1H), 6.74 (d, J = 7.8 Hz, 1H), 6.34 − 6.28 (m, 1H), 5.39 (s, 2H), 3.92 (s, 3H), 3.85 − 3.76 (m, 1H), 2.35 − 2.24 (m, 2H), 2.22 − 2.10 (m, 2H), 2.05 − 1.93 (m, 1H), 1.87 − 1.76 (m, 1H), 1.16 (s, 9H). MS (ESI) m/e [M + H]⁺ = 483.4. |
| C22 | 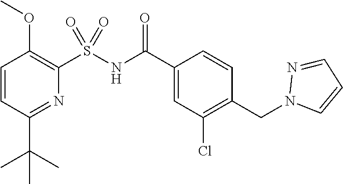 [SMILES: COc1ccc(C(C)(C)C)nc1S(=O)(=O)NC(=O)c1ccc(Cn2cccn2)c(Cl)c1] | 4-((1H- pyrazol-1- yl)methyl)-N- ((6-(tert- butyl)-3- methoxypyridin- 2- yl)sulfonyl)-3- chlorobenzamide | ¹H NMR (400 MHz, DMSO-d₆) δ 12.44 (s, 1H), 8.00 (s, 1H), 7.87 (d, J = 2.0 Hz, 1H), 7.78 (d, J = 8.0 Hz, 1H), 7.67 − 7.60 (m, 2H), 7.51 (d, J = 2.0 Hz, 1H), 6.86 (d, J = 8.0 Hz, 1H), 6.31 (t, J = 2.0 Hz, 1H), 5.49 (s, 2H), 3.86 (s, 3H), 1.11 (s, 9H). MS (ESI) m/e [M + H]⁺ = 463. |
| C23 | 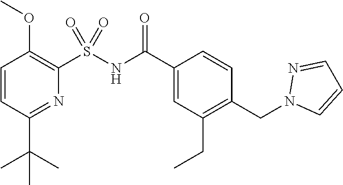 [SMILES: CCc1cc(C(=O)NS(=O)(=O)c2nc(C(C)(C)C)ccc2OC)ccc1Cn1cccn1] | 4-((1H- pyrazol-1- yl)methyl)-N- ((6-(tert- butyl)-3- methoxypyridin- 2- yl)sulfonyl)-3- ethylbenzamide | ¹H NMR (400 MHz, DMSO-d₆) δ12.26 (s, 1H), 7.79 (d, J = 2.0 Hz, 1H), 7.77 (d, J = 2.0 Hz, 1H), 7.69 (d, J = 8.7 Hz, 1H), 7.66-7.59 (m, 2H), 7.47 (d, J = 2.0 Hz, 1H), 6.84 (d, J = 8.1 Hz, 1H), 6.28 (t, J = 2.0 Hz, 1H), 5.43 (s, 2H), 3.88 (s, 3H), 2.75-2.67 (m, 2H), 1.15-1.08 (m, 12H). MS (ESI) m/e [M + H]⁺ = 457. |
| C24 | 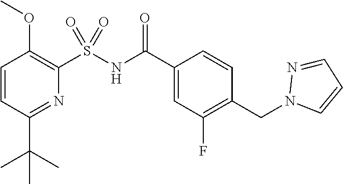 [SMILES: COc1ccc(C(C)(C)C)nc1S(=O)(=O)NC(=O)c1ccc(Cn2cccn2)c(F)c1] | 4-((1H- pyrazol-1- yl)methyl)-N- ((6-(tert- butyl)-3- methoxypyridin- 2- yl)sulfonyl)-3- fluorobenzamide | ¹H NMR (400 MHz, DMSO-d₆) δ 12.41 (brs, 1H), 7.85 − 7.84 (m, 1H), 7.75 − 7.68 (m, 3H), 7.63 (d, J = 8.0 Hz, 1H), 7.47 (s, 1H), 7.15 − 7.11 (m, 1H), 6.28 − 6.27 (m, 1H), 5.45 (s, 2H), 3.88 (s, 3H), 1.11 (s, 9H). MS (ESI) m/e [M + H]⁺ = 447. |
| C25 | 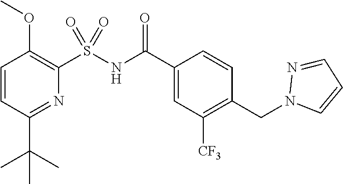 [SMILES: COc1ccc(C(C)(C)C)nc1S(=O)(=O)NC(=O)c1ccc(Cn2cccn2)c(C(F)(F)F)c1] | 4-((1H- pyrazol-1- yl)methyl)-N- ((6-(tert- butyl)-3- methoxypyridin-2- yl)sulfonyl)-3- (trifluoromethyl) benzamide | ¹H NMR (400 MHz, DMSO-d₆) δ 12.60 (brs, 1H), 8.30 (s, 1H), 8.07 (d, J = 8.0 Hz, 1H), 7.91 − 7.90 (m, 1H), 7.67 − 7.63 (m, 2H), 7.54 − 7.53 (m, 1H), 6.83 (d, J = 8.0 Hz, 1H), 6.35 − 6.34 (m, 1H), 5.61 (s, 2H), 3.88 (s, 3H), 1.10 (s, 9H). MS (ESI) m/e [M + H]⁺ = 497. |
| C26 | 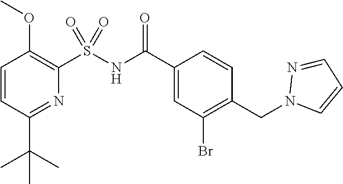 [SMILES: COc1ccc(C(C)(C)C)nc1S(=O)(=O)NC(=O)c1ccc(Cn2cccn2)c(Br)c1] | 4-((1H- pyrazol-1- yl)methyl)-3- bromo-N-((6- (tert-butyl)-3- methoxypyridin- 2- yl)sulfonyl) benzamide | ¹H NMR (400 MHz, DMSO-d₆) δ 12.44 (s, 1H), 8.17 (s, 1H), 7.88 (d, J = 1.9 Hz, 1H), 7.82 (d, J = 8.1, 1H), 7.70 − 7.60 (m, 2H), 7.52 (d, J = 1.3 Hz, 1H), 6.77 (d, J = 8.1 Hz, 1H), 6.32 (t, J = 2.0 Hz, 1H), 5.46 (s, 2H), 3.88 (s, 3H), 1.11 (s, 9H). MS (ESI) m/e [M + H]⁺ = 507. |
| C27 | 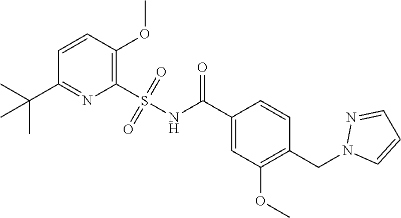 [SMILES: COc1cc(C(=O)NS(=O)(=O)c2nc(C(C)(C)C)ccc2OC)ccc1Cn1cccn1] | 4-((1H- pyrazol-1- yl)methyl)-N- ((6-(tert- butyl)-3- methoxypyridin-2- yl)sulfonyl)-3- methoxy- benzamide | ¹H NMR (400 MHz, DMSO-d₆) δ 12.30 (s, 1H), 7.78 (d, J = 1.9 Hz, 1H), 7.69 (d, J = 8.8 Hz, 1H), 7.63 (d, J = 8.7 Hz, 1H), 7.55 (d, J = 1.3 Hz, 1H), 7.46 (d, J = 1.3 Hz, 1H), 7.41 (d, J = 7.9, 1H), 6.74 (d, J = 7.9 Hz, 1H), 6.27 (t, J = 2.0 Hz, 1H), 5.32 (s, 2H), 3.88 (s, 3H), 3.87 (s, 3H), 1.12 (s, 9H). MS (ESI) m/e [M + H]⁺ = 459. |
| C28 | 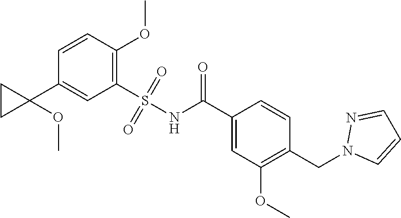 [SMILES: COc1cc(C(=O)NS(=O)(=O)c2cc(C3(OC)CC3)ccc2OC)ccc1Cn1cccn1] | 4-((1H- pyrazol-1- yl)methyl)-3- methoxy-N- ((2-methoxy- 5-(1- methoxy- cyclopropyl) phenyl) sulfonyl) benzamide | ¹H NMR (400 MHz, DMSO-d₆) δ 12.51 (s, 1H), 7.81 (d, J = 2.3 Hz, 1H), 7.75 (d, J = 2.1 Hz, 1H), 7.51 (d, J = 1.3 Hz, 1H), 7.49 − 7.40 (m, 2H), 7.37 (d, J = 7.9, 1H), 7.16 (d, J = 8.7 Hz, 1H), 6.73 (d, J = 7.9 Hz, 1H), 6.25 (t, J = 2.0 Hz, 1H), 5.29 (s, 2H), 3.87 (s, 3H), 3.81 (s, 3H), 3.12 (s, 3H), 1.14 (t, J = 5.1 Hz, 2H), 0.92 (t, J = 5.4 Hz, 2H). MS (ESI) m/e [M + H]⁺ = 472. |
| C29 | 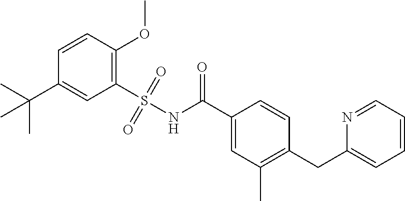 [SMILES: COc1ccc(C(C)(C)C)cc1S(=O)(=O)NC(=O)c1ccc(Cc2ccccn2)c(C)c1] | N-((5-(tert- butyl)-2- methoxyphenyl) sulfonyl)-3- methyl-4- (pyridin-2- ylmethyl) benzamide | ¹H NMR (400 MHz, DMSO-d₆) δ 12.28 (br s, 1H), 8.43 (d, J = 4.1 Hz, 1H), 7.81 (d, J = 2.5 Hz, 1H), 7.71 − 7.65 (m, 2H), 7.65 − 7.57 (m, 2H), 7.25 − 7.13 (m, 3H), 7.07 (br s, 1H), 4.10 (s, 2H), 3.75 (s, 3H), 2.23 (s, 3H), 1.26 (s, 9H). MS (ESI) m/e [M + H]⁺ = 453.4. |
| C30 | 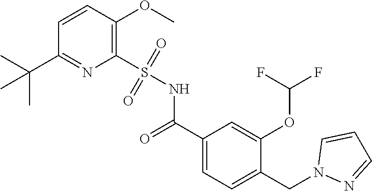 [SMILES: COc1ccc(C(C)(C)C)nc1S(=O)(=O)NC(=O)c1ccc(Cn2cccn2)c(OC(F)F)c1] | 4-((1H- pyrazol-1- yl)methyl)-N- ((6-(tert- butyl)-3- methoxypyridin- 2- yl)sulfonyl)-3- (difluoromethoxy) benzamide | ¹H NMR (400 MHz, DMSO-d₆) δ 12.47 (s, 1H), 7.86 (d, J = 2.0 Hz, 1H), 7.77 − 7.63 (m, 4H), 7.52 (d, J = 1.3 Hz, 1H), 7.31 (t, J = 73.4 Hz, 1H), 6.99 (d, J = 8.4 Hz, 1H), 6.33 (t, J = 2.0 Hz, 1H), 5.45 (s, 2H), 3.91 (s, 3H), 1.15 (s, 9H). MS (ESI) m/e [M + H]⁺ = 495.3. |
| C31 | 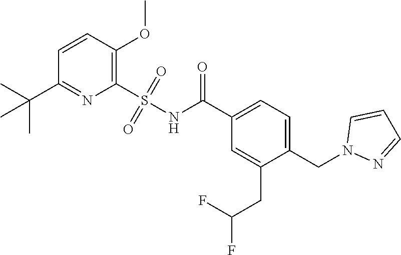 [SMILES: COc1ccc(C(C)(C)C)nc1S(=O)(=O)NC(=O)c1ccc(Cn2cccn2)c(CC(F)F)c1] | 4-((1H- pyrazol-1- yl)methyl)-N- ((6-(tert- butyl)-3- methoxypyridin- 2- yl)sulfonyl)-3- (2,2- difluoroethyl) benzamide | ¹H NMR (400 MHz, DMSO-d₆) δ 12.33 (s, 1H), 7.89 − 7.86 (m, 1H), 7.86 − 7.83 (m, 1H), 7.79 (d, J = 7.6 Hz, 1H), 7.73 − 7.58 (m, 2H), 7.52 (s, 1H), 6.98 (d, J = 8.1 Hz, 1H), 6.34 − 6.28 (m, 1H), 6.21 − 5.99 (m, 1H), 5.51 (s, 2H), 3.89 (s, 3H), 3.47 − 3.37 (m, J = 14.2 Hz, 2H), 1.16 (s, 9H). MS (ESI) m/e [M + H]⁺ = 493.3. |
| C32 | 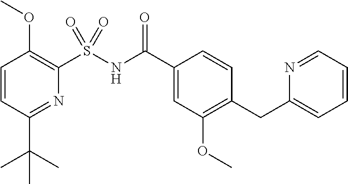 [SMILES: COc1cc(C(=O)NS(=O)(=O)c2nc(C(C)(C)C)ccc2OC)ccc1Cc1ccccn1] | N-((6-(tert- butyl)-3- methoxypyridin- 2- yl)sulfonyl)-3- methoxy-4- (pyridin-2- ylmethyl) benzamide | ¹H NMR (400 MHz, DMSO-d₆) δ 12.23 (s, 1H), 8.41 (d, J = 4.0 Hz, 1H), 7.77 − 7.53 (m, 3H), 7.50 (s, 1H), 7.41 (d, J = 7.7 Hz, 1H), 7.23 − 7.05 (m, 3H), 4.04 (s, 2H), 3.85 (s, 3H), 3.77 (s, 3H), 1.11 (s, 9H). MS (ESI) m/e [M + H]⁺ = 470. |
| C33 | 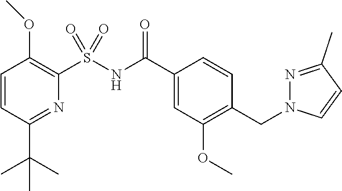 [SMILES: COc1cc(C(=O)NS(=O)(=O)c2nc(C(C)(C)C)ccc2OC)ccc1Cn1ccc(C)n1] | N-((6-(tert- butyl)-3- methoxypyridin- 2- yl)sulfonyl)-3- methoxy-4- ((3-methyl- 1H-pyrazol-1- yl)methyl) benzamide | ¹H NMR (400 MHz, DMSO-d₆) δ 12.28 (s, 1H), 7.73 − 7.58 (m, 3H), 7.56 − 7.50 (m, 1H), 7.39 (t, J = 6.8 Hz, 1H), 6.76 (d, J = 7.9 Hz, 1H), 6.01 (d, J = 1.8 Hz, 1H), 5.20 (s, 2H), 3.86 (s, 6H), 2.10 (s, 3H), 1.10 (d, J = 2.1 Hz, 9H). MS (ESI) m/e [M + H]⁺ = 473. |
| C34 | 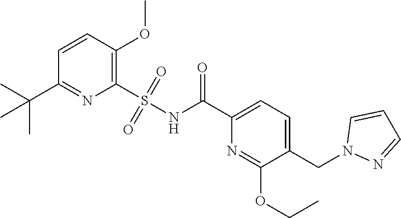 [SMILES: CCOc1nc(C(=O)NS(=O)(=O)c2nc(C(C)(C)C)ccc2OC)ccc1Cn1cccn1] | 5-((1H- pyrazol-1- yl)methyl)-N- ((6-(tert- butyl)-3- methoxypyridin- 2- yl)sulfonyl)-6- ethoxy- picolinamide | ¹H NMR (400 MHz, DMSO-d₆) δ 11.71 (s, 1H), 7.85 (d, J = 2.0 Hz, 1H), 7.74 (d, J = 8.8 Hz, 1H), 7.67 (d, J = 8.5 Hz, 1H), 7.55 (d, J = 7.5 Hz, 1H), 7.51 (d, J = 1.3 Hz, 1H), 7.26 (d, J = 7.6 Hz, 1H), 6.32 (t, J = 2.0 Hz, 1H), 5.37 (s, 2H), 4.61 (q, J = 6.8 Hz, 2H), 3.93 (s, 3H), 1.36 (t, J = 7.1 Hz, 3H), 1.09 (s, 9H). MS (ESI) m/e [M + H]⁺ = 474.3. |
| C35 | 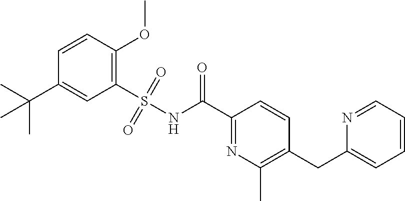 [SMILES: COc1ccc(C(C)(C)C)cc1S(=O)(=O)NC(=O)c1ccc(Cc2ccccn2)c(C)n1] | N-((5-(tert- butyl)-2- methoxyphenyl) sulfonyl)-6- methyl-5- (pyridin-2- ylmethyl) picolinamide | ¹H NMR (400 MHz, DMSO-d₆) δ 11.34 (s, 1H), 8.41 (d, J = 4.8 Hz, 1H), 7.81 (d, J = 2.5 Hz, 1H), 7.74 − 7.61 (m, 4H), 7.25 (d, J = 7.4 Hz, 1H), 7.19 (dd, J = 7.4, 5.4 Hz, 1H), 7.10 (d, J = 8.6 Hz, 1H), 4.19 (s, 2H), 3.75 (s, 3H), 2.51 (s, 3H), 1.24 (s, 9H). MS (ESI) m/e [M + H]⁺ = 454.3. |
| C36 | 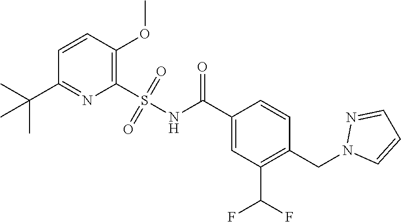 [SMILES: COc1ccc(C(C)(C)C)nc1S(=O)(=O)NC(=O)c1ccc(Cn2cccn2)c(C(F)F)c1] | 4-((1H- pyrazol-1- yl)methyl)-N- ((6-(tert- butyl)-3- methoxypyridin- 2- yl)sulfonyl)-3- (difluoromethyl) benzamide | ¹H NMR (400 MHz, DMSO-d₆) δ 12.51 (s, 1H), 8.12 (s, 1H), 7.95 (d, J = 8.1 Hz, 1H), 7.86 (d, J = 2.0 Hz, 1H), 7.67 (d, J = 8.8 Hz, 1H), 7.61 (d, J = 8.7 Hz, 1H), 7.52 − 7.46 (m, 1H), 7.40 − 7.20 (m, 1H), 6.90 (d, J = 8.1 Hz, 1H), 6.29 (t, J = 2.0 Hz, 1H), 5.57 (s, 2H), 3.86 (s, 3H), 1.07 (s, 9H). MS (ESI) m/e [M + H]⁺ = 479. |

### Example C37: Synthesis of 5-((3-amino-1H-pyrazol-1-yl)methyl)-N-((6-(tert-butyl)-3-methoxypyridin-2-yl)sulfonyl)-6-methoxypicolinamide

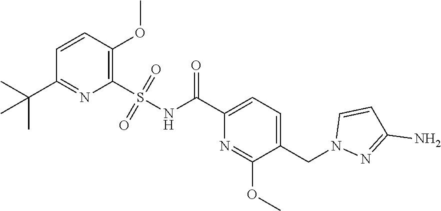 [SMILES: COc1ccc(C(C)(C)C)nc1S(=O)(=O)NC(=O)c1ccc(Cn2ccc(N)n2)c(OC)n1]

### Step 1: tert-butyl (1-((6-(((6-(tert-butyl)-3-methoxypyridin-2-yl)sulfonyl)carbamoyl)-2-methoxypyridin-3-yl)methyl)-1H-pyrazol-3-yl)carbamate

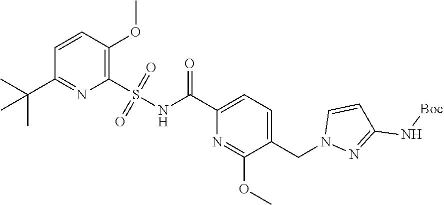 [SMILES: COc1ccc(C(C)(C)C)nc1S(=O)(=O)NC(=O)c1ccc(Cn2ccc(NC(=O)OC(C)(C)C)n2)c(OC)n1]

A solution of 5-((3-((tert-butoxycarbonyl)amino)-1H-pyrazol-1-yl)methyl)-6-methoxypicolinic acid (30 mg, 0.09 mmol), 6-(tert-butyl)-3-methoxypyridine-2-sulfonamide (21 mg, 0.09 mmol), DMAP (33 mg, 0.27 mmol) and EDCI (26 mg, 0.14 mmol) in DCM (2 mL) was stirred at rt for overnight. Upon completion of the reaction, the mixture was concentrated, and the residue was purified by C18 column (mobile phase, ACN in water (0.1% FA), 20% to 100%) to give the product (15 mg, 29%). MS (ESI) m/e [M+H]+=574.9.

### Step 2: 5-((3-amino-1H-pyrazol-1-yl)methyl)-N-((6-(tert-butyl)-3-methoxypyridin-2-yl)sulfonyl)-6-methoxypicolinamide

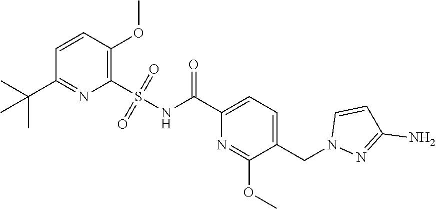 [SMILES: COc1ccc(C(C)(C)C)nc1S(=O)(=O)NC(=O)c1ccc(Cn2ccc(N)n2)c(OC)n1]

To a solution of tert-butyl (1-((6-(((6-(tert-butyl)-3-methoxypyridin-2-yl)sulfonyl)carbamoyl)-2-methoxypyridin-3-yl)methyl)-1H-pyrazol-3-yl)carbamate (15 mg, 0.03 mmol) in DCM (4 mL) was added TFA (1 mL) and the solution was stirred at rt for 4h. Upon completion of the reaction, the mixture was concentrated, and the residue was purified by C18 column (mobile phase, ACN in water (0.1% FA), 20% to 100%) to give the product (15 mg, 29%). 1H NMR (400 MHz, DMSO-d6) δ 11.82 (s, 1H), 7.74-7.70 (m, 1H), 7.68-7.64 (m, 1H), 7.57 (d, J=7.4 Hz, 1H), 7.47 (d, J=2.2 Hz, 1H), 7.25 (d, J=7.4 Hz, 1H), 5.46 (d, J=2.2 Hz, 1H), 5.07 (s, 2H), 4.64 (brs, 2H), 4.09 (s, 3H), 3.92 (s, 3H), 1.11 (s, 9H). MS (ESI) m/e [M+H]+=475.3.

Examples C38 below (Table 4) was synthesized starting from the corresponding starting materials according to the similar procedures described as those of Example C37.

TABLE 4

| xample | Compound | Chemical Name | 1H NMR data LC/MS m/z (M + H) |
|---|---|---|---|
| C38 | 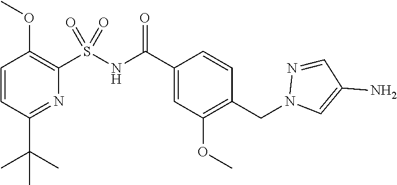 [SMILES: COc1cc(C(=O)NS(=O)(=O)c2nc(C(C)(C)C)ccc2OC)ccc1Cn1cc(N)cn1] | 4-((4-amino-1H-pyrazol-1- yl)methyl)-N-((6-(tert- butyl)-3-methoxypyridin-2- yl)sulfonyl)-3- methoxybenzamide | 1H NMR (400 MHz, DMSO-d₆) δ 7.58 − 7.47 (m, 3H), 7.38 (d, J = 7.5 Hz, 1H), 7.07 (s, 1H), 6.93 (s, 1H), 6.66 (d, J = 7.9 Hz, 1H), 5.09 (s, 2H), 3.82 (s, 3H), 3.80 (s, 3H), 1.12 (s, 9H). MS (ESI) m/e [M + H]+ = 474. |

### Example C39: Synthesis of 5-((1H-pyrazol-1-yl)methyl)-6-methoxy-N-((7-methoxy-4,4-dimethylchroman-8-yl)sulfonyl)picolinamide

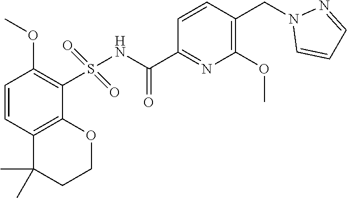 [SMILES: COc1ccc2c(c1S(=O)(=O)NC(=O)c1ccc(Cn3cccn3)c(OC)n1)OCCC2(C)C]

A mixture of 5-((1H-pyrazol-1-yl)methyl)-6-methoxypicolinic acid (15 mg, 0.06 mmol), 7-methoxy-4,4-dimethylchromane-8-sulfonamide (17 mg, 0.06 mmol), EDCI (25 mg, 0.13 mmol) and DMAP (24 mg, 0.19 mmol) was added into DCM (3 mL). The resulting reaction was stirred at room temperature for 16 hrs. Upon completion of the reaction, DCM was removed in vacuo. The residue was applied onto Prep-HPLC (column: Sunfire C18; mobile phase: [H2O (0.1% FA)-ACN]; gradient: 48%-63% B over 11.0 min) to give the title compound (7 mg, 23%). 1H NMR (400 MHz, DMSO-d6) δ 11.23 (brs, 1H), 7.81-7.80 (m, 1H), 7.55-7.43 (m, 3H), 7.16 (d, J=8.0 Hz, 1H), 6.66 (d, J=8.0 Hz, 1H), 6.27-6.26 (m, 1H), 5.32 (s, 2H), 4.06-4.04 (m, 5H), 3.68 (s, 3H), 1.67-1.65 (m, 2H), 1.18 (s, 6H). MS (ESI) m/e [M+1]+487.

### Example C40: Synthesis of 5-((1H-pyrazol-1-yl)methyl)-N-((5-(tert-butyl)-2-methoxyphenyl)sulfonyl)-6-methoxypicolinamide

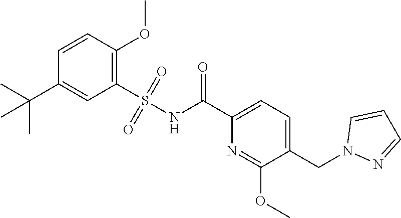 [SMILES: COc1ccc(C(C)(C)C)cc1S(=O)(=O)NC(=O)c1ccc(Cn2cccn2)c(OC)n1]

A mixture of 5-((1H-pyrazol-1-yl)methyl)-6-methoxypicolinic acid (23 mg, 0.1 mmol), 5-(tert-butyl)-2-methoxybenzenesulfonamide (24 mg, 0.1 mmol), DMAP (37 mg, 0.3 mmol) and EDCI (29 mg, 0.15 mmol) in DCM (2 mL) was stirred at rt for overnight. Upon completion of the reaction, the mixture was concentrated, and the residue was purified by C18 column (mobile phase, ACN in water (0.1% FA), 20% to 100%) to give the product (10 mg, 22%). 1H NMR (400 MHz, DMSO-d6) δ 11.75 (s, 1H), 7.85 (d, J=2.5 Hz, 1H), 7.84-7.82 (m, 1H), 7.73-7.57 (m, 1H), 7.53-7.47 (m, 2H), 7.23-7.06 (m, 2H), 6.30 (t, J=2.0 Hz, 1H), 5.34 (s, 2H), 4.08 (s, 3H), 3.79 (s, 3H), 1.29 (s, 9H). MS (ESI) m/e [M+H]+=459.3.

### Example C41: Synthesis of 5-((1H-pyrazol-1-yl)methyl)-N-((6-(tert-butyl)-3-methoxypyridin-2-yl)sulfonyl)-6-methoxypicolinamide

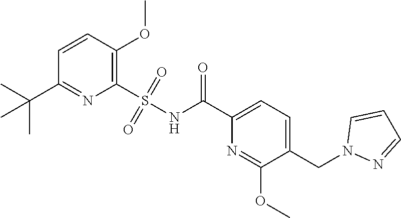 [SMILES: COc1ccc(C(C)(C)C)nc1S(=O)(=O)NC(=O)c1ccc(Cn2cccn2)c(OC)n1]

A mixture of 5-((1H-pyrazol-1-yl)methyl)-6-methoxypicolinic acid (23 mg, 0.1 mmol), 6-(tert-butyl)-3-methoxypyridine-2-sulfonamide (24 mg, 0.1 mmol), DMAP (37 mg, 0.3 mmol) and EDCI (29 mg, 0.15 mmol) in DCM (2 mL) was stirred at rt for 4h. Upon completion of the reaction, the mixture was concentrated, and the residue was purified by C18 column (mobile phase, ACN in water (0.1% FA), 20% to 100%) to give the product (7 mg, 15%). 1H NMR (400 MHz, DMSO-d6) δ 11.80 (s, 1H), 7.89-7.83 (m, 1H), 7.77-7.60 (m, 2H), 7.58-7.50 (m, 2H), 7.23-7.16 (m, 1H), 6.33-6.29 (m, 1H), 5.36 (s, 2H), 4.07 (s, 3H), 3.89 (s, 3H), 1.11 (s, 9H). MS (ESI) m/e [M+H]+=460.3.

### Example C42: Synthesis of 5-((1H-pyrazol-1-yl)methyl)-6-methoxy-N-((6-methoxy-2,2-dimethyl-2,3-dihydrobenzofuran-7-yl)sulfonyl)picolinamide

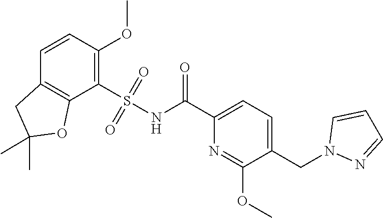 [SMILES: COc1ccc2c(c1S(=O)(=O)NC(=O)c1ccc(Cn3cccn3)c(OC)n1)OC(C)(C)C2]

A mixture of 5-((1H-pyrazol-1-yl)methyl)-6-methoxypicolinic acid (23 mg, 0.1 mmol), 6-methoxy-2,2-dimethyl-2,3-dihydrobenzofuran-7-sulfonamide (25 mg, 0.1 mmol), DMAP (37 mg, 0.3 mmol) and EDCI (29 mg, 0.15 mmol) in DCM (2 mL) was stirred at 45° C. for 4h. Upon completion of the reaction, the mixture was concentrated, and the residue was purified by C18 column (mobile phase, ACN in water (0.1% FA), 20% to 100%) to give the product (10 mg, 21%). 1H NMR (400 MHz, DMSO-d6) δ 11.39 (s, 1H), 7.84 (d, J=2.0 Hz, 1H), 7.56 (d, J=7.4 Hz, 1H), 7.52-7.50 (m, 1H), 7.31-7.26 (m, 1H), 7.20 (d, J=7.6 Hz, 1H), 6.54 (d, J=8.3 Hz, 1H), 6.33-6.29 (m, 1H), 5.36 (s, 2H), 4.09 (s, 3H), 3.75 (s, 3H), 2.90 (s, 2H), 1.24 (s, 6H). MS (ESI) m/e [M+H]+=473.3.

### Example C43: Synthesis of 5-((1H-pyrazol-1-yl)methyl)-6-methoxy-N-((6-methoxy-3,3-dimethyl-2,3-dihydrobenzofuran-7-yl)sulfonyl)picolinamide

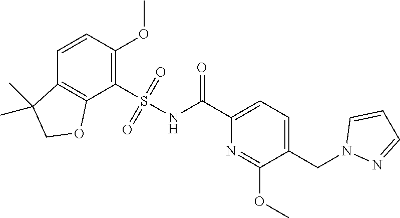 [SMILES: COc1ccc2c(c1S(=O)(=O)NC(=O)c1ccc(Cn3cccn3)c(OC)n1)OCC2(C)C]

A mixture of 5-((1H-pyrazol-1-yl)methyl)-6-methoxypicolinic acid (23 mg, 0.1 mmol), 6-methoxy-3,3-dimethyl-2,3-dihydrobenzofuran-7-sulfonamide (25 mg, 0.1 mmol), DMAP (37 mg, 0.3 mmol) and EDCI (29 mg, 0.15 mmol) in DCM (2 mL) was stirred at 45° C. for 4h. Upon completion of the reaction, the mixture was concentrated, and the residue was purified by C18 column (mobile phase, ACN in water (0.1% FA), 20% to 100%) to give the product (14 mg, 30%). 1H NMR (400 MHz, DMSO-d6) δ 11.57 (s, 1H), 7.85 (d, J=2.0 Hz, 1H), 7.55-7.50 (m, 2H), 7.41-7.32 (m, 1H), 7.19 (d, J=7.5 Hz, 1H), 6.60 (d, J=7.9 Hz, 1H), 6.32 (t, J=2.0 Hz, 1H), 5.36 (s, 2H), 4.28 (s, 2H), 4.10 (s, 3H), 3.74 (s, 3H), 1.26 (s, 6H). MS (ESI) m/e [M+H]+=473.3.

### Example C44: Synthesis of 4-((1H-pyrazol-1-yl)methyl)-N-((6-methoxy-2,2-dimethyl-2,3-dihydrobenzofuran-7-yl)sulfonyl)-3-vinylbenzamide

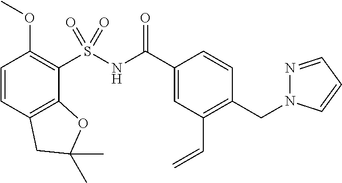 [SMILES: C=Cc1cc(C(=O)NS(=O)(=O)c2c(OC)ccc3c2OC(C)(C)C3)ccc1Cn1cccn1]

A solution of 4-((1H-pyrazol-1-yl)methyl)-3-vinylbenzoic acid (70 mg, 0.31 mmol), 6-methoxy-2,2-dimethyl-2,3-dihydrobenzofuran-7-sulfonamide (80 mg, 0.31 mmol), EDCI (119 mg, 0.62 mmol) and DMAP (114 mg, 0.93 mmol) in DCM (5 mL) was stirred at rt for overnight. Upon completion of the reaction, the reaction was concentrated, water was added, the mixture was extracted with MeOH/DCM, the organic layer was concentrated, and the crude was purified by silica gel column chromatography (MeOH/DCM=1:10) to give the crude product (70 mg). The crude product was further purified by Prep-HPLC (Waters Sunfire C18, 43%-63% ACN in H2O/0.11% FA) to give the product (12 mg). 1H NMR (400 MHz, DMSO-d6) δ 12.25 (s, 1H), 8.16 (s, 1H), 7.80-7.73 (m, 1H), 7.70 (d, J=8.1, 1H), 7.45 (d, J=1.3 Hz, 1H), 7.28 (d, J=6.4 Hz, 1H), 7.12 (d, J=17.3, 1H), 6.90 (d, J=8.0 Hz, 1H), 6.52 (d, J=6.6 Hz, 1H), 6.26 (t, J=1.8 Hz, 1H), 5.93 (d, J=17.4 Hz, 1H), 5.54-5.36 (m, 3H), 3.72 (s, 3H), 2.87 (s, 2H), 1.23 (s, 6H). MS (ESI) m/e [M+H]+=468.

### Example C45: Synthesis of 4-((1H-pyrazol-1-yl)methyl)-3-methoxy-N-((6-methoxy-2,2-dimethyl-2,3-dihydrobenzofuran-7-yl)sulfonyl)benzamide

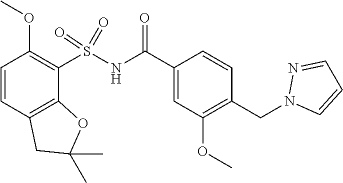 [SMILES: COc1cc(C(=O)NS(=O)(=O)c2c(OC)ccc3c2OC(C)(C)C3)ccc1Cn1cccn1]

A solution of 4-((1H-pyrazol-1-yl)methyl)-3-methoxybenzoic acid (35 mg, 0.15 mmol), 6-methoxy-2,2-dimethyl-2,3-dihydrobenzofuran-7-sulfonamide (39 mg, 0.15 mmol), EDCI (58 mg, 0.3 mmol) and DMAP (55 mg, 0.45 mmol) in DCM (5 mL) was stirred at rt for overnight. Upon completion of the reaction, the reaction was concentrated, water was added, the mixture was extracted with MeOH/DCM, the organic layer was concentrated, and the crude was purified by Prep-HPLC (Waters Sunfire C18, 40%-60% ACN in H2O/0.1% FA) to give the product (35 mg, 49%). 1H NMR (400 MHz, DMSO-d6) δ 12.18 (s, 1H), 7.76 (d, J=2.0 Hz, 1H), 7.56 (d, J=1.1 Hz, 1H), 7.45 (d, J=1.3 Hz, 1H), 7.41 (d, J=7.9, 1H), 7.28 (d, J=8.1 Hz, 1H), 6.72 (d, J=7.9 Hz, 1H), 6.51 (d, J=8.3 Hz, 1H), 6.26 (t, J=2.0 Hz, 1H), 5.31 (s, 2H), 3.88 (s, 3H), 3.72 (s, 3H), 2.88 (s, 2H), 1.26 (s, 6H). MS (ESI) m/e [M+H]+=472.

### Example C46: Synthesis of 5-((1H-pyrazol-1-yl)methyl)-6-cyclopropyl-N-((6-methoxy-2,2-dimethyl-2,3-dihydrobenzofuran-7-yl)sulfonyl)picolinamide

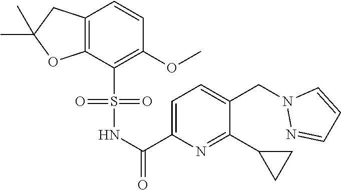 [SMILES: COc1ccc2c(c1S(=O)(=O)NC(=O)c1ccc(Cn3cccn3)c(C3CC3)n1)OC(C)(C)C2]

A solution of 5-((1H-pyrazol-1-yl)methyl)-6-cyclopropylpicolinic acid (30 mg, 0.12 mmol), 2,2-dimethyl-2,3-dihydrobenzofuran-7-sulfonamide (28 mg, 0.12 mmol), EDCI (47 mg, 0.25 mmol) and DMAP (45 mg, 0.37 mmol) in DCM (5 mL) was stirred at rt for overnight. Upon completion of the reaction, the reaction was concentrated, water was added, the mixture was extracted with MeOH/DCM, the organic layer was concentrated, and the crude was purified by Prep-HPLC (Waters Sunfire C18, 48%-68% ACN in H2O/FA) to give the product (22 mg, 37%). 1H NMR (400 MHz, DMSO-d6) δ 10.95 (s, 1H), 7.83 (d, J=2.0 Hz, 1H), 7.68 (d, J=7.9 Hz, 1H), 7.48 (d, J=1.3 Hz, 1H), 7.31-7.21 (m, 2H), 6.52 (d, J=8.3 Hz, 1H), 6.28 (t, J=2.0 Hz, 1H), 5.62 (s, 2H), 3.71 (s, 3H), 2.84 (s, 2H), 2.40-2.32 (m, 1H), 1.18-1.08 (m, 8H), 1.03-0.95 (m, 2H). MS (ESI) m/e [M+H]+=483.

### Example C47: Synthesis of 5-((1H-pyrazol-1-yl)methyl)-6-cyclopropyl-N-((6-methoxy-3,3-dimethyl-2,3-dihydrobenzofuran-7-yl)sulfonyl)picolinamide

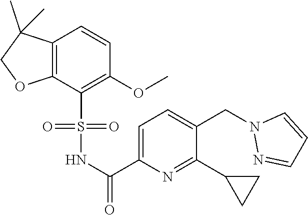 [SMILES: COc1ccc2c(c1S(=O)(=O)NC(=O)c1ccc(Cn3cccn3)c(C3CC3)n1)OCC2(C)C]

A solution of 5-((1H-pyrazol-1-yl)methyl)-6-cyclopropylpicolinic acid (30 mg, 0.12 mmol), 3,3-dimethyl-2,3-dihydrobenzofuran-7-sulfonamide (28 mg, 0.12 mmol), EDCI (47 mg, 0.25 mmol) and DMAP (45 mg, 0.37 mmol) in DCM (5 mL) was stirred at rt for overnight. Upon completion of the reaction, the reaction was concentrated, water was added, the mixture was extracted with MeOH/DCM, the organic layer was concentrated, and the crude was purified by Prep-HPLC (Waters Sunfire C18, 48%-68% ACN in H2O/FA) to give the product (23 mg, 39%). 1H NMR (400 MHz, DMSO-d6) δ 11.16 (s, 1H), 7.84 (d, J=2.0 Hz, 1H), 7.64 (d, J=7.9 Hz, 1H), 7.48 (d, J=1.3 Hz, 1H), 7.33 (d, J=8.3 Hz, 1H), 7.23 (d, J=7.9 Hz, 1H), 6.56 (d, J=8.4 Hz, 1H), 6.28 (t, J=2.0 Hz, 1H), 5.61 (s, 2H), 4.24 (s, 2H), 3.69 (s, 3H), 2.39-2.32 (m, 1H), 1.21 (s, 6H), 1.17-1.13 (m, 2H), 1.02-0.96 (m, 2H). MS (ESI) m/e [M+H]+=483.

### Example C48: Synthesis of 5-((1H-pyrazol-1-yl)methyl)-N-((6-(tert-butyl)-3-methoxypyridin-2-yl)sulfonyl)-6-cyclopropylpicolinamide

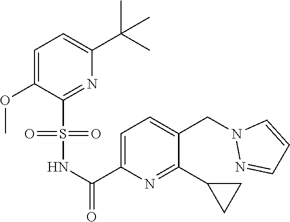 [SMILES: COc1ccc(C(C)(C)C)nc1S(=O)(=O)NC(=O)c1ccc(Cn2cccn2)c(C2CC2)n1]

A solution of 5-((1H-pyrazol-1-yl)methyl)-6-cyclopropylpicolinic acid (30 mg, 0.12 mmol), 6-(tert-butyl)-3-methoxypyridine-2-sulfonamide (30 mg, 0.12 mmol), EDCI (47 mg, 0.25 mmol) and DMAP (45 mg, 0.37 mmol) in DCM (5 mL) was stirred at rt for overnight. Upon completion of the reaction, the reaction was concentrated, water was added, the mixture was extracted with MeOH/DCM, the organic layer was concentrated, and the crude was purified by Prep-HPLC (Waters Sunfire C18, 53%-73% ACN in H2O/FA) to give the product (14 mg, 24%). 1H NMR (400 MHz, DMSO-d6) δ 11.43 (s, 1H), 7.84 (d, J=2.1 Hz, 1H), 7.72-7.59 (m, 3H), 7.49 (d, J=1.4 Hz, 1H), 7.24 (d, J=7.9 Hz, 1H), 6.29 (t, J=2.0 Hz, 1H), 5.63 (s, 2H), 3.88 (s, 3H), 2.38-2.30 (m, 1H), 1.26-1.16 (m, 2H), 1.01 (s, 9H), 0.96-0.91 (m, 2H). MS (ESI) m/e [M+H]+=470.

### Example C49: Synthesis of 4-((1H-pyrazol-1-yl)methyl)-3-methoxy-N-((6-methoxy-3,3-dimethyl-2,3-dihydrobenzofuran-7-yl)sulfonyl)benzamide

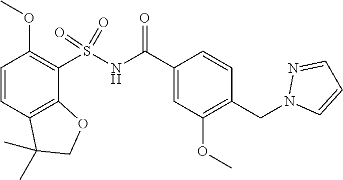 [SMILES: COc1cc(C(=O)NS(=O)(=O)c2c(OC)ccc3c2OCC3(C)C)ccc1Cn1cccn1]

A solution of 4-((1H-pyrazol-1-yl)methyl)-3-methoxybenzoic acid (30 mg, 0.13 mmol), 6-methoxy-3,3-dimethyl-2,3-dihydrobenzofuran-7-sulfonamide (33 mg, 0.13 mmol), EDCI (50 mg, 0.26 mmol) and DMAP (48 mg, 0.39 mmol) in DCM (5 mL) was stirred at rt for overnight. Upon completion of the reaction, the reaction was concentrated, water was added, the mixture was extracted with MeOH/DCM, the organic layer was concentrated, and the crude was purified by Prep-HPLC (Waters Sunfire C18, 43%-58% ACN in H2O/FA) to give the product (20 mg, 33%). 1H NMR (400 MHz, DMSO-d6) δ 11.43 (s, 1H), 7.84 (d, J=2.1 Hz, 1H), 7.72-7.59 (m, 3H), 7.49 (d, J=1.4 Hz, 1H), 7.24 (d, J=7.9 Hz, 1H), 6.29 (t, J=2.0 Hz, 1H), 5.63 (s, 2H), 3.88 (s, 3H), 2.38-2.30 (m, 1H), 1.26-1.16 (m, 2H), 1.01 (s, 9H), 0.96-0.91 (m, 2H). MS (ESI) m/e [M+H]+=470.

### Example C50: Synthesis of 4-((1H-pyrazol-1-yl)methyl)-N-((6-methoxy-3,3-dimethyl-2,3-dihydrobenzofuran-7-yl)sulfonyl)-3-vinylbenzamide

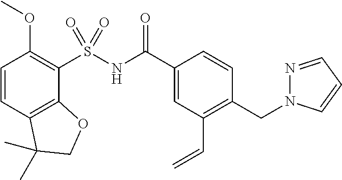 [SMILES: C=Cc1cc(C(=O)NS(=O)(=O)c2c(OC)ccc3c2OCC3(C)C)ccc1Cn1cccn1]

A solution of 4-((1H-pyrazol-1-yl)methyl)-3-vinylbenzoic acid (60 mg, 0.26 mmol), 6-methoxy-3,3-dimethyl-2,3-dihydrobenzofuran-7-sulfonamide (68 mg, 0.26 mmol), EDCI (101 mg, 0.53 mmol) and DMAP (97 mg, 0.79 mmol) in DCM (5 mL) was stirred at rt for overnight. Upon completion of the reaction, the reaction was concentrated, water was added, the mixture was extracted with MeOH/DCM, the organic layer was concentrated, and the crude was purified by silica gel column chromatography (MeOH/DCM=1:20) to give the crude product (40 mg). The crude (10 mg) was purified by Prep-HPLC (Waters Sunfire C18, 45%-65% ACN in H₂O/FA) to give the product (2 mg). 1H NMR (400 MHz, DMSO-d6) δ 12.30 (s, 1H), 8.11-8.03 (m, 1H), 7.75 (s, 1H), 7.66 (d, J=8.0, 1H), 7.43 (d, J=1.4 Hz, 1H), 7.36-7.24 (m, 1H), 7.10 (dd, J=17.3, 11.0 Hz, 1H), 6.90 (d, J=8.1 Hz, 1H), 6.54 (d, J=6.7 Hz, 1H), 6.24 (t, J=1.8 Hz, 1H), 5.89 (d, J=16.5 Hz, 1H), 5.47-5.40 (m, 3H), 4.23 (s, 2H), 3.69 (s, 3H), 1.21 (s, 6H). MS (ESI) m/e [M+H]+=468.

### Example C51: Synthesis of 4-((1H-pyrazol-1-yl)methyl)-3-ethyl-N-((6-methoxy-3,3-dimethyl-2,3-dihydrobenzofuran-7-yl)sulfonyl)benzamide

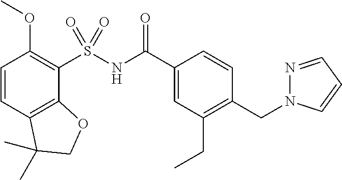 [SMILES: CCc1cc(C(=O)NS(=O)(=O)c2c(OC)ccc3c2OCC3(C)C)ccc1Cn1cccn1]

To a solution of 4-((1H-pyrazol-1-yl)methyl)-N-((6-methoxy-3,3-dimethyl-2,3-dihydrobenzofuran-7-yl)sulfonyl)-3-vinylbenzamide (30 mg, 0.13 mmol) in MeOH (10 mL) was added Pd/C (30 mg), and then H2 was introduced, the resulting mixture was stirred at rt for 2h. Upon completion of the reaction, the reaction was filtered out and concentrated, the crude was purified by Prep-HPLC (Waters Sunfire C18, 45%-65% ACN in H2O/FA) to give the product (6 mg, 20%). 1H NMR (400 MHz, DMSO-d6) δ 12.20 (s, 1H), 7.76 (d, J=2.0 Hz, 1H), 7.71 (d, J=1.4 Hz, 1H), 7.59 (d, J=8.0, 1H), 7.44 (d, J=1.6 Hz, 1H), 7.31 (d, J=8.2 Hz, 1H), 6.81 (d, J=8.1 Hz, 1H), 6.55 (d, J=8.3 Hz, 1H), 6.25 (t, J=2.0 Hz, 1H), 5.39 (s, 2H), 4.24 (s, 2H), 3.70 (s, 3H), 2.68 (q, J=7.5 Hz, 2H), 1.21 (s, 6H), 1.10 (t, J=7.5 Hz, 3H). MS (ESI) m/e [M+H]+=470.

### Example C52: Synthesis of 4-((1H-pyrazol-1-yl)methyl)-3-ethyl-N-((6-methoxy-2,2-dimethyl-2,3-dihydrobenzofuran-7-yl)sulfonyl)benzamide

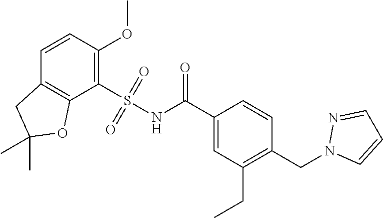 [SMILES: CCc1cc(C(=O)NS(=O)(=O)c2c(OC)ccc3c2OC(C)(C)C3)ccc1Cn1cccn1]

Examples C52 was synthesized starting from the corresponding starting materials according to the similar procedures described as those of Example C51.

1H NMR (400 MHz, DMSO-d6) δ 12.13 (s, 1H), 7.82-7.73 (m, 2H), 7.64 (d, J=8.1, 1H), 7.46 (d, J=1.3 Hz, 1H), 7.28 (d, J=8.1 Hz, 1H), 6.82 (d, J=8.1 Hz, 1H), 6.51 (d, J=8.3 Hz, 1H), 6.27 (t, J=2.0 Hz, 1H), 5.41 (s, 2H), 3.72 (s, 3H), 2.88 (s, 2H), 2.69 (q, J=7.5 Hz, 2H), 1.25 (s, 6H), 1.12 (t, J=7.5 Hz, 3H). MS (ESI) m/e [M+H]+=470.

### Example C53: Synthesis of 5-((1H-pyrazol-1-yl)methyl)-6-ethyl-N-((6-methoxy-3,3-dimethyl-2,3-dihydrobenzofuran-7-yl)sulfonyl)picolinamide

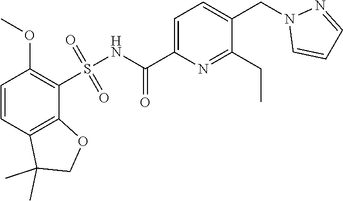 [SMILES: CCc1nc(C(=O)NS(=O)(=O)c2c(OC)ccc3c2OCC3(C)C)ccc1Cn1cccn1]

A solution of 6-methoxy-3,3-dimethyl-2,3-dihydrobenzofuran-7-sulfonamide (26 mg, 0.10 mmol), 5-((1H-pyrazol-1-yl)methyl)-6-ethylpicolinic acid (23 mg, 0.10 mmol), EDCI (36 mg, 0.20 mmol) and DMAP (36 mg, 0.30 mmol) in DCM (1 mL) was stirred at rt for overnight. Upon completion of the reaction, the mixture was concentrated. The crude was purified by Combi-Flash (Column=C18 spherical 20-35 um; mobile phase: [water (0.1% FA)-ACN], B %=5%-80%; 6.0 min) to give the product (10 mg, 21%). 1H NMR (400 MHz, DMSO-d6) δ 11.22 (s, 1H), 7.90-7.84 (m, 1H), 7.78 (d, J=7.9 Hz, 1H), 7.55-7.48 (m, 1H), 7.41-7.33 (m, 1H), 7.31 (d, J=7.8 Hz, 1H), 6.59 (d, J=8.1 Hz, 1H), 6.35-6.31 (m, 1H), 5.54 (s, 2H), 4.29 (s, 2H), 3.72 (s, 3H), 2.96-2.86 (m, 2H), 1.26 (s, 6H), 1.26-1.20 (m, 3H). MS (ESI) m/e [M+H]+=471.3.

### Example C54: Synthesis of 5-((1H-pyrazol-1-yl)methyl)-6-methoxy-N-((3-methoxy-6-(1,1,1-trifluoro-2-methylpropan-2-yl)pyridin-2-yl)sulfonyl)picolinamide

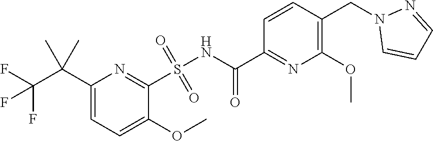 [SMILES: COc1ccc(C(C)(C)C(F)(F)F)nc1S(=O)(=O)NC(=O)c1ccc(Cn2cccn2)c(OC)n1]

A solution of 3-methoxy-6-(1,1,1-trifluoro-2-methylpropan-2-yl)pyridine-2-sulfonamide (30 mg, 0.1 mmol), 5-((1H-pyrazol-1-yl)methyl)-6-methoxypicolinic acid (23 mg, 0.1 mmol), EDCI (38.4 mg, 0.2 mmol) and DMAP (24.4 mg, 0.2 mmol) in DCM (5 mL) was stirred at r.t overnight. The solvent was removed, and the residue was purified by Prep-HPLC (column: Sunfire C18; mobile phase: [H2O (0.1% FA)-ACN]; gradient: 55%-70% B over 11.0 min) to give the desired product (26 mg, 50.6%). 1H NMR (400 MHz, DMSO-d6) δ 11.94 (s, 1H), 7.92-7.73 (m, 3H), 7.48 (dd, J=10.6, 4.5 Hz, 2H), 7.16 (d, J=7.5 Hz, 1H), 6.27 (t, J=2.0 Hz, 1H), 5.33 (s, 2H), 4.09 (s, 3H), 3.93 (s, 3H), 1.32 (s, 6H). MS (ESI) m/e [M+H]+=514.

### Example C55: Synthesis of 5-((1H-pyrazol-1-yl)methyl)-6-methoxy-N-((7-methoxy-3,3-dimethylchroman-8-yl)sulfonyl)picolinamide

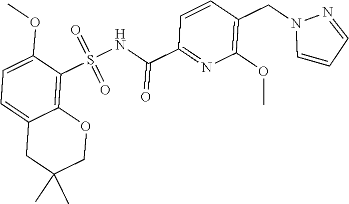 [SMILES: COc1ccc2c(c1S(=O)(=O)NC(=O)c1ccc(Cn3cccn3)c(OC)n1)OCC(C)(C)C2]

A mixture of 5-((1H-pyrazol-1-yl)methyl)-6-methoxypicolinic acid (23 mg, 0.1 mmol), 7-methoxy-3,3-dimethylchromane-8-sulfonamide (27 mg, 0.1 mmol), DMAP (37 mg, 0.3 mmol) and EDCI (29 mg, 0.15 mmol) in DCM (2 mL) was stirred at 45° C. for 4 h. After cooling to rt, the mixture was concentrated, and the residue was purified by C18 column (mobile phase, ACN in water (0.1% FA), 10% to 70%) to give the product (25 mg, 51%). 1H NMR (400 MHz, DMSO-d6) δ 11.38 (s, 1H), 7.85 (d, J=2.0 Hz, 1H), 7.55-7.49 (m, 2H), 7.24-7.17 (m, 2H), 6.68 (d, J=8.6 Hz, 1H), 6.32 (t, J=2.0 Hz, 1H), 5.37 (s, 2H), 4.10 (s, 3H), 3.76 (s, 3H), 3.69 (s, 2H), 2.46 (s, 2H), 0.81 (s, 6H). MS (ESI) m/e [M+H]+=487.3.

### Example C56: Synthesis of 5-((1H-pyrazol-1-yl)methyl)-6-methoxy-N-((2-methoxy-5-(1-methoxycyclopropyl)phenyl)sulfonyl)picolinamide

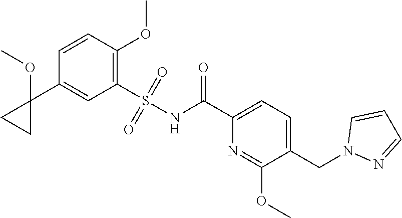 [SMILES: COc1ccc(C2(OC)CC2)cc1S(=O)(=O)NC(=O)c1ccc(Cn2cccn2)c(OC)n1]

A mixture of 5-((1H-pyrazol-1-yl)methyl)-6-methoxypicolinic acid (23 mg, 0.1 mmol), 2-methoxy-5-(1-methoxycyclopropyl)benzenesulfonamide (26 mg, 0.1 mmol), DMAP (37 mg, 0.3 mmol) and EDCI (29 mg, 0.15 mmol) in DCM (2 mL) was stirred at 45° C. for 6 h. After cooled to rt, the mixture was concentrated, and the residue was purified by C18 column (mobile phase, ACN in water (0.1% FA), 10% to 70%) to give the product (21 mg, 44%). 1H NMR (400 MHz, DMSO-d6) δ 11.83 (s, 1H), 7.87-7.82 (m, 2H), 7.52-7.47 (m, 3H), 7.22-7.17 (m, 2H), 6.31 (t, J=2.0 Hz, 1H), 5.35 (s, 2H), 4.12 (s, 3H), 3.84 (s, 3H), 3.15 (s, 3H), 1.19-1.14 (m, 2H), 0.97-0.93 (m, 2H). MS (ESI) m/e [M+H]+=473.3.

### Example C57: Synthesis of 5-((1H-pyrazol-1-yl)methyl)-6-methoxy-N-((2-methoxy-5-(1-methoxy-2-methylpropan-2-yl)phenyl)sulfonyl)picolinamide

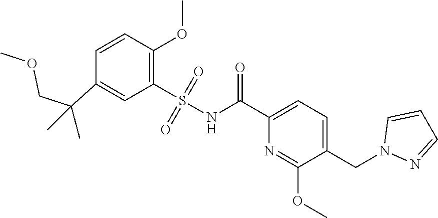 [SMILES: COCC(C)(C)c1ccc(OC)c(S(=O)(=O)NC(=O)c2ccc(Cn3cccn3)c(OC)n2)c1]

A mixture of 5-((1H-pyrazol-1-yl)methyl)-6-methoxypicolinic acid (13 mg, 0.055 mmol), 2-methoxy-5-(1-methoxy-2-methylpropan-2-yl)benzenesulfonamide (15 mg, 0.055 mmol), DMAP (20 mg, 0.164 mmol) and EDCI (16 mg, 0.082 mmol) in DCM (2 mL) was stirred at 45° C. for 16 h. After cooling to rt, the mixture was concentrated, and the residue was purified by C18 column (mobile phase, ACN in water (0.1% FA), 20% to 80%) to give the product (10 mg, 37%). 1H NMR (400 MHz, DMSO-d6) δ 11.68 (s, 1H), 7.86-7.80 (m, 2H), 7.70-7.64 (m, 1H), 7.53-7.48 (m, 2H), 7.18 (d, J=16.3 Hz, 2H), 6.32-6.28 (m, 1H), 5.35 (s, 2H), 4.12 (s, 3H), 3.82 (s, 3H), 3.35 (s, 3H), 3.29 (s, 2H), 1.26 (s, 6H). MS (ESI) m/e [M+H]+=489.3.

### Example C58: Synthesis of 5-((1H-pyrazol-1-yl)methyl)-6-methoxy-N-((6-methoxy-2,3-dihydrobenzofuran-7-yl)sulfonyl)picolinamide

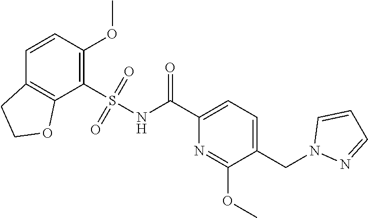 [SMILES: COc1ccc2c(c1S(=O)(=O)NC(=O)c1ccc(Cn3cccn3)c(OC)n1)OCC2]

A mixture of 5-((1H-pyrazol-1-yl)methyl)-6-methoxypicolinic acid (12 mg, 0.05 mmol), 6-methoxy-2,3-dihydrobenzofuran-7-sulfonamide (12 mg, 0.05 mmol), DMAP (12 mg, 0.1 mmol) and EDCI (14 mg, 0.075 mmol) in DCM (2 mL) was stirred at 45° C. for 2 h. After cooling to rt, the mixture was concentrated, and the residue was purified by C18 column (mobile phase, ACN in water (0.1% FA), 10% to 80%) to give the product (10 mg, 45%). 1H NMR (400 MHz, DMSO-d6) δ 11.41 (s, 1H), 7.84 (d, J=2.1 Hz, 1H), 7.54-7.49 (m, 2H), 7.36 (d, J=8.2 Hz, 1H), 7.20 (d, J=7.5 Hz, 1H), 6.54 (d, J=8.2 Hz, 1H), 6.32-6.29 (m, 1H), 5.35 (s, 2H), 4.60 (t, J=8.8 Hz, 2H), 4.09 (s, 3H), 3.73 (s, 3H), 3.12 (t, J=8.8 Hz, 2H). MS (ESI) m/e [M+H]+=445.3.

### Example C59: Synthesis of 5-((1H-pyrazol-1-yl)methyl)-6-methoxy-N-((2-methoxy-5-(1-(trifluoromethyl)cyclopropyl)phenyl)sulfonyl)picolinamide

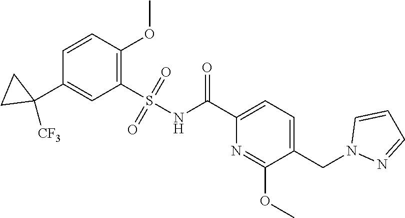 [SMILES: COc1ccc(C2(C(F)(F)F)CC2)cc1S(=O)(=O)NC(=O)c1ccc(Cn2cccn2)c(OC)n1]

A mixture of 5-((1H-pyrazol-1-yl)methyl)-6-methoxypicolinic acid (23 mg, 0.1 mmol), 2-methoxy-5-(1-(trifluoromethyl)cyclopropyl)benzenesulfonamide (30 mg, 0.1 mmol), DMAP (37 mg, 0.3 mmol) and EDCI (29 mg, 0.15 mmol) in DCM (2 mL) was stirred at 45° C. for 2 h. After cooling to rt, the mixture was concentrated, and the residue was purified by C18 column (mobile phase, ACN in water (0.1% FA), 15% to 70%) to give the product (36 mg, 70%). 1H NMR (400 MHz, DMSO-d6) δ 11.94 (s, 1H), 7.96-7.92 (m, 1H), 7.85-7.82 (m, 1H), 7.75 (d, J=7.7 Hz, 1H), 7.52-7.47 (m, 2H), 7.25 (d, J=8.3 Hz, 1H), 7.18 (d, J=7.4 Hz, 1H), 6.33-6.29 (m, 1H), 5.35 (s, 2H), 4.12 (s, 3H), 3.86 (s, 3H), 1.42-1.35 (m, 2H), 1.16-1.10 (m, 2H). MS (ESI) m/e [M+H]+=511.2.

### Example C60: Synthesis of 5-((1H-pyrazol-1-yl)methyl)-6-methoxy-N-((6-methoxy-2H-spiro[benzofuran-3,1′-cyclobutan]-7-yl)sulfonyl)picolinamide

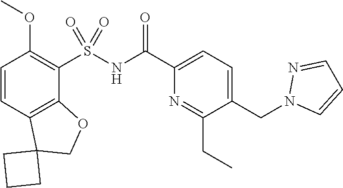 [SMILES: CCc1nc(C(=O)NS(=O)(=O)c2c(OC)ccc3c2OCC32CCC2)ccc1Cn1cccn1]

A solution of 6-methoxy-2H-spiro[benzofuran-3,1′-cyclobutane]-7-sulfonamide (27 mg, 0.10 mmol), 5-((1H-pyrazol-1-yl)methyl)-6-methoxypicolinic acid (23 mg, 0.10 mmol), EDCI (38 mg, 0.20 mmol) and DMAP (36 mg, 0.30 mmol) in DCM (1 mL) was stirred at r.t overnight. Upon completion of the reaction, the mixture was concentrated and purified by (Column=C18 spherical 20-35 μm; mobile phase: [water (HCOOH)-ACN], B %=5%-80%; 7.0 min) to give the product (20 mg, 41%). 1H NMR (400 MHz, DMSO-d6) δ 11.52 (s, 1H), 7.85 (d, J=2.0 Hz, 1H), 7.64 (d, J=8.2 Hz, 1H), 7.55-7.46 (m, 2H), 7.23-7.16 (m, 1H), 6.68-6.62 (m, 1H), 6.31 (t, J=2.0 Hz, 1H), 5.36 (s, 2H), 4.65 (s, 2H), 4.11 (s, 3H), 3.75 (s, 3H), 2.35-2.20 (m, 4H), 2.00-1.86 (m, 2H). MS (ESI) m/e [M+H]+=485.3.

### Example C61: Synthesis of 5-((1H-pyrazol-1-yl)methyl)-6-methoxy-N-((6-methoxy-3,3-dimethyl-2,3-dihydrobenzofuran-5-yl)sulfonyl)picolinamide

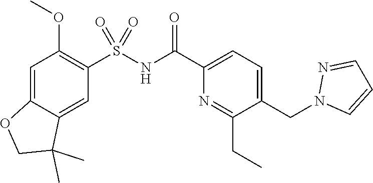 [SMILES: CCc1nc(C(=O)NS(=O)(=O)c2cc3c(cc2OC)OCC3(C)C)ccc1Cn1cccn1]

A solution of 6-methoxy-3,3-dimethyl-2,3-dihydrobenzofuran-5-sulfonamide (26 mg, 0.10 mmol), 5-((1H-pyrazol-1-yl)methyl)-6-methoxypicolinic acid (23 mg, 0.10 mmol), EDCI (38 mg, 0.20 mmol) and DMAP (36 mg, 0.30 mmol) in DCM (1 mL) was stirred at r.t overnight. Upon completion of the reaction, the mixture was concentrated and purified by (Column=C18 spherical 20-35 um; mobile phase: [water (HCOOH)-ACN], B %=5%-80%; 7.0 min) to give the product (10 mg, 21%). 1H NMR (400 MHz, DMSO-d6) δ 11.49 (s, 1H), 7.84 (d, J=2.0 Hz, 1H), 7.70-7.65 (m, 1H), 7.53-7.48 (m, 2H), 7.19 (d, J=7.5 Hz, 1H), 6.72-6.64 (m, 1H), 6.33-6.28 (m, 1H), 5.35 (s, 2H), 4.35 (s, 2H), 4.10 (s, 3H), 3.78 (s, 3H), 1.32 (s, 6H). MS (ESI) m/e [M+H]+=473.3.

Examples C62-C76 below (Table 9) were synthesized starting from the corresponding starting materials according to the similar procedures described as those of Example C61.

TABLE 9

| Example | Compound | Chemical Name | 1H NMR data LC/MS m/z (M + H) |
|---|---|---|---|
| C62 | 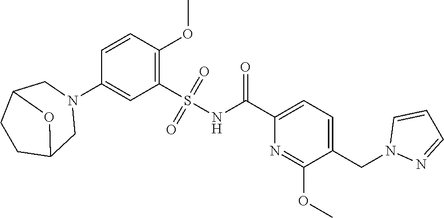 [SMILES: COc1ccc(N2CC3CCC(C2)O3)cc1S(=O)(=O)NC(=O)c1ccc(Cn2cccn2)c(OC)n1] | 5-((1H-pyrazol-1- yl)methyl)-N- ((5-(8-oxa-3- azabicyclo[3.2.1] octan-3- yl)-2- methoxyphenyl) sulfonyl)- 6-methoxy- picolinamide | 1H NMR (400 MHz, DMSO-d6) δ 11.69 (s, 1H), 7.84 (d, J = 2.0 Hz, 1H), 7.52 − 7.49 (m, 2H), 7.30 (d, J = 2.8 Hz, 1H), 7.21 − 7.15 (m, 2H), 7.13 − 7.09 (m, 1H), 6.31 (t, J = 2.0 Hz, 1H), 5.36 (s, 2H), 4.44 (m, 2H), 4.12 (s, 3H), 3.75 (s, 3H), 3.36 − 3.35 (m, 1H), 3.33 − 3.31 (m, 1H), 2.83 − 2.76 (m, 2H), 1.89 − 1.81 (m, 4H). MS (ESI) m/e [M + H]+ = 514.3. |
| C63 | 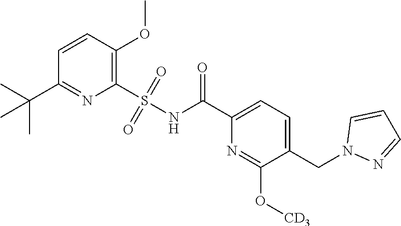 [SMILES: [2H]C([2H])([2H])Oc1nc(C(=O)NS(=O)(=O)c2nc(C(C)(C)C)ccc2OC)ccc1Cn1cccn1] | 5-((1H-pyrazol-1- yl)methyl)-N- ((6-(tert- butyl)-3- methoxypyridin-2- yl)sulfonyl)-6- (methoxy- d3)picolinamide | 1H NMR (400 MHz, DMSO-d6) δ 11.76 (s, 1H), 7.85 (d, J = 1.8 Hz, 1H), 7.75 − 7.69 (m, 1H), 7.68 − 7.62 (m, 1H), 7.55 (d, J = 7.5 Hz, 1H), 7.53 − 7.50 (m, 1H), 7.24 − 7.19 (m, 1H), 6.33 − 6.30 (m, 1H), 5.36 (s, 2H), 3.92 (s, 3H), 1.10 (s, 9H). MS (ESI) m/e [M + H]+ = 463.3. |
| C64 | 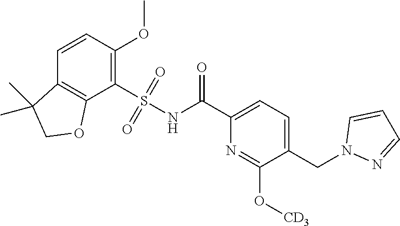 [SMILES: [2H]C([2H])([2H])Oc1nc(C(=O)NS(=O)(=O)c2c(OC)ccc3c2OCC3(C)C)ccc1Cn1cccn1] | 5-((1H-pyrazol-1- yl)methyl)-6- (methoxy-d3)- N-((6-methoxy- 3,3- dimethyl-2,3- dihydrobenzofuran- 7- yl)sulfonyl) picolinamide | 1H NMR (400 MHz, DMSO-d6) δ 11.54 (s, 1H), 7.85 (d, J = 2.0 Hz, 1H), 7.53 (d, J = 7.5 Hz, 1H), 7.51 (d, J = 2.0 Hz, 1H), 7.37 (d, J = 7.5 Hz, 1H), 7.22 − 7.17 (m, 1H), 6.60 (d, J = 8.4 Hz, 1H), 6.32 (t, J = 2.0 Hz, 1H), 5.36 (s, 2H), 4.29 (s, 2H), 3.74 (s, 3H), 1.26 (s, 6H). MS (ESI) m/e [M + H]+ = 476.3. |
| C65 | 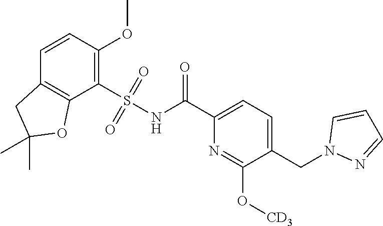 [SMILES: [2H]C([2H])([2H])Oc1nc(C(=O)NS(=O)(=O)c2c(OC)ccc3c2OC(C)(C)C3)ccc1Cn1cccn1] | 5-((1H-pyrazol-1- yl)methyl)-6- (methoxy-d3)- N-((6-methoxy-2,2- dimethyl-2,3- dihydrobenzofuran- 7- yl)sulfonyl) picolinamide | 1H NMR (400 MHz, DMSO-d6) δ 11.34 (s, 1H), 7.85 (d, J = 2.0 Hz, 1H), 7.57 (d, J = 7.6 Hz, 1H), 7.51 (d, J = 2.0 Hz, 1H), 7.33 (d, J = 7.6 Hz, 1H), 7.22 (d, J = 8.0 Hz, 1H), 6.57 (d, J = 8.0 Hz, 1H), 6.32 (t, J = 2.0 Hz, 1H), 5.37 (s, 2H), 3.77 (s, 3H), 2.91 (s, 2H), 1.24 (s, 6H). MS (ESI) m/e [M + H]+ = 476.3. |
| C66 | 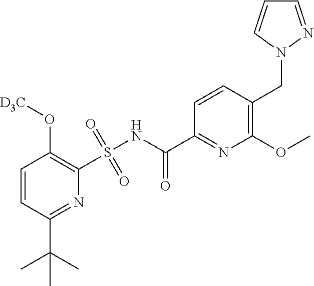 [SMILES: [2H]C([2H])([2H])Oc1ccc(C(C)(C)C)nc1S(=O)(=O)NC(=O)c1ccc(Cn2cccn2)c(OC)n1] | 5-((1H-pyrazol-1- yl)methyl)- N-((6-(tert- butyl)-3-(methoxy- d3)pyridin-2- yl)sulfonyl)- 6- methoxy- picolinamide | 1H NMR (400 MHz, DMSO-d6) δ 11.77 (s, 1H), 7.87 − 7.84 (m, 1H), 7.75 − 7.70 (m, 1H), 7.69 − 7.63 (m, 1H), 7.56 (d, J = 7.4 Hz, 1H), 7.52 (br s, 1H), 7.22 (d, J = 7.4 Hz, 1H), 6.33 − 6.31 (m, 1H), 5.38 (s, 2H), 4.10 (s, 3H), 1.10 (s, 9H). MS (ESI) m/e [M + H]+ = 463.3. |
| C67 | 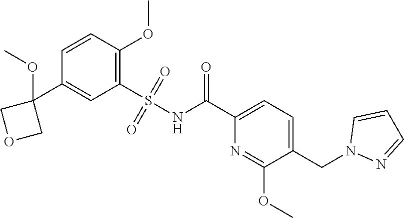 [SMILES: COc1ccc(C2(OC)COC2)cc1S(=O)(=O)NC(=O)c1ccc(Cn2cccn2)c(OC)n1] | 5-((1H-pyrazol-1- yl)methyl)- 6-methoxy-N- ((2-methoxy-5-(3- methoxyoxetan-3- yl)phenyl)sulfonyl) picolinamide | 1H NMR (400 MHz, DMSO-d6) δ 11.93 (s, 1H), 7.91 − 7.89 (m, 1H), 7.85 − 7.83 (m, 1H), 7.81 − 7.76 (m, 1H), 7.51 − 7.48 (m, 2H), 7.31 (d, J = 8.8 Hz, 1H), 7.18 (d, J = 7.6 Hz, 1H), 6.30 (t, J = 2.0 Hz, 1H), 5.35 (s, 2H), 4.81 (d, J = 7.2 Hz, 2H), 4.72 (d, J = 7.2 Hz, 2H), 4.13 (s, 3H), 3.88 (s, 3H), 3.06 (s, 3H). MS (ESI) m/e [M + H]+ = 489.2. |
| C68 | 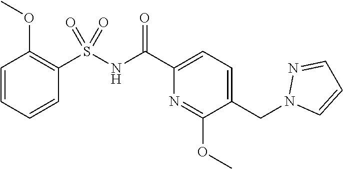 [SMILES: COc1ccccc1S(=O)(=O)NC(=O)c1ccc(Cn2cccn2)c(OC)n1] | 5-((1H-pyrazol-1- yl)methyl)-6- methoxy-N- ((2- methoxyphenyl) sulfonyl) picolinamide | 1H NMR (400 MHz, DMSO-d6) δ 11.70 (s, 1H), 7.87 (br d, J = 6.8 Hz, 1H), 7.79 (d, J = 2.0 Hz, 1H), 7.63 (t, J = 7.8 Hz, 1H), 7.47 − 7.41 (m, 2H), 7.23 − 7.14 (m, 2H), 7.11 (t, J = 7.8 Hz, 1H), 6.26 (t, J = 2.0 Hz, 1H), 5.31 (s, 2H), 4.08 (s, 3H), 3.80 (s, 3H). MS (ESI) m/e [M + H]+ = 403.4. |
| C69 | 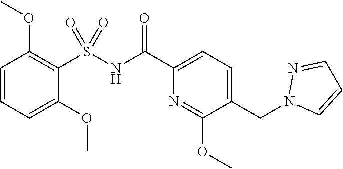 [SMILES: COc1cccc(OC)c1S(=O)(=O)NC(=O)c1ccc(Cn2cccn2)c(OC)n1] | 5-((1H-pyrazol-1- yl)methyl)-N-((2,6- dimethoxyphenyl) sulfonyl)- 6- methoxy- picolinamide | 1H NMR (400 MHz, DMSO-d6) δ 11.34 (s, 1H), 7.80 (d, J = 2.0 Hz, 1H), 7.54 − 7.43 (m, 3H), 7.16 (d, J = 7.5 Hz, 1H), 6.74 (d, J = 8.5 Hz, 2H), 6.27 (t, J = 2.0 Hz, 1H), 5.32 (s, 2H), 4.07 (s, 3H), 3.73 (s, 6H). MS (ESI) m/e [M + H]+ = 433.3. |
| C70 | 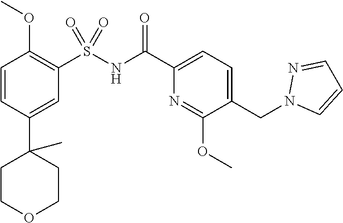 [SMILES: COc1ccc(C2(C)CCOCC2)cc1S(=O)(=O)NC(=O)c1ccc(Cn2cccn2)c(OC)n1] | 5-((1H-pyrazol-1- yl)methyl)-6- methoxy-N- ((2-methoxy-5-(4- methyltetrahydro- 2H- pyran-4- yl)phenyl)sulfonyl) picolinamide | 1H NMR (400 MHz, DMSO-d6) δ 11.69 (s, 1H), 7.83 − 7.75 (m, 2H), 7.64 (br dd, J = 8.7, 2.0 Hz, 1H), 7.49 −7.43 (m, 2H), 7.20 − 7.10 (m, 2H), 6.26 (t, J = 2.0 Hz, 1H), 5.31 (s, 2H), 4.08 (s, 3H), 3.80 (s, 3H), 3.67 − 3.56 (m, 2H), 3.53 − 3.39 (m, 2H), 1.96 − 1.83 (m, 2H), 1.73 − 1.63 (m, 2H), 1.20 (s, 3H). MS (ESI) m/e [M + H]+ = 501.3. |
| C71 | 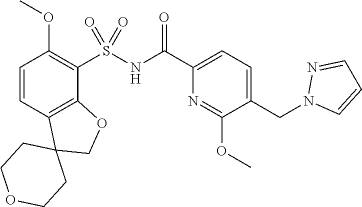 [SMILES: COc1ccc2c(c1S(=O)(=O)NC(=O)c1ccc(Cn3cccn3)c(OC)n1)OCC21CCOCC1] | 5-((1H-pyrazol-1- yl)methyl)-6- methoxy-N- ((6-methoxy- 2′,3′,5′,6′- tetrahydro-2H- spiro[benzofuran- 3,4′- pyran]-7- yl)sulfonyl) picolinamide | 1H NMR (400 MHz, DMSO-d6) δ 11.50 (s, 1H), 7.80 (d, J = 2.0 Hz, 1H), 7.48 (d, J = 7.5 Hz, 1H), 7.46 (d, J = 2.0 Hz, 1H), 7.40 (d, J = 8.4 Hz, 1H), 7.16 (d, J = 7.5 Hz, 1H), 6.57 (d, J = 8.4 Hz, 1H), 6.27 (t, J = 2.0 Hz, 1H), 5.32 (s, 2H), 4.53 (s, 2H), 4.06 (s, 3H), 3.79 (dd, J = 11.8, 3.6 Hz, 2H), 3.70 (s, 3H), 3.35 (t, J = 11.8 Hz, 2H), 1.82 (td, J = 13.0, 3.6 Hz, 2H), 1.50 (d, J = 13.0 Hz, 2H). MS (ESI) m/e [M + H]+ = 515.3. |
| C72 | 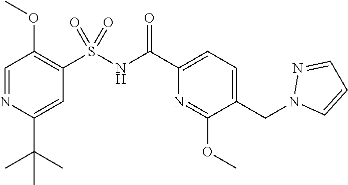 [SMILES: COc1cnc(C(C)(C)C)cc1S(=O)(=O)NC(=O)c1ccc(Cn2cccn2)c(OC)n1] | 5-((1H-pyrazol-1- yl)methyl)- N-((2-(tert- butyl)-5- methoxypyridin-4- yl)sulfonyl)-6- methoxy- picolinamide | 1H NMR (400 MHz, DMSO-d6) δ 12.25 (s, 1H), 8.56 (s, 1H), 7.79 (d, J = 2.0 Hz, 1H), 7.70 (s, 1H), 7.50 − 7.42 (m, 2H), 7.14 (d, J = 7.5 Hz, 1H), 6.26 (t, J = 2.0 Hz, 1H), 5.31 (s, 2H), 4.08 (s, 3H), 3.90 (s, 3H), 1.28 (s, 9H). MS (ESI) m/e [M + H]+ = 460.4. |
| C73 | 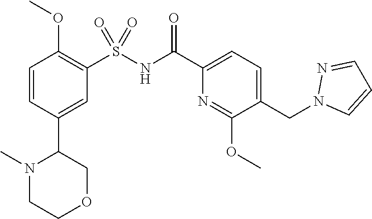 [SMILES: COc1ccc(C2COCCN2C)cc1S(=O)(=O)NC(=O)c1ccc(Cn2cccn2)c(OC)n1] | 5-((1H-pyrazol-1- yl)methyl)- 6-methoxy-N- ((2-methoxy-5-(4- methylmorpholin-3- yl)phenyl)sulfonyl) picolinamide | 1H NMR (400 MHz, DMSO-d6) δ 11.76 (s, 1H), 7.91 (s, 1H), 7.83 (d, J = 2.0 Hz, 1H), 7.61 (br d, J = 7.8 Hz, 1H), 7.54 − 7.49 (m, 1H), 7.48 (s, 1H), 7.19 (d, J = 7.8 Hz, 2H), 6.30 (t, J = 2.0 Hz, 1H), 5.35 (s, 2H), 4.11 (s, 3H), 3.83 (br s, 4H), 3.68 − 3.58 (m, 2H), 3.15 (br d, 2H), 2.88 − 2.82 (m, 1H), 2.36 − 2.25 (m, 1H), 2.00 (s, 3H). MS (ESI) m/e [M + H]+ = 502.3. |
| C74 | 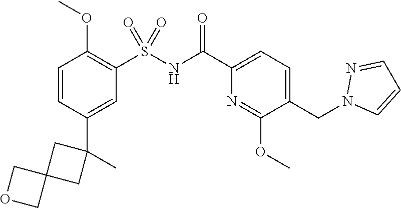 [SMILES: COc1ccc(C2(C)CC3(COC3)C2)cc1S(=O)(=O)NC(=O)c1ccc(Cn2cccn2)c(OC)n1] | 5-((1H-pyrazol-1- yl)methyl)- 6-methoxy-N- ((2-methoxy-5- (6-methyl- 2-oxaspiro[3.3] heptan-6- yl)phenyl)sulfonyl) picolinamide | 1H NMR (400 MHz, DMSO-d6) δ 11.68 (s, 1H), 7.79 (d, J = 2.0 Hz, 1H), 7.60 (d, J = 2.4 Hz, 1H), 7.51 − 7.35 (m, 3H), 7.14 (dd, J = 14.4, 8.1 Hz, 2H), 6.26 (t, J = 2.0 Hz, 1H), 5.31 (s, 2H), 4.69 (s, 2H), 4.41 (s, 2H), 4.08 (s, 3H), 3.77 (s, 3H), 2.43 − 2.33 (m, 4H), 1.22 (s, 3H). MS (ESI) m/e [M + H]+ = 513.3. |
| C75 | 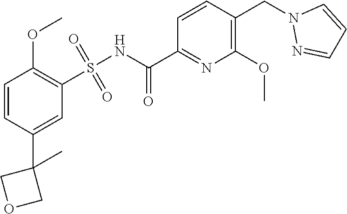 [SMILES: COc1ccc(C2(C)COC2)cc1S(=O)(=O)NC(=O)c1ccc(Cn2cccn2)c(OC)n1] | 5-((1H-pyrazol-1- yl)methyl)-6- methoxy-N- ((2-methoxy-5-(3- methyloxetan-3- yl)phenyl)sulfonyl) picolinamide | 1H NMR (400 MHz, DMSO-d6) δ 11.84 (s, 1H), 7.86 − 7.83 (m, 1H), 7.77 − 7.75 (m, 1H), 7.63 − 7.58 (m, 1H), 7.53 − 7.49 (m, J = 7.3 Hz, 2H), 7.24 (d, J = 8.4 Hz, 1H), 7.19 (d, J = 7.3 Hz, 1H), 6.31 (t, J = 2.0 Hz, 1H), 5.36 (s, 2H), 4.76 (d, J = 5.8 Hz, 2H), 4.59 (d, J = 5.8 Hz, 2H), 4.13 (s, 3H), 3.85 (s, 3H), 1.65 (s, 3H). MS (ESI) m/e [M + H]+ = 473.4. |
| C76 | 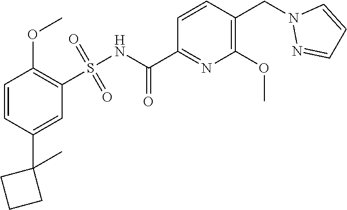 [SMILES: COc1ccc(C2(C)CCC2)cc1S(=O)(=O)NC(=O)c1ccc(Cn2cccn2)c(OC)n1] | 5-((1H-pyrazol-1- yl)methyl)-6- methoxy-N- ((2-methoxy-5-(1- methylcyclobutyl) phenyl) sulfonyl) picolinamide | 1H NMR (400 MHz, DMSO-d6) δ 11.70 (s, 1H), 7.79 (d, J = 2.0 Hz, 1H), 7.60 (d, J = 2.0 Hz, 1H), 7.51 − 7.34 (m, 3H), 7.18 − 7.07 (m, 2H), 6.26 (t, J = 2.0 Hz, 1H), 5.30 (s, 2H), 4.07 (s, 3H), 3.77 (s, 3H), 2.26 − 2.18 (m, 2H), 2.08 − 1.96 (m, 3H), 1.80 − 1.71 (m, 1H), 1.37 (s, 3H). MS (ESI) m/e [M + H]+ = 471. |

### Example C77: Synthesis of 5-((1H-pyrazol-1-yl)methyl)-N-((5-(2-oxabicyclo[2.1.1]hexan-4-yl)-2-methoxyphenyl)sulfonyl)-6-methoxypicolinamide

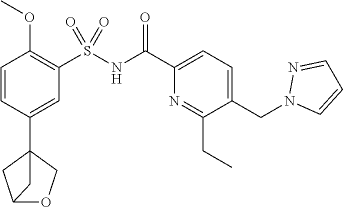 [SMILES: CCc1nc(C(=O)NS(=O)(=O)c2cc(C34COC(C3)C4)ccc2OC)ccc1Cn1cccn1]

A mixture of 5-(2-oxabicyclo[2.1.1]hexan-4-yl)-2-methoxybenzenesulfonamide (54 mg, 0.2 mmol), 5-((1H-pyrazol-1-yl)methyl)-6-methoxypicolinic acid (47 mg, 0.2 mmol), EDCI (76 mg, 0.4 mmol) and DMAP (73 mg, 0.3 mmol) in DCM (2 mL) was stirred at r.t overnight. Upon completion of the reaction, the mixture was concentrated and purified by prep-HPLC (Column=C18 spherical 20-35 μm; mobile phase: [water (HCOOH)-ACN], B %=5%-80%; 7.0 min) to give the desired product (39 mg, 40% yield). 1H NMR: (400 MHz, DMSO-d6) δ 11.83 (s, 1H), 7.83-7.77 (m, 1H), 7.74-7.70 (m, 1H), 7.59 (d, J=8.8 Hz, 1H), 7.51-7.43 (m, 2H), 7.19 (d, J=8.8 Hz, 1H), 7.15 (d, J=7.5 Hz, 1H), 6.29-6.26 (m, 1H), 5.32 (s, 2H), 4.56 (s, 1H), 4.10 (s, 3H), 3.80 (s, 3H), 3.73 (s, 2H), 2.04-2.00 (m, 2H), 1.83-1.79 (m, 2H). MS (ESI) m/e [M+H]+=485.2.

### Example C78: Synthesis of 5-((1H-pyrazol-1-yl)methyl)-6-methoxy-N-((7-methoxy-3,4-dihydro-2H-2,4-methanochroman-8-yl)sulfonyl)picolinamide

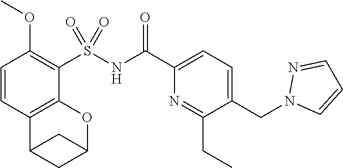 [SMILES: CCc1nc(C(=O)NS(=O)(=O)c2c(OC)ccc3c2OC2CC3C2)ccc1Cn1cccn1]

A mixture of 7-methoxy-3,4-dihydro-2H-2,4-methanochromane-8-sulfonamide (50 mg, 0.18 mmol), 5-((1H-pyrazol-1-yl)methyl)-6-methoxypicolinic acid (47 mg, 0.2 mmol), 2-chloromethylpyridnium iodide (CMPI, 102 mg, 0.4 mmol), DMAP (52 mg, 0.4 mmol) and TEA (45 mg, 0.4 mmol) in DCM (10 mL) was stirred at r.t for 4 h. Upon completion of the reaction, Sat. NH4Cl (aq.) was added to the reaction mixture. The aqueous phase was extracted with DCM. The combined organic phases were washed with brine, dried over anhydrous Na2SO4, concentrated. The residue was purified by pre-HPLC (Column=C18 spherical 20-35 μm; mobile phase: [water (HCOOH)-ACN], B %=5%-80%; 7.0 min) to give the desired product (30 mg, 35% yield). 1H NMR: (400 MHz, DMSO-d6) δ 11.41 (s, 1H), 7.85 (d, J=1.8 Hz, 1H), 7.58-7.48 (m, 2H), 7.28-7.14 (m, 2H), 6.56 (d, J=8.1 Hz, 1H), 6.34-6.28 (m, 1H), 5.36 (s, 2H), 5.08-4.97 (m, 1H), 4.10 (s, 3H), 3.74 (s, 3H), 3.45-3.36 (m, 1H), 2.42-2.30 (m, 2H), 1.39-1.29 (m, 2H). MS(ESI) m/z [M+H]+=471.1.

### Example C79: Synthesis of 5-((1H-pyrazol-1-yl)methyl)-N-((6-(bicyclo[1.1.1]pentan-1-yl)-3-methoxypyridin-2-yl)sulfonyl)-6-methoxypicolinamide

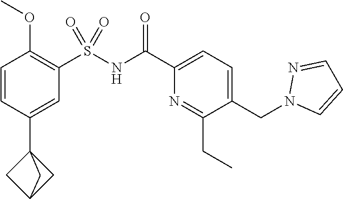 [SMILES: CCc1nc(C(=O)NS(=O)(=O)c2cc(C34CC(C3)C4)ccc2OC)ccc1Cn1cccn1]

A mixture of 6-(bicyclo[1.1.1]pentan-1-yl)-3-methoxypyridine-2-sulfonamide (50 mg, 0.18 mmol), 5-((1H-pyrazol-1-yl)methyl)-6-methoxypicolinic acid (47 mg, 0.2 mmol), 2-chloromethylpyridnium iodide (CMPI, 102 mg, 0.4 mmol), DMAP (52 mg, 0.4 mmol) and TEA (45 mg, 0.4 mmol) in DCM (10 mL) was stirred at r.t for 4 h. Upon completion of the reaction, Sat. NH4Cl (aq.) was added to the reaction mixture. The aqueous phase was extracted with DCM. The combined organic phases were washed with brine, dried over anhydrous Na2SO4, concentrated. The residue was purified by pre-HPLC (Column=C18 spherical 20-35 μm; mobile phase: [water (HCOOH)-ACN], B %=5%-80%; 7.0 min) to give the desired product (25 mg, 30% yield). 1H NMR: (400 MHz, DMSO-d6) δ 11.72 (s, 1H), 7.87-7.82 (m, 1H), 7.77-7.64 (m, 1H), 7.57 (d, J=6.7 Hz, 1H), 7.53-7.42 (m, 2H), 7.24 (d, J=6.7 Hz, 1H), 6.31 (br, 1H), 5.37 (s, 2H), 4.08 (s, 3H), 3.87 (s, 3H), 2.43-2.38 (m, 1H), 2.00-1.80 (m, 6H). MS(ESI) m/z [M+H]⁺=470.1.

### Assay A: KAT6A and KAT6B Biochemical Assay

Enzymatic reactions of KAT6A and KAT6B were performed using Fluorescence Resonance Energy Transfer (TR-FRET) assay, which measures acetylation of a synthetic, biotinylated histone-H4 peptide by the KAT6A and KAT6B enzymes. Recombinant human KAT6A protein (497-780) were expressed in BL21 cell using an E. coli expression system. Recombinant human KAT6B protein (657-1069) was expressed in sf9 cell using a baculovirus expression system. Histone H4 peptide (SEQ ID NO: 1—SGRGKGGKGLGKGGAKRHRKV-GGK-Biotin), which was used as substrate, was synthesized by GL Biochem, Shanghai, China. Acetyl coenzyme A was purchased from Sigma-Aldrich (#A-2056).

Detailed information for enzyme, H4 and AcCoA are listed in Table 5.

TABLE 5

| | Final concentration in reaction | | |
|---|---|---|---|
| Enzyme | Enzyme (nM) | H4 (nM) | AcCoA (nM) |
| KAT6A | 5 | 2000 | 300 |
| KAT6B | 5 | 1000 | 100 |

Testing for the inhibition activities of various compounds disclosed herein was carried out at room temperature in assay buffer containing 100 mM Tris/HCl Ph7.8, 15 mM NaCl, 0.01% chicken egg white albumin, 1 mM DTT and 0.01% Tween-20. Compounds in DMSO were dispensed into wells of a black, 384-well plate (Corning 4514) using ECHO555 (Beckman). The final concentration ranges of the test compounds were 0.0625-1,000 nM, 0.625-10,000 nM, or 0.313-5,000 nM. Five μL of KAT6A or KAT6B enzyme solution were added to wells, and the plate was incubated for 1 hour at room temperature (rt). After incubation for 1 hour at rt, 5 μL H4 and AcCoA substrate solutions were added to the wells to initiate reaction, and the plate was incubated. After 2 hours reaction, 5 μL detection reagent were added to the wells, and the plate was incubated for 2 hours. After two hours incubation, detection reagent containing 5 nM Anti-histone H4 antibody (ABCAM #ab177790), 100 μM formic acid (sigma #06473-100ML), 0.1 test/μL streptavidin-XL665 (PerkinElmer #610SAXLG), 0.05 test/μL PAb anti-rabbit IgG-Eu (PerkinElmer μ61PARKLA) was added to the wells. TR-FRET was measured on a microplate reader (PHERAstar FSX, BMG labtech). The IC₅₀s were calculated based on inhibition of enzyme activity in the presence of increasing concentrations of test compounds, and the results are shown in Table 6. In Table 6, I≤5 nM, 5 nM<II≤50 nM.

TABLE 6
IC50 values of examples in biochemical
KAT6A and KAT6B assays.

Biochemical assay

| Example | KAT6A IC50, nM | KAT6B IC50, nM |
|---|---|---|
| C1 | I | I |
| C2 | I | I |
| C3 | I | I |
| C4 | I | I |
| C5 | I | I |
| C6 | I | I |
| C7 | I | I |
| C8 | I | I |
| C9 | II | II |
| C10 | I | I |
| C11 | I | I |
| C12 | II | II |
| C13 | I | I |
| C14 | I | I |
| C15 | I | I |
| C16 | I | I |
| C17 | I | I |
| C18 | I | I |
| C19 | I | I |
| C20 | I | I |
| C21 | I | I |
| C22 | I | I |
| C23 | I | I |
| C24 | I | I |
| C25 | I | I |
| C26 | I | I |
| C27 | I | I |
| C28 | I | I |
| C29 | I | I |
| C30 | I | I |
| C31 | I | I |
| C32 | I | I |
| C33 | I | I |
| C34 | I | I |
| C35 | I | I |
| C36 | I | I |
| C37 | I | I |
| C38 | I | I |
| C39 | I | I |
| C40 | I | I |
| C41 | I | I |
| C42 | I | I |
| C43 | I | I |
| C44 | I | I |
| C45 | I | I |
| C46 | I | I |
| C47 | I | I |
| C48 | I | I |
| C49 | I | I |
| C50 | I | I |
| C51 | I | I |
| C52 | I | I |
| C53 | I | I |
| C54 | I | I |
| C55 | I | I |
| C56 | I | I |
| C57 | I | I |
| C58 | I | I |
| C59 | I | I |
| C60 | I | I |
| C61 | I | I |
| C62 | I | I |
| C63 | I | I |
| C64 | I | I |
| C65 | I | I |
| C66 | I | I |
| C67 | I | I |
| C68 | II | I |
| C69 | I | I |
| C70 | I | I |
| C71 | I | I |
| C72 | I | I |
| C73 | I | I |
| C74 | I | I |
| C75 | I | I |
| C76 | I | I |
| C77 | II | I |
| C78 | I | I |
| C79 | I | I |

Assay B: Histone H3 lysine 23 Acetylation Assay

Compounds were tested for their capacity to suppress the histone H3K23 acetylation in the following high content analysis assay.

The cell line U-2 OS (ATCC HTB-96) was seeded at a density of 2000 cells per well in black 384 well cell culture/imaging microplates in 45 μl McCoy's 5A medium with 810 fetal bovine serum. After overnight incubation in 37° C., 5% CO2 incubator, cells were then treated with a 9-point dilution series of compounds and a blank control group with medium containing 0.1% DMSO.

Following a 24 hrs exposure to the compounds, the cells were fixed with 4% immunohistochemical fixative for 20 mins at room temperature, washed with DPBS with 2% fetal bovine serum, and permed with Perm Buffer III for 30 min on ice, washed with DPBS with 2% fetal bovine serum. Then the cells were blocked with Odyssey blocking buffer containing 0.1% Tween 20. 20 ul Anti-Histone H3 (acetyl K23) antibody (Abcam, ab177275) in odyssey blocking buffer containing 0.1% Tween20 was added and incubated overnight at 4° C. After incubation, the cells were washed for 3 times and a secondary antibody Anti-rabbit IgG (H+L), F(ab′)2 Fragment (Alexa Fluor® 647 Conjugate) (CST, 4414S) were added into cells for one hour. The cells were washed and 1 ug/ml DAPI (Thermo, 62248) were added for 10 min. Then cells were washed and read on the PerkinElmer Operetta CLS high content analysis system. The nuclei were identified by DAPI and H3K23ac level was calculated from the Alexa Fluor 647 intensity in the nuclei. Inhibition rate of H3K23ac was calculated as the mean intensity of cells per well relative to blank controls on the same plate.

Detailed information for reagents are listed in table 7.

TABLE 7

| | | Catalogue |
|---|---|---|
| Materials | Supplier | Number |
| U-2 OS | ATCC | HTB-96 |
| McCoy's 5A medium | Gibco | 16600-082 |
| Black 384 well cell culture/imaging | Agilent | 204628-100 |
| microplates | | |
| Fetal bovine serum | Gibco | 10099-141C |
| 4% immunohistochemical fixative | Lablead | P4500 |
| Perm Buffer III | BD Bioscience | 558050 |
| DPBS | Gibco | 14190-144 |
| Odyssey blocking buffer | LI-COR BioScience | 927-60001 |
| Tween 20 | Sigma-Aldrich | P1379 |
| Anti-Histone H3 (acetyl K23) | Abcam | ab177275 |
| antibody | | |
| Anti-rabbit IgG (H + L), | Cell Signaling | 4414S |
| F(ab′)2 Fragment (Alexa | Technology | |
| Fluor ® 647 Conjugate) | | |
| DAPI | Thermo Fisher | 62248 |
| | Scientific | |

In Table 8, I≤10 nM, 10 nM<II≤100 nM.

TABLE 8
IC₅₀ values of examples in U-2 OS H3K23ac assays.

| Example | IC50 nM |
|---|---|
| C1 | I |
| C2 | I |
| C3 | I |
| C4 | I |
| C5 | I |
| C6 | I |
| C7 | I |
| C8 | I |
| C9 | II |
| C10 | I |
| C11 | I |
| C12 | II |
| C13 | I |
| C14 | I |
| C15 | I |
| C16 | I |
| C17 | II |
| C18 | I |
| C19 | II |
| C20 | I |
| C21 | I |
| C22 | I |
| C23 | I |
| C24 | II |
| C25 | I |
| C26 | I |
| C27 | I |
| C28 | I |
| C29 | I |
| C30 | I |
| C31 | II |
| C32 | I |
| C33 | I |
| C34 | I |
| C35 | I |
| C36 | I |
| C37 | I |
| C38 | I |
| C39 | I |
| C40 | I |
| C41 | I |
| C42 | I |
| C43 | I |
| C44 | I |
| C45 | I |
| C46 | I |
| C47 | I |
| C48 | I |
| C49 | I |
| C50 | I |
| C51 | I |
| C52 | I |
| C53 | I |
| C54 | I |
| C55 | I |
| C56 | I |
| C57 | I |
| C58 | I |
| C59 | I |
| C60 | I |
| C61 | I |
| C62 | I |
| C63 | I |
| C64 | I |
| C65 | I |
| C66 | I |
| C67 | I |
| C68 | I |
| C69 | I |
| C70 | I |
| C71 | I |
| C72 | I |
| C73 | I |
| C74 | I |
| C75 | I |
| C76 | I |
| C77 | I |
| C78 | I |
| C79 | I |

1-30. (canceled)

31. A pharmaceutical composition comprising the compound of claim 33, or a pharmaceutically acceptable salt, deuterated analog, N-oxide, or tautomer thereof, and at least one pharmaceutically acceptable carrier or excipient.

32. A method of treating breast cancer, comprising administering to a subject in need thereof a therapeutically effective amount of the compound of claim 33, or a pharmaceutically acceptable salt, deuterated analog, N-oxide, or tautomer thereof.

33. A compound selected from: 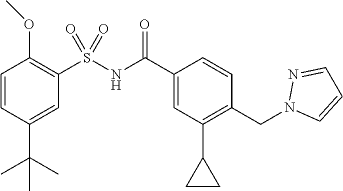 [SMILES: COc1ccc(C(C)(C)C)cc1S(=O)(=O)NC(=O)c1ccc(Cn2cccn2)c(C2CC2)c1] 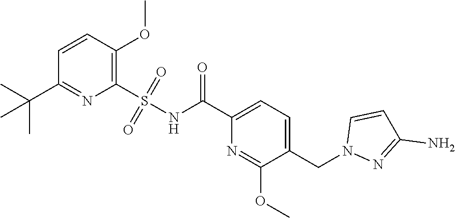 [SMILES: COc1ccc(C(C)(C)C)nc1S(=O)(=O)NC(=O)c1ccc(Cn2ccc(N)n2)c(OC)n1] 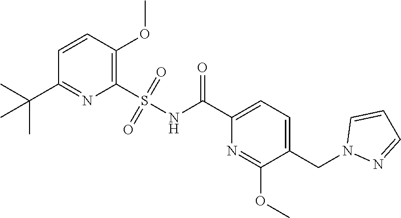 [SMILES: COc1ccc(C(C)(C)C)nc1S(=O)(=O)NC(=O)c1ccc(Cn2cccn2)c(OC)n1] 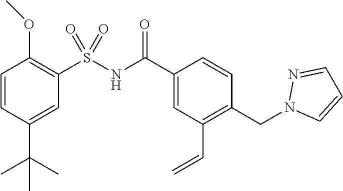 [SMILES: C=Cc1cc(C(=O)NS(=O)(=O)c2cc(C(C)(C)C)ccc2OC)ccc1Cn1cccn1] 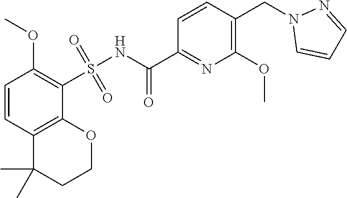 [SMILES: COc1ccc2c(c1S(=O)(=O)NC(=O)c1ccc(Cn3cccn3)c(OC)n1)OCCC2(C)C] 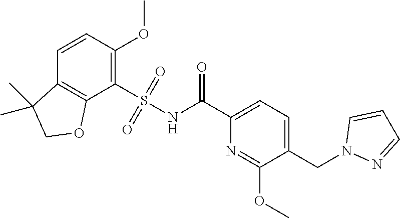 [SMILES: COc1ccc2c(c1S(=O)(=O)NC(=O)c1ccc(Cn3cccn3)c(OC)n1)OCC2(C)C] 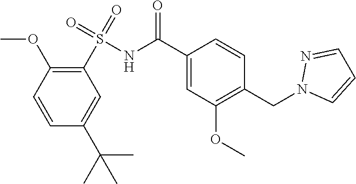 [SMILES: COc1cc(C(=O)NS(=O)(=O)c2cc(C(C)(C)C)ccc2OC)ccc1Cn1cccn1] 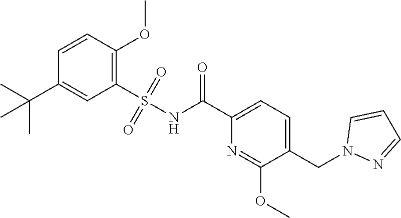 [SMILES: COc1ccc(C(C)(C)C)cc1S(=O)(=O)NC(=O)c1ccc(Cn2cccn2)c(OC)n1] 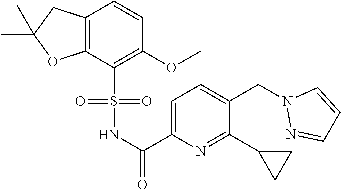 [SMILES: COc1ccc2c(c1S(=O)(=O)NC(=O)c1ccc(Cn3cccn3)c(C3CC3)n1)OC(C)(C)C2]
or a pharmaceutically acceptable salt, deuterated analog, N-oxide, or tautomer thereof.

34. The compound of claim 33, wherein the compound is 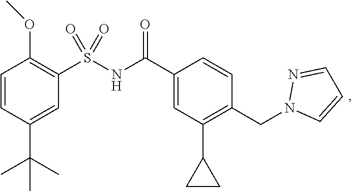 [SMILES: COc1ccc(C(C)(C)C)cc1S(=O)(=O)NC(=O)c1ccc(Cn2cccn2)c(C2CC2)c1]
or a pharmaceutically acceptable salt, deuterated analog, N-oxide, or tautomer thereof.

35. The compound of claim 33, wherein the compound is 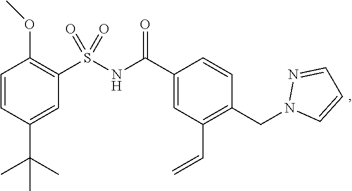 [SMILES: C=Cc1cc(C(=O)NS(=O)(=O)c2cc(C(C)(C)C)ccc2OC)ccc1Cn1cccn1]
or a pharmaceutically acceptable salt, deuterated analog, N-oxide, or tautomer thereof.

36. The compound of claim 33, wherein the compound is 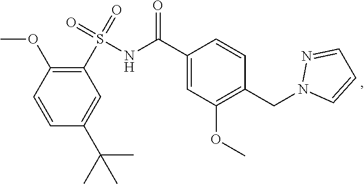 [SMILES: COc1cc(C(=O)NS(=O)(=O)c2cc(C(C)(C)C)ccc2OC)ccc1Cn1cccn1]
or a pharmaceutically acceptable salt, deuterated analog, N-oxide, or tautomer thereof.

37. The compound of claim 33, wherein the compound is 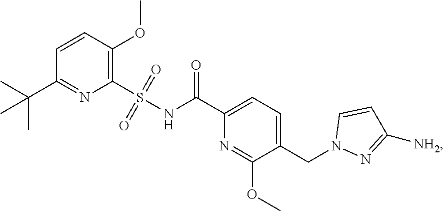 [SMILES: COc1ccc(C(C)(C)C)nc1S(=O)(=O)NC(=O)c1ccc(Cn2ccc(N)n2)c(OC)n1]
or a pharmaceutically acceptable salt, deuterated analog, N-oxide, or tautomer thereof.

38. The compound of claim 33, wherein the compound is 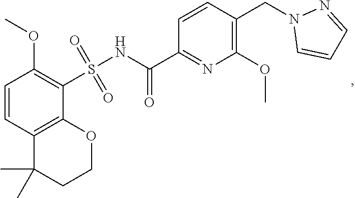 [SMILES: COc1ccc2c(c1S(=O)(=O)NC(=O)c1ccc(Cn3cccn3)c(OC)n1)OCCC2(C)C]
or a pharmaceutically acceptable salt, deuterated analog, N-oxide, or tautomer thereof.

39. The compound of claim 33, wherein the compound is 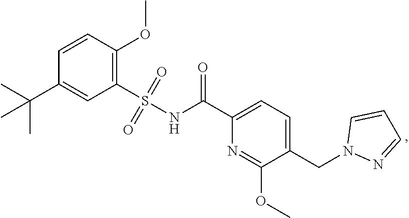 [SMILES: COc1ccc(C(C)(C)C)cc1S(=O)(=O)NC(=O)c1ccc(Cn2cccn2)c(OC)n1]
or a pharmaceutically acceptable salt, deuterated analog, N-oxide, or tautomer thereof.

40. The compound of claim 33, wherein the compound is 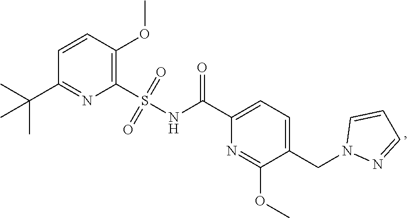 [SMILES: COc1ccc(C(C)(C)C)nc1S(=O)(=O)NC(=O)c1ccc(Cn2cccn2)c(OC)n1]
or a pharmaceutically acceptable salt, deuterated analog, N-oxide, or tautomer thereof.

41. The compound of claim 33, wherein the compound is 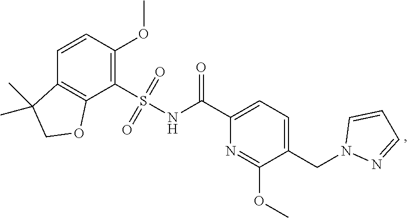 [SMILES: COc1ccc2c(c1S(=O)(=O)NC(=O)c1ccc(Cn3cccn3)c(OC)n1)OCC2(C)C]
or a pharmaceutically acceptable salt, deuterated analog, N-oxide, or tautomer thereof.

42. The compound of claim 33, wherein the compound is 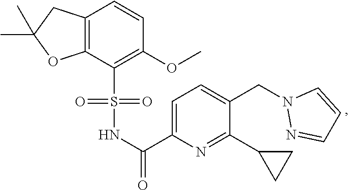 [SMILES: COc1ccc2c(c1S(=O)(=O)NC(=O)c1ccc(Cn3cccn3)c(C3CC3)n1)OC(C)(C)C2]
or a pharmaceutically acceptable salt, deuterated analog, N-oxide, or tautomer thereof.

43. The compound of claim 33, wherein the compound is 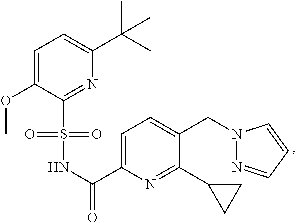 [SMILES: COc1ccc(C(C)(C)C)nc1S(=O)(=O)NC(=O)c1ccc(Cn2cccn2)c(C2CC2)n1]
or a pharmaceutically acceptable salt, deuterated analog, N-oxide, or tautomer thereof.

44. The compound of claim 33, wherein the compound is 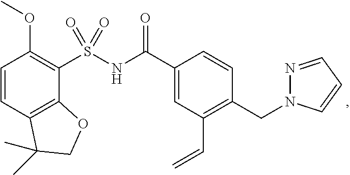 [SMILES: C=Cc1cc(C(=O)NS(=O)(=O)c2c(OC)ccc3c2OCC3(C)C)ccc1Cn1cccn1]
or a pharmaceutically acceptable salt, deuterated analog, N-oxide, or tautomer thereof.

45. The compound of claim 33, wherein the compound is 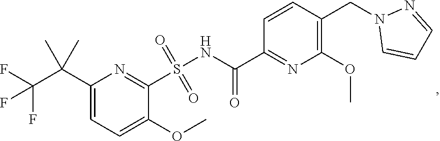 [SMILES: COc1ccc(C(C)(C)C(F)(F)F)nc1S(=O)(=O)NC(=O)c1ccc(Cn2cccn2)c(OC)n1]
or a pharmaceutically acceptable salt, deuterated analog, N-oxide, or tautomer thereof.

46. The compound of claim 33, wherein the compound is 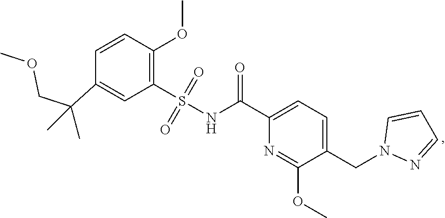 [SMILES: COCC(C)(C)c1ccc(OC)c(S(=O)(=O)NC(=O)c2ccc(Cn3cccn3)c(OC)n2)c1]
or a pharmaceutically acceptable salt, deuterated analog, N-oxide, or tautomer thereof.

47. The compound of claim 33, wherein the compound is 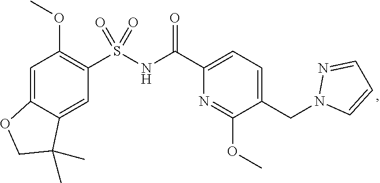 [SMILES: COc1cc2c(cc1S(=O)(=O)NC(=O)c1ccc(Cn3cccn3)c(OC)n1)C(C)(C)CO2]
or a pharmaceutically acceptable salt, deuterated analog, N-oxide, or tautomer thereof.

48. The compound of claim 33, wherein the compound is 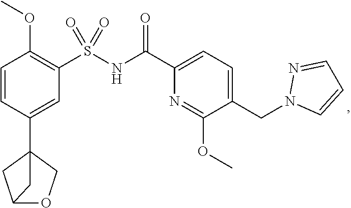 [SMILES: COc1ccc(C23COC(C2)C3)cc1S(=O)(=O)NC(=O)c1ccc(Cn2cccn2)c(OC)n1]
or a pharmaceutically acceptable salt, deuterated analog, N-oxide, or tautomer thereof.

49. The compound of claim 33, wherein the compound is 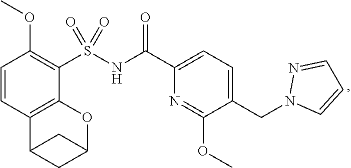 [SMILES: COc1ccc2c(c1S(=O)(=O)NC(=O)c1ccc(Cn3cccn3)c(OC)n1)OC1CC2C1]
or a pharmaceutically acceptable salt, deuterated analog, N-oxide, or tautomer thereof.

50. The compound of claim 33, wherein the compound is 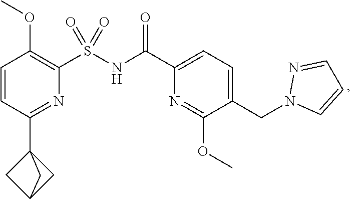 [SMILES: COc1ccc(C23CC(C2)C3)nc1S(=O)(=O)NC(=O)c1ccc(Cn2cccn2)c(OC)n1]
or a pharmaceutically acceptable salt, deuterated analog, N-oxide, or tautomer thereof.


SEQUENCE LISTING (1 sequence)

SEQ ID NO: 1 - protein, 21 aa, synthetic construct
SGRGKGGKGLGKGGAKRHRKV